(19) United States
(12) Patent Application Publication
(10) Pub. No.: US 2022/0403473 A1
(43) Pub. Date: Dec. 22, 2022

(54) METHODS FOR DETECTING COLORECTAL CANCER

(71) Applicant: EPIGENOMICS AG, Berlin (DE)

(72) Inventors: Jörn LEWIN, Berlin (DE); Denise KOTTWITZ, Berlin (DE)

(21) Appl. No.: 17/785,302

(22) PCT Filed: Dec. 16, 2020
(86) PCT No.: PCT/EP2020/086498

§ 371 Date: Jun. 14, 2022

(30) Foreign Application Priority Data
Dec. 16, 2019 (EP) 19216669.2

(51) Int. Cl. C12Q 1/6886

(52) U.S. Cl. CPC C12Q 1/6886; C12Q 2600/118; C12Q 2600/154; C12Q 2600/16

(57) ABSTRACT

The present invention relates to the field of pharmacogenomics and in particular to detecting the presence or absence of methylated genomic DNA derived from colorectal cancer cells in biological samples such as body fluids that contain circulating DNA from the cancer cells. This detection is useful for an early and reliable diagnosis of colorectal cancer and the invention provides methods and oligonucleotides suitable for this purpose.

### FIELD OF THE INVENTION

The present invention relates to the field of pharmacogenomics and in particular to detecting the presence or absence of methylated genomic DNA derived from colorectal cancer cells in biological samples such as body fluids that contain circulating DNA from the cancer cells. This detection is useful for an early and reliable diagnosis of colorectal cancer and the invention provides methods and oligonucleotides suitable for this purpose.

### BACKGROUND OF THE INVENTION

Colorectal cancer (CRC) encompasses tumors originating from the colon and rectum. It is the third most common cancer worldwide, but the second most common cancer killer. When colorectal cancer is found at an early stage, the 5-year relative survival rate is about 90%. At advanced stages, however, colorectal cancer is not curable. Conventional CRC screening involved either visual exams or stool-based tests. Visual exams look at the structure of the colon and rectum for abnormal areas using a scope put into the rectum (e.g. colonoscopy or sigmoidoscopy) or non-invasive imaging techniques (e.g. x-ray or CR colonography (virtual colonoscopy)). Stool tests such as FIT (Fecal immunochemical test) or gFOBT (Guaiac-based fecal occult blood test) usually detect blood or polyps in stool samples. Stool tests have relatively low sensitivity and specificity and are also problematic with regard to participants' compliance, satisfaction and intention to be rescreened. Invasive visual exams are uncomfortable and incur a risk of bleeding, tears and infection. Therefore, they are often avoided by at-risk subjects. Non-invasive imaging techniques expose the subjects to radiation and often miss small polyps.

DNA methylation patterns are largely modified in cancer cells and can therefore be used to distinguish cancer cells from normal tissues. As such, DNA methylation patterns are being used to diagnose all sorts of cancers. One of the challenges is identifying genes or genomic regions that (i) are abnormally methylated in CRC and (ii) provide for a diagnostic power that is suitable for detecting CRC, i.e. which provide for a sufficient sensitivity and specificity.

It was the goal of the inventors to provide further genes or genomic regions that are abnormally methylated in CRC and that also have good and ideally improved sensitivity and/or specificity. It was also the goal of the inventors to provide combinations of such genes or genomic regions that are particularly suitable for detecting CRC. Particular emphasis was thereby put on detection using body fluid samples, since their use allows minimally invasive screening of large, e.g. at-risk, populations.

The less advanced CRC is, the better the treatment options and the chances of curing the patient are. Thus, it is highly desirable to diagnose it as early and reliably as possible with tests subjects do not hesitate to undergo.

### SUMMARY OF THE INVENTION

In a first aspect, the present invention relates to a method of detecting DNA methylation, comprising the step of detecting DNA methylation within at least one genomic DNA polynucleotide selected from the group consisting of polynucleotides having a sequence comprised in SEQ ID NO: 16 (mADCYAP1), SEQ ID NO: 56 and/or SEQ ID NO: 61 (mANKRD13B), SEQ ID NO: 41 and/or SEQ ID NO: 46 (mCLEC14A), SEQ ID NO: 71 (mCRMP1), SEQ ID NO: 81 and/or SEQ ID NO: 86 (mEYA4), SEQ ID NO: 31 (mKHDRBS2), SEQ ID NO: 96 and/or SEQ ID NO: 101 (mMSC), SEQ ID NO: 111 and/or SEQ ID NO: 116 (mNGFR), SEQ ID NO: 126 (mNKX2), SEQ ID NO: 141 and/or SEQ ID NO: 146 (mRASSF2), SEQ ID NO: 1 (mSEPT9), SEQ ID NO: 161 (mSND1), SEQ ID NO: 171 (mTBX18), SEQ ID NO: 186 and/or SEQ ID NO: 191 (mTFAP2E), SEQ ID NO: 201 and/or SEQ ID NO: 206 (mTMEFF2), or SEQ ID NO: 216 (mVAX1) in a subject's biological sample comprising genomic DNA, wherein the genomic DNA may comprise DNA derived from colorectal cancer (CRC) cells.

In a second aspect, the invention relates to a method for detecting the presence or absence of CRC in a subject, comprising detecting DNA methylation according to the method of the first aspect, wherein the presence of detected methylated genomic DNA indicates the presence of CRC and the absence of detected methylated genomic DNA indicates the absence of CRC.

In a third aspect, the present invention relates to an oligonucleotide selected from the group consisting of a primer and a probe, comprising a sequence that is substantially identical to a stretch of contiguous nucleotides of one of SEQ ID NOs 17-20 (mADCYAP1), 57-60 and/or 62-65 (mANKRD13B), 42-45 and/or 47-50 (mCLEC14A), 72-75 (mCRMP1), 82-85 and/or 87-90 (mEYA4), 32-35 (mKHDRBS2), 97-100 and/or 102-105 (mMSC), 112-115 and/or 117-120 (mNGFR), 127-130 (mNKX2), 142-145 and/or 147-150 (mRASSF2), 2-5 (mSEPT9), 162-165 (mSND1), 172-175 (mTBX18), 187-190 and/or 192-195 (mTFAP2E), 202-205 and/or 207-210 (mTMEFF2), or 217-220 (mVAX1).

In a fourth aspect, the present invention relates to a kit comprising at least a first and a second oligonucleotide of the third aspect.

In a fifth aspect, the present invention relates to the use of the method of the first aspect, of the oligonucleotide of the third aspect or of the kit the fourth aspect for the detection of CRC or for monitoring a subject having an increased risk of developing CRC, suspected of having CRC or that has had CRC.

In a sixth aspect, the present invention relates to the method of the first or the second aspect, or the use of the fifth aspect, comprising a step of treating CRC of a subject for which the DNA methylation is detected in its biological sample.

### LEGENDS TO THE FIGURES

FIG. 1: Map of target regions. See Table 3 for an explanation of the SEQ ID NOs.

FIG. 2: Single marker performance and methylation differences. Grey squares show comethylation for marker B-P (CoM number of completely methylated fragments in relation to all amplified DNA in an assay as detected by reads matching an assay) normalized to a range of 0 to 1 in a linear scale by greyscale color or in a logarithmic scale by size as laid out in the legend at the bottom. Positivity of marker A measured in triplicate realtime PCR (x/3 pos Septin 9 as measured by the Epi proColon diagnostic test) is shown as number from 0 to 3. Plasma samples for 105 colorectal cancer patients (CRC) and 69 individuals with no evidence of disease (NED) are vertically grouped into their two diagnostic groups. Numbers at the bottom are area under the curves from responder operator characteristic curves. Grey bars and numbers on the right are the sum of all fully methylated molecules (rounded to 1000) as amplified in the PCR and normalized by total amount of amplified DNA measured for a sample. Markers are A: mSEPT9, B: mADCYAP1, C: mKHDRBS2, D: mCLEC14A, E: mANKRD13B, F: mCRMP1, G: mEYA4, H: mMSC, I: mNGFR, J: mNKX2, K: mRASSF2, L: mSND1, M: mTBX18, N: mTFAP2E, O: mTMEFF2; P: mVAX1.

FIG. 3: Responder operator curves (ROCs) for sixteen markers and two exemplary marker combinations by logistic regression analysis. The curves show the relation of the sensitivity (y-axis) to the specificity (x-axis). Areas under the curve (AUC) are written at the bottom right of the plotting area. Markers are A: mSEPT9, B: mADCYAP1, C: mKHDRBS2, D: mCLEC14A, E: mANKRD13B, F: mCRMP1, G: mEYA4, H: mMSC, I: mNGFR, J: mNKX2, K: mRASSF2, L: mSND1, M: mTBX18, N: mTFAP2E, O: mTMEFF2; P: mVAX1.

### DETAILED DESCRIPTION OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention, which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Kolbl, H. eds. (1995), Helvetica Chimica Acta, CH-4010 Basel, Switzerland).

Several documents are cited throughout the text of this specification. Each of the documents cited herein (including all patents, patent applications, scientific publications, manufacturers' specifications, instructions etc.), whether supra or infra, is hereby incorporated by reference in its entirety.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments, which combine the explicitly described embodiments with any number of the disclosed and/or preferred elements. Furthermore, any permutations and combinations of all described elements in this application should be considered disclosed by the description of the present application unless the context indicates otherwise.

Throughout this specification and the claims which follow, unless the context requires otherwise, the word “comprise”, and variations such as “comprises” and “comprising”, are to be understood to imply the inclusion of a stated integer or step or group of integers or steps but not the exclusion of any other integer or step or group of integer or step. In preferred embodiments, “comprise” can mean “consist of”. As used in this specification and the appended claims, the singular forms “a”, “an”, and “the” include plural referents, unless the content clearly dictates otherwise.

### Aspects of the Invention and Particular Embodiments Thereof

In a first aspect, the present invention relates to a method of detecting DNA methylation, comprising the step of detecting DNA methylation within at least one genomic DNA polynucleotide selected from the group consisting of polynucleotides having a sequence comprised in SEQ ID NO: 16 (mADCYAP1), SEQ ID NO: 56 and/or SEQ ID NO: 61 (mANKRD13B), SEQ ID NO: 41 and/or SEQ ID NO: 46 (mCLEC14A), SEQ ID NO: 71 (mCRMP1), SEQ ID NO: 81 and/or SEQ ID NO: 86 (mEYA4), SEQ ID NO: 31 (mKHDRBS2), SEQ ID NO: 96 and/or SEQ ID NO: 101 (mMSC), SEQ ID NO: 111 and/or SEQ ID NO: 116 (mNGFR), SEQ ID NO: 126 (mNKX2), SEQ ID NO: 141 and/or SEQ ID NO: 146 (mRASSF2), SEQ ID NO: 1 (mSEPT9), SEQ ID NO: 161 (mSND1), SEQ ID NO: 171 (mTBX18), SEQ ID NO: 186 and/or SEQ ID NO: 191 (mTFAP2E), SEQ ID NO: 201 and/or SEQ ID NO: 206 (mTMEFF2), or SEQ ID NO: 216 (mVAX1) in a subject's biological sample comprising genomic DNA. Specifically, the genomic DNA may comprise DNA derived from colorectal cancer (CRC) cells. Preferably, the genomic DNA, in particular the genomic DNA derived from CRC cells, is cell-free DNA. The phrase “the genomic DNA may comprise DNA derived from colorectal cancer (CRC) cells” does, in a preferred embodiment, mean that the subject has an increased risk of CRC, is suspected of having CRC or has had CRC (i.e. has been treated to remove any detectable sign of CRC, but is suspected to relapse).

Preferably, the method is an in vitro method.

In a preferred embodiment,
the polynucleotide having a sequence comprised in SEQ ID NO: 16 has a sequence comprised in SEQ ID NO: 21, preferably in SEQ ID NO: 26,
the polynucleotide having a sequence comprised in SEQ ID NO: 56 and/or SEQ ID NO: 61 has a sequence comprised in SEQ ID NO: 66,
the polynucleotide having a sequence comprised in SEQ ID NO: 41 and/or SEQ ID NO: 46 has a sequence comprised in SEQ ID NO: 51,
the polynucleotide having a sequence comprised in SEQ ID NO: 71 has a sequence comprised in SEQ ID NO: 76,
the polynucleotide having a sequence comprised in SEQ ID NO: 81 and/or SEQ ID NO: 86 has a sequence comprised in SEQ ID NO: 91,
the polynucleotide having a sequence comprised in SEQ ID NO: 31 has a sequence comprised in SEQ ID NO: 36,
the polynucleotide having a sequence comprised in SEQ ID NO: 96 and/or SEQ ID NO: 101 has a sequence comprised in SEQ ID NO: 106,
the polynucleotide having a sequence comprised in SEQ ID NO: 111 and/or SEQ ID NO: 116 has a sequence comprised in SEQ ID NO: 121,
the polynucleotide having a sequence comprised in SEQ ID NO: 126 has a sequence comprised in SEQ ID NO: 131, preferably in SEQ ID NO: 136,
the polynucleotide having a sequence comprised in SEQ ID NO: 141 and/or SEQ ID NO: 146 has a sequence comprised in SEQ ID NO: 151,
the polynucleotide having a sequence comprised in SEQ ID NO: 1 has a sequence comprised in SEQ ID NO: 6, preferably in SEQ ID NO: 11,
the polynucleotide having a sequence comprised in SEQ ID NO: 161 has a sequence comprised in SEQ ID NO: 156, preferably in SEQ ID NO: 166,
the polynucleotide having a sequence comprised in SEQ ID NO: 171 has a sequence comprised in SEQ ID NO: 176, preferably in SEQ ID NO: 181,
the polynucleotide having a sequence comprised in SEQ ID NO: 186 and/or SEQ ID NO: 191 has a sequence comprised in SEQ ID NO: 196,
the polynucleotide having a sequence comprised in SEQ ID NO: 201 and/or SEQ ID NO: 206 has a sequence comprised in SEQ ID NO: 211, and/or
the polynucleotide having a sequence comprised in SEQ ID NO: 216 has a sequence comprised in SEQ ID NO: 221.

Preferably, DNA methylation is detected within at least two, more preferably at least three (or at least 4, 5, 6, 7, 8, 9, 10, 11, 12, 13, 14, 15 or in all, wherein larger numbers are preferred to smaller numbers) genomic DNA polynucleotides selected from said group (each polynucleotide corresponding to a different methylation marker). In specific preferred embodiments, methylation is detected for a combination of two markers according to Table 1 or three markers according to Table 2 (the tables showing advantageous AUC values), and optionally one or more further markers of the group consisting of mADCYAP1, mANKRD13B, mCLEC14A, mCRMP1, mEYA4, mKHDRBS2, mMSC, mNGFR, mNKX2, mRASSF2, mSEPT9, mSND1, mTBX18, mTFAP2E, mTMEFF2 and mVAX1 (sequences recited as above, including preferred ones). Of the combinations recited in Table 1, those are particularly preferred for which an AUC of at least 0.80, preferably at least 0.84, 0.86, 0.88, 0.90, or 0.92, more preferably at least 0.93 is shown in Table 1. Of the combinations recited in Table 2, those are particularly preferred for which an AUC of at least 0.85, preferably at least 0.87, 0.89, 0.9, 0.91, or 0.92, more preferably at least 0.93 or 0.94 is shown in Table 2.

The sequence the polynucleotide has is also referred to herein as the target region or target DNA and may be the sequence of the entire SEQ ID NO, or may be a sequence with a length as specified below in the section “Definitions and further embodiments of the invention”.

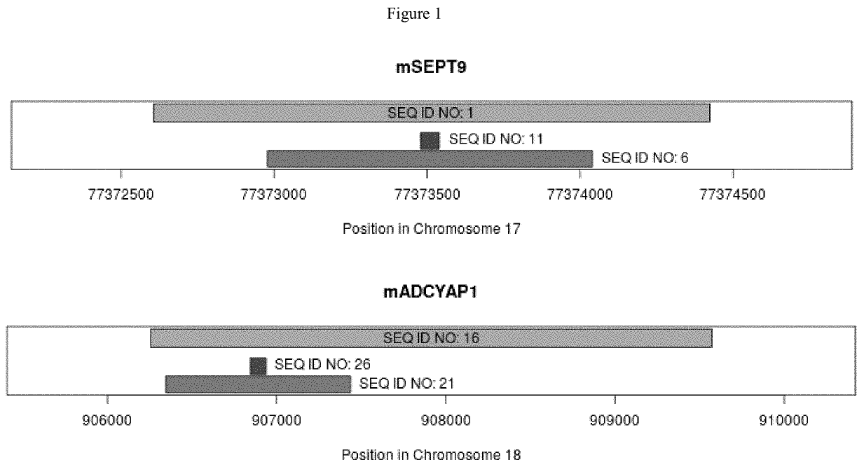

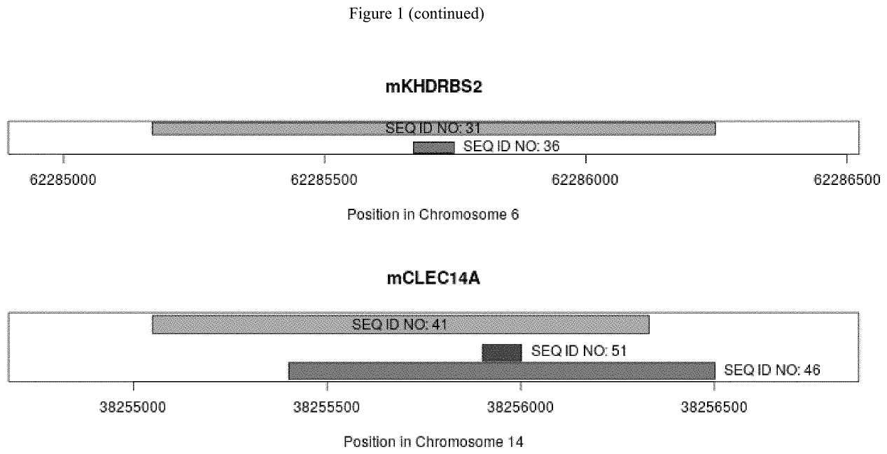

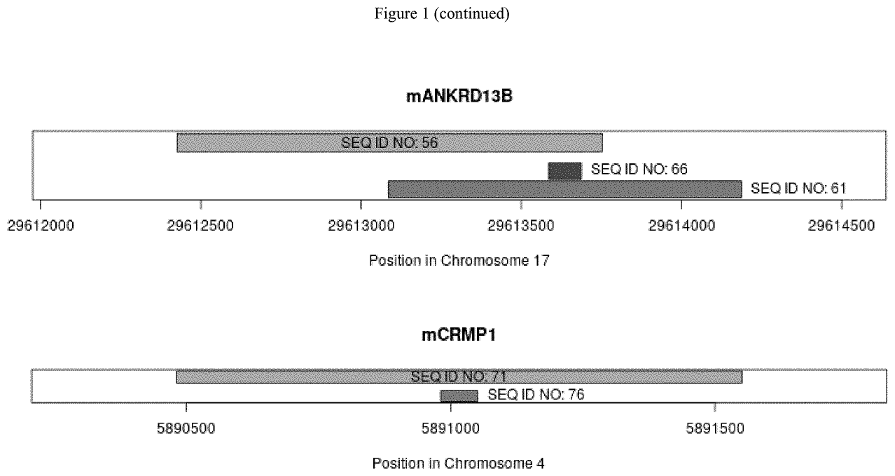

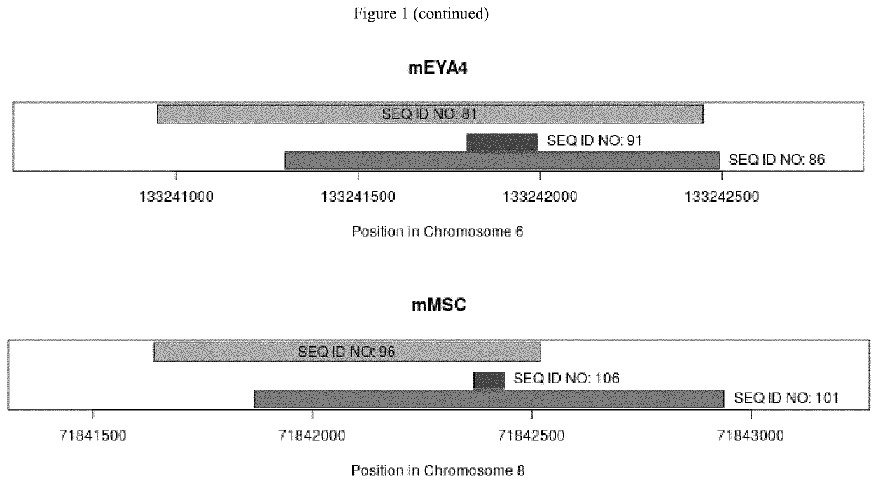

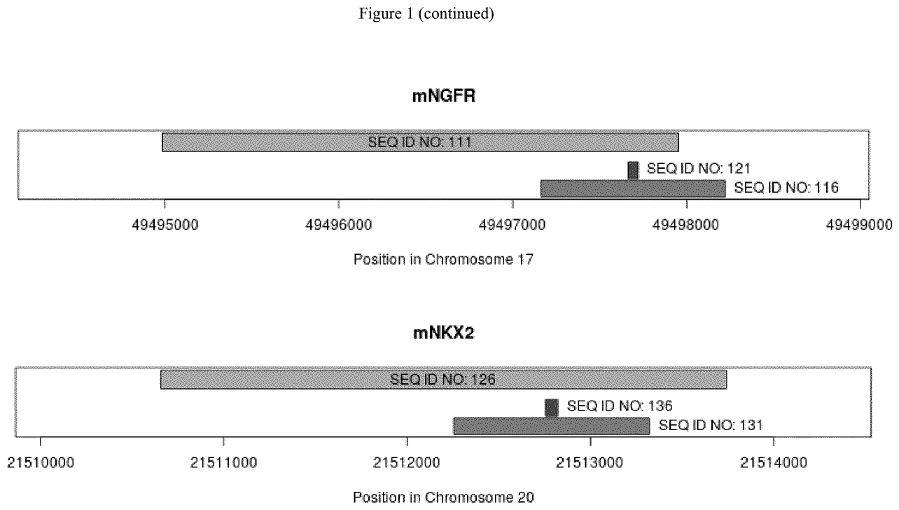

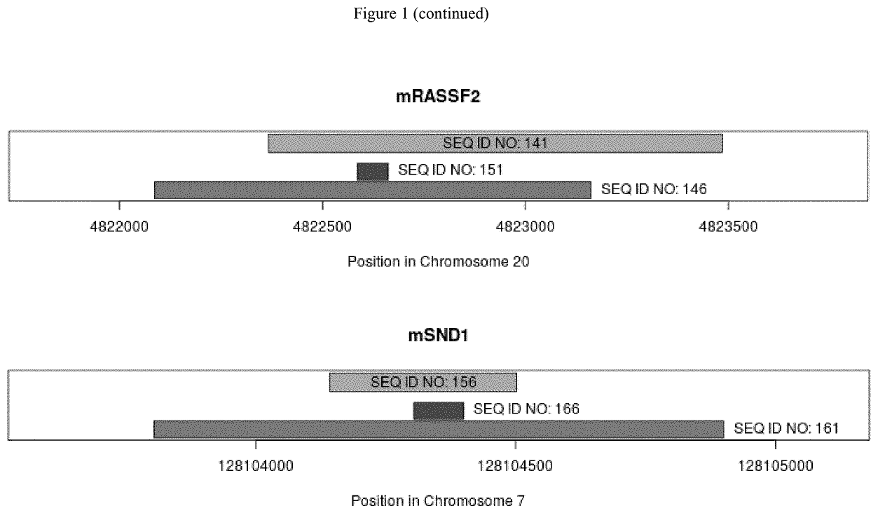

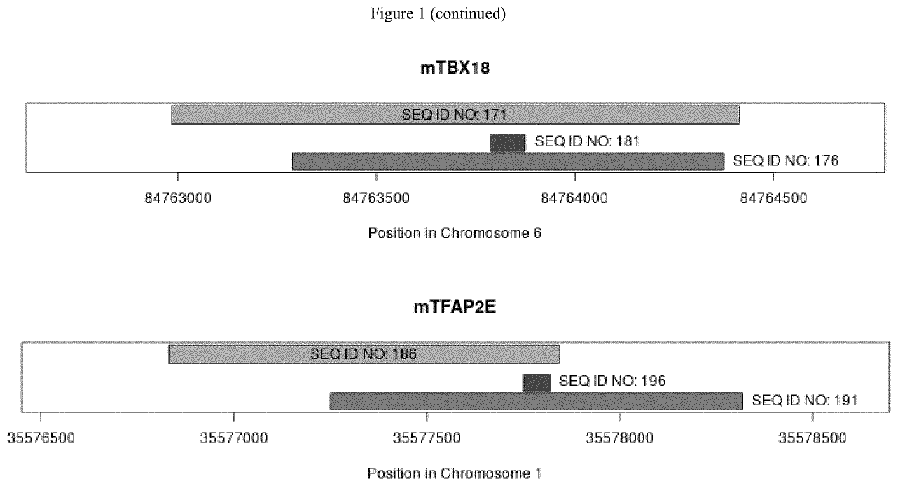

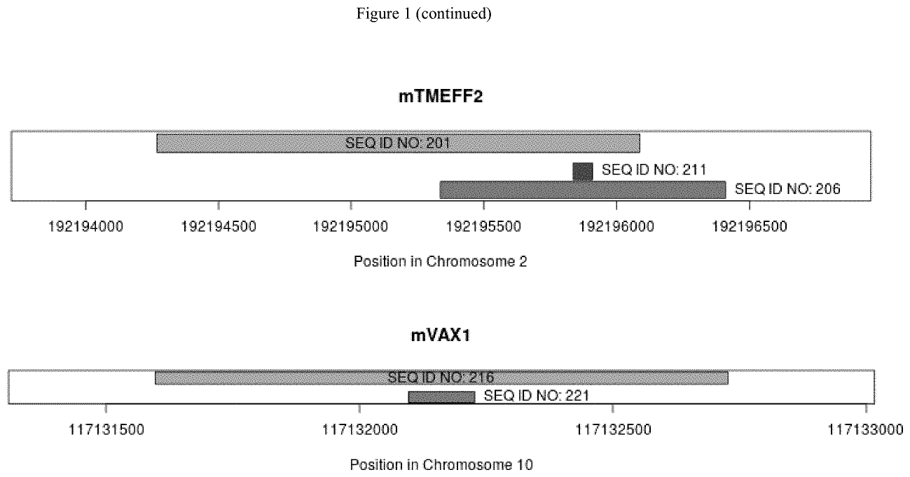

In this specification, the target DNAs are also referred to using the designations mSEPT9, mADCYAP1, mKHDRBS2, mCLEC14A, mANKRD13B, mCRMP1, mEYA4, mMSC, mNGFR, mNKX2, mRASSF2, mSND1, mTBX18, mTFAP2E, mTMEFF2 and mVAX1, which are the different methylation markers of the invention. In these, the first letter “m” means “methylation marker”, and the capital letters refer to the gene the target DNA resides in (the corresponding genomic region is provided in Table 3). When using these designations only without indicating specific SEQ ID NOs, it is referred to the SEQ ID NOs which correspond to the designation according to FIG. 1 and Table 3, with the order of preference indicated in the first and second aspects of the invention.

In a preferred embodiment, the genomic target DNA (the DNA region within which methylation is detected) comprises at least one CpG dinucleotide, preferably at least 2, 3, 4, or 5, most preferably at least 6 (e.g. at least 10, 15 or 30) CpG dinucleotides. Generally, the methylation of at least one CpG dinucleotide comprised in the genomic DNA is detected, preferably of at least 2, 3, 4, or 5, most preferably at least 6 (e.g. at least 10, 15 or 30) CpG dinucleotides. Furthermore, the methylation of usually all CpG dinucleotides comprised in the genomic target DNA is detected. Nevertheless, it is possible that the methylation detection of a part of the CpG dinucleotides is omitted (a part meaning up to 3, 2 or preferably 1, but never all), for example if the species the subject belongs to (preferably human) has a single polynucleotide polymorphism (SNP) at one or both positions of the CpG dinucleotide.

In one embodiment, the method of the first aspect comprises the steps of

(a) converting cytosine unmethylated in the 5-position to uracil or another base that does not hybridize to guanine in the genomic DNA of the biological sample; and
(b) detecting DNA methylation within the genomic DNA by detecting unconverted cytosine in the converted DNA of step (a).

A preferred way of carrying out the method comprises the steps of

(a) converting cytosine unmethylated in the 5-position to uracil or another base that does not hybridize to guanine in the genomic DNA;
(b) amplifying methylation-specifically a region of the converted DNA;
(c) detecting the presence or absence of DNA amplified in step (b);
wherein the presence or absence of amplified DNA indicates the presence or absence, respectively, of methylated genomic DNA.

In a preferred embodiment, step b) of amplifying comprises the use of at least one oligonucleotide according to the fourth aspect, preferably as a primer. More preferably, it comprises the use of oligonucleotides as comprised in the kit of the fifth aspect.

In a preferred embodiment of the method of the first aspect, the detecting of the DNA methylation comprises determining the amount of methylated genomic DNA. Any means known in the art can be used to detect DNA methylation or determine its amount (see also below for art-known and preferred means). It is preferred that methylation is detected or the amount of methylated genomic DNA is determined by sequencing, in particular next-generation-sequencing (NGS), by real-time PCR or by digital PCR.

Markers mADCYAP1, mANKRD13B, mCLEC14A, mCRMP1, mEYA4, mKHDRBS2, mMSC, mNGFR, mNKX2, mRASSF2, mSEPT9, mSND1, mTBX18, mTFAP2E, mTMEFF2 and mVAX1 show consistent comethylation and, thus, the amount of methylation can be determined simply by counting the number of methylated sequences (reads) when determining the amount of methylation by sequencing.

In a preferred embodiment, the biological sample is a colon or rectum tissue sample or a liquid biopsy, preferably a blood sample, a sample comprising cell-free DNA from blood (e.g. a urine sample), a blood-derived sample or a saliva sample.

In another preferred embodiment, the subject has an increased risk of developing CRC, is suspected of having CRC, has had CRC or has CRC.

The term “colorectal cancer (CRC)”, also known as bowel cancer and colon cancer and also referred to herein as the “cancer of the specification”, is used in the broadest sense and refers to all cancers that start in the colon or in the rectum. It includes the subtypes adenocarcinoma (cancer starting in cells that make mucus to lubricate the inside of the colon and rectum), carcinoid tumor (cancer starting from the interstitial cells of Cajal in the wall of the colon), lymphoma starting in the colon or rectum, and sarcoma starting in blood vessels, muscle layers, or other connective tissues in the wall of the colon and rectum. The most common and preferred CRC with regard to the invention is adenocarcinoma.

A “colon or rectum tissue sample” is a tissue sample from any tissue in which CRC can occur. In one embodiment, if the subject has cancer, it is a CRC tissue sample.

Depending on what the method of the first aspect is to be used for, the term “subject” may have different limitations. For example, if the method is to be used for detecting CRC or screening subjects for CRC, the subject is not known to have CRC, i.e. it may or may not have CRC. In this example, the subject preferably has an increased risk of developing or is suspected to have CRC, or has had CRC (i.e. has been cured of detectable CRC). “Increased risk” means that one or more risk factors for cancer generally or for the CRC can be attributed to the subject, preferably as defined by the American Cancer Society for cancer generally or for CRC. Examples of risk factors for CRC are: heavy alcohol use (more than 3 or 4 alcohol units a day for men, or more than 2 or 3 alcohol units a day for women; an alcohol unit is defined as 10 ml (8 g) of pure alcohol), tobacco consumption (in particular smoking, but also including smokeless tobacco), being overweight (Body Mass Index (BMI) of 25 to 29.9) or obese (BMI of 30 or more), especially having a larger waistline, physical inactivity (exercise (sports) for less than 150, preferably 75 minutes per week beyond usual (non-sport) daily activities), diet rich in red meats (such as beef, pork, lamb or liver) and processed meats, age of 50 or older, personal history of colorectal polyps, colorectal cancer and/or inflammatory bowel disease (e.g. ulcerative colitis or Crohn's disease), a familial history of colorectal cancer or adenomatous polyps (preferably first degree relative (parent, sibling or child), more preferably diagnosed at age 45 or younger and/or more than one first degree relative affected), having an inherited syndrome increased CRC risk such as preferably Lynch syndrome (hereditary non-polyposis colorectal cancer or HNPCC) or familial adenomatous polyposis (FAP), but also Peutz-Jeghers syndrome (PJS) or MYH-associated polyposis (MAP), racial and ethnic background with increased risk (e.g. African Americans or Ashkenazi Jews), and having type 2 diabetes.

Definitions and embodiments described below, in particular under the header ‘Definitions and further embodiments of the invention’ apply to the method of the first aspect.

In a second aspect, the invention relates to a method for detecting the presence or absence of CRC in a subject, comprising detecting DNA methylation according to the method of the first aspect, wherein the presence of detected methylated genomic DNA indicates the presence of CRC and the absence of detected methylated genomic DNA indicates the absence of CRC. Thus, the method of the second aspect useful as a method for diagnosis of CRC. The method is also useful as a method for screening a population of subjects for CRC.

Preferably, the method is an in vitro method.

The cancer may be of any subtype and stage as defined below, i.e. the presence or absence of any subtype and/or stage can be detected.

In a preferred embodiment, the presence of a significant amount of methylated genomic DNA, or of an amount larger than in a control, indicates the presence of CRC, and the absence of a significant amount of methylated genomic DNA, or of an amount equal to or smaller than in a control, indicates the absence of CRC.

In a particular embodiment, the method of the second aspect further comprises confirming the detection of CRC by using one or more further means for detecting CRC. The further means may be a cancer marker (or “biomarker”) or a conventional (non-marker) detection means. The cancer marker can for example be a DNA methylation marker, a mutation marker (e.g. SNP), an antigen marker, a protein marker, a miRNA marker, a cancer specific metabolite, or an expression marker (e.g. RNA or protein expression). The conventional means can for example be a biopsy (e.g. visual biopsy examination with or without staining methods for example for protein or expression markers), an imaging technique (e.g. X-ray imaging, CT scan, CR colonography, nuclear imaging such as PET and SPECT, ultrasound, magnetic resonance imaging (MM), thermography, or endoscopy, colonoscopy or sigmoidoscopy) or a physical, e.g. tactile examination. It is preferred that it is a colonoscopy, preferably involving a biopsy or other means that removes and examines a solid tissue sample of the subject from the tissue for which CRC is indicated (i.e. no liquid tissue such as blood).

In a preferred embodiment, the method of the second aspect is for monitoring a subject having an increased risk of developing CRC, suspected of having or developing CRC or that has had CRC, comprising detecting DNA methylation repeatedly, wherein the presence of detected methylated genomic DNA indicates the presence of CRC and the absence of detected methylated genomic DNA indicates the absence of CRC. Preferably, the detecting of the DNA methylation comprises determining the amount of methylated genomic DNA, wherein an increased amount of methylated genomic DNA in one or more repeated detections of DNA methylation indicates the presence of CRC and a constant or decreased amount in repeated detections of DNA methylation indicates the absence of CRC.

Definitions given and embodiments described with respect to the first aspect apply also to the second aspect, in as far as they are applicable. Also, definitions and embodiments described below, in particular under the header ‘Definitions and further embodiments of the invention’ apply to the method of the second aspect.

In a third aspect, the present invention relates to an oligonucleotide selected from the group consisting of a primer and a probe, comprising a sequence that is substantially identical to a stretch of contiguous nucleotides of one of SEQ ID NOs 17-20 (mADCYAP1), one of 57-60 and/or one of 62-65 (mANKRD13B), one of 42-45 and/or one of 47-50 (mCLEC14A), one of 72-75 (mCRMP1), one of 82-85 and/or one of 87-90 (mEYA4), one of 32-35 (mKHDRBS2), one of 97-100 and/or one of 102-105 (mMSC), one of 112-115 and/or one of 117-120 (mNGFR), one of 127-130 (mNKX2), one of 142-145 and/or one of 147-150 (mRASSF2), one of 2-5 (mSEPT9), one of 162-165 (mSND1), one of 172-175 (mTBX18), one of 187-190 and/or one of 192-195 (mTFAP2E), one of 202-205 and/or one of 207-210 (mTMEFF2), or one of 217-220 (mVAX1).

In a preferred embodiment,
the sequence that is substantially identical to a stretch of contiguous nucleotides of one of SEQ ID NOs 17-20 is substantially identical to a stretch of contiguous nucleotides of—one of SEQ ID NOs 22-25, preferably one of SEQ ID NOs 27-30,
the sequence that is substantially identical to a stretch of contiguous nucleotides of one of SEQ ID NOs 57-60 and/or one of SEQ ID NOs 62-65 is substantially identical to a stretch of contiguous nucleotides of one of SEQ ID NOs 67-70,
the sequence that is substantially identical to a stretch of contiguous nucleotides of one of SEQ ID NOs 42-45 and/or one of SEQ ID NOs 47-50 is substantially identical to a stretch of contiguous nucleotides of one of SEQ ID NOs 52-55,
the sequence that is substantially identical to a stretch of contiguous nucleotides of one of SEQ ID NOs 72-75 is substantially identical to a stretch of contiguous nucleotides of one of SEQ ID NOs 77-80,
the sequence that is substantially identical to a stretch of contiguous nucleotides of one of SEQ ID NOs 82-85 and/or one of SEQ ID NOs 87-90 is substantially identical to a stretch of contiguous nucleotides of one of SEQ ID NOs 92-95,
the sequence that is substantially identical to a stretch of contiguous nucleotides of one of SEQ ID NOs 32-35 is substantially identical to a stretch of contiguous nucleotides of one of SEQ ID NOs 37-40,
the sequence that is substantially identical to a stretch of contiguous nucleotides of one of SEQ ID NOs 97-100 and/or one of SEQ ID NOs 102-105 is substantially identical to a stretch of contiguous nucleotides of one of SEQ ID NOs 107-110,
the sequence that is substantially identical to a stretch of contiguous nucleotides of one of SEQ ID NOs 112-115 and/or one of SEQ ID NOs 117-120 is substantially identical to a stretch of contiguous nucleotides of one of SEQ ID NOs 122-125,
the sequence that is substantially identical to a stretch of contiguous nucleotides of one of SEQ ID NOs 127-130 is substantially identical to a stretch of contiguous nucleotides of one of SEQ ID NOs 132-135, preferably one of SEQ ID NOs 137-140,
the sequence that is substantially identical to a stretch of contiguous nucleotides of one of SEQ ID NOs 142-145 and/or one of SEQ ID NOs 147-150 is substantially identical to a stretch of contiguous nucleotides of one of SEQ ID NOs 152-155,
the sequence that is substantially identical to a stretch of contiguous nucleotides of one of SEQ ID NOs 2-5 is substantially identical to a stretch of contiguous nucleotides of one of SEQ

ID NOs 7-10, preferably one of SEQ ID NOs 12-15,
the sequence that is substantially identical to a stretch of contiguous nucleotides of one of SEQ ID NOs 162-165 is substantially identical to a stretch of contiguous nucleotides of one of SEQ ID NOs 157-160, preferably one of SEQ ID NOs 167-170,
the sequence that is substantially identical to a stretch of contiguous nucleotides of one of SEQ ID NOs 172-175 is substantially identical to a stretch of contiguous nucleotides of one of SEQ ID NOs 177-180, preferably one of SEQ ID NOs 182-185,
the sequence that is substantially identical to a stretch of contiguous nucleotides of one of SEQ ID NOs 187-190 and/or one of SEQ ID NOs 192-195 is substantially identical to a stretch of contiguous nucleotides of one of SEQ ID NOs 197-200,
the sequence that is substantially identical to a stretch of contiguous nucleotides of one of SEQ ID NOs 202-205 and/or one of SEQ ID NOs 207-210 is substantially identical to a stretch of contiguous nucleotides of one of SEQ ID NOs 212-215, and/or
the sequence that is substantially identical to a stretch of contiguous nucleotides of one of SEQ ID NOs 217-220 is substantially identical to a stretch of contiguous nucleotides of one of SEQ ID NOs 222-225.

Herein, a sequence that is substantially identical to a stretch of contiguous nucleotides of two (or more) SEQ ID NOs, e.g. of one of SEQ ID NOs 17-20 and of one of SEQ ID NOs 22-25 or e.g. of one of SEQ ID NOs 57-60 and/or one of SEQ ID NOs 62-65, is identical to two (or more) corresponding SEQ ID NOs. “Corresponding” means of the same type of the same methylation marker (e.g. mADCYAP1) according to Table 3 (the types are genomic reference, C to T (bis1), rc C to T (bis1), G to A (bis2 rc) and G to A (bis2 rc) rc).

Generally, the oligonucleotide is bisulfite-specific. Preferably, the oligonucleotide is methylation-specific, more preferably positive methylation-specific.

The oligonucleotide may be a primer or a probe oligonucleotide, preferably it is a primer oligonucleotide. A probe preferably has one or more modifications selected from the group consisting of a detectable label and a quencher, and/or a length of 5-40 nucleotides. A primer preferably has a priming region with a length of 10-40 nucleotides.

Definitions given and embodiments described with respect to the first and second aspect apply also to the third aspect, in as far as they are applicable. Also, definitions and embodiments described below, in particular under the header ‘Definitions and further embodiments of the invention’ apply to the oligonucleotide of the third aspect.

In a fourth aspect, the present invention relates to a kit comprising at least a first and a second oligonucleotide of the third aspect.

In a preferred embodiment, the first and second oligonucleotides are primers forming a primer pair suitable for amplification of DNA having a sequence comprised in one of SEQ ID NOs 17-20 (mADCYAP1), one of SEQ ID NOs 57-60 and/or one of SEQ ID NOs 62-65 (mANKRD13B), one of SEQ ID NOs 42-45 and/or one of SEQ ID NOs 47-50 (mCLEC14A), one of SEQ ID NOs 72-75 (mCRMP1), one of SEQ ID NOs 82-85 and/or one of SEQ ID NOs 87-90 (mEYA4), one of SEQ ID NOs 32-35 (mKHDRBS2), one of SEQ ID NOs 97-100 and/or one of SEQ ID NOs 102-105 (mMSC), one of SEQ ID NOs 112-115 and/or one of SEQ ID NOs 117-120 (mNGFR), one of SEQ ID NOs 127-130 (mNKX2), one of SEQ ID NOs 142-145 and/or one of SEQ ID NOs 147-150 (mRASSF2), one of SEQ ID NOs 2-5 (mSEPT9), one of SEQ ID NOs 162-165 (mSND1), one of SEQ ID NOs 172-175 (mTBX18), one of SEQ ID NOs 187-190 and/or one of SEQ ID NOs 192-195 (mTFAP2E), one of SEQ ID NOs 202-205 and/or one of SEQ ID NOs 207-210 (mTMEFF2), or one of SEQ ID NOs 217-220 (mVAX1).

Preferably,
the sequence comprised in one of SEQ ID NOs 17-20 is comprised in one of SEQ ID NOs 22-25, preferably one of SEQ ID NOs 27-30,
the sequence comprised in one of SEQ ID NOs 57-60 and/or one of SEQ ID NOs 62-65 is comprised in one of SEQ ID NOs 67-70,
the sequence comprised in one of SEQ ID NOs 42-45 and/or one of SEQ ID NOs 47-50 is comprised in one of SEQ ID NOs 52-55,
the sequence comprised in one of SEQ ID NOs 72-75 is comprised in one of SEQ ID NOs 77-80,
the sequence comprised in one of SEQ ID NOs 82-85 and/or one of SEQ ID NOs 87-90 is comprised in one of SEQ ID NOs 92-95,
the sequence comprised in one of SEQ ID NOs 32-35 is comprised in one of SEQ ID NOs 37-40,
the sequence comprised in one of SEQ ID NOs 97-100 and/or one of SEQ ID NOs 102-105 is comprised in one of SEQ ID NOs 107-110,
the sequence comprised in one of SEQ ID NOs 112-115 and/or one of SEQ ID NOs 117-120 is comprised in one of SEQ ID NOs 122-125,
the sequence comprised in one of SEQ ID NOs 127-130 is comprised in one of SEQ ID NOs 132-135, preferably one of SEQ ID NOs 137-140,
the sequence comprised in one of SEQ ID NOs 142-145 and/or one of SEQ ID NOs 147-150 is comprised in one of SEQ ID NOs 152-155,
the sequence comprised in one of SEQ ID NOs 2-5 is comprised in one of SEQ ID NOs 7-10, preferably one of SEQ ID NOs 12-15,
the sequence comprised in one of SEQ ID NOs 162-165 is comprised in one of SEQ ID NOs 157-160, preferably one of SEQ ID NOs 167-170,
the sequence comprised in one of SEQ ID NOs 172-175 is comprised in one of SEQ ID NOs 177-180, preferably one of SEQ ID NOs 182-185,
the sequence comprised in one of SEQ ID NOs 187-190 and/or one of SEQ ID NOs 192-195 is comprised in one of SEQ ID NOs 197-200,
the sequence comprised in one of SEQ ID NOs 202-205 and/or one of SEQ ID NOs 207-210 is comprised in one of SEQ ID NOs 212-215, and/or
the sequence comprised in one of SEQ ID NOs 217-220 is comprised in one of SEQ ID NOs 222-225.

Herein, a sequence that is comprised in two (or more) SEQ ID NOs, e.g. of one of SEQ ID NOs 17-20 and of one of SEQ ID NOs 22-25 or e.g. of one of SEQ ID NOs 57-60 and/or one of SEQ ID NOs 62-65, is comprised to two (or more) corresponding SEQ ID NOs. “Corresponding” means of the same type of the same methylation marker according to Table 3.

In another preferred embodiment, the kit comprises polynucleotides forming at least two, preferably at least three (or at least 4, 5, 6, 7, 8, 9, 10, 11, 12, 13, 14, 15 or at least 16, wherein larger numbers are preferred to smaller numbers) such primer pairs, wherein each primer pair is suitable for amplification of DNA having a sequence of a different marker selected from the group consisting of mADCYAP1, mANKRD13B, mCLEC14A, mCRMP1, mEYA4, mKHDRBS2, mMSC, mNGFR, mNKX2, mRASSF2, mSEPT9, mSND1, mTBX18, mTFAP2E, mTMEFF2 and mVAX1.

In specific preferred embodiments, the kit comprises polynucleotides forming primer pairs for markers of a combination of two markers according to Table 1 or three markers according to Table 2 (for which advantageous AUC values are shown), and optionally one or more further marker of the group consisting of mADCYAP1, mANKRD13B, mCLEC14A, mCRMP1, mEYA4, mKHDRBS2, mMSC, mNGFR, mNKX2, mRASSF2, mSEPT9, mSND1, mTBX18, mTFAP2E, mTMEFF2 and mVAX1.

Of the combinations recited in Table 1, those are particularly preferred for which an AUC of at least 0.80, preferably at least 0.84, 0.86, 0.88, 0.90, or 0.92, more preferably at least 0.93 is shown in Table 1. Of the combinations recited in Table 2, those are particularly preferred for which an AUC of at least 0.85, preferably at least 0.87, 0.89, 0.9, 0.91, or 0.92, more preferably at least 0.93 or 0.94 is shown in Table 2.

Definitions given and embodiments described with respect to the first, second and third aspect apply also to the fourth aspect, in as far as they are applicable. Also, definitions and embodiments described below, in particular under the header ‘Definitions and further embodiments of the invention’ apply to the kit of the fourth aspect.

In a fifth aspect, the present invention relates to the use of the method of the first aspect, of the oligonucleotide of the third aspect or of the kit the fourth aspect for the detection of CRC or for monitoring a subject having an increased risk of developing CRC, suspected of having or developing CRC or who has had CRC. Preferably, the use is an in vitro use.

Definitions given and embodiments described with respect to the first, second, third and fourth aspect apply also to the fifth aspect, in as far as they are applicable. Also, definitions and embodiments described below, in particular under the header ‘Definitions and further embodiments of the invention’ apply to the use of the fifth aspect.

In a sixth aspect, the present invention relates to the method of the first or the second aspect, or the use of the fifth aspect, comprising a step of treating CRC of a subject for which the DNA methylation is detected in its biological sample. In other words, the method of the sixth aspect can be described as a method of treatment, comprising the method of the first or the second aspect, or the use of the fifth aspect and a step of treating CRC of a subject for which the DNA methylation is detected in its biological sample. It can also be described as a method of treatment, comprising treating CRC in a subject for which DNA methylation has been detected according to the method of the first or the second aspect, or the use of the fifth aspect.

Definitions given and embodiments described with respect to the first, second, third, fourth and fifth aspect apply also to the sixth aspect, in as far as they are applicable. Also, definitions and embodiments described below, in particular under the header ‘Definitions and further embodiments of the invention’ apply to the method of the sixth aspect.

TABLE 1
Combinations of at least two markers comprising markers 1 and 2

| Marker 1 | Marker 2 | AUC |
|---|---|---|
| mSEPT9 | mADCYAP1 | 0.920 |
| mSEPT9 | mKHDRBS2 | 0.929 |
| mSEPT9 | mCLEC14A | 0.911 |
| mSEPT9 | mANKRD13B | 0.931 |
| mSEPT9 | mCRMP1 | 0.932 |
| mSEPT9 | mEYA4 | 0.908 |
| mSEPT9 | mMSC | 0.922 |
| mSEPT9 | mNGFR | 0.876 |
| mSEPT9 | mNKX2 | 0.918 |
| mSEPT9 | mRASSF2 | 0.912 |
| mSEPT9 | mSND1 | 0.926 |
| mSEPT9 | mTBX18 | 0.904 |
| mSEPT9 | mTFAP2E | 0.910 |
| mSEPT9 | mTMEFF2 | 0.925 |
| mSEPT9 | mVAX1 | 0.897 |
| mADCYAP1 | mKHDRBS2 | 0.881 |
| mADCYAP1 | mCLEC14A | 0.857 |
| mADCYAP1 | mANKRD13B | 0.898 |
| mADCYAP1 | mCRMP1 | 0.870 |
| mADCYAP1 | mEYA4 | 0.858 |
| mADCYAP1 | mMSC | 0.867 |
| mADCYAP1 | mNGFR | 0.865 |
| mADCYAP1 | mNKX2 | 0.856 |
| mADCYAP1 | mRASSF2 | 0.866 |
| mADCYAP1 | mSND1 | 0.887 |
| mADCYAP1 | mTBX18 | 0.852 |
| mADCYAP1 | mTFAP2E | 0.868 |
| mADCYAP1 | mTMEFF2 | 0.860 |
| mADCYAP1 | mVAX1 | 0.854 |
| mKHDRBS2 | mCLEC14A | 0.864 |
| mKHDRBS2 | mANKRD13B | 0.905 |
| mKHDRBS2 | mCRMP1 | 0.867 |
| mKHDRBS2 | mEYA4 | 0.852 |
| mKHDRBS2 | mMSC | 0.882 |
| mKHDRBS2 | mNGFR | 0.864 |
| mKHDRBS2 | mNKX2 | 0.876 |
| mKHDRBS2 | mRASSF2 | 0.878 |
| mKHDRBS2 | mSND1 | 0.887 |
| mKHDRBS2 | mTBX18 | 0.843 |
| mKHDRBS2 | mTFAP2E | 0.878 |
| mKHDRBS2 | mTMEFF2 | 0.867 |
| mKHDRBS2 | mVAX1 | 0.846 |
| mCLEC14A | mANKRD13B | 0.879 |
| mCLEC14A | mCRMP1 | 0.854 |
| mCLEC14A | mEYA4 | 0.828 |
| mCLEC14A | mMSC | 0.837 |
| mCLEC14A | mNGFR | 0.840 |
| mCLEC14A | mNKX2 | 0.837 |
| mCLEC14A | mRASSF2 | 0.838 |
| mCLEC14A | mSND1 | 0.855 |
| mCLEC14A | mTBX18 | 0.811 |
| mCLEC14A | mTFAP2E | 0.846 |
| mCLEC14A | mTMEFF2 | 0.826 |
| mCLEC14A | mVAX1 | 0.816 |
| mANKRD13B | mCRMP1 | 0.900 |
| mANKRD13B | mEYA4 | 0.875 |
| mANKRD13B | mMSC | 0.865 |
| mANKRD13B | mNGFR | 0.872 |
| mANKRD13B | mNKX2 | 0.883 |
| mANKRD13B | mRASSF2 | 0.868 |
| mANKRD13B | mSND1 | 0.871 |
| mANKRD13B | mTBX18 | 0.863 |
| mANKRD13B | mTFAP2E | 0.891 |
| mANKRD13B | mTMEFF2 | 0.880 |
| mANKRD13B | mVAX1 | 0.868 |
| mCRMP1 | mEYA4 | 0.864 |
| mCRMP1 | mMSC | 0.857 |
| mCRMP1 | mNGFR | 0.860 |
| mCRMP1 | mNKX2 | 0.854 |
| mCRMP1 | mRASSF2 | 0.853 |
| mCRMP1 | mSND1 | 0.880 |
| mCRMP1 | mTBX18 | 0.837 |
| mCRMP1 | mTFAP2E | 0.876 |
| mCRMP1 | mTMEFF2 | 0.837 |
| mCRMP1 | mVAX1 | 0.851 |
| mEYA4 | mMSC | 0.802 |
| mEYA4 | mNGFR | 0.822 |
| mEYA4 | mNKX2 | 0.849 |
| mEYA4 | mRASSF2 | 0.798 |
| mEYA4 | mSND1 | 0.822 |
| mEYA4 | mTBX18 | 0.770 |
| mEYA4 | mTFAP2E | 0.854 |
| mEYA4 | mTMEFF2 | 0.822 |
| mEYA4 | mVAX1 | 0.787 |
| mMSC | mNGFR | 0.829 |
| mMSC | mNKX2 | 0.838 |
| mMSC | mRASSF2 | 0.825 |
| mMSC | mSND1 | 0.850 |
| mMSC | mTBX18 | 0.816 |
| mMSC | mTFAP2E | 0.822 |
| mMSC | mTMEFF2 | 0.833 |
| mMSC | mVAX1 | 0.803 |
| mNGFR | mNKX2 | 0.842 |
| mNGFR | mRASSF2 | 0.813 |
| mNGFR | mSND1 | 0.841 |
| mNGFR | mTBX18 | 0.800 |
| mNGFR | mTFAP2E | 0.835 |
| mNGFR | mTMEFF2 | 0.843 |
| mNGFR | mVAX1 | 0.782 |
| mNKX2 | mRASSF2 | 0.851 |
| mNKX2 | mSND1 | 0.863 |
| mNKX2 | mTBX18 | 0.818 |
| mNKX2 | mTFAP2E | 0.861 |
| mNKX2 | mTMEFF2 | 0.839 |
| mNKX2 | mVAX1 | 0.835 |
| mRASSF2 | mSND1 | 0.842 |
| mRASSF2 | mTBX18 | 0.825 |
| mRASSF2 | mTFAP2E | 0.832 |
| mRASSF2 | mTMEFF2 | 0.823 |
| mRASSF2 | mVAX1 | 0.806 |
| mSND1 | mTBX18 | 0.833 |
| mSND1 | mTFAP2E | 0.851 |
| mSND1 | mTMEFF2 | 0.859 |
| mSND1 | mVAX1 | 0.824 |
| mTBX18 | mTFAP2E | 0.837 |
| mTBX18 | mTMEFF2 | 0.814 |
| mTBX18 | mVAX1 | 0.772 |
| mTFAP2E | mTMEFF2 | 0.858 |
| mTFAP2E | mVAX1 | 0.819 |
| mTMEFF2 | mVAX1 | 0.820 |

TABLE 2
Combinations of at least three markers
comprising markers 1, 2 and 3

| Marker 1 | Marker 2 | Marker 3 | AUC |
|---|---|---|---|
| mSEPT9 | mADCYAP1 | mKHDRBS2 | 0.938 |
| mSEPT9 | mADCYAP1 | mCLEC14A | 0.916 |
| mSEPT9 | mADCYAP1 | mANKRD13B | 0.929 |
| mSEPT9 | mADCYAP1 | mCRMP1 | 0.930 |
| mSEPT9 | mADCYAP1 | mEYA4 | 0.919 |
| mSEPT9 | mADCYAP1 | mMSC | 0.924 |
| mSEPT9 | mADCYAP1 | mNGFR | 0.918 |
| mSEPT9 | mADCYAP1 | mNKX2 | 0.919 |
| mSEPT9 | mADCYAP1 | mRASSF2 | 0.920 |
| mSEPT9 | mADCYAP1 | mSND1 | 0.930 |
| mSEPT9 | mADCYAP1 | mTBX18 | 0.919 |
| mSEPT9 | mADCYAP1 | mTFAP2E | 0.920 |
| mSEPT9 | mADCYAP1 | mTMEFF2 | 0.923 |
| mSEPT9 | mADCYAP1 | mVAX1 | 0.917 |
| mSEPT9 | mKHDRBS2 | mCLEC14A | 0.919 |
| mSEPT9 | mKHDRBS2 | mANKRD13B | 0.940 |
| mSEPT9 | mKHDRBS2 | mCRMP1 | 0.933 |
| mSEPT9 | mKHDRBS2 | mEYA4 | 0.926 |
| mSEPT9 | mKHDRBS2 | mMSC | 0.934 |
| mSEPT9 | mKHDRBS2 | mNGFR | 0.925 |
| mSEPT9 | mKHDRBS2 | mNKX2 | 0.934 |
| mSEPT9 | mKHDRBS2 | mRASSF2 | 0.933 |
| mSEPT9 | mKHDRBS2 | mSND1 | 0.941 |
| mSEPT9 | mKHDRBS2 | mTBX18 | 0.926 |
| mSEPT9 | mKHDRBS2 | mTFAP2E | 0.930 |
| mSEPT9 | mKHDRBS2 | mTMEFF2 | 0.932 |
| mSEPT9 | mKHDRBS2 | mVAX1 | 0.925 |
| mSEPT9 | mCLEC14A | mANKRD13B | 0.913 |
| mSEPT9 | mCLEC14A | mCRMP1 | 0.913 |
| mSEPT9 | mCLEC14A | mEYA4 | 0.916 |
| mSEPT9 | mCLEC14A | mMSC | 0.911 |
| mSEPT9 | mCLEC14A | mNGFR | 0.905 |
| mSEPT9 | mCLEC14A | mNKX2 | 0.909 |
| mSEPT9 | mCLEC14A | mRASSF2 | 0.897 |
| mSEPT9 | mCLEC14A | mSND1 | 0.918 |
| mSEPT9 | mCLEC14A | mTBχ18 | 0.902 |
| mSEPT9 | mCLEC14A | mTFAP2E | 0.914 |
| mSEPT9 | mCLEC14A | mTMEFF2 | 0.912 |
| mSEPT9 | mCLEC14A | mVAX1 | 0.908 |
| mSEPT9 | mANKRD13B | mCRMP1 | 0.940 |
| mSEPT9 | mANKRD13B | mEYA4 | 0.931 |
| mSEPT9 | mANKRD13B | mMSC | 0.930 |
| mSEPT9 | mANKRD13B | mNGFR | 0.927 |
| mSEPT9 | mANKRD13B | mNKX2 | 0.933 |
| mSEPT9 | mANKRD13B | mRASSF2 | 0.926 |
| mSEPT9 | mANKRD13B | mSND1 | 0.929 |
| mSEPT9 | mANKRD13B | mTBX18 | 0.925 |
| mSEPT9 | mANKRD13B | mTFAP2E | 0.932 |
| mSEPT9 | mANKRD13B | mTMEFF2 | 0.935 |
| mSEPT9 | mANKRD13B | mVAX1 | 0.926 |
| mSEPT9 | mCRMP1 | mEYA4 | 0.932 |
| mSEPT9 | mCRMP1 | mMSC | 0.926 |
| mSEPT9 | mCRMP1 | mNGFR | 0.926 |
| mSEPT9 | mCRMP1 | mNKX2 | 0.928 |
| mSEPT9 | mCRMP1 | mRASSF2 | 0.925 |
| mSEPT9 | mCRMP1 | mSND1 | 0.931 |
| mSEPT9 | mCRMP1 | mTBX18 | 0.920 |
| mSEPT9 | mCRMP1 | mTFAP2E | 0.928 |
| mSEPT9 | mCRMP1 | mTMEFF2 | 0.921 |
| mSEPT9 | mCRMP1 | mVAX1 | 0.928 |
| mSEPT9 | mEYA4 | mMSC | 0.923 |
| mSEPT9 | mEYA4 | mNGFR | 0.901 |
| mSEPT9 | mEYA4 | mNKX2 | 0.922 |
| mSEPT9 | mEYA4 | mRASSF2 | 0.914 |
| mSEPT9 | mEYA4 | mSND1 | 0.926 |
| mSEPT9 | mEYA4 | mTBX18 | 0.909 |
| mSEPT9 | mEYA4 | mTFAP2E | 0.919 |
| mSEPT9 | mEYA4 | mTMEFF2 | 0.923 |
| mSEPT9 | mEYA4 | mVAX1 | 0.903 |
| mSEPT9 | mMSC | mNGFR | 0.921 |
| mSEPT9 | mMSC | mNKX2 | 0.924 |
| mSEPT9 | mMSC | mRASSF2 | 0.923 |
| mSEPT9 | mMSC | mSND1 | 0.926 |
| mSEPT9 | mMSC | mTBX18 | 0.918 |
| mSEPT9 | mMSC | mTFAP2E | 0.918 |
| mSEPT9 | mMSC | mTMEFF2 | 0.923 |
| mSEPT9 | mMSC | mVAX1 | 0.922 |
| mSEPT9 | mNGFR | mNKX2 | 0.913 |
| mSEPT9 | mNGFR | mRASSF2 | 0.907 |
| mSEPT9 | mNGFR | mSND1 | 0.923 |
| mSEPT9 | mNGFR | mTBX18 | 0.903 |
| mSEPT9 | mNGFR | mTFAP2E | 0.902 |
| mSEPT9 | mNGFR | mTMEFF2 | 0.918 |
| mSEPT9 | mNGFR | mVAX1 | 0.890 |
| mSEPT9 | mNKX2 | mRASSF2 | 0.923 |
| mSEPT9 | mNKX2 | mSND1 | 0.934 |
| mSEPT9 | mNKX2 | mTBX18 | 0.909 |
| mSEPT9 | mNKX2 | mTFAP2E | 0.920 |
| mSEPT9 | mNKX2 | mTMEFF2 | 0.922 |
| mSEPT9 | mNKX2 | mVAX1 | 0.915 |
| mSEPT9 | mRASSF2 | mSND1 | 0.925 |
| mSEPT9 | mRASSF2 | mTBX18 | 0.911 |
| mSEPT9 | mRASSF2 | mTFAP2E | 0.912 |
| mSEPT9 | mRASSF2 | mTMEFF2 | 0.923 |
| mSEPT9 | mRASSF2 | mVAX1 | 0.914 |
| mSEPT9 | mSND1 | mTBX18 | 0.925 |
| mSEPT9 | mSND1 | mTFAP2E | 0.924 |
| mSEPT9 | mSND1 | mTMEFF2 | 0.928 |
| mSEPT9 | mSND1 | mVAX1 | 0.927 |
| mSEPT9 | mTBX18 | mTFAP2E | 0.910 |
| mSEPT9 | mTBX18 | mTMEFF2 | 0.913 |
| mSEPT9 | mTBX18 | mVAX1 | 0.904 |
| mSEPT9 | mTFAP2E | mTMEFF2 | 0.928 |
| mSEPT9 | mTFAP2E | mVAX1 | 0.910 |
| mSEPT9 | mTMEFF2 | mVAX1 | 0.919 |
| mADCYAP1 | mKHDRBS2 | mCLEC14A | 0.876 |
| mADCYAP1 | mKHDRBS2 | mANKRD13B | 0.911 |
| mADCYAP1 | mKHDRBS2 | mCRMP1 | 0.880 |
| mADCYAP1 | mKHDRBS2 | mEYA4 | 0.882 |
| mADCYAP1 | mKHDRBS2 | mMSC | 0.885 |
| mADCYAP1 | mKHDRBS2 | mNGFR | 0.886 |
| mADCYAP1 | mKHDRBS2 | mNKX2 | 0.882 |
| mADCYAP1 | mKHDRBS2 | mRASSF2 | 0.879 |
| mADCYAP1 | mKHDRBS2 | mSND1 | 0.902 |
| mADCYAP1 | mKHDRBS2 | mTBχ18 | 0.874 |
| mADCYAP1 | mKHDRBS2 | mTFAP2E | 0.893 |
| mADCYAP1 | mKHDRBS2 | mTMEFF2 | 0.876 |
| mADCYAP1 | mKHDRBS2 | mVAX1 | 0.880 |
| mADCYAP1 | mCLECUA | mANKRD13B | 0.888 |
| mADCYAP1 | mCLECUA | mCRMP1 | 0.864 |
| mADCYAP1 | mCLECUA | mEYA4 | 0.856 |
| mADCYAP1 | mCLECUA | mMSC | 0.868 |
| mADCYAP1 | mCLECUA | mNGFR | 0.860 |
| mADCYAP1 | mCLECUA | mNKX2 | 0.857 |
| mADCYAP1 | mCLECUA | mRASSF2 | 0.861 |
| mADCYAP1 | mCLECUA | mSND1 | 0.888 |
| mADCYAP1 | mCLEC14A | mTBX18 | 0.849 |
| mADCYAP1 | mCLEC14A | mTFAP2E | 0.868 |
| mADCYAP1 | mCLEC14A | mTMEFF2 | 0.857 |
| mADCYAP1 | mCLEC14A | mVAX1 | 0.851 |
| mADCYAP1 | mANKRD13B | mCRMP1 | 0.903 |
| mADCYAP1 | mANKRD13B | mEYA4 | 0.899 |
| mADCYAP1 | mANKRD13B | mMSC | 0.898 |
| mADCYAP1 | mANKRD13B | mNGFR | 0.893 |
| mADCYAP1 | mANKRD13B | mNKX2 | 0.892 |
| mADCYAP1 | mANKRD13B | mRASSF2 | 0.890 |
| mADCYAP1 | mANKRD13B | mSND1 | 0.907 |
| mADCYAP1 | mANKRD13B | mTBX18 | 0.889 |
| mADCYAP1 | mANKRD13B | mTFAP2E | 0.897 |
| mADCYAP1 | mANKRD13B | mTMEFF2 | 0.897 |
| mADCYAP1 | mANKRD13B | mVAX1 | 0.898 |
| mADCYAP1 | mCRMP1 | mEYA4 | 0.868 |
| mADCYAP1 | mCRMP1 | mMSC | 0.872 |
| mADCYAP1 | mCRMP1 | mNGFR | 0.873 |
| mADCYAP1 | mCRMP1 | mNKX2 | 0.865 |
| mADCYAP1 | mCRMP1 | mRASSF2 | 0.869 |
| mADCYAP1 | mCRMP1 | mSND1 | 0.890 |
| mADCYAP1 | mCRMP1 | mTBX18 | 0.862 |
| mADCYAP1 | mCRMP1 | mTFAP2E | 0.879 |
| mADCYAP1 | mCRMP1 | mTMEFF2 | 0.859 |
| mADCYAP1 | mCRMP1 | mVAX1 | 0.869 |
| mADCYAP1 | mEYA4 | mMSC | 0.866 |
| mADCYAP1 | mEYA4 | mNGFR | 0.863 |
| mADCYAP1 | mEYA4 | mNKX2 | 0.856 |
| mADCYAP1 | mEYA4 | mRASSF2 | 0.866 |
| mADCYAP1 | mEYA4 | mSND1 | 0.888 |
| mADCYAP1 | mEYA4 | mTBX18 | 0.850 |
| mADCYAP1 | mEYA4 | mTFAP2E | 0.866 |
| mADCYAP1 | mEYA4 | mTMEFF2 | 0.860 |
| mADCYAP1 | mEYA4 | mVAX1 | 0.854 |
| mADCYAP1 | mMSC | mNGFR | 0.872 |
| mADCYAP1 | mMSC | mNKX2 | 0.871 |
| mADCYAP1 | mMSC | mRASSF2 | 0.869 |
| mADCYAP1 | mMSC | mSND1 | 0.897 |
| mADCYAP1 | mMSC | mTBX18 | 0.862 |
| mADCYAP1 | mMSC | mTFAP2E | 0.874 |
| mADCYAP1 | mMSC | mTMEFF2 | 0.866 |
| mADCYAP1 | mMSC | mVAX1 | 0.865 |
| mADCYAP1 | mNGFR | mNKX2 | 0.862 |
| mADCYAP1 | mNGFR | mRASSF2 | 0.870 |
| mADCYAP1 | mNGFR | mSND1 | 0.893 |
| mADCYAP1 | mNGFR | mTBX18 | 0.857 |
| mADCYAP1 | mNGFR | mTFAP2E | 0.874 |
| mADCYAP1 | mNGFR | mTMEFF2 | 0.866 |
| mADCYAP1 | mNGFR | mVAX1 | 0.862 |
| mADCYAP1 | mNKX2 | mRASSF2 | 0.864 |
| mADCYAP1 | mNKX2 | mSND1 | 0.890 |
| mADCYAP1 | mNKX2 | mTBX18 | 0.855 |
| mADCYAP1 | mNKX2 | mTFAP2E | 0.868 |
| mADCYAP1 | mNKX2 | mTMEFF2 | 0.859 |
| mADCYAP1 | mNKX2 | mVAX1 | 0.854 |
| mADCYAP1 | mRASSF2 | mSND1 | 0.885 |
| mADCYAP1 | mRASSF2 | mTBX18 | 0.863 |
| mADCYAP1 | mRASSF2 | mTFAP2E | 0.872 |
| mADCYAP1 | mRASSF2 | mTMEFF2 | 0.861 |
| mADCYAP1 | mRASSF2 | mVAX1 | 0.867 |
| mADCYAP1 | mSND1 | mTBX18 | 0.884 |
| mADCYAP1 | mSND1 | mTFAP2E | 0.888 |
| mADCYAP1 | mSND1 | mTMEFF2 | 0.888 |
| mADCYAP1 | mSND1 | mVAX1 | 0.888 |
| mADCYAP1 | mTBX18 | mTFAP2E | 0.871 |
| mADCYAP1 | mTBX18 | mTMEFF2 | 0.855 |
| mADCYAP1 | mTBX18 | mVAX1 | 0.847 |
| mADCYAP1 | mTFAP2E | mTMEFF2 | 0.873 |
| mADCYAP1 | mTFAP2E | mVAX1 | 0.866 |
| mADCYAP1 | mTMEFF2 | mVAX1 | 0.860 |
| mKHDRBS2 | mCLEC14A | mANKRD13B | 0.893 |
| mKHDRBS2 | mCLEC14A | mCRMP1 | 0.868 |
| mKHDRBS2 | mCLEC14A | mEYA4 | 0.861 |
| mKHDRBS2 | mCLEC14A | mMSC | 0.884 |
| mKHDRBS2 | mCLEC14A | mNGFR | 0.870 |
| mKHDRBS2 | mCLEC14A | mNKX2 | 0.874 |
| mKHDRBS2 | mCLEC14A | mRASSF2 | 0.875 |
| mKHDRBS2 | mCLEC14A | mSND1 | 0.895 |
| mKHDRBS2 | mCLEC14A | mTBX18 | 0.854 |
| mKHDRBS2 | mCLEC14A | mTFAP2E | 0.882 |
| mKHDRBS2 | mCLEC14A | mTMEFF2 | 0.863 |
| mKHDRBS2 | mCLEC14A | mVAX1 | 0.858 |
| mKHDRBS2 | mANKRD13B | mCRMP1 | 0.907 |
| mKHDRBS2 | mANKRD13B | mEYA4 | 0.904 |
| mKHDRBS2 | mANKRD13B | mMSC | 0.899 |
| mKHDRBS2 | mANKRD13B | mNGFR | 0.900 |
| mKHDRBS2 | mANKRD13B | mNKX2 | 0.907 |
| mKHDRBS2 | mANKRD13B | mRASSF2 | 0.905 |
| mKHDRBS2 | mANKRD13B | mSND1 | 0.907 |
| mKHDRBS2 | mANKRD13B | mTBX18 | 0.884 |
| mKHDRBS2 | mANKRD13B | mTFAP2E | 0.906 |
| mKHDRBS2 | mANKRD13B | mTMEFF2 | 0.896 |
| mKHDRBS2 | mANKRD13B | mVAX1 | 0.900 |
| mKHDRBS2 | mCRMP1 | mEYA4 | 0.865 |
| mKHDRBS2 | mCRMP1 | mMSC | 0.878 |
| mKHDRBS2 | mCRMP1 | mNGFR | 0.874 |
| mKHDRBS2 | mCRMP1 | mNKX2 | 0.875 |
| mKHDRBS2 | mCRMP1 | mRASSF2 | 0.877 |
| mKHDRBS2 | mCRMP1 | mSND1 | 0.895 |
| mKHDRBS2 | mCRMP1 | mTBX18 | 0.852 |
| mKHDRBS2 | mCRMP1 | mTFAP2E | 0.882 |
| mKHDRBS2 | mCRMP1 | mTMEFF2 | 0.857 |
| mKHDRBS2 | mCRMP1 | mVAX1 | 0.861 |
| mKHDRBS2 | mEYA4 | mMSC | 0.882 |
| mKHDRBS2 | mEYA4 | mNGFR | 0.860 |
| mKHDRBS2 | mEYA4 | mNKX2 | 0.873 |
| mKHDRBS2 | mEYA4 | mRASSF2 | 0.872 |
| mKHDRBS2 | mEYA4 | mSND1 | 0.893 |
| mKHDRBS2 | mEYA4 | mTBX18 | 0.843 |
| mKHDRBS2 | mEYA4 | mTFAP2E | 0.874 |
| mKHDRBS2 | mEYA4 | mTMEFF2 | 0.861 |
| mKHDRBS2 | mEYA4 | mVAX1 | 0.848 |
| mKHDRBS2 | mMSC | mNGFR | 0.884 |
| mKHDRBS2 | mMSC | mNKX2 | 0.891 |
| mKHDRBS2 | mMSC | mRASSF2 | 0.882 |
| mKHDRBS2 | mMSC | mSND1 | 0.899 |
| mKHDRBS2 | mMSC | mTBX18 | 0.871 |
| mKHDRBS2 | mMSC | mTFAP2E | 0.884 |
| mKHDRBS2 | mMSC | mTMEFF2 | 0.875 |
| mKHDRBS2 | mMSC | mVAX1 | 0.881 |
| mKHDRBS2 | mNGFR | mNKX2 | 0.882 |
| mKHDRBS2 | mNGFR | mRASSF2 | 0.880 |
| mKHDRBS2 | mNGFR | mSND1 | 0.891 |
| mKHDRBS2 | mNGFR | mTBX18 | 0.849 |
| mKHDRBS2 | mNGFR | mTFAP2E | 0.879 |
| mKHDRBS2 | mNGFR | mTMEFF2 | 0.874 |
| mKHDRBS2 | mNGFR | mVAX1 | 0.861 |
| mKHDRBS2 | mNKX2 | mRASSF2 | 0.884 |
| mKHDRBS2 | mNKX2 | mSND1 | 0.904 |
| mKHDRBS2 | mNKX2 | mTBX18 | 0.862 |
| mKHDRBS2 | mNKX2 | mTFAP2E | 0.889 |
| mKHDRBS2 | mNKX2 | mTMEFF2 | 0.874 |
| mKHDRBS2 | mNKX2 | mVAX1 | 0.870 |
| mKHDRBS2 | mRASSF2 | mSND1 | 0.893 |
| mKHDRBS2 | mRASSF2 | mTBX18 | 0.865 |
| mKHDRBS2 | mRASSF2 | mTFAP2E | 0.892 |
| mKHDRBS2 | mRASSF2 | mTMEFF2 | 0.868 |
| mKHDRBS2 | mRASSF2 | mVAX1 | 0.874 |
| mKHDRBS2 | mSND1 | mTBX18 | 0.881 |
| mKHDRBS2 | mSND1 | mTFAP2E | 0.892 |
| mKHDRBS2 | mSND1 | mTMEFF2 | 0.887 |
| mKHDRBS2 | mSND1 | mVAX1 | 0.887 |
| mKHDRBS2 | mTBX18 | mTFAP2E | 0.867 |
| mKHDRBS2 | mTBX18 | mTMEFF2 | 0.854 |
| mKHDRBS2 | mTBX18 | mVAX1 | 0.840 |
| mKHDRBS2 | mTFAP2E | mTMEFF2 | 0.882 |
| mKHDRBS2 | mTFAP2E | mVAX1 | 0.872 |
| mKHDRBS2 | mTMEFF2 | mVAX1 | 0.861 |
| mCLEC14A | mANKRD13B | mCRMP1 | 0.892 |
| mCLEC14A | mANKRD13B | mEYA4 | 0.877 |
| mCLEC14A | mANKRD13B | mMSC | 0.879 |
| mCLEC14A | mANKRD13B | mNGFR | 0.879 |
| mCLEC14A | mANKRD13B | mNKX2 | 0.868 |
| mCLEC14A | mANKRD13B | mRASSF2 | 0.877 |
| mCLEC14A | mANKRD13B | mSND1 | 0.885 |
| mCLEC14A | mANKRD13B | mTBX18 | 0.870 |
| mCLEC14A | mANKRD13B | mTFAP2E | 0.881 |
| mCLEC14A | mANKRD13B | mTMEFF2 | 0.872 |
| mCLEC14A | mANKRD13B | mVAX1 | 0.876 |
| mCLEC14A | mCRMP1 | mEYA4 | 0.855 |
| mCLEC14A | mCRMP1 | mMSC | 0.859 |
| mCLEC14A | mCRMP1 | mNGFR | 0.860 |
| mCLEC14A | mCRMP1 | mNKX2 | 0.853 |
| mCLEC14A | mCRMP1 | mRASSF2 | 0.861 |
| mCLEC14A | mCRMP1 | mSND1 | 0.879 |
| mCLEC14A | mCRMP1 | mTBX18 | 0.844 |
| mCLEC14A | mCRMP1 | mTFAP2E | 0.866 |
| mCLEC14A | mCRMP1 | mTMEFF2 | 0.837 |
| mCLEC14A | mCRMP1 | mVAX1 | 0.853 |
| mCLEC14A | mEYA4 | mMSC | 0.833 |
| mCLEC14A | mEYA4 | mNGFR | 0.843 |
| mCLEC14A | mEYA4 | mNKX2 | 0.841 |
| mCLEC14A | mEYA4 | mRASSF2 | 0.836 |
| mCLEC14A | mEYA4 | mSND1 | 0.855 |
| mCLEC14A | mEYA4 | mTBχ18 | 0.810 |
| mCLEC14A | mEYA4 | mTFAP2E | 0.852 |
| mCLEC14A | mEYA4 | mTMEFF2 | 0.826 |
| mCLEC14A | mEYA4 | mVAX1 | 0.825 |
| mCLEC14A | mMSC | mNGFR | 0.847 |
| mCLEC14A | mMSC | mNKX2 | 0.851 |
| mCLEC14A | mMSC | mRASSF2 | 0.848 |
| mCLEC14A | mMSC | mSND1 | 0.872 |
| mCLEC14A | mMSC | mTBX18 | 0.843 |
| mCLEC14A | mMSC | mTFAP2E | 0.851 |
| mCLEC14A | mMSC | mTMEFF2 | 0.842 |
| mCLEC14A | mMSC | mVAX1 | 0.836 |
| mCLEC14A | mNGFR | mNKX2 | 0.848 |
| mCLEC14A | mNGFR | mRASSF2 | 0.843 |
| mCLEC14A | mNGFR | mSND1 | 0.857 |
| mCLEC14A | mNGFR | mTBχ18 | 0.829 |
| mCLEC14A | mNGFR | mTFAP2E | 0.855 |
| mCLEC14A | mNGFR | mTMEFF2 | 0.846 |
| mCLEC14A | mNGFR | mVAX1 | 0.830 |
| mCLEC14A | mNKX2 | mRASSF2 | 0.849 |
| mCLEC14A | mNKX2 | mSND1 | 0.869 |
| mCLEC14A | mNKX2 | mTBX18 | 0.826 |
| mCLEC14A | mNKX2 | mTFAP2E | 0.855 |
| mCLEC14A | mNKX2 | mTMEFF2 | 0.836 |
| mCLEC14A | mNKX2 | mVAX1 | 0.834 |
| mCLEC14A | mRASSF2 | mSND1 | 0.859 |
| mCLEC14A | mRASSF2 | mTBX18 | 0.839 |
| mCLEC14A | mRASSF2 | mTFAP2E | 0.854 |
| mCLEC14A | mRASSF2 | mTMEFF2 | 0.834 |
| mCLEC14A | mRASSF2 | mVAX1 | 0.835 |
| mCLEC14A | mSND1 | mTBX18 | 0.858 |
| mCLEC14A | mSND1 | mTFAP2E | 0.868 |
| mCLEC14A | mSND1 | mTMEFF2 | 0.864 |
| mCLEC14A | mSND1 | mVAX1 | 0.855 |
| mCLEC14A | mTBX18 | mTFAP2E | 0.846 |
| mCLEC14A | mTBX18 | mTMEFF2 | 0.830 |
| mCLEC14A | mTBX18 | mVAX1 | 0.811 |
| mCLEC14A | mTFAP2E | mTMEFF2 | 0.849 |
| mCLEC14A | mTFAP2E | mVAX1 | 0.842 |
| mCLEC14A | mTMEFF2 | mVAX1 | 0.823 |
| mANKRD13B | mCRMP1 | mEYA4 | 0.902 |
| mANKRD13B | mCRMP1 | mMSC | 0.889 |
| mANKRD13B | mCRMP1 | mNGFR | 0.889 |
| mANKRD13B | mCRMP1 | mNKX2 | 0.895 |
| mANKRD13B | mCRMP1 | mRASSF2 | 0.894 |
| mANKRD13B | mCRMP1 | mSND1 | 0.904 |
| mANKRD13B | mCRMP1 | mTBχ18 | 0.883 |
| mANKRD13B | mCRMP1 | mTFAP2E | 0.903 |
| mANKRD13B | mCRMP1 | mTMEFF2 | 0.886 |
| mANKRD13B | mCRMP1 | mVAX1 | 0.900 |
| mANKRD13B | mEYA4 | mMSC | 0.861 |
| mANKRD13B | mEYA4 | mNGFR | 0.875 |
| mANKRD13B | mEYA4 | mNKX2 | 0.883 |
| mANKRD13B | mEYA4 | mRASSF2 | 0.866 |
| mANKRD13B | mEYA4 | mSND1 | 0.869 |
| mANKRD13B | mEYA4 | mTBχ18 | 0.859 |
| mANKRD13B | mEYA4 | mTFAP2E | 0.888 |
| mANKRD13B | mEYA4 | mTMEFF2 | 0.877 |
| mANKRD13B | mEYA4 | mVAX1 | 0.876 |
| mANKRD13B | mMSC | mNGFR | 0.870 |
| mANKRD13B | mMSC | mNKX2 | 0.874 |
| mANKRD13B | mMSC | mRASSF2 | 0.865 |
| mANKRD13B | mMSC | mSND1 | 0.872 |
| mANKRD13B | mMSC | mTBX18 | 0.862 |
| mANKRD13B | mMSC | mTFAP2E | 0.867 |
| mANKRD13B | mMSC | mTMEFF2 | 0.869 |
| mANKRD13B | mMSC | mVAX1 | 0.865 |
| mANKRD13B | mNGFR | mNKX2 | 0.874 |
| mANKRD13B | mNGFR | mRASSF2 | 0.868 |
| mANKRD13B | mNGFR | mSND1 | 0.872 |
| mANKRD13B | mNGFR | mTBX18 | 0.867 |
| mANKRD13B | mNGFR | mTFAP2E | 0.883 |
| mANKRD13B | mNGFR | mTMEFF2 | 0.880 |
| mANKRD13B | mNGFR | mVAX1 | 0.872 |
| mANKRD13B | mNKX2 | mRASSF2 | 0.877 |
| mANKRD13B | mNKX2 | mSND1 | 0.888 |
| mANKRD13B | mNKX2 | mTBX18 | 0.870 |
| mANKRD13B | mNKX2 | mTFAP2E | 0.885 |
| mANKRD13B | mNKX2 | mTMEFF2 | 0.880 |
| mANKRD13B | mNKX2 | mVAX1 | 0.881 |
| mANKRD13B | mRASSF2 | mSND1 | 0.879 |
| mANKRD13B | mRASSF2 | mTBχ18 | 0.867 |
| mANKRD13B | mRASSF2 | mTFAP2E | 0.874 |
| mANKRD13B | mRASSF2 | mTMEFF2 | 0.867 |
| mANKRD13B | mRASSF2 | mVAX1 | 0.864 |
| mANKRD13B | mSND1 | mTBχ18 | 0.874 |
| mANKRD13B | mSND1 | mTFAP2E | 0.873 |
| mANKRD13B | mSND1 | mTMEFF2 | 0.886 |
| mANKRD13B | mSND1 | mVAX1 | 0.873 |
| mANKRD13B | mTBX18 | mTFAP2E | 0.870 |
| mANKRD13B | mTBX18 | mTMEFF2 | 0.866 |
| mANKRD13B | mTBX18 | mVAX1 | 0.863 |
| mANKRD13B | mTFAP2E | mTMEFF2 | 0.885 |
| mANKRD13B | mTFAP2E | mVAX1 | 0.889 |
| mANKRD13B | mTMEFF2 | mVAX1 | 0.877 |
| mCRMP1 | mEYA4 | mMSC | 0.855 |
| mCRMP1 | mEYA4 | mNGFR | 0.864 |
| mCRMP1 | mEYA4 | mNKX2 | 0.855 |
| mCRMP1 | mEYA4 | mRASSF2 | 0.853 |
| mCRMP1 | mEYA4 | mSND1 | 0.877 |
| mCRMP1 | mEYA4 | mTBX18 | 0.834 |
| mCRMP1 | mEYA4 | mTFAP2E | 0.875 |
| mCRMP1 | mEYA4 | mTMEFF2 | 0.836 |
| mCRMP1 | mEYA4 | mVAX1 | 0.863 |
| mCRMP1 | mMSC | mNGFR | 0.860 |
| mCRMP1 | mMSC | mNKX2 | 0.857 |
| mCRMP1 | mMSC | mRASSF2 | 0.854 |
| mCRMP1 | mMSC | mSND1 | 0.877 |
| mCRMP1 | mMSC | mTBX18 | 0.846 |
| mCRMP1 | mMSC | mTFAP2E | 0.857 |
| mCRMP1 | mMSC | mTMEFF2 | 0.842 |
| mCRMP1 | mMSC | mVAX1 | 0.857 |
| mCRMP1 | mNGFR | mNKX2 | 0.859 |
| mCRMP1 | mNGFR | mRASSF2 | 0.854 |
| mCRMP1 | mNGFR | mSND1 | 0.878 |
| mCRMP1 | mNGFR | mTBX18 | 0.846 |
| mCRMP1 | mNGFR | mTFAP2E | 0.871 |
| mCRMP1 | mNGFR | mTMEFF2 | 0.847 |
| mCRMP1 | mNGFR | mVAX1 | 0.848 |
| mCRMP1 | mNKX2 | mRASSF2 | 0.862 |
| mCRMP1 | mNKX2 | mSND1 | 0.889 |
| mCRMP1 | mNKX2 | mTBX18 | 0.840 |
| mCRMP1 | mNKX2 | mTFAP2E | 0.871 |
| mCRMP1 | mNKX2 | mTMEFF2 | 0.842 |
| mCRMP1 | mNKX2 | mVAX1 | 0.853 |
| mCRMP1 | mRASSF2 | mSND1 | 0.877 |
| mCRMP1 | mRASSF2 | mTBX18 | 0.852 |
| mCRMP1 | mRASSF2 | mTFAP2E | 0.869 |
| mCRMP1 | mRASSF2 | mTMEFF2 | 0.840 |
| mCRMP1 | mRASSF2 | mVAX1 | 0.859 |
| mCRMP1 | mSND1 | mTBX18 | 0.879 |
| mCRMP1 | mSND1 | mTFAP2E | 0.879 |
| mCRMP1 | mSND1 | mTMEFF2 | 0.873 |
| mCRMP1 | mSND1 | mVAX1 | 0.881 |
| mCRMP1 | mTBX18 | mTFAP2E | 0.861 |
| mCRMP1 | mTBX18 | mTMEFF2 | 0.830 |
| mCRMP1 | mTBX18 | mVAX1 | 0.836 |
| mCRMP1 | mTFAP2E | mTMEFF2 | 0.856 |
| mCRMP1 | mTFAP2E | mVAX1 | 0.870 |
| mCRMP1 | mTMEFF2 | mVAX1 | 0.837 |
| mEYA4 | mMSC | mNGFR | 0.831 |
| mEYA4 | mMSC | mNKX2 | 0.839 |
| mEYA4 | mMSC | mRASSF2 | 0.826 |
| mEYA4 | mMSC | mSND1 | 0.849 |
| mEYA4 | mMSC | mTBX18 | 0.813 |
| mEYA4 | mMSC | mTFAP2E | 0.824 |
| mEYA4 | mMSC | mTMEFF2 | 0.834 |
| mEYA4 | mMSC | mVAX1 | 0.802 |
| mEYA4 | mNGFR | mNKX2 | 0.850 |
| mEYA4 | mNGFR | mRASSF2 | 0.813 |
| mEYA4 | mNGFR | mSND1 | 0.839 |
| mEYA4 | mNGFR | mTBX18 | 0.805 |
| mEYA4 | mNGFR | mTFAP2E | 0.850 |
| mEYA4 | mNGFR | mTMEFF2 | 0.845 |
| mEYA4 | mNGFR | mVAX1 | 0.806 |
| mEYA4 | mNKX2 | mRASSF2 | 0.850 |
| mEYA4 | mNKX2 | mSND1 | 0.864 |
| mEYA4 | mNKX2 | mTBX18 | 0.820 |
| mEYA4 | mNKX2 | mTFAP2E | 0.865 |
| mEYA4 | mNKX2 | mTMEFF2 | 0.839 |
| mEYA4 | mNKX2 | mVAX1 | 0.845 |
| mEYA4 | mRASSF2 | mSND1 | 0.841 |
| mEYA4 | mRASSF2 | mTBX18 | 0.825 |
| mEYA4 | mRASSF2 | mTFAP2E | 0.837 |
| mEYA4 | mRASSF2 | mTMEFF2 | 0.823 |
| mEYA4 | mRASSF2 | mVAX1 | 0.808 |
| mEYA4 | mSND1 | mTBX18 | 0.830 |
| mEYA4 | mSND1 | mTFAP2E | 0.851 |
| mEYA4 | mSND1 | mTMEFF2 | 0.856 |
| mEYA4 | mSND1 | mVAX1 | 0.820 |
| mEYA4 | mTBX18 | mTFAP2E | 0.835 |
| mEYA4 | mTBX18 | mTMEFF2 | 0.813 |
| mEYA4 | mTBX18 | mVAX1 | 0.773 |
| mEYA4 | mTFAP2E | mTMEFF2 | 0.857 |
| mEYA4 | mTFAP2E | mVAX1 | 0.843 |
| mEYA4 | mTMEFF2 | mVAX1 | 0.819 |
| mMSC | mNGFR | mNKX2 | 0.850 |
| mMSC | mNGFR | mRASSF2 | 0.833 |
| mMSC | mNGFR | mSND1 | 0.860 |
| mMSC | mNGFR | mTBX18 | 0.841 |
| mMSC | mNGFR | mTFAP2E | 0.841 |
| mMSC | mNGFR | mTMEFF2 | 0.850 |
| mMSC | mNGFR | mVAX1 | 0.825 |
| mMSC | mNKX2 | mRASSF2 | 0.848 |
| mMSC | mNKX2 | mSND1 | 0.874 |
| mMSC | mNKX2 | mTBX18 | 0.847 |
| mMSC | mNKX2 | mTFAP2E | 0.850 |
| mMSC | mNKX2 | mTMEFF2 | 0.849 |
| mMSC | mNKX2 | mVAX1 | 0.834 |
| mMSC | mRASSF2 | mSND1 | 0.858 |
| mMSC | mRASSF2 | mTBX18 | 0.843 |
| mMSC | mRASSF2 | mTFAP2E | 0.840 |
| mMSC | mRASSF2 | mTMEFF2 | 0.832 |
| mMSC | mRASSF2 | mVAX1 | 0.823 |
| mMSC | mSND1 | mTBX18 | 0.857 |
| mMSC | mSND1 | mTFAP2E | 0.855 |
| mMSC | mSND1 | mTMEFF2 | 0.867 |
| mMSC | mSND1 | mVAX1 | 0.850 |
| mMSC | mTBX18 | mTFAP2E | 0.841 |
| mMSC | mTBX18 | mTMEFF2 | 0.834 |
| mMSC | mTBX18 | mVAX1 | 0.813 |
| mMSC | mTFAP2E | mTMEFF2 | 0.846 |
| mMSC | mTFAP2E | mVAX1 | 0.819 |
| mMSC | mTMEFF2 | mVAX1 | 0.832 |
| mNGFR | mNKX2 | mRASSF2 | 0.848 |
| mNGFR | mNKX2 | mSND1 | 0.867 |
| mNGFR | mNKX2 | mTBX18 | 0.828 |
| mNGFR | mNKX2 | mTFAP2E | 0.860 |
| mNGFR | mNKX2 | mTMEFF2 | 0.847 |
| mNGFR | mNKX2 | mVAX1 | 0.834 |
| mNGFR | mRASSF2 | mSND1 | 0.849 |
| mNGFR | mRASSF2 | mTBX18 | 0.833 |
| mNGFR | mRASSF2 | mTFAP2E | 0.844 |
| mNGFR | mRASSF2 | mTMEFF2 | 0.843 |
| mNGFR | mRASSF2 | mVAX1 | 0.813 |
| mNGFR | mSND1 | mTBX18 | 0.847 |
| mNGFR | mSND1 | mTFAP2E | 0.864 |
| mNGFR | mSND1 | mTMEFF2 | 0.867 |
| mNGFR | mSND1 | mVAX1 | 0.839 |
| mNGFR | mTBX18 | mTFAP2E | 0.848 |
| mNGFR | mTBX18 | mTMEFF2 | 0.834 |
| mNGFR | mTBX18 | mVAX1 | 0.803 |
| mNGFR | mTFAP2E | mTMEFF2 | 0.862 |
| mNGFR | mTFAP2E | mVAX1 | 0.829 |
| mNGFR | mTMEFF2 | mVAX1 | 0.838 |
| mNKX2 | mRASSF2 | mSND1 | 0.868 |
| mNKX2 | mRASSF2 | mTBX18 | 0.848 |
| mNKX2 | mRASSF2 | mTFAP2E | 0.862 |
| mNKX2 | mRASSF2 | mTMEFF2 | 0.843 |
| mNKX2 | mRASSF2 | mVAX1 | 0.848 |
| mNKX2 | mSND1 | mTBX18 | 0.868 |
| mNKX2 | mSND1 | mTFAP2E | 0.870 |
| mNKX2 | mSND1 | mTMEFF2 | 0.876 |
| mNKX2 | mSND1 | mVAX1 | 0.863 |
| mNKX2 | mTBX18 | mTFAP2E | 0.850 |
| mNKX2 | mTBX18 | mTMEFF2 | 0.833 |
| mNKX2 | mTBX18 | mVAX1 | 0.824 |
| mNKX2 | mTFAP2E | mTMEFF2 | 0.860 |
| mNKX2 | mTFAP2E | mVAX1 | 0.857 |
| mNKX2 | mTMEFF2 | mVAX1 | 0.835 |
| mRASSF2 | mSND1 | mTBX18 | 0.852 |
| mRASSF2 | mSND1 | mTFAP2E | 0.860 |
| mRASSF2 | mSND1 | mTMEFF2 | 0.856 |
| mRASSF2 | mSND1 | mVAX1 | 0.841 |
| mRASSF2 | mTBχ18 | mTFAP2E | 0.850 |
| mRASSF2 | mTBχ18 | mTMEFF2 | 0.833 |
| mRASSF2 | mTBχ18 | mVAX1 | 0.823 |
| mRASSF2 | mTFAP2E | mTMEFF2 | 0.851 |
| mRASSF2 | mTFAP2E | mVAX1 | 0.831 |
| mRASSF2 | mTMEFF2 | mVAX1 | 0.821 |
| mSND1 | mTBX18 | mTFAP2E | 0.850 |
| mSND1 | mTBX18 | mTMEFF2 | 0.854 |
| mSND1 | mTBX18 | mVAX1 | 0.834 |
| mSND1 | mTFAP2E | mTMEFF2 | 0.865 |
| mSND1 | mTFAP2E | mVAX1 | 0.851 |
| mSND1 | mTMEFF2 | mVAX1 | 0.859 |
| mTBX18 | mTFAP2E | mTMEFF2 | 0.850 |
| mTBX18 | mTFAP2E | mVAX1 | 0.833 |
| mTBX18 | mTMEFF2 | mVAX1 | 0.813 |
| mTFAP2E | mTMEFF2 | mVAX1 | 0.846 |

### Definitions and Further Embodiments of the Invention

The specification uses a variety of terms and phrases, which have certain meanings as defined below. Preferred meanings are to be construed as preferred embodiments of the aspects of the invention described herein. As such, they and also further embodiments described in the following can be combined with any embodiment of the aspects of the invention and in particular any preferred embodiment of the aspects of the invention described above.

The term “methylated” as used herein refers to a biochemical process involving the addition of a methyl group to cytosine DNA nucleotides. DNA methylation at the 5 position of cytosine, especially in promoter regions, can have the effect of reducing gene expression and has been found in every vertebrate examined. In adult non-gamete cells, DNA methylation typically occurs in a CpG site. The term “CpG site” or “CpG dinucleotide”, as used herein, refers to regions of DNA where a cytosine nucleotide occurs next to a guanine nucleotide in the linear sequence of bases along its length. “CpG” is shorthand for “C-phosphate-G”, that is cytosine and guanine separated by only one phosphate; phosphate links any two nucleosides together in DNA. The “CpG” notation is used to distinguish this linear sequence from the CG base-pairing of cytosine and guanine. Cytosines in CpG dinucleotides can be methylated to form 5-methylcytosine. The term “CpG site” or “CpG site of genomic DNA” is also used with respect to the site of a former (unmethylated) CpG site in DNA in which the unmethylated C of the CpG site was converted to another as described herein (e.g. by bisulfite to uracil). The application provides the genomic sequence of each relevant DNA region as well as the bisulfite converted sequences of each converted strand. CpG sites referred to are always the positions of the CpG sites of the genomic sequence, even if the converted sequence does no longer contain these CpG sites due to the conversion. Specifically, methylation in the context of the present invention means hypermethylation. The term “hypermethylation” refers to an aberrant methylation pattern or status (i.e. the presence or absence of methylation of one or more nucleotides), wherein one or more nucleotides, preferably C(s) of a CpG site(s), are methylated compared to the same genomic DNA of a control, i.e. from a non-cancer cell of the subject or a subject not suffering or having suffered from the cancer the subject is treated for, preferably any cancer (healthy control). The term “control” can also refer to the methylation status, pattern or amount which is the average or median known of or determined from a group of at least 5, preferably at least 10 subjects. In particular, it refers to an increased presence of 5-mCyt at one or a plurality of CpG dinucleotides within a DNA sequence of a test DNA sample, relative to the amount of 5-mCyt found at corresponding CpG dinucleotides within a (healthy) control DNA sample, both samples preferably being of the same type, e.g. both blood plasma, both blood serum, both saliva, or both urine. Hypermethylation as a methylation status/pattern can be determined at one or more CpG site(s). If more than one CpG site is used, hypermethylation can be determined at each site separately or as an average of the CpG sites taken together. Alternatively, all assessed CpG sites must be methylated (comethylation) such that the requirement hypermethylation is fulfilled.

The term “detecting DNA methylation” as used herein refers to at least qualitatively analysing for the presence or absence of methylated target DNA. “Target DNA” refers to a sequence within the genomic DNA polynucleotide (region) that is generally limited in length, but is preferably a length suitable for PCR amplification, e.g. at least 30 to 1000, more preferably 50 to 300 and even more preferably 75 to 200 or 75 to 150 nucleotides long. This includes primer binding sites if the target region is amplified using primers. Methylation is preferably determined at 1 or more, 2 or more, 3 or more, 4 or more, or 5 or more, most preferably 6 or more (e.g. 10 or more, 15 or more, or 30 or more) CpG sites of the target DNA. Usually, the CpG sites analysed are comethylated in cancer, such that also CpG sites of neighbouring DNA are methylated and can be analysed in addition or instead. “At least qualitatively” means that also a quantitative determination of methylated target DNA, if present, can be performed. In fact, it is preferred that detecting of the DNA methylation comprises determining the amount of methylated genomic DNA.

DNA methylation can be detected or its amount can be determined by various means known in the art, e.g. autoradiography, silver staining or ethidium bromide staining, methylation sensitive single nucleotide extension (MS-SNUPE), methyl-binding proteins, antibodies for methylated DNA, methylation-sensitive restriction enzymes etc., preferably by sequencing, e.g. next-generation-sequencing (NGS), or by real-time PCR, e.g. multiplex real-time PCR, or by digital PCR (dPCR). In particular if 3 or more (e.g. 4 or more or 5 or more) different target DNAs (i.e. markers) are examined in parallel, it is preferred that the presence or absence of methylated DNA is detected by sequencing, preferably by NGS.

In a real-time PCR, this is done by detecting a methylation-specific oligonucleotide probe during amplifying the converted (e.g. bisulfite converted) target DNA methylation-specifically using methylation-specific primers or a methylation-specific blocker with methylation-specific primers or preferably methylation-unspecific primers.

Digital PCR (dPCR) is a quantitative PCR in which a PCR reaction mixture is partitioned into individual compartments (e.g. wells or water-in-oil emulsion droplets) resulting in either 1 or 0 targets being present in each compartment. Following PCR amplification, the number of positive vs negative reactions is determined and the quantification is by derived from this result statistically, preferably using Poisson statistics. A preferred dPCR is BEAMing (Beads, Emulsion, Amplification, Magnetics), in which DNA templates (which may be pre-amplified) are amplified using primers bound to magnetic beads present compartmentalized in water-in-oil emulsion droplets. Amplification results in the beads being covered with amplified DNA. The beads are then pooled and amplification is analysed, e.g. using methylation-specific fluorescent probes which can be analyzed by flow cytometry. See for instance Yokoi et al. (Int J Sci. 2017 April; 18(4):735). Applied to methylation analysis, the method is also known as Methyl BEAMing.

A detection by sequencing is preferably a detection by NGS. Therein, the converted methylated target DNA is amplified, preferably methylation-specifically (the target DNA is amplified if it is methylated, in other words if cytosines of the CpG sites are not converted). This can be achieved by bisulfite-specific primers which are methylation-specific. Then, the amplified sequences are sequenced and subsequently counted. The ratio of sequences derived from converted methylated DNA (identified in the sequences by CpG sites) and sequences derived from converted unmethylated DNA is calculated, resulting in a (relative) amount of methylated target DNA.

The term “next-generation-sequencing” (NGS, also known as 2^{nd} or 3^{rd} generation sequencing) refers to a sequencing the bases of a small fragment of DNA are sequentially identified from signals emitted as each fragment is re-synthesized from a DNA template strand. NGS extends this process across millions of reactions in a massively parallel fashion, rather than being limited to a single or a few DNA fragments. This advance enables rapid sequencing of the amplified DNA, with the latest instruments capable of producing hundreds of gigabases of data in a single sequencing run. See, e.g., Shendure and Ji, Nature Biotechnology 26, 1135-1145 (2008) or Mardis, Annu Rev Genomics Hum Genet. 2008; 9:387-402. Suitable NGS platforms are available commercially, e.g. the Roche 454 platform, the Roche 454 Junior platform, the Illumina HiSeq or MiSeq platforms, or the Life Technologies SOLiD 5500 or Ion Torrent platforms.

Generally, a quantification (e.g. determining the amount of methylated target DNA) may be absolute, e.g. in pg per mL or ng per mL sample, copies per mL sample, number of PCR cycles etc., or it may be relative, e.g. 10 fold higher than in a control sample or as percentage of methylation of a reference control (preferably fully methylated DNA). Determining the amount of methylated target DNA in the sample may comprise normalizing for the amount of total DNA in the sample. Normalizing for the amount of total DNA in the test sample preferably comprises calculating the ratio of the amount of methylated target DNA and (i) the amount of DNA of a reference site or (ii) the amount of total DNA of the target (e.g. the amount of methylated target DNA plus the amount of unmethylated target DNA, the latter preferably measured on the reverse strand). A reference site can be any genomic site and does not have to be a gene. It is preferred that the number of occurrences of the sequence of the reference site is stable or expected to be stable (i.e. constant) over a large population (e.g. is not in a repeat, i.e. in repetitive DNA). The reference site can, for instance be a housekeeping gene such as beta-Actin.

As mentioned above, the amount of methylated target DNA in the sample may be expressed as the proportion of the amount of methylated target DNA relative to the amount of methylated target DNA (reference control) in a reference sample comprising substantially fully methylated genomic DNA. Preferably, determining the proportion of methylated target DNA comprises determining the amount of methylated DNA of the same target in a reference sample, inter sample normalization of total methylated DNA, preferably by using the methylation unspecific measurement of a reference site, and dividing the ratio derived from the test sample by the corresponding ratio derived from the reference sample. The proportion can be expressed as a percentage or PMR (Percentage of Methylated Reference) by multiplying the result of the division by 100. The determination of the PMR is described in detail in Ogino et al. (JMD May 2006, Vol. 8, No. 2).

The term “amplifying” or “generating an amplicon” as used herein refers to an increase in the number of copies of the target nucleic acid and its complementary sequence, or particularly a region thereof. The target can be a double-stranded or single-stranded DNA template. The amplification may be performed by using any method known in the art, typically with a polymerase chain reaction (PCR). An “amplicon” is a double-stranded fragment of DNA according to said defined region. The amplification is preferably performed by methylation-specific PCR (i.e. an amplicon is produced depending on whether one or more CpG sites are converted or not) using (i) methylation-specific primers, or (ii) primers which are methylation-unspecific, but specific to bisulfite-converted DNA (i.e. hybridize only to converted DNA by covering at least one converted C not in a CpG context). Methylation-specificity with (ii) is achieved by using methylation-specific blocker oligonucleotides, which hybridize specifically to converted or non-converted CpG sites and thereby terminate the PCR polymerization. For example, the step of amplifying comprises a real-time PCR, in particular HeavyMethyl™ or HeavyMethyl™-MethyLight™.

The term “genomic DNA” as used herein refers to chromosomal DNA and is used to distinguish from coding DNA. As such, it includes exons, introns as well as regulatory sequences, in particular promoters, belonging to a gene.

The phrase “converting, in DNA, cytosine unmethylated in the 5-position to uracil or another base that does not hybridize to guanine” as used herein refers to a process of chemically treating the DNA in such a way that all or substantially all of the unmethylated cytosine bases are converted to uracil bases, or another base which is dissimilar to cytosine in terms of base pairing behaviour, while the 5-methylcytosine bases remain unchanged. The conversion of unmethylated, but not methylated, cytosine bases within the DNA sample is conducted with a converting agent. The term “converting agent” as used herein relates to a reagent capable of converting an unmethylated cytosine to uracil or to another base that is detectably dissimilar to cytosine in terms of hybridization properties. The converting agent is preferably a bisulfite such as disulfite, or hydrogen sulfite. The reaction is performed according to standard procedures (Frommer et al., 1992, Proc Natl Acad Sci USA 89:1827-31; Olek, 1996, Nucleic Acids Res 24:5064-6; EP 1394172). It is also possible to conduct the conversion enzymatically, e.g by use of methylation specific cytidine deaminases. Most preferably, the converting agent is sodium bisulfite, ammonium bisulfite or bisulfite.

The term “bisulfite-specific” means specific for bisulfite-converted DNA. Bisulfite-converted DNA is DNA in which at least one C not in a CpG context (e.g. of a CpC, CpA or CpT dinucleotide), preferably all, has/have been converted into a T or U (chemically converted into U, which by DNA amplification becomes T). With respect to an oligonucleotide, it means that the oligonucleotide covers or hybridizes to at least one nucleotide derived from conversion of a C not in a CpG context (e.g. of a CpC, CpA or CpT dinucleotide) or its complement into a T.

The term “methylation-specific” as used herein refers generally to the dependency from the presence or absence of CpG methylation.

The term “methylation-specific” as used herein with respect to an oligonucleotide means that the oligonucleotide does or does not anneal to a single-strand of DNA (in which cytosine unmethylated in the 5-position has been converted to uracil or another base that does not hybridize to guanine, and where it comprises at least one CpG site before conversion) without a mismatch regarding the position of the C in the at least one CpG site, depending on whether the C of the at least one CpG sites was unmethylated or methylated prior to the conversion, i.e. on whether the C has been converted or not. The methylation-specificity can be either positive (the oligonucleotide anneals without said mismatch if the C was not converted) or negative (the oligonucleotide anneals without said mismatch if the C was converted). To prevent annealing of the oligonucleotide contrary to its specificity, it preferably covers at least 2, 3, 4, 5 or 6 and preferably 3 to 6 CpG sites before conversion.

The term “methylation-unspecific” as used herein refers generally to the independency from the presence or absence of CpG methylation.

The term “methylation-unspecific” as used herein with respect to an oligonucleotide means that the oligonucleotide does anneal to a single-strand of DNA (in which cytosine unmethylated in the 5-position has been converted to uracil or another base that does not hybridize to guanine, and where it may or may not comprise at least one CpG site before conversion) irrespective of whether the C of the at least one CpG site was unmethylated or methylated prior to the conversion, i.e. of whether the C has been converted or not. In one case, the region of the single-strand of DNA the oligonucleotide anneals to does not comprise any CpG sites (before and after conversion) and the oligonuclotide is methylation-unspecific solely for this reason. While a methylation-unspecific oligonucleotide may cover one or more CpG dinucleotides, it does so with mismatches and/or spacers. The term “mismatch” as used herein refers to base-pair mismatch in DNA, more specifically a base-pair that is unable to form normal base-pairing interactions (i.e., other than “A” with “T” or “U”, or “G” with “C”).

Methylation is detected within the at least one genomic DNA polynucleotide, i.e. in a particular region of the DNA according to the SEQ ID NO referred to (the “target DNA”). The term “target DNA” as used herein refers to a genomic nucleotide sequence at a specific chromosomal location. In the context of the present invention, it is typically a genetic marker that is known to be methylated in the state of disease (for example in cancer cells vs. non-cancer cells). A genetic marker can be a coding or non-coding region of genomic DNA.

The term “region of the target DNA” or “region of the converted DNA” as used herein refers to a part of the target DNA which is to be analysed. Preferably, the region is at least 40, 50, 60, 70, 80, 90, 100, 150, or 200 or 300 base pairs (bp) long and/or not longer than 500, 600, 700, 800, 900 or 1000 bp (e.g. 25-500, 50-250 or 75-150 bp). In particular, it is a region comprising at least 3, 4, 5, 6, 7, 8, 9, 10, 11, 12, 13, 14, 15 or 16 CpG sites of the genomic DNA. The target DNAs of the invention are given in FIG. 1 and Table 3.

For an amplification of the target region with at least one methylation-specific primer, it is preferred that the at least one methylation-specific primer covers at least 1, at least 2 or preferably at least 3 CpG sites (e.g. 2-8 or preferably 3-6 CpG sites) of the target region. Preferably, at least 1, at least 2 or preferably at least 3 CpG sites of these CpG sites are covered by the 3′ third of the primer (and/or one of these CpG sites is covered by the 3′ end of the primer (last three nucleotides of the primer).

The term “covering a CpG site” as used herein with respect to an oligonucleotide refers to the oligonucleotide annealing to a region of DNA comprising this CpG site, before or after conversion of the C of the CpG site (i.e. the CpG site of the corresponding genomic DNA when it is referred to a bisulfite converted sequence). The annealing may, with respect to the CpG site (or former CpG site if the C was converted), be methylation-specific or methylation-unspecific as described herein.

The term “annealing”, when used with respect to an oligonucleotide, is to be understood as a bond of an oligonucleotide to an at least substantially complementary sequence along the lines of the Watson-Crick base pairings in the sample DNA, forming a duplex structure, under moderate or stringent hybridization conditions. When it is used with respect to a single nucleotide or base, it refers to the binding according to Watson-Crick base pairings, e.g. C-G, A-T and A-U. Stringent hybridization conditions involve hybridizing at 68° C. in 5×SSC/5×Denhardt's solution/1.0% SDS, and washing in 0.2×SSC/0.1% SDS at room temperature, or involve the art-recognized equivalent thereof (e.g., conditions in which a hybridization is carried out at 60° C. in 2.5×SSC buffer, followed by several washing steps at 37° C. in a low buffer concentration, and remains stable). Moderate conditions involve washing in 3×SSC at 42° C., or the art-recognized equivalent thereof. The parameters of salt concentration and temperature can be varied to achieve the optimal level of identity between the probe and the target nucleic acid. Guidance regarding such conditions is available in the art, for example, by Sambrook et al., 1989, Molecular Cloning, A Laboratory Manual, Cold Spring Harbor Press, N.Y.; and Ausubel et al. (eds.), 1995, Current Protocols in Molecular Biology, (John Wiley & Sons, N.Y.) at Unit 2.10.

The cancer of the specification includes the following stages (as defined by the corresponding TNM classification(s) in brackets) of the cancer and each of its subtypes: stage 0 (Tis, N0, M0), stage I (T1, N0, M0), stage II (T2, N0, M0), stage III (T3, N0, M0; or T1 to T3, N1, M0), stage IVA (T4a, N0 or N1, M0; or T1 to T4a, N2, M0), stage IVB (T4b, any N, M0 or any T, N3, M0), and stage IVC (any T, any N, M1). The TNM classification is a staging system for malignant cancer. As used herein the term “TNM classification” refers to the 6^{th} edition of the TNM stage grouping as defined in Sobin et al. (International Union Against Cancer (UICC), TNM Classification of Malignant tumors, 6th ed. New York; Springer, 2002, pp. 191-203).

The term “subject” as used herein refers to a human individual.

The term “biological sample” as used herein refers to material obtained from a subject and comprises genomic DNA from all chromosomes, preferably genomic DNA covering the whole genome. Preferably, the sample comprises cell-free genomic DNA (including the target DNA), preferably circulating genomic DNA. If a subject has cancer, the cell-free (preferably circulating) genomic DNA comprises cell-free (preferably circulating) genomic DNA from cancer cells, i.e. preferably ctDNA.

The term “liquid biopsy” as used herein refers to a body fluid sample comprising cell-free (preferably circulating) genomic DNA. It is envisaged that it is a body liquid in which cell-free (preferably circulating) genomic DNA from cells of the cancer of the specification can be found if the subject has the cancer. A “blood-derived sample” is any sample that is derived by in vitro processing from blood, e.g. plasma or serum. “A sample comprising cell-free DNA from blood” can be any such sample. For example, urine comprises cell-free DNA from blood.

The term “cell-free DNA” as used herein or its synonyms “cfDNA”, and “extracellular DNA”, “circulating DNA” and “free circulating DNA” refers to DNA that is not comprised within an intact cell in the respective body fluid which is the sample or from which the sample is derived, but which is free in the body liquid sample. Cell-free DNA usually is genomic DNA that is fragmented as described below.

The term “circulating DNA” or “free circulating DNA” as used herein refers to cell-free DNA in a body liquid (in particular blood) which circulates in the body.

The term “circulating tumor DNA” or “ctDNA” as used herein refers to circulating DNA that is derived from a tumor (i.e. cell-free DNA derived from tumor cells).

Typically, in samples comprising the target DNA, especially extracellular target DNA, from cancer cells, there is also target DNA from non-cancer cells which is not methylated contrary to the target DNA from cancer cells. Usually, said target DNA from non-cancer cells exceeds the amount from diseased cells by at least 10-fold, at least 100-fold, at least 1,000-fold or at least 10,000-fold. Generally, the genomic DNA comprised in the sample is at least partially fragmented. “At least partially fragmented” means that at least the extracellular DNA, in particular at least the extracellular target DNA, from cancer cells, is fragmented. The term “fragmented genomic DNA” refers to pieces of DNA of the genome of a cell, in particular a cancer cell, that are the result of a partial physical, chemical and/or biological break-up of the lengthy DNA into discrete fragments of shorter length. Particularly, “fragmented” means fragmentation of at least some of the genomic DNA, preferably the target DNA, into fragments shorter than 1,500 bp, 1,300 bp, 1,100 bp, 1,000 bp, 900 bp, 800 bp, 700 bp, 600 bp, 500 bp, 400 bp, 300 bp, 200 bp or 100 bp. “At least some” in this respect means at least 5%, 10%, 20%, 30%, 40%, 50% or 75%.

The term “cancer cell” as used herein refers to a cell that acquires a characteristic set of functional capabilities during their development, particularly one or more of the following: the ability to evade apoptosis, self-sufficiency in growth signals, insensitivity to anti-growth signals, tissue invasion/metastasis, significant growth potential, and/or sustained angiogenesis. The term is meant to encompass both pre-malignant and malignant cancer cells.

The term “a significant amount of methylated genomic DNA” as used herein refers to an amount of at least X molecules of the methylated target DNA per ml of the sample used, preferably per ml of blood, serum or plasma. X may be as low as 1 and is usually a value between and including 1 and 50, in particular at least 2, 3, 4, 5, 10, 15, 20, 25, 30 or 40. For determination whether there is such a significant amount, the methylated target DNA may be, but does not necessarily have to be quantified. The determination, if no quantification is performed, may also be made by comparison to a standard, for example a standard comprising genomic DNA and therein a certain amount of fully methylated DNA, e.g. the equivalence of X genomes, wherein X is as above. The term may also refer to an amount of at least Y % of methylated target DNA in the sample (wherein the sum of methylated and unmethylated target DNA is 100%), wherein Y may be as low as 0.05 and is usually a value between and including 0.05 and 5, preferably 0.05 and 1 and more preferably 0.05 and 0.5. For example, Y may be at least 0.05, 0.1, 0.2, 0.3, 0.5, 1.0, 1.5, 2.0, 2.5, 3.0, 4.0 or 5.0.

The term “tumor DNA” or “tumor DNA of a cancer cell” as used herein refers simply to DNA of a cancer cell. It is used only to distinguish DNA of a cancer cell more clearly from other DNA referred to herein. Thus, unless ambiguities are introduced, the term “DNA of a cancer cell” may be used instead.

The term “is indicative for” or “indicates” as used herein refers to an act of identifying or specifying the thing to be indicated. As will be understood by persons skilled in the art, such assessment normally may not be correct for 100% of the subjects, although it preferably is correct. The term, however, requires that a correct indication can be made for a statistically significant part of the subjects. Whether a part is statistically significant can be determined easily by the person skilled in the art using several well-known statistical evaluation tools, for example, determination of confidence intervals, determination of p values, Student's t-test, Mann-Whitney test, etc. Details are provided in Dowdy and Wearden, Statistics for Research, John Wiley & Sons, New York 1983. The preferred confidence intervals are at least 50%, at least 60%, at least 70%, at least 80%, at least 90%, at least 95%. The p values are preferably 0.05, 0.01, or 0.005.

The phrase “method for detecting the presence or absence” as used herein with regard to the cancer of the specification refers to a determination whether the subject has the cancer or not. As will be understood by persons skilled in the art, such assessment normally may not be correct for 100% of the subjects, although it preferably is correct. The term, however, requires that a correct indication can be made for a statistically significant part of the subjects. For a description of statistic significance and suitable confidence intervals and p values, see above.

The term “diagnosis” as used herein refers to a determination whether a subject does or does not have cancer. A diagnosis by methylation analysis of the target DNA as described herein may be supplemented with a further means as described herein to confirm the cancer detected with the methylation analysis. As will be understood by persons skilled in the art, the diagnosis normally may not be correct for 100% of the subjects, although it preferably is correct. The term, however, requires that a correct diagnosis can be made for a statistically significant part of the subjects. For a description of statistic significance and suitable confidence intervals and p values, see above.

The phrase “screening a population of subjects” as used herein with regard to the cancer of the specification refers to the use of the method of the first aspect with samples of a population of subjects. Preferably, the subjects have an increased risk for, are suspected of having, or have had the cancer. In particular, one or more of the risk factors recited herein can be attributed to the subjects of the population. In a specific embodiment, the same one or more risk factors can be attributed to all subjects of the population. For example, the population may consist of subjects characterized by heavy alcohol use and/or tobacco consumption. It is to be understood that the term “screening” refers to a diagnosis as described above for subjects of the population, and is preferably confirmed using a further means as described herein. As will be understood by persons skilled in the art, the screening result normally may not be correct for 100% of the subjects, although it preferably is correct. The term, however, requires that a correct screening result can be achieved for a statistically significant part of the subjects. For a description of statistic significance and suitable confidence intervals and p values, see above.

The term “monitoring” as used herein refers to the accompaniment of a diagnosed cancer during a treatment procedure or during a certain period of time, typically during at least 1 week, 2 weeks, 3 weeks, 4 weeks, 1 month, 2 months, 3 months, 4 months, 5 months, 6 months, 1 year, 2 years, 3 years, 5 years, 10 years, or any other period of time. The term “accompaniment” means that states of and, in particular, changes of these states of a cancer may be detected based on the amount of methylated target DNA, particular based on changes in the amount in any type of periodical time segment, determined e.g., daily or 1, 2, 3, 4, 5, 6, 7, 8, 9, 10, 11, 12, 13, 14 or 15 times per month (no more than one determination per day) over the course of the treatment, which may be up to 1, 2, 3, 4, 5, 6, 7, 8, 9, 10, 11, 12, 15 or 24 months. Amounts or changes in the amounts can also be determined at treatment specific events, e.g. before and/or after every treatment cycle or drug/therapy administration. A cycle is the time between one round of treatment until the start of the next round. Cancer treatment is usually not a single treatment, but a course of treatments. A course usually takes between 3 to 6 months, but can be more or less than that. During a course of treatment, there are usually between 4 to 8 cycles of treatment. Usually a cycle of treatment includes a treatment break to allow the body to recover. As will be understood by persons skilled in the art, the result of the monitoring normally may not be correct for 100% of the subjects, although it preferably is correct. The term, however, requires that a correct result of the monitoring can be achieved for a statistically significant part of the subjects. For a description of statistic significance and suitable confidence intervals and p values, see above.

“Substantially identical” means that an oligonucleotide does not need to be 100% identical to a reference sequence but can comprise mismatches and/or spacers as defined herein. It is preferred that a substantially identical oligonucleotide, if not 100% identical, comprises 1 to 3, i.e. 1, 2 or 3 mismatches and/or spacers, preferably one mismatch or spacer per oligonucleotide, such that the intended annealing does not fail due to the mismatches and/or spacers. To enable annealing despite mismatches and/or spacers, it is preferred that an oligonucleotide does not comprise more than 1 mismatch per 10 nucleotides (rounded up if the first decimal is 5 or higher, otherwise rounded down) of the oligonucleotide.

The mismatch or a spacer is preferably a mismatch with or a spacer covering an SNP in the genomic DNA of the subject. A mismatch with an SNP is preferably not complementary to any nucleotide at this position in the subject's species. The term “SNP” as used herein refers to the site of an SNP, i.e. a single nucleotide polymorphism, at a particular position in the (preferably human) genome that varies among a population of individuals. SNPs of the genomic DNA the present application refers to are known in the art and can be found in online databases such as db SNP of NCBI (http://www.ncbi.nlm.nih.gov/snp).

The term “spacer” as used herein refers to a non-nucleotide spacer molecule, which increases, when joining two nucleotides, the distance between the two nucleotides to about the distance of one nucleotide (i.e. the distance the two nucleotides would be apart if they were joined by a third nucleotide). Non-limiting examples for spacers are Inosine, d-Uracil, halogenated bases, Amino-dT, C3, C12, Spacer 9, Spacer 18, and dSpacer.

The term “oligonucleotide” as used herein refers to a linear oligomer of 5 to 50 ribonucleotides or preferably deoxyribonucleotides. Preferably, it has the structure of a single-stranded DNA fragment. The “stretch of contiguous nucleotides” referred to herein preferably is as long as the oligonucleotide.

The term “primer oligonucleotide” as used herein refers to a single-stranded oligonucleotide sequence comprising at its 3′ end a priming region which is substantially complementary to a nucleic acid sequence sought to be copied (the template) and serves as a starting point for synthesis of a primer extension product. Preferably, the priming region is 10 to 40 nucleotides, more preferably 15-30 nucleotides and most preferably 19 to 25 nucleotides in length. The “stretch of contiguous nucleotides” referred to herein preferably corresponds to the priming region. The primer oligonucleotide may further comprise, at the 5′ end of the primer oligonucleotide, an overhang region. The overhang region consists of a sequence which is not complementary to the original template, but which is in a subsequent amplification cycle incorporated into the template by extension of the opposite strand. The overhang region has a length that does not prevent priming by the priming region (e.g. annealing of the primer via the priming region to the template). For example, it may be 1-200 nucleotides, preferably 4-100 or 4-50, more preferably 4-25 or most preferably 4-15 nucleotides long. The overhang region usually comprises one or more functional domains, i.e. it has a sequence which encodes (not in the sense of translation into a polypeptide) a function which is or can be used for the method of the first aspect. Examples of functional domains are restriction sites, ligation sites, universal priming sites (e.g. for NGS), annealing sites (not for annealing to the template to be amplified by extension of the priming region, but to other oligonucleotides), and index (barcode) sites. The overhang region does not comprise a “stretch of contiguous nucleotides” as referred to herein with respect to the methylation markers of the invention. It is, as indicated above, understood by the skilled person that the sequence of an overhang region incorporated into a new double-strand generated by amplification. Therefore, the overhang region could be considered part of the priming region for further amplification of the new double-strand. However, the term “priming region” is used herein to distinguish a region that is the priming region of the initial template, i.e. which has a sequence that substantially corresponds to a methylation marker sequence of Table 3, from an overhang region with respect to the same methylation marker sequence.

It is also understood by the skilled person that the term “template” in the context of amplification of bisulfite converted DNA comprises not only double-stranded DNA, but also a single strand that is the result of bisulfite conversion of genomic DNA (rendering it non-complementary to its previous opposite strand). In the first round of amplification, only one of the primers of a primer pair binds to this single-strand and is extended, thereby creating a new complementary opposite strand to which the other primer of the primer pair can bind. Table 3 provides the sequences of the strands that are the result of bisulfite conversion of the genomic DNA of the methylation markers of the invention (bis1 and bis2), as well as corresponding new complementary opposite strands in 5′-3′ orientation (rc).

The term “primer pair” as used herein refers to two oligonucleotides, namely a forward and a reverse primer, that have, with respect to a double-stranded nucleic acid molecule (including a single strand that is the result of bisulfite conversion plus the new complementary opposite strand to be created as explained above), sequences that are (at least substantially) identical to one strand each such that they each anneal to the complementary strand of the strand they are (at least substantially) identical to. The term “forward primer” refers to the primer which is (at least substantially) identical to the forward strand (as defined by the direction of the genomic reference sequence) of the double-stranded nucleic acid molecule, and the term “reverse primer” refers to the primer which is (at least substantially) identical to the reverse complementary strand of the forward strand in the double-stranded nucleic acid molecule. The distance between the sites where forward and reverse primer anneal to their template depends on the length of the amplicon the primers are supposed to allow generating. Typically, with respect to the present invention it is between 40 and 1000 bp. Preferred amplicon sizes are specified herein. In case of single-stranded DNA template that is to be amplified using a pair of primers, only one of the primers anneals to the single strand in the first amplification cycle. The other primer then binds to the newly generated complementary strand such that the result of amplification is a double-stranded DNA fragment.

The term “blocker” as used herein refers to a molecule which binds in a methylation-specific manner to a single-strand of DNA (i.e. it is specific for either the converted methylated or preferably for the converted unmethylated DNA or the amplified DNA derived from it) and prevents amplification of the DNA by binding to it, for example by preventing a primer to bind or by preventing primer extension where it binds. Non-limiting examples for blockers are sequence and/or methylation specific antibodies (blocking e.g. primer binding or the polymerase) and in particular blocker oligonucleotides.

A “blocker oligonucleotide” may be a blocker that prevents the extension of the primer located upstream of the blocker oligonucleotide. It comprises nucleosides/nucleotides having a backbone resistant to the 5′ nuclease activity of the polymerase. This may be achieved, for example, by comprising peptide nucleic acid (PNA), locked nucleic acid (LNA), Morpholino, glycol nucleic acid (GNA), threose nucleic acid (TNA), bridged nucleic acids (BNA), N3′-P5′ phosphoramidate (NP) oligomers, minor groove binder-linked-oligonucleotides (MGB-linked oligonucleotides), phosphorothioate (PS) oligomers, CrC₄alkylphosphonate oligomers, phosphoramidates, β-phosphodiester oligonucleotides, a-phosphodiester oligonucleotides or a combination thereof. Alternatively, it may be a non-extendable oligonucleotide with a binding site on the DNA single-strand that overlaps with the binding site of a primer oligonucleotide. When the blocker is bound, the primer cannot bind and therefore the amplicon is not generated. When the blocker is not bound, the primer-binding site is accessible and the amplicon is generated. For such an overlapping blocker, it is preferable that the affinity of the blocker is higher than the affinity of the primer for the DNA. A blocker oligonucleotide is typically 15 to 50, preferably 20 to 40 and more preferably 25 to 35 nucleotides long. “At least one blocker” refers in particular to a number of 1, 2, 3, 4 or 5 blockers, more particularly to 1-2 or 1-3 blockers. Also, a blocker oligonucleotide cannot by itself act as a primer (i.e. cannot be extended by a polymerase) due to a non-extensible 3′ end.

The term “probe oligonucleotide” or “probe” as used herein refers to an oligonucleotide that is used to detect an amplicon by annealing to one strand of the amplicon, usually not where any of the primer oligonucleotides binds (i.e. not to a sequence segment of the one strand which overlaps with a sequence segment a primer oligonucleotide anneals to). Preferably it anneals without a mismatch or spacer, in other words it is preferably complementary to one strand of the amplicon. A probe oligonucleotide is preferably 5-40 nucleotides, more preferably 10 to 25 and most preferably 15 to 20 nucleotides long. The “stretch of contiguous nucleotides” referred to herein preferably is as long as the probe oligonucleotide. Usually, the probe is linked, preferably covalently linked, to at least one detectable label which allows detection of the amplicon and/or at least one quencher which allows quenching the signal of a (preferably the) detectable label. The term “detectable label” as used herein does not exhibit any particular limitation. The detectable label may be selected from the group consisting of radioactive labels, luminescent labels, fluorescent dyes, compounds having an enzymatic activity, magnetic labels, antigens, and compounds having a high binding affinity for a detectable label. For example, fluorescent dyes linked to a probe may serve as a detection label, e.g. in a real-time PCR. Suitable radioactive markers are P-32, S-35, I-125, and H-3, suitable luminescent markers are chemiluminescent compounds, preferably luminol, and suitable fluorescent markers are preferably dansyl chloride, fluorcein-5-isothiocyanate, and 4-fluor-7-nitrobenz-2-aza-1,3 diazole, in particular 6-Carboxyfluorescein (FAM), 6-Hexachlorofluorescein (HEX), 5(6)-Carboxytetramethylrhodamine (TAMRA), 5(6)-Carboxy-X-Rhodamine (ROX), Cyanin-5-Fluorophor (Cy5) and derivates thereof; suitable enzyme markers are horseradish peroxidase, alkaline phosphatase, a-galactosidase, acetylcholinesterase, or biotin. A probe may also be linked to a quencher. The term “quencher” as used herein refers to a molecule that deactivates or modulates the signal of a corresponding detectable label, e.g. by energy transfer, electron transfer, or by a chemical mechanism as defined by IUPAC (see compendium of chemical terminology 2^{nd} ed. 1997). In particular, the quencher modulates the light emission of a detectable label that is a fluorescent dye. In some cases, a quencher may itself be a fluorescent molecule that emits fluorescence at a characteristic wavelength distinct from the label whose fluorescence it is quenching. In other cases, the quencher does not itself fluoresce (i.e., the quencher is a “dark acceptor”). Such quenchers include, for example, dabcyl, methyl red, the QSY diarylrhodamine dyes, and the like.

The term “treatment” or “treating” with respect to cancer as used herein refers to a therapeutic treatment, wherein the goal is to reduce progression of cancer. Beneficial or desired clinical results include, but are not limited to, release of symptoms, reduction of the length of the disease, stabilized pathological state (specifically not deteriorated), slowing down of the disease's progression, improving the pathological state and/or remission (both partial and total), preferably detectable. A successful treatment does not necessarily mean cure, but it can also mean a prolonged survival, compared to the expected survival if the treatment is not applied. In a preferred embodiment, the treatment is a first line treatment, i.e. the cancer was not treated previously. Cancer treatment involves a treatment regimen.

The term “treatment regimen” as used herein refers to how the subject is treated in view of the disease and available procedures and medication. Non-limiting examples of cancer treatment regimens are chemotherapy, surgery and/or irradiation or combinations thereof. The early detection of cancer the present invention enables allows in particular for a surgical treatment, especially for a curative resection. In particular, the term “treatment regimen” refers to administering one or more anti-cancer agents or therapies as defined below. The term “anti-cancer agent or therapy” as used herein refers to chemical, physical or biological agents or therapies, or surgery, including combinations thereof, with antiproliferative, antioncogenic and/or carcinostatic properties.

A chemical anti-cancer agent or therapy may be selected from the group consisting of alkylating agents, antimetabolites, plant alkaloyds and terpenoids and topoisomerase inhibitors. Preferably, the alkylating agents are platinum-based compounds. In one embodiment, the platinum-based compounds are selected from the group consisting of cisplatin, oxaliplatin, eptaplatin, lobaplatin, nedaplatin, carboplatin, iproplatin, tetraplatin, lobaplatin, DCP, PLD-147, JM1 18, JM216, JM335, and satraplatin.

A physical anti-cancer agent or therapy may be selected from the group consisting of radiation therapy (e.g. curative radiotherapy, adjuvant radiotherapy, palliative radiotherapy, teleradiotherapy, brachytherapy or metabolic radiotherapy), phototherapy (using, e.g. hematoporphoryn or photofrin II), and hyperthermia.

Surgery may be a curative resection, palliative surgery, preventive surgery or cytoreductive surgery. Typically, it involves an excision, e.g. intracapsular excision, marginal, extensive excision or radical excision as described in Baron and Valin (Rec. Med. Vet, Special Canc. 1990; 11(166):999-1007).

A biological anti-cancer agent or therapy may be selected from the group consisting of antibodies (e.g. antibodies stimulating an immune response destroying cancer cells such as retuximab or alemtuzubab, antibodies stimulating an immune response by binding to receptors of immune cells an inhibiting signals that prevent the immune cell to attack “own” cells, such as ipilimumab, antibodies interfering with the action of proteins necessary for tumor growth such as bevacizumab, cetuximab or panitumumab, or antibodies conjugated to a drug, preferably a cell-killing substance like a toxin, chemotherapeutic or radioactive molecule, such as Y-ibritumomab tiuxetan, I-tositumomab or ado-trastuzumab emtansine), cytokines (e.g. interferons or interleukins such as INF-alpha and IL-2), vaccines (e.g. vaccines comprising cancer-associated antigens, such as sipuleucel-T), oncolytic viruses (e.g. naturally oncolytic viruses such as reovirus, Newcastle disease virus or mumps virus, or viruses genetically engineered viruses such as measles virus, adenovirus, vaccinia virus or herpes virus preferentially targeting cells carrying cancer-associated antigens), gene therapy agents (e.g. DNA or RNA replacing an altered tumor suppressor, blocking the expression of an oncogene, improving a subject's immune system, making cancer cells more sensitive to chemotherapy, radiotherapy or other treatments, inducing cellular suicide or conferring an anti-angiogenic effect) and adoptive T cells (e.g. subject-harvested tumor-invading T-cells selected for antitumor activity, or subject-harvested T-cells genetically modified to recognize a cancer-associated antigen).

In one embodiment, the one or more anti-cancer drugs is/are selected from the group consisting of Abiraterone Acetate, ABVD, ABVE, ABVE-PC, AC, AC-T, ADE, Ado-Trastuzumab Emtansine, Afatinib Dimaleate, Aldesleukin, Alemtuzumab, Aminolevulinic Acid, Anastrozole, Aprepitant, Arsenic Trioxide, Asparaginase Erwinia chrysanthemi, Axitinib, Azacitidine, BEACOPP, Belinostat, Bendamustine Hydrochloride, BEP, Bevacizumab, Bexarotene, Bicalutamide, Bleomycin, Bortezomib, Bosutinib, Brentuximab Vedotin, Busulfan, Cabazitaxel, Cabozantinib-S-Malate, CAFCapecitabine, CAPDX, Carboplatin, CARBOPLATIN-TAXOL, Carfilzomib, Carmustine, Carmustine Implant, Ceritinib, Cetuximab, Chlorambucil, CHLORAMBUCIL-PREDNISONE, CHOP, Cisplatin, Clofarabine, CMF, COPP, COPP-ABV, Crizotinib, CVP, Cyclophosphamide, Cytarabine, Cytarabine, Liposomal, Dabrafenib, Dacarbazine, Dactinomycin, Dasatinib, Daunorubicin Hydrochloride, Decitabine, Degarelix, Denileukin Diftitox, Denosumab, Dexrazoxane Hydrochloride, Docetaxel, Doxorubicin Hydrochloride, Doxorubicin Hydrochloride Liposome, Eltrombopag Olamine, Enzalutamide, Epirubicin Hydrochloride, EPOCH, Eribulin Mesylate, Erlotinib Hydrochloride, Etoposide Phosphate, Everolimus, Exemestane, FEC, Filgrastim, Fludarabine Phosphate, Fluorouracil, FU-LV, Fulvestrant, Gefitinib, Gemcitabine Hydrochloride, GEMCITABINE-CISPLATIN, GEMCITABINE-OXALIPLATIN, Gemtuzumab Ozogamicin, Glucarpidase, Goserelin Acetate, HPV Bivalent Vaccine, Recombinant HPV Quadrivalent Vaccine, Hyper-CVAD, Ibritumomab Tiuxetan, Ibrutinib, ICE, Idelalisib, Ifosfamide, Imatinib, Mesylate, Imiquimod, Iodine 131 Tositumomab and Tositumomab, Ipilimumab, Irinotecan Hydrochloride, Ixabepilone, Lapatinib Ditosylate, Lenalidomide, Letrozole, Leucovorin Calcium, Leuprolide Acetate, Liposomal Cytarabine, Lomustine, Mechlorethamine Hydrochloride, Megestrol Acetate, Mercaptopurine, Mesna, Methotrexate, Mitomycin C, Mitoxantrone Hydrochloride, MOPP, Nelarabine, Nilotinib, Obinutuzumab, Ofatumumab, Omacetaxine Mepesuccinate, OEPA, OFF, OPPA, Oxaliplatin, Paclitaxel, Paclitaxel Albumin-stabilized Nanoparticle Formulation, PAD, Palifermin, Palonosetron Hydrochloride, Pamidronate Di sodium, Panitumumab, Pazopanib Hydrochloride, Pegaspargase, Peginterferon Alfa-2b, Pembrolizumab, Pemetrexed Disodium, Pertuzumab, Plerixafor, Pomalidomide, Ponatinib Hydrochloride, Pralatrexate, Prednisone, Procarbazine Hydrochloride, Radium 223 Dichloride, Raloxifene Hydrochloride, Ramucirumab, Rasburicase, R-CHOP, R-CVP, Recombinant HPV Bivalent Vaccine, Recombinant HPV Quadrivalent Vaccine, Recombinant Interferon Alfa-2b, Regorafenib, Rituximab, Romidepsin, Romiplostim, Ruxolitinib Phosphate, Siltuximab, Sipuleucel-T, Sorafenib Tosylate, STANFORD V, Sunitinib Malate, TAC, Talc, Tamoxifen Citrate, Temozolomide, Temsirolimus, Thalidomide, Topotecan Hydrochloride, Toremifene, Tositumomab and I 131 Iodine Tositumomab, TPF, Trametinib, Trastuzumab, Vandetanib, VAMP, VeIP, Vemurafenib, Vinblastine Sulfate, Vincristine Sulfate, Vincristine Sulfate Liposome, Vinorelbine Tartrate, Vismodegib, Vorinostat, XELOX, Ziv-Aflibercept, and Zoledronic Acid.

### SEQ IDs Referred to in the Application

The present application refers to SEQ ID NOs 1-255. An overview and explanation of these SED IDs is given in the following Table 3.

TABLE 3
SEQ ID NOs of the specification, m as first letter of the gene name
means methylated, rc means reverse complement, C to T or G to A means
converted by bisulfite conversion of cytosines outside of CpG context
into uracil and replaced by thymidine in subsequent amplification,
bis1 refers to the bisulfite converted forward strand (as recited
in the SEQ ID of the respective genomic DNA) and bis2 to the bisulfite
converted reverse complement strand of the forward strand (reverse
complement of the SEQ ID of the respective genomic DNA), whereby
the direction of the strand is defined by the direction of the genomic
reference sequence as e.g. obtained from the genome build (GRCh38).
For a mapping of the sequences, see FIG. 1.
mSEPT9 Assay + CpG island 17: 77372606-77374424

| | |
|---|---|
| SEQ ID NO: 1 | genomic reference |
| SEQ ID NO: 2 | C to T (bis1) |
| SEQ ID NO: 3 | rc C to T (bis1) |
| SEQ ID NO: 4 | G to A (bis2 rc) |
| SEQ ID NO: 5 | G to A (bis2 rc) rc |

mSEPT9 Extended Assay 17: 77372979-77374040

| | |
|---|---|
| SEQ ID NO: 6 | genomic reference |
| SEQ ID NO: 7 | C to T (bis1) |
| SEQ ID NO: 8 | rc C to T (bis1) |
| SEQ ID NO: 9 | G to A (bis2 rc) |
| SEQ ID NO: 10 | G to A (bis2 rc) rc |

mSEPT9 Assay 17: 77373479-77373540

| | |
|---|---|
| SEQ ID NO: 11 | genomic reference |
| SEQ ID NO: 12 | C to T (bis1) |
| SEQ ID NO: 13 | rc C to T (bis1) |
| SEQ ID NO: 14 | G to A (bis2 rc) |
| SEQ ID NO: 15 | G to A (bis2 rc) rc |

mADCYAP1 Assay + CpG island 18: 906256-909573

| | |
|---|---|
| SEQ ID NO: 16 | genomic reference |
| SEQ ID NO: 17 | C to T (bis1) |
| SEQ ID NO: 18 | rc C to T (bis1) |
| SEQ ID NO: 19 | G to A (bis2 rc) |
| SEQ ID NO: 20 | G to A (bis2 rc) rc |

mADCYAP1 Extended Assay 18: 906345-907438

| | |
|---|---|
| SEQ ID NO: 21 | genomic reference |
| SEQ ID NO: 22 | C to T (bis1) |
| SEQ ID NO: 23 | rc C to T (bis1) |
| SEQ ID NO: 24 | G to A (bis2 rc) |
| SEQ ID NO: 25 | G to A (bis2 rc) rc |

mADCYAP1 Assay 18: 906845-906938

| | |
|---|---|
| SEQ ID NO: 26 | genomic reference |
| SEQ ID NO: 27 | C to T (bis1) |
| SEQ ID NO: 28 | rc C to T (bis1) |
| SEQ ID NO: 29 | G to A (bis2 rc) |
| SEQ ID NO: 30 | G to A (bis2 rc) rc |

mKHDRBS2 Extended Assay 6: 62285170-62286248

| | |
|---|---|
| SEQ ID NO: 31 | genomic reference |
| SEQ ID NO: 32 | C to T (bis1) |
| SEQ ID NO: 33 | rc C to T (bis1) |
| SEQ ID NO: 34 | G to A (bis2 rc) |
| SEQ ID NO: 35 | G to A (bis2 rc) rc |

mKHDRBS2 Assay 6: 62285670-62285748

| | |
|---|---|
| SEQ ID NO: 36 | genomic reference |
| SEQ ID NO: 37 | C to T (bis1) |
| SEQ ID NO: 38 | rc C to T (bis1) |
| SEQ ID NO: 39 | G to A (bis2 rc) |
| SEQ ID NO: 40 | G to A (bis2 rc) rc |

mCLEC14A Assay + CpG island 14: 38255049-38256332

| | |
|---|---|
| SEQ ID NO: 41 | genomic reference |
| SEQ ID NO: 42 | C to T (bis1) |
| SEQ ID NO: 43 | rc C to T (bis1) |
| SEQ ID NO: 44 | G to A (bis2 rc) |
| SEQ ID NO: 45 | G to A (bis2 rc) rc |

mCLEC14A Extended Assay 14: 38255401-38256502

| | |
|---|---|
| SEQ ID NO: 46 | genomic reference |
| SEQ ID NO: 47 | C to T (bis1) |
| SEQ ID NO: 48 | rc C to T (bis1) |
| SEQ ID NO: 49 | G to A (bis2 rc) |
| SEQ ID NO: 50 | G to A (bis2 rc) rc |

mCLEC14A Assay 14: 38255901-38256002

| | |
|---|---|
| SEQ ID NO: 51 | genomic reference |
| SEQ ID NO: 52 | C to T (bis1) |
| SEQ ID NO: 53 | rc C to T (bis1) |
| SEQ ID NO: 54 | G to A (bis2 rc) |
| SEQ ID NO: 55 | G to A (bis2 rc) rc |

mANKRD13B Assay + CpG island 17: 29612426-29613752

| | |
|---|---|
| SEQ ID NO: 56 | genomic reference |
| SEQ ID NO: 57 | C to T (bis1) |
| SEQ ID NO: 58 | rc C to T (bis1) |
| SEQ ID NO: 59 | G to A (bis2 rc) |
| SEQ ID NO: 60 | G to A (bis2 rc) rc |

mANKRD13B Extended Assay 17: 29613085-29614187

| | |
|---|---|
| SEQ ID NO: 61 | genomic reference |
| SEQ ID NO: 62 | C to T (bis1) |
| SEQ ID NO: 63 | rc C to T (bis1) |
| SEQ ID NO: 64 | G to A (bis2 rc) |
| SEQ ID NO: 65 | G to A (bis2 rc) rc |

mANKRD13B Assay 17: 29613585-29613687

| | |
|---|---|
| SEQ ID NO: 66 | genomic reference |
| SEQ ID NO: 67 | C to T (bis1) |
| SEQ ID NO: 68 | rc C to T (bis1) |
| SEQ ID NO: 69 | G to A (bis2 rc) |
| SEQ ID NO: 70 | G to A (bis2 rc) rc |

mCRMP1 Extended Assay 4: 5890481-5891551

| | |
|---|---|
| SEQ ID NO: 71 | genomic reference |
| SEQ ID NO: 72 | C to T (bis1) |
| SEQ ID NO: 73 | rc C to T (bis1) |
| SEQ ID NO: 74 | G to A (bis2 rc) |
| SEQ ID NO: 75 | G to A (bis2 rc) rc |

mCRMP1 Assay 4: 5890981-5891051

| | |
|---|---|
| SEQ ID NO: 76 | genomic reference |
| SEQ ID NO: 77 | C to T (bis1) |
| SEQ ID NO: 78 | rc C to T (bis1) |
| SEQ ID NO: 79 | G to A (bis2 rc) |
| SEQ ID NO: 80 | G to A (bis2 rc) rc |

mEYA4 Assay + CpG island 6: 133240948-133242448

| | |
|---|---|
| SEQ ID NO: 81 | genomic reference |
| SEQ ID NO: 82 | C to T (bis1) |
| SEQ ID NO: 83 | rc C to T (bis1) |
| SEQ ID NO: 84 | G to A (bis2 rc) |
| SEQ ID NO: 85 | G to A (bis2 rc) rc |

mEYA4 Extended Assay 6: 133241300-133242493

| | |
|---|---|
| SEQ ID NO: 86 | genomic reference |
| SEQ ID NO: 87 | C to T (bis1) |
| SEQ ID NO: 88 | rc C to T (bis1) |
| SEQ ID NO: 89 | G to A (bis2 rc) |
| SEQ ID NO: 90 | G to A (bis2 rc) rc |

mEYA4 Assay 6: 133241800-133241993

| | |
|---|---|
| SEQ ID NO: 91 | genomic reference |
| SEQ ID NO: 92 | C to T (bis1) |
| SEQ ID NO: 93 | rc C to T (bis1) |
| SEQ ID NO: 94 | G to A (bis2 rc) |
| SEQ ID NO: 95 | G to A (bis2 rc) rc |

mMSC Assay + CpG island 8: 71841639-71842520

| | |
|---|---|
| SEQ ID NO: 96 | genomic reference |
| SEQ ID NO: 97 | C to T (bis1) |
| SEQ ID NO: 98 | rc C to T (bis1) |
| SEQ ID NO: 99 | G to A (bis2 rc) |
| SEQ ID NO: 100 | G to A (bis2 rc) rc |

mMSC Extended Assay 8: 71841868-71842937

| | |
|---|---|
| SEQ ID NO: 101 | genomic reference |
| SEQ ID NO: 102 | C to T (bis1) |
| SEQ ID NO: 103 | rc C to T (bis1) |
| SEQ ID NO: 104 | G to A (bis2 rc) |
| SEQ ID NO: 105 | G to A (bis2 rc) rc |

mMSC Assay 8: 71842368-71842437

| | |
|---|---|
| SEQ ID NO: 106 | genomic reference |
| SEQ ID NO: 107 | C to T (bis1) |
| SEQ ID NO: 108 | rc C to T (bis1) |
| SEQ ID NO: 109 | G to A (bis2 rc) |
| SEQ ID NO: 110 | G to A (bis2 rc) rc |

mNGFR Assay + CpG island 17: 49494983-49497954

| | |
|---|---|
| SEQ ID NO: 111 | genomic reference |
| SEQ ID NO: 112 | C to T (bis1) |
| SEQ ID NO: 113 | rc C to T (bis1) |
| SEQ ID NO: 114 | G to A (bis2 rc) |
| SEQ ID NO: 115 | G to A (bis2 rc) rc |

mNGFR Extended Assay 17: 49497163-49498222

| | |
|---|---|
| SEQ ID NO: 116 | genomic reference |
| SEQ ID NO: 117 | C to T (bis1) |
| SEQ ID NO: 118 | rc C to T (bis1) |
| SEQ ID NO: 119 | G to A (bis2 rc) |
| SEQ ID NO: 120 | G to A (bis2 rc) rc |

mNGFR Assay 17: 49497663-49497722

| | |
|---|---|
| SEQ ID NO: 121 | genomic reference |
| SEQ ID NO: 122 | C to T (bis1) |
| SEQ ID NO: 123 | rc C to T (bis1) |
| SEQ ID NO: 124 | G to A (bis2 rc) |
| SEQ ID NO: 125 | G to A (bis2 rc) rc |

mNKX2 Assay + CpG island 20: 21510655-21513742

| | |
|---|---|
| SEQ ID NO: 126 | genomic reference |
| SEQ ID NO: 127 | C to T (bis1) |
| SEQ ID NO: 128 | rc C to T (bis1) |
| SEQ ID NO: 129 | G to A (bis2 rc) |
| SEQ ID NO: 130 | G to A (bis2 rc) rc |

mNKX2 Extended Assay 20: 21512255-21513321

| | |
|---|---|
| SEQ ID NO: 131 | genomic reference |
| SEQ ID NO: 132 | C to T (bis1) |
| SEQ ID NO: 133 | rc C to T (bis1) |
| SEQ ID NO: 134 | G to A (bis2 rc) |
| SEQ ID NO: 135 | G to A (bis2 rc) rc |

mNKX2 Assay 20: 21512755-21512821

| | |
|---|---|
| SEQ ID NO: 136 | genomic reference |
| SEQ ID NO: 137 | C to T (bis1) |
| SEQ ID NO: 138 | rc C to T (bis1) |
| SEQ ID NO: 139 | G to A (bis2 rc) |
| SEQ ID NO: 140 | G to A (bis2 rc) rc |

mRASSF2 Assay + CpG island 20: 4822367-4823486

| | |
|---|---|
| SEQ ID NO: 141 | genomic reference |
| SEQ ID NO: 142 | C to T (bis1) |
| SEQ ID NO: 143 | rc C to T (bis1) |
| SEQ ID NO: 144 | G to A (bis2 rc) |
| SEQ ID NO: 145 | G to A (bis2 rc) rc |

mRASSF2 Extended Assay 20: 4822086-4823162

| | |
|---|---|
| SEQ ID NO: 146 | genomic reference |
| SEQ ID NO: 147 | C to T (bis1) |
| SEQ ID NO: 148 | rc C to T (bis1) |
| SEQ ID NO: 149 | G to A (bis2 rc) |
| SEQ ID NO: 150 | G to A (bis2 rc) rc |

mRASSF2 Assay 20: 4822586-4822662

| | |
|---|---|
| SEQ ID NO: 151 | genomic reference |
| SEQ ID NO: 152 | C to T (bis1) |
| SEQ ID NO: 153 | rc C to T (bis1) |
| SEQ ID NO: 154 | G to A (bis2 rc) |
| SEQ ID NO: 155 | G to A (bis2 rc) rc |

mSND1 Assay + CpG island 7: 128104142-128104502

| | |
|---|---|
| SEQ ID NO: 156 | genomic reference |
| SEQ ID NO: 157 | C to T (bis1) |
| SEQ ID NO: 158 | rc C to T (bis1) |
| SEQ ID NO: 159 | G to A (bis2 rc) |
| SEQ ID NO: 160 | G to A (bis2 rc) rc |

mSND1 Extended Assay 7: 128103804-128104900

| | |
|---|---|
| SEQ ID NO: 161 | genomic reference |
| SEQ ID NO: 162 | C to T (bis1) |
| SEQ ID NO: 163 | rc C to T (bis1) |
| SEQ ID NO: 164 | G to A (bis2 rc) |
| SEQ ID NO: 165 | G to A (bis2 rc) rc |

mSND1 Assay 7: 128104304-128104400

| | |
|---|---|
| SEQ ID NO: 166 | genomic reference |
| SEQ ID NO: 167 | C to T (bis1) |
| SEQ ID NO: 168 | rc C to T (bis1) |
| SEQ ID NO: 169 | G to A (bis2 rc) |
| SEQ ID NO: 170 | G to A (bis2 rc) rc |

mTBX18 Assay + CpG island 6: 84762984-84764414

| | |
|---|---|
| SEQ ID NO: 171 | genomic reference |
| SEQ ID NO: 172 | C to T (bis1) |
| SEQ ID NO: 173 | rc C to T (bis1) |
| SEQ ID NO: 174 | G to A (bis2 rc) |
| SEQ ID NO: 175 | G to A (bis2 rc) rc |

mTBX18 Extended Assay 6: 84763288-84764374

| | |
|---|---|
| SEQ ID NO: 176 | genomic reference |
| SEQ ID NO: 177 | C to T (bis1) |
| SEQ ID NO: 178 | rc C to T (bis1) |
| SEQ ID NO: 179 | G to A(bis2 rc) |
| SEQ ID NO: 180 | G to A (bis2 rc) rc |

mTBX18 Assay 6: 84763788-84763874

| | |
|---|---|
| SEQ ID NO: 181 | genomic reference |
| SEQ ID NO: 182 | C to T (bis1) |
| SEQ ID NO: 183 | rc C to T (bis1) |
| SEQ ID NO: 184 | G to A (bis2 rc) |
| SEQ ID NO: 185 | G to A (bis2 rc) rc |

mTFAP2E Assay + CpG island 1: 35576831-35577843

| | |
|---|---|
| SEQ ID NO: 186 | genomic reference |
| SEQ ID NO: 187 | C to T (bis1) |
| SEQ ID NO: 188 | rc C to T (bis1) |
| SEQ ID NO: 189 | G to A (bis2 rc) |
| SEQ ID NO: 190 | G to A (bis2 rc) rc |

mTFAP2E Extended Assay 1: 35577250-35578318

| | |
|---|---|
| SEQ ID NO: 191 | genomic reference |
| SEQ ID NO: 192 | C to T (bis1) |
| SEQ ID NO: 193 | rc C to T (bis1) |
| SEQ ID NO: 194 | G to A (bis2 rc) |
| SEQ ID NO: 195 | G to A (bis2 rc) rc |

mTFAP2E Assay 1: 35577750-35577818

| | |
|---|---|
| SEQ ID NO: 196 | genomic reference |
| SEQ ID NO: 197 | C to T (bis1) |
| SEQ ID NO: 198 | rc C to T (bis1) |
| SEQ ID NO: 199 | Gto A (bis2 rc) |
| SEQ ID NO: 200 | G to A (bis2 rc) rc |

mTMEFF2 Assay + CpG island 2: 192194269-192196086

| | |
|---|---|
| SEQ ID NO: 201 | genomic reference |
| SEQ ID NO: 202 | C to T (bis1) |
| SEQ ID NO: 203 | rc C to T (bis1) |
| SEQ ID NO: 204 | G to A (bis2 rc) |
| SEQ ID NO: 205 | G to A (bis2 rc) rc |

mTMEFF2 Extended Assay 2: 192195336-192196409

| | |
|---|---|
| SEQ ID NO: 206 | genomic reference |
| SEQ ID NO: 207 | C to T (bis1) |
| SEQ ID NO: 208 | rc C to T (bis1) |
| SEQ ID NO: 209 | G to A (bis2 rc) |
| SEQ ID NO: 210 | G to A (bis2 rc) rc |

mTMEFF2 Assay 2: 192195836-192195909

| | |
|---|---|
| SEQ ID NO: 211 | genomic reference |
| SEQ ID NO: 212 | C to T (bis1) |
| SEQ ID NO: 213 | rc C to T (bis1) |
| SEQ ID NO: 214 | G to A (bis2 rc) |
| SEQ ID NO: 215 | G to A (bis2 rc) rc |

mVAX1 Extended Assay 10: 117131597-117132727

| | |
|---|---|
| SEQ ID NO: 216 | genomic reference |
| SEQ ID NO: 217 | C to T (bis1) |
| SEQ ID NO: 218 | rc C to T (bis1) |
| SEQ ID NO: 219 | G to A (bis2 rc) |
| SEQ ID NO: 220 | G to A (bis2 rc) rc |

mVAX1 Assay 10: 117132097-117132227

| | |
|---|---|
| SEQ ID NO: 221 | genomic reference |
| SEQ ID NO: 222 | C to T (bis1) |
| SEQ ID NO: 223 | rc C to T (bis1) |
| SEQ ID NO: 224 | G to A (bis2 rc) |
| SEQ ID NO: 225 | G to A (bis2 rc) rc |
| SEQ ID NO: 226 | mADCYAP1-F |
| SEQ ID NO: 227 | mKHDRBS2-F |
| SEQ ID NO: 228 | mCLEC14A-F |
| SEQ ID NO: 229 | mANKRD13B-F |
| SEQ ID NO: 230 | mCRMP1-F |
| SEQ ID NO: 231 | mEYA4-F |
| SEQ ID NO: 232 | mMSC-F |
| SEQ ID NO: 233 | mNGFR-F |
| SEQ ID NO: 234 | mNKX2-F |
| SEQ ID NO: 235 | mRASSF2-F |
| SEQ ID NO: 236 | mSND1-F |
| SEQ ID NO: 237 | mTBX18-F |
| SEQ ID NO: 238 | mTFAP2E-F |
| SEQ ID NO: 239 | mTMEFF2-F |
| SEQ ID NO: 240 | mVAX1-F |
| SEQ ID NO: 241 | mADCYAP1-R |
| SEQ ID NO: 242 | mKHDRBS2-R |
| SEQ ID NO: 243 | mCLEC14A-R |
| SEQ ID NO: 244 | mANKRD13B-R |
| SEQ ID NO: 245 | mCRMP1-R |
| SEQ ID NO: 246 | mEYA4-R |
| SEQ ID NO: 247 | mMSC-R |
| SEQ ID NO: 248 | mNGFR-R |
| SEQ ID NO: 249 | mNKX2-R |
| SEQ ID NO: 250 | mRASSF2-R |
| SEQ ID NO: 251 | mSND1-R |
| SEQ ID NO: 252 | mTBX18-R |
| SEQ ID NO: 253 | mTFAP2E-R |
| SEQ ID NO: 254 | mTMEFF2-R |
| SEQ ID NO: 255 | mVAX1-R |

The invention is described by way of the following examples which are to be construed as merely illustrative and not limitative of the scope of the invention.

### Example 1

### Material and Methods

Blood plasma samples from colorectal cancer (CRC) patients and healthy individuals (no evidence of disease, NED) were collected as defined in the instructions for use (IFU) of the Epi proColon 2.0 kit (Epigenomics AG). Briefly, for EDTA plasma was prepared by two centrifugation steps. Until processing plasma samples were stored at −70° C.

DNA extraction from plasma samples and bisulfite conversion of DNA was performed with the Plasma Quick kit according to the pre-analytic workflow as defined in the instructions for use (IFU) of the Epi proColon 2.0 kit (Epigenomics AG).

The PCR was set up with bisulfite DNA yield of an equivalent of about 1 ml plasma in a ready to use multiplex PCR kit (QIAGEN® Multiplex PCR) according to manufactures protocol. PCR oligos (sequences as shown in Table 3) were modified with a 5′ phosphate for NGS library preparation. The multiplex PCR profile used a protocol as follows: degeneration at 94° C. for 30 seconds, annealing at 56° C. for 90 seconds, extension step of 30 seconds at 72° C.; 45 cycles.

The PCR product was sequenced paired end with an Illumina MiSeq using a read length of 150 bp.

Fastq files were trimmed to insertions between sequencing adaptors, paired sequences were merged, and sequences filtered for those flanked by primers on both sides reflecting molecules amplified by PCR, called Inserts. Inserts that showed more cytosine that guanine outside of CpG context were turned to their reverse complement to enable assessment of methylation by taking cytosine positions of CpGs into account exclusively. Such inserts were aligned to reference sequences of the assays to assess DNA-methylation: For each assay/sample combination any methylation pattern at CpG sites was assessed by counting occurrence of cytosines and thymidines at CpG positions. Comethylation was calculated as number of insert sequences with cytosine in all CpG positions divided by total number of all inserts found for a sample, normalized by the length of the inserts.

Septin-9 methylation was determined using the Epi proColon 2.0 kit (Epigenomics AG) with the oligos of the kit.

### Results

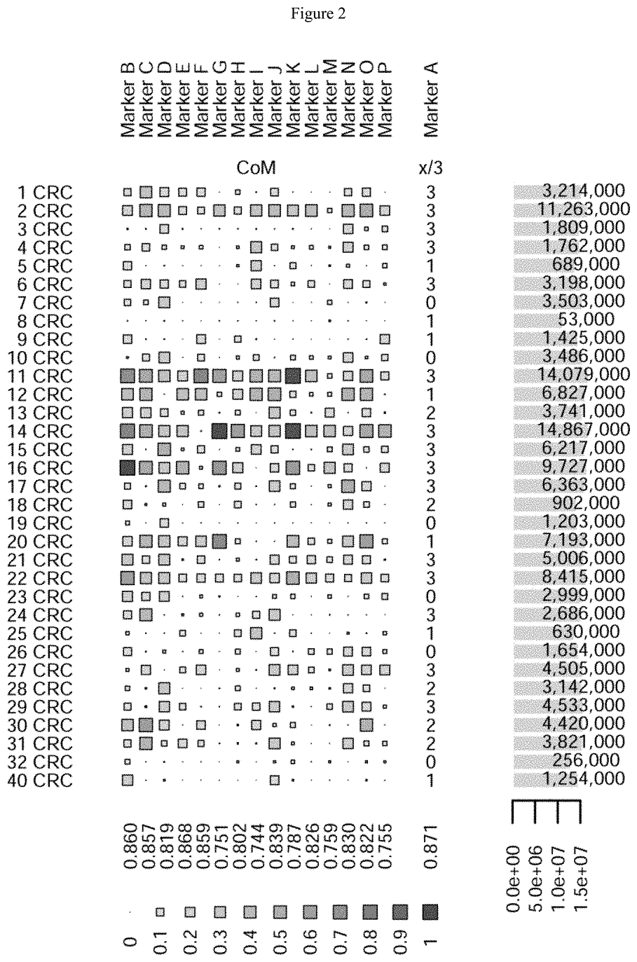

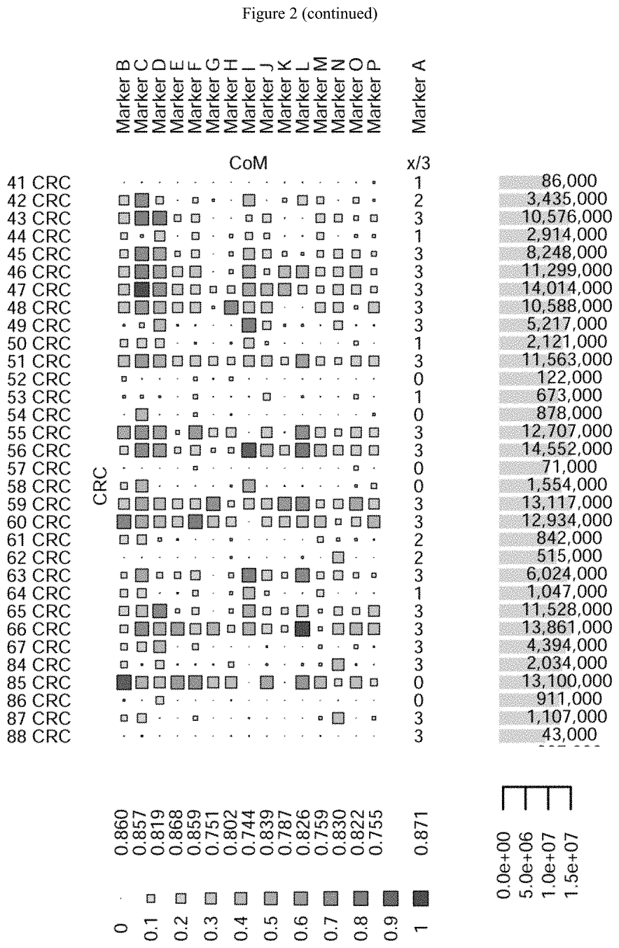

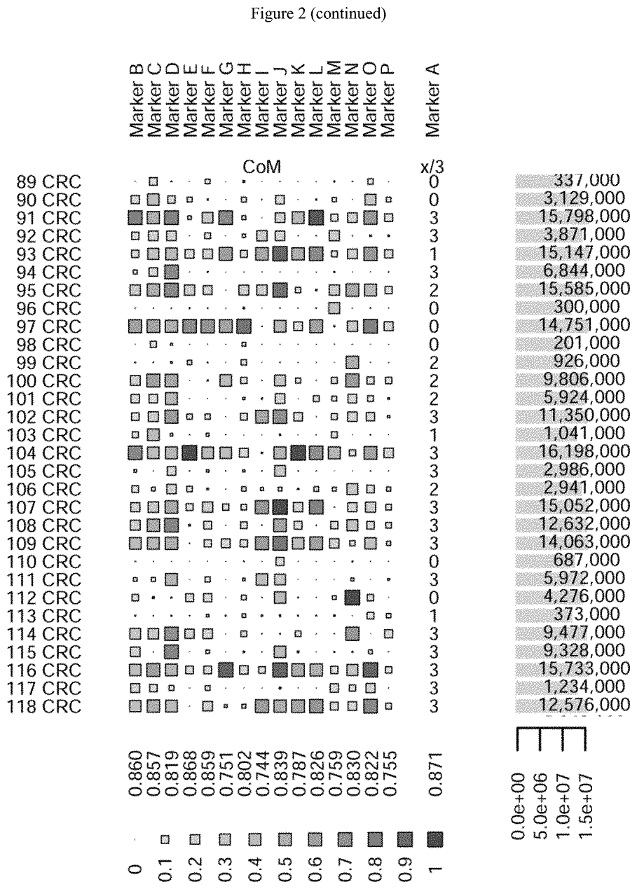

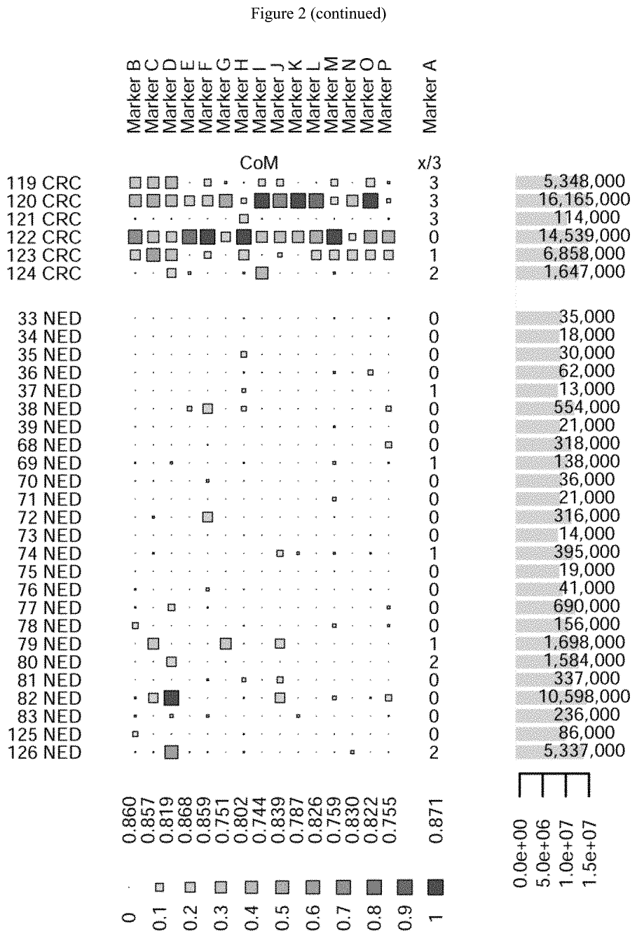

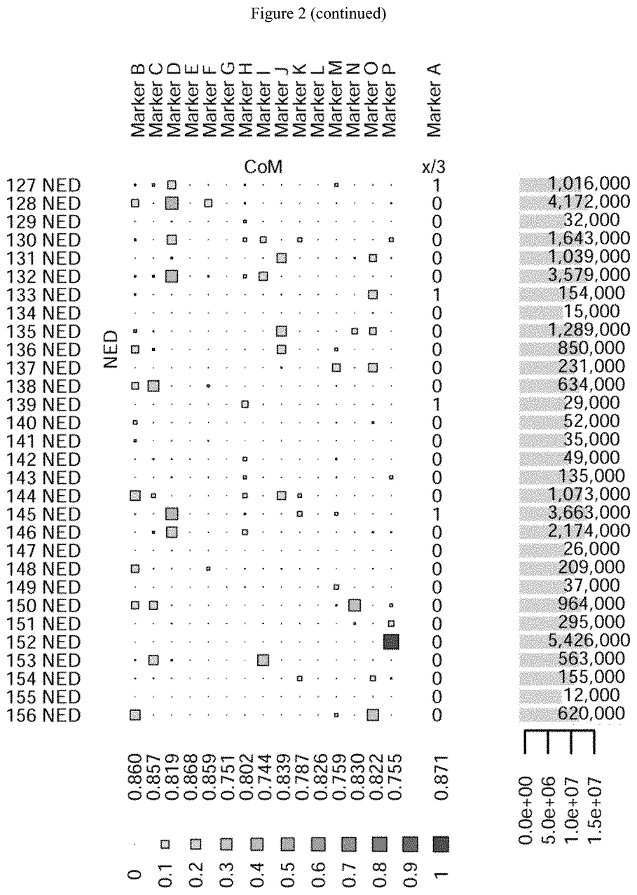

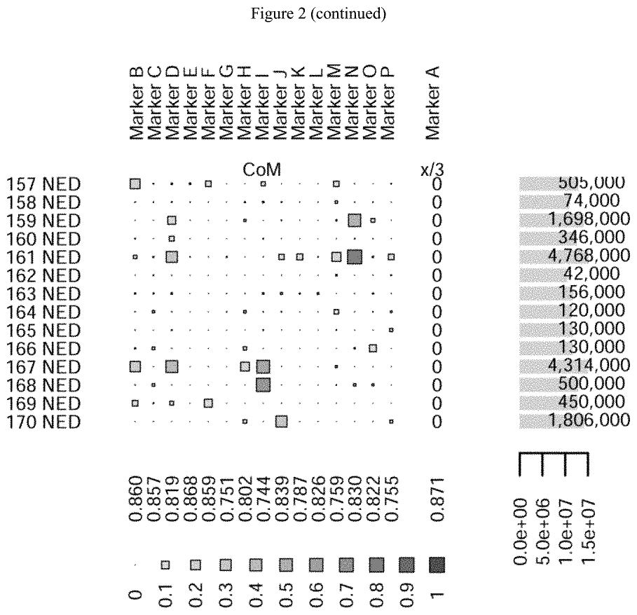

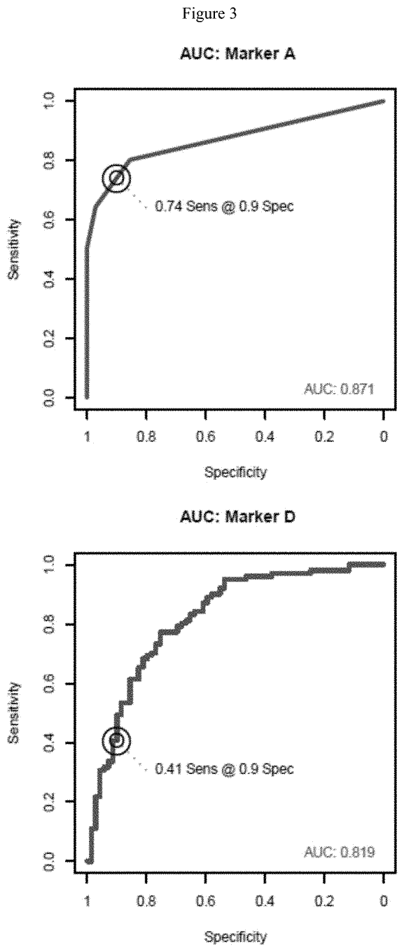

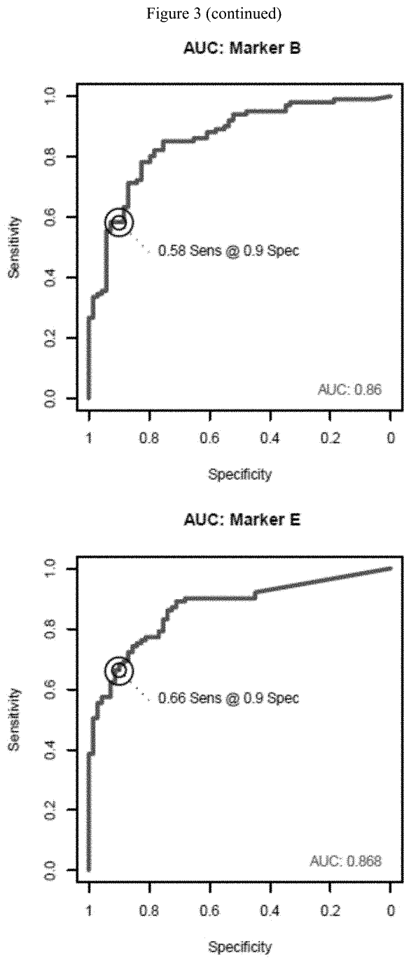

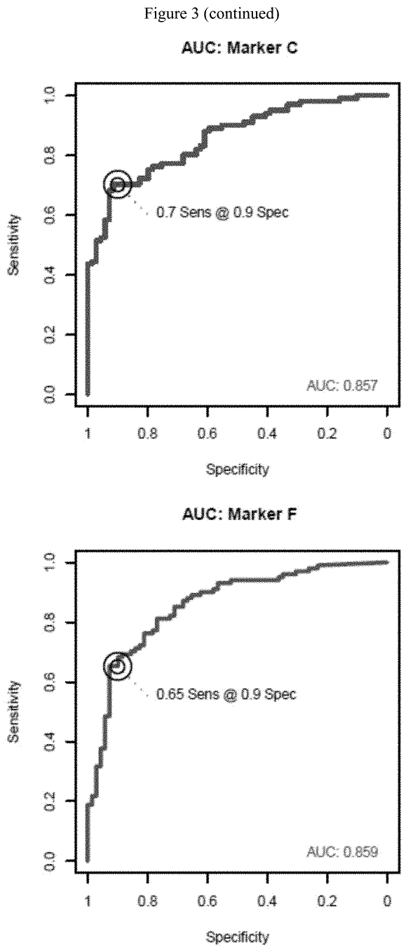

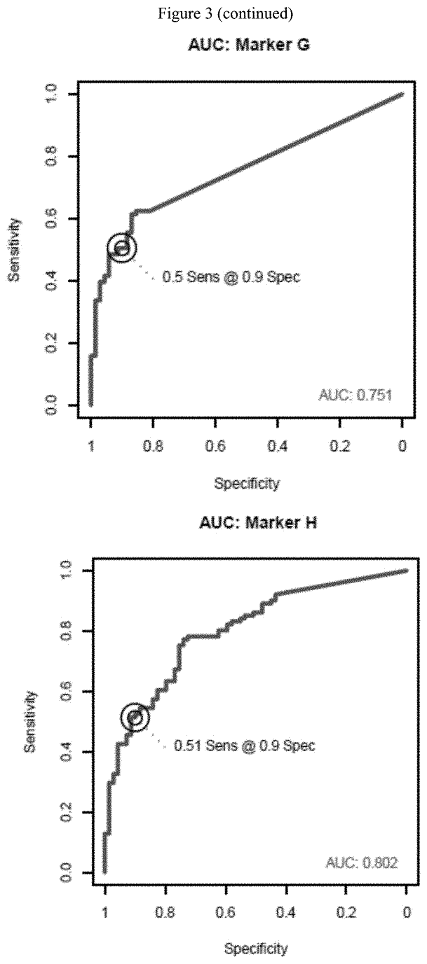

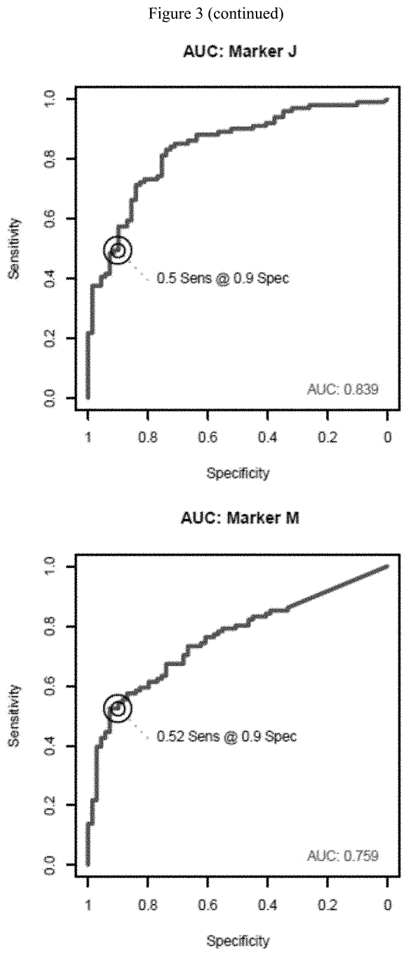

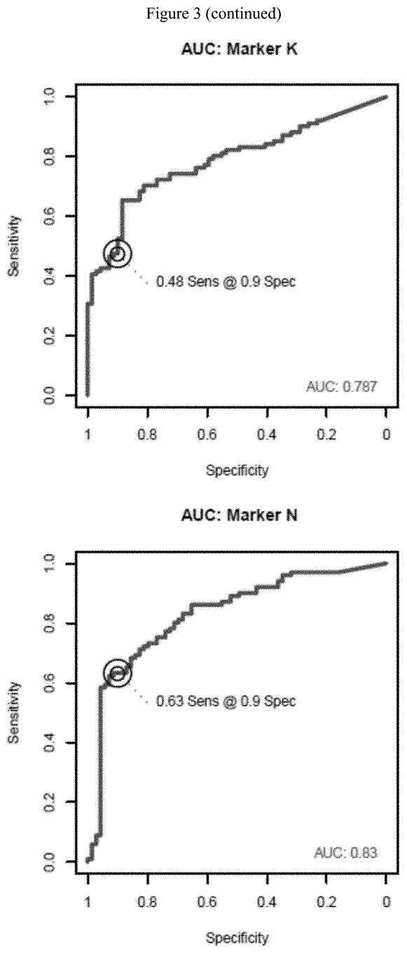

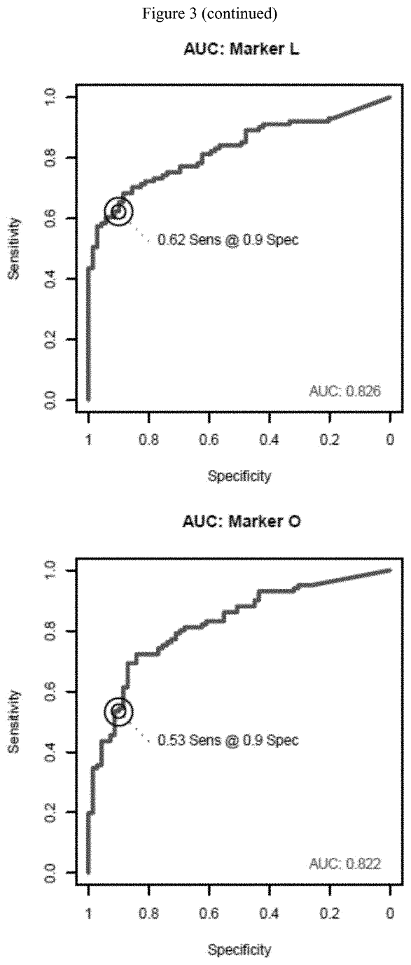

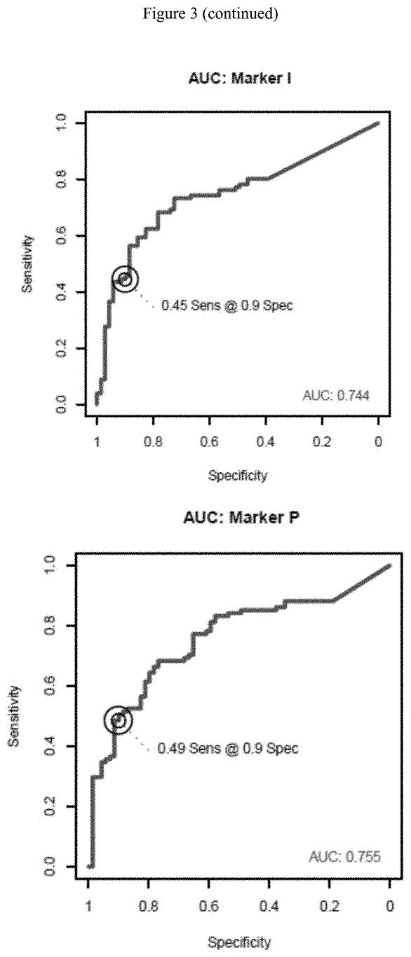

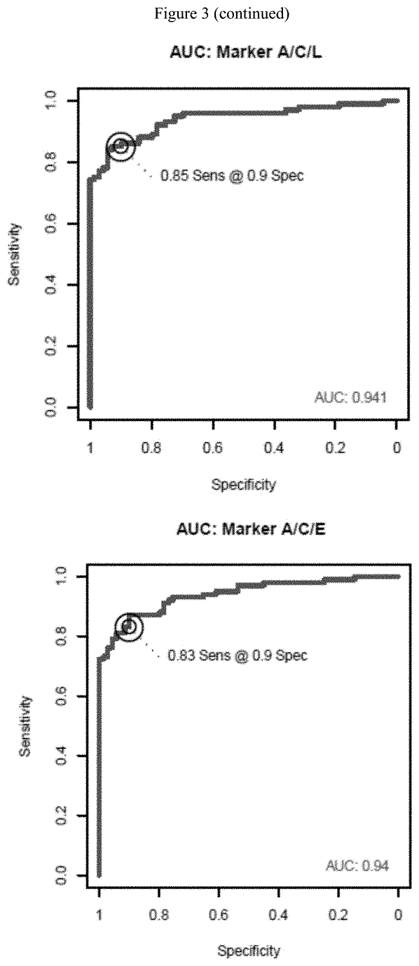

The univariate comparison of DNA-methylation levels found in blood plasma from CRC patients and healthy individuals (NED) for the set of preselected cancer-markers showed that cancer specific methylation patterns from free circulating tumor cell DNA (ctDNA) can be used to distinguish both groups (summarized in FIG. 2 and in Table 4). The performance as determined by areas under the curves (AUC) of responder operator characteristic (ROC) was higher than even 0.8 for most markers, with good sensitivities at specificity of 90% (FIG. 3). All markers (mADCYAP1, mKHDRBS2, mCLEC14A, mFOXL2, mHOXA9, mNKX2-2, mSND1, mTFAP2E, mSOX2 and mVAX1) had methylation patterns with high grade of comethylation (methylation state of all CpGs within the region assessed is identical in the same molecule), which enables using the amount of reads from molecules with all CpGs methylated to reflect the amount of ctDNA molecules in the template. Within the data set, combination of two or three markers using logistic regression is able to increase the performance above AUC of 0.90 (see FIG. 3 and Tables 1 and 2).

TABLE 4
Data from single marker performance on 105 CRC vs. 69 NED samples (Sample IDs by type and
number) for different types of data. “N.c.c.” stands for “N comethylated copies”
and means the number of reads found containing the exact sequence expected from completely
methylated molecule. “N.p.E.t.” stands for “N of positive Epi proColon triplicates”
and means number of real-time PCR with amplification curves out of three replicates of a mSept9
real-time PCR according to the instructions for use of the commercially available Epi proColon 2.0 kit.

| | mSEPT9 | mADCYAP1 | mKHDRBS2 | mCLEC14A | mANKRD13B | mCRMP1 | mEYA4 | mMSC |
|---|---|---|---|---|---|---|---|---|
| Sample | N.p.E.T. | N.c.c. | N.c.c. | N.c.c. | N.c.c. | N.c.c. | N.c.c. | N.c.c. |
| 1 CRC | 3 | 130210 | 640505 | 1335283 | 122685 | 170883 | 0 | 10397 |
| 2 CRC | 3 | 309946 | 814491 | 3598017 | 119915 | 105606 | 227647 | 27693 |
| 3 CRC | 3 | 7174 | 4946 | 1210290 | 3645 | 1335 | 1406 | 639 |
| 4 CRC | 3 | 69247 | 172288 | 261235 | 16912 | 28344 | 0 | 4520 |
| 5 CRC | 1 | 186096 | 887 | 20219 | 349 | 100 | 0 | 3307 |
| 6 CRC | 3 | 126569 | 323209 | 1081483 | 77582 | 328157 | 0 | 0 |
| 7 CRC | 0 | 97250 | 61714 | 2518514 | 0 | 892 | 0 | 312 |
| 8 CRC | 1 | 2771 | 1473 | 24370 | 815 | 146 | 1093 | 466 |
| 9 CRC | 1 | 206485 | 3655 | 22216 | 0 | 210623 | 0 | 20712 |
| 10 CRC | 0 | 11550 | 144263 | 2406230 | 511 | 102760 | 311 | 5752 |
| 11 CRC | 3 | 1090968 | 993945 | 3094813 | 244839 | 1068097 | 393359 | 73506 |
| 12 CRC | 1 | 562424 | 842295 | 8585 | 402386 | 494890 | 20387 | 69242 |
| 13 CRC | 2 | 352314 | 409620 | 857981 | 18614 | 56262 | 1964 | 12249 |
| 14 CRC | 3 | 1185216 | 918196 | 2761382 | 368450 | 3324 | 671038 | 205303 |
| 15 CRC | 3 | 338550 | 2756 | 4292543 | 14707 | 115983 | 402 | 7454 |
| 16 CRC | 3 | 1537637 | 970207 | 2479642 | 545115 | 21332 | 438436 | 78180 |
| 17 CRC | 3 | 89165 | 6606 | 3706334 | 55640 | 46458 | 3373 | 18694 |
| 18 CRC | 2 | 216514 | 14981 | 147064 | 313 | 69845 | 0 | 23432 |
| 19 CRC | 0 | 38476 | 590 | 1149786 | 642 | 1158 | 0 | 275 |
| 20 CRC | 1 | 271025 | 808951 | 2834722 | 186896 | 351911 | 488092 | 12747 |
| 21 CRC | 3 | 457524 | 166132 | 2495348 | 6203 | 130171 | 1245 | 5628 |
| 22 CRC | 3 | 919568 | 587758 | 2653826 | 117920 | 169652 | 71664 | 33943 |
| 23 CRC | 0 | 354741 | 230763 | 1852651 | 953 | 20081 | 2813 | 0 |
| 24 CRC | 3 | 176458 | 777981 | 13553 | 10602 | 23899 | 1239 | 6405 |
| 25 CRC | 1 | 27799 | 2704 | 11436 | 57489 | 733 | 364 | 23595 |
| 26 CRC | 0 | 174883 | 4836 | 147216 | 1231 | 34026 | 341 | 12434 |
| 27 CRC | 3 | 24699 | 315873 | 3285 | 60257 | 262836 | 0 | 5100 |
| 28 CRC | 2 | 46290 | 10998 | 2669810 | 0 | 0 | 1565 | 11527 |
| 29 CRC | 3 | 116121 | 14148 | 2129732 | 74674 | 3044 | 987 | 23582 |
| 30 CRC | 2 | 536776 | 1144609 | 1807690 | 837 | 189783 | 0 | 1909 |
| 31 CRC | 2 | 130514 | 832507 | 420883 | 157850 | 81606 | 1262 | 1661 |
| 32 CRC | 0 | 54038 | 1425 | 26730 | 392 | 1175 | 0 | 2935 |
| 33 NED | 0 | 493 | 1485 | 1692 | 0 | 0 | 421 | 835 |
| 34 NED | 0 | 806 | 1428 | 5172 | 396 | 177 | 0 | 0 |
| 35 NED | 0 | 1434 | 585 | 2632 | 640 | 0 | 0 | 16616 |
| 36 NED | 0 | 1657 | 1452 | 2053 | 0 | 175 | 0 | 1461 |
| 37 NED | 1 | 744 | 706 | 243 | 0 | 0 | 0 | 7503 |
| 38 NED | 0 | 1476 | 1545 | 8045 | 37691 | 266418 | 0 | 11814 |
| 39 NED | 0 | 2597 | 2095 | 3169 | 0 | 197 | 0 | 0 |
| 40 CRC | 1 | 369860 | 1587 | 63665 | 125 | 589 | 302 | 0 |
| 41 CRC | 1 | 1387 | 4615 | 11826 | 1042 | 1068 | 580 | 105 |
| 42 CRC | 2 | 297746 | 1314914 | 841976 | 1600 | 61093 | 6699 | 137 |
| 43 CRC | 3 | 432726 | 1463009 | 6950635 | 82897 | 162953 | 335 | 285 |
| 44 CRC | 1 | 108916 | 26890 | 2143725 | 1618 | 108247 | 548 | 6768 |
| 45 CRC | 3 | 185396 | 1354379 | 4779047 | 50269 | 148691 | 0 | 9431 |
| 46 CRC | 3 | 417873 | 1394869 | 5059301 | 260161 | 478819 | 602 | 9474 |
| 47 CRC | 3 | 468177 | 1837545 | 5901388 | 311829 | 258146 | 42272 | 33308 |
| 48 CRC | 3 | 466336 | 1191165 | 3924883 | 281106 | 383830 | 7931 | 244785 |
| 49 CRC | 3 | 12275 | 66437 | 3561289 | 7858 | 5755 | 251 | 629 |
| 50 CRC | 1 | 149872 | 343921 | 1333434 | 729 | 11168 | 393 | 2535 |
| 51 CRC | 3 | 527139 | 1162192 | 4219362 | 235458 | 370523 | 108803 | 33793 |
| 52 CRC | 0 | 45322 | 4048 | 12836 | 333 | 30382 | 2658 | 7682 |
| 53 CRC | 1 | 21285 | 28890 | 66618 | 638 | 41924 | 1091 | 730 |
| 54 CRC | 0 | 1414 | 760821 | 12549 | 1023 | 33140 | 361 | 2019 |
| 55 CRC | 3 | 820197 | 1255749 | 5008842 | 27290 | 891700 | 92819 | 66184 |
| 56 CRC | 3 | 191816 | 1345219 | 5282742 | 69456 | 287729 | 18951 | 15954 |
| 57 CRC | 0 | 3601 | 1016 | 1951 | 91 | 29853 | 0 | 0 |
| 58 CRC | 0 | 116677 | 808589 | 12161 | 2079 | 2338 | 864 | 2792 |
| 59 CRC | 3 | 447285 | 1015944 | 3389294 | 255333 | 359968 | 474024 | 21091 |
| 60 CRC | 3 | 1184851 | 968223 | 3504147 | 318529 | 1137256 | 235291 | 67102 |
| 61 CRC | 2 | 200757 | 338845 | 181443 | 8392 | 920 | 315 | 3657 |
| 62 CRC | 2 | 1346 | 3050 | 6460 | 747 | 211 | 0 | 2948 |
| 63 CRC | 3 | 85286 | 996085 | 474068 | 77324 | 245175 | 0 | 12311 |
| 64 CRC | 1 | 150323 | 293977 | 15591 | 6801 | 68736 | 0 | 5519 |
| 65 CRC | 3 | 279620 | 659170 | 6661400 | 30171 | 109653 | 0 | 9509 |
| 66 CRC | 3 | 162827 | 1327043 | 4269119 | 684212 | 395520 | 315326 | 28486 |
| 67 CRC | 3 | 105594 | 336452 | 3768395 | 310 | 81494 | 328 | 178 |
| 68 NED | 0 | 238 | 1645 | 5093 | 178 | 3468 | 0 | 0 |
| 69 NED | 1 | 6781 | 2115 | 87595 | 0 | 576 | 0 | 1488 |
| 70 NED | 0 | 2574 | 3627 | 3728 | 345 | 18596 | 219 | 592 |
| 71 NED | 0 | 129 | 962 | 1002 | 0 | 787 | 0 | 0 |
| 72 NED | 0 | 1074 | 16353 | 2566 | 0 | 290556 | 0 | 0 |
| 73 NED | 0 | 732 | 1164 | 3275 | 268 | 1557 | 0 | 633 |
| 74 NED | 1 | 568 | 12027 | 5834 | 283 | 162 | 0 | 0 |
| 75 NED | 0 | 2641 | 617 | 5458 | 676 | 0 | 460 | 0 |
| 76 NED | 0 | 8581 | 566 | 1481 | 834 | 21867 | 0 | 671 |
| 77 NED | 0 | 8110 | 120 | 595940 | 0 | 6382 | 0 | 0 |
| 78 NED | 0 | 81787 | 736 | 3001 | 0 | 0 | 0 | 935 |
| 79 NED | 1 | 0 | 537851 | 1733 | 543 | 1111 | 202139 | 583 |
| 80 NED | 2 | 1121 | 1726 | 1575687 | 0 | 0 | 1033 | 0 |
| 81 NED | 0 | 0 | 1404 | 1413 | 0 | 10503 | 0 | 7899 |
| 82 NED | 0 | 11275 | 333393 | 8798637 | 254 | 298 | 0 | 1415 |
| 83 NED | 0 | 12412 | 576 | 155520 | 0 | 16353 | 0 | 0 |
| 84 CRC | 3 | 105281 | 12302 | 1175410 | 4998 | 6598 | 2940 | 15141 |
| 85 CRC | 0 | 1460109 | 839565 | 2949397 | 726260 | 965455 | 223028 | 126940 |
| 86 CRC | 0 | 11895 | 2372 | 889095 | 894 | 688 | 0 | 1151 |
| 87 CRC | 3 | 90111 | 290212 | 15109 | 281 | 40890 | 0 | 0 |
| 88 CRC | 3 | 2882 | 12380 | 11164 | 701 | 1094 | 0 | 1002 |
| 89 CRC | 0 | 630 | 202677 | 25610 | 0 | 50858 | 0 | 2723 |
| 90 CRC | 0 | 197200 | 666751 | 865792 | 44685 | 5745 | 0 | 13105 |
| 91 CRC | 3 | 1214069 | 900804 | 6851374 | 24834 | 399533 | 495101 | 8397 |
| 92 CRC | 3 | 141430 | 381240 | 1758146 | 319 | 74142 | 0 | 6464 |
| 93 CRC | 1 | 148405 | 592114 | 3577316 | 179098 | 270485 | 410810 | 27100 |
| 94 CRC | 3 | 40563 | 265056 | 6517692 | 0 | 4633 | 0 | 0 |
| 95 CRC | 2 | 413834 | 952447 | 6552500 | 300508 | 228603 | 0 | 79263 |
| 96 CRC | 0 | 2202 | 1881 | 14126 | 3642 | 2658 | 313 | 2251 |
| 97 CRC | 0 | 923857 | 1074415 | 4246015 | 828968 | 957811 | 385426 | 288743 |
| 98 CRC | 0 | 0 | 111243 | 76210 | 225 | 505 | 0 | 8229 |
| 99 CRC | 2 | 4741 | 3396 | 65869 | 36373 | 3543 | 0 | 15450 |
| 100 CRC | 2 | 330514 | 1119397 | 4810619 | 0 | 5482 | 255776 | 21200 |
| 101 CRC | 2 | 217016 | 256470 | 3796054 | 358 | 178 | 0 | 3596 |
| 102 CRC | 3 | 158907 | 413250 | 5441276 | 53398 | 59780 | 0 | 27411 |
| 103 CRC | 1 | 88408 | 724423 | 125007 | 1494 | 22312 | 584 | 0 |
| 104 CRC | 3 | 1166346 | 693014 | 3805765 | 1151714 | 555155 | 248085 | 53609 |
| 105 CRC | 3 | 21009 | 1398 | 1285936 | 1596 | 16839 | 776 | 3425 |
| 106 CRC | 2 | 117168 | 42755 | 1707311 | 55404 | 10498 | 569 | 10588 |
| 107 CRC | 3 | 257542 | 518594 | 4725293 | 93399 | 365657 | 45487 | 16966 |
| 108 CRC | 3 | 348437 | 949268 | 6744598 | 8469 | 148910 | 493 | 25403 |
| 109 CRC | 3 | 598882 | 782401 | 4428971 | 1428 | 126208 | 103710 | 30885 |
| 110 CRC | 0 | 2873 | 1022 | 1911 | 0 | 174 | 0 | 802 |
| 111 CRC | 3 | 33562 | 47116 | 3845760 | 0 | 61990 | 0 | 4524 |
| 112 CRC | 0 | 105967 | 14978 | 33072 | 134702 | 185681 | 0 | 12671 |
| 113 CRC | 1 | 5738 | 3006 | 31334 | 2170 | 19078 | 2379 | 7452 |
| 114 CRC | 3 | 456222 | 496229 | 6216284 | 215464 | 279893 | 0 | 17698 |
| 115 CRC | 3 | 304598 | 1476 | 6738505 | 370 | 29864 | 0 | 2288 |
| 116 CRC | 3 | 562215 | 1121488 | 3846111 | 119946 | 151298 | 621424 | 47350 |
| 117 CRC | 3 | 247118 | 184525 | 340440 | 698 | 52473 | 0 | 0 |
| 118 CRC | 3 | 550968 | 925975 | 3531247 | 276 | 293353 | 10941 | 9017 |
| 119 CRC | 3 | 433734 | 601777 | 3505886 | 660 | 107614 | 7188 | 810 |
| 120 CRC | 3 | 628943 | 919026 | 3115231 | 231072 | 354870 | 361170 | 13980 |
| 121 CRC | 3 | 5988 | 3114 | 21379 | 423 | 1209 | 0 | 45214 |
| 122 CRC | 0 | 1088602 | 632005 | 2182382 | 975053 | 1398265 | 126820 | 337622 |
| 123 CRC | 1 | 333778 | 1084817 | 3079982 | 792 | 103554 | 0 | 75676 |
| 124 CRC | 2 | 3152 | 475 | 1336429 | 14867 | 818 | 0 | 2586 |
| 125 NED | 0 | 62110 | 775 | 10728 | 138 | 1266 | 0 | 1233 |
| 126 NED | 2 | 5174 | 5988 | 5274078 | 3239 | 4330 | 0 | 1876 |
| 127 NED | 1 | 9782 | 18545 | 958748 | 942 | 1916 | 0 | 1618 |
| 128 NED | 0 | 126870 | 2588 | 3903531 | 152 | 115193 | 0 | 1562 |
| 129 NED | 0 | 1547 | 0 | 21344 | 142 | 517 | 0 | 4314 |
| 130 NED | 0 | 9876 | 930 | 1402281 | 0 | 0 | 0 | 6904 |
| 131 NED | 0 | 1384 | 1714 | 60458 | 172 | 157 | 335 | 0 |
| 132 NED | 0 | 10060 | 14671 | 3452848 | 531 | 7107 | 0 | 5468 |
| 133 NED | 1 | 9209 | 1370 | 9367 | 0 | 563 | 0 | 189 |
| 134 NED | 0 | 143 | 390 | 9473 | 132 | 0 | 0 | 0 |
| 135 NED | 0 | 21535 | 6294 | 5892 | 0 | 169 | 0 | 0 |
| 136 NED | 0 | 127381 | 14901 | 2264 | 0 | 0 | 0 | 463 |
| 137 NED | 0 | 201 | 604 | 6695 | 0 | 1130 | 0 | 0 |
| 138 NED | 0 | 106892 | 503060 | 7348 | 165 | 13185 | 0 | 0 |
| 139 NED | 1 | 216 | 1465 | 1454 | 0 | 765 | 0 | 20164 |
| 140 NED | 0 | 31627 | 2313 | 7580 | 160 | 0 | 0 | 0 |
| 141 NED | 0 | 14560 | 270 | 12681 | 0 | 3633 | 0 | 0 |
| 142 NED | 0 | 1746 | 8144 | 21571 | 1383 | 498 | 0 | 7119 |
| 143 NED | 0 | 1201 | 1812 | 26654 | 718 | 1450 | 0 | 5392 |
| 144 NED | 0 | 299736 | 42233 | 2331 | 129 | 175 | 0 | 9685 |
| 145 NED | 1 | 4124 | 703 | 3558582 | 0 | 310 | 0 | 2072 |
| 146 NED | 0 | 1538 | 17095 | 2104543 | 0 | 800 | 0 | 11913 |
| 147 NED | 0 | 784 | 1848 | 14921 | 659 | 867 | 0 | 0 |
| 148 NED | 0 | 145071 | 4400 | 11829 | 151 | 22042 | 0 | 693 |
| 149 NED | 0 | 847 | 951 | 4746 | 152 | 0 | 554 | 781 |
| 150 NED | 0 | 129073 | 204439 | 13299 | 0 | 395 | 0 | 0 |
| 151 NED | 0 | 996 | 585 | 20928 | 0 | 212 | 265 | 0 |
| 152 NED | 0 | 0 | 790 | 4919 | 0 | 231 | 0 | 0 |
| 153 NED | 0 | 4846 | 318912 | 42160 | 795 | 0 | 0 | 0 |
| 154 NED | 0 | 3473 | 5025 | 10222 | 0 | 259 | 0 | 0 |
| 155 NED | 0 | 0 | 328 | 5144 | 0 | 0 | 0 | 0 |
| 156 NED | 0 | 308439 | 1394 | 1338 | 158 | 0 | 0 | 407 |
| 157 NED | 0 | 310530 | 4429 | 47301 | 7177 | 67211 | 0 | 0 |
| 158 NED | 0 | 2880 | 3272 | 24805 | 1747 | 910 | 215 | 1179 |
| 159 NED | 0 | 575 | 312 | 933638 | 511 | 0 | 0 | 3145 |
| 160 NED | 0 | 3880 | 606 | 330504 | 173 | 453 | 0 | 149 |
| 161 NED | 0 | 30345 | 7715 | 2717696 | 2043 | 934 | 2350 | 0 |
| 162 NED | 0 | 631 | 4540 | 9226 | 0 | 273 | 0 | 0 |
| 163 NED | 0 | 6978 | 6570 | 52830 | 311 | 696 | 551 | 267 |
| 164 NED | 0 | 1936 | 20662 | 12581 | 166 | 0 | 1188 | 4064 |
| 165 NED | 0 | 1972 | 247 | 27258 | 132 | 255 | 0 | 0 |
| 166 NED | 0 | 6536 | 25566 | 8550 | 0 | 139 | 0 | 6159 |
| 167 NED | 0 | 371707 | 741 | 3516126 | 138 | 864 | 0 | 50167 |
| 168 NED | 0 | 2089 | 24171 | 6465 | 0 | 283 | 201 | 0 |
| 169 NED | 0 | 74322 | 5761 | 207348 | 0 | 157775 | 0 | 0 |
| 170 NED | 0 | 238 | 1113 | 2898 | 0 | 128 | 0 | 7157 |

| | mNGFR | mNKX2 | mRASSF2 | mSND1 | mTBX18 | mTFAP2E | mTMEFF2 | mVAX1 |
|---|---|---|---|---|---|---|---|---|
| Sample | N.c.c. | N.c.c. | N.c.c. | N.c.c. | N.c.c. | N.c.c. | N.c.c. | N.c.c. |
| 1 CRC | 1411 | 556447 | 1845 | 0 | 0 | 134790 | 109688 | 0 |
| 2 CRC | 218375 | 2169392 | 672944 | 858394 | 22736 | 652274 | 464643 | 1000614 |
| 3 CRC | 1872 | 7703 | 7007 | 1578 | 0 | 279033 | 19150 | 263153 |
| 4 CRC | 203449 | 365490 | 33296 | 51803 | 7197 | 334701 | 28904 | 185091 |
| 5 CRC | 149203 | 3926 | 132364 | 1421 | 4724 | 24950 | 611 | 161014 |
| 6 CRC | 126528 | 585470 | 45950 | 151168 | 0 | 226391 | 67926 | 57937 |
| 7 CRC | 872 | 797164 | 121 | 399 | 22130 | 397 | 3705 | 0 |
| 8 CRC | 554 | 11553 | 603 | 794 | 5070 | 1989 | 357 | 760 |
| 9 CRC | 2174 | 1609 | 1184 | 0 | 0 | 1883 | 1426 | 953482 |
| 10 CRC | 32198 | 9720 | 81964 | 50366 | 10655 | 281168 | 11412 | 337090 |
| 11 CRC | 305426 | 2359409 | 2277480 | 799358 | 24455 | 272007 | 482432 | 599396 |
| 12 CRC | 296364 | 2773542 | 142645 | 46409 | 20246 | 717139 | 417195 | 13340 |
| 13 CRC | 0 | 1661786 | 100360 | 5784 | 120529 | 1761 | 114500 | 27688 |
| 14 CRC | 184024 | 1839900 | 2267830 | 838657 | 282727 | 247849 | 506915 | 2585797 |
| 15 CRC | 129115 | 472033 | 3767 | 3485 | 28876 | 412501 | 39211 | 355260 |
| 16 CRC | 0 | 756196 | 1460597 | 146471 | 249074 | 178824 | 920 | 864524 |
| 17 CRC | 1426 | 1416433 | 91518 | 1239 | 12785 | 761261 | 151573 | 0 |
| 18 CRC | 1020 | 7080 | 97521 | 1066 | 12873 | 263270 | 44083 | 2919 |
| 19 CRC | 0 | 5229 | 1140 | 625 | 0 | 985 | 983 | 3179 |
| 20 CRC | 874 | 4717 | 882871 | 151188 | 20900 | 425704 | 548710 | 203893 |
| 21 CRC | 1556 | 878171 | 174368 | 282018 | 36714 | 238160 | 106391 | 25942 |
| 22 CRC | 178040 | 1002423 | 1147958 | 553349 | 90724 | 123432 | 235642 | 528953 |
| 23 CRC | 648 | 131917 | 69247 | 1130 | 16189 | 859 | 29625 | 287168 |
| 24 CRC | 53808 | 1617417 | 562 | 462 | 1600 | 1724 | 693 | 0 |
| 25 CRC | 207546 | 8203 | 163590 | 2206 | 0 | 13105 | 2135 | 109559 |
| 26 CRC | 0 | 631978 | 4602 | 114416 | 32295 | 240249 | 125194 | 129859 |
| 27 CRC | 1750 | 1547616 | 462049 | 112034 | 5986 | 407918 | 173576 | 1122203 |
| 28 CRC | 261 | 18893 | 29486 | 28971 | 4754 | 256374 | 62653 | 0 |
| 29 CRC | 60059 | 1402229 | 12983 | 1993 | 36116 | 436645 | 133495 | 86758 |
| 30 CRC | 108644 | 104391 | 88773 | 0 | 2141 | 1692 | 428880 | 3596 |
| 31 CRC | 2533 | 1601016 | 33709 | 658 | 293 | 364320 | 34994 | 157156 |
| 32 CRC | 0 | 54925 | 47913 | 2926 | 7711 | 244 | 8460 | 46954 |
| 33 NED | 264 | 3083 | 864 | 0 | 2657 | 298 | 426 | 22879 |
| 34 NED | 0 | 3408 | 0 | 579 | 0 | 729 | 694 | 4247 |
| 35 NED | 522 | 2897 | 1279 | 623 | 0 | 0 | 700 | 1643 |
| 36 NED | 1057 | 5535 | 823 | 1263 | 7450 | 2253 | 35999 | 899 |
| 37 NED | 0 | 0 | 888 | 324 | 0 | 1761 | 146 | 697 |
| 38 NED | 691 | 4410 | 2069 | 1444 | 0 | 1555 | 1112 | 215372 |
| 39 NED | 515 | 5147 | 748 | 0 | 5766 | 565 | 184 | 0 |
| 40 CRC | 0 | 791963 | 14724 | 123 | 700 | 340 | 5346 | 5165 |
| 41 CRC | 378 | 6607 | 5041 | 5077 | 331 | 1256 | 1622 | 45378 |
| 42 CRC | 245513 | 3671 | 60188 | 440297 | 64538 | 2053 | 48218 | 46812 |
| 43 CRC | 38846 | 736083 | 4910 | 2031 | 120887 | 168652 | 30580 | 380917 |
| 44 CRC | 55634 | 283436 | 4314 | 6065 | 63452 | 867 | 14633 | 89115 |
| 45 CRC | 227372 | 784822 | 80211 | 30403 | 56245 | 236244 | 104037 | 201092 |
| 46 CRC | 365920 | 278957 | 939885 | 1036510 | 113027 | 287957 | 361986 | 294037 |
| 47 CRC | 349143 | 2370748 | 1136628 | 341044 | 66028 | 373776 | 163767 | 360692 |
| 48 CRC | 213500 | 1761423 | 0 | 1345 | 172347 | 346611 | 18364 | 1573875 |
| 49 CRC | 525016 | 739019 | 22402 | 21935 | 1629 | 224734 | 3329 | 24277 |
| 50 CRC | 149718 | 97593 | 2721 | 1421 | 0 | 814 | 25025 | 1670 |
| 51 CRC | 199186 | 1501715 | 207779 | 1355760 | 104628 | 117265 | 203384 | 1215568 |
| 52 CRC | 815 | 3017 | 789 | 4003 | 1111 | 980 | 486 | 7542 |
| 53 CRC | 1664 | 451042 | 113 | 20125 | 1506 | 2594 | 26906 | 7515 |
| 54 CRC | 0 | 1945 | 454 | 897 | 0 | 279 | 358 | 62974 |
| 55 CRC | 393 | 1513907 | 7796 | 1616398 | 173462 | 125821 | 246621 | 860279 |
| 56 CRC | 614728 | 2597750 | 442368 | 1884675 | 331326 | 296788 | 206539 | 965590 |
| 57 CRC | 148 | 1477 | 0 | 265 | 154 | 0 | 26021 | 6696 |
| 58 CRC | 331578 | 9081 | 7714 | 8102 | 663 | 1555 | 9958 | 239680 |
| 59 CRC | 178393 | 1583437 | 1517665 | 1584498 | 94141 | 277387 | 506666 | 1411501 |
| 60 CRC | 0 | 1079933 | 666439 | 783818 | 273511 | 75499 | 156376 | 2482572 |
| 61 CRC | 0 | 2221 | 573 | 503 | 41012 | 27378 | 11568 | 24231 |
| 62 CRC | 1219 | 2030 | 1881 | 20019 | 0 | 474547 | 327 | 685 |
| 63 CRC | 515616 | 1322864 | 124870 | 1667521 | 65247 | 249279 | 41930 | 146409 |
| 64 CRC | 252578 | 170200 | 1428 | 4337 | 64304 | 216 | 1191 | 11479 |
| 65 CRC | 319493 | 567784 | 169919 | 839969 | 19287 | 198840 | 75234 | 1587796 |
| 66 CRC | 312885 | 1117017 | 152832 | 2273131 | 20508 | 476950 | 392058 | 1932932 |
| 67 CRC | 0 | 34932 | 276 | 806 | 16103 | 437 | 21459 | 27036 |
| 68 NED | 0 | 1929 | 315 | 346 | 0 | 0 | 0 | 304430 |
| 69 NED | 0 | 698 | 548 | 0 | 11530 | 303 | 0 | 26390 |
| 70 NED | 0 | 1353 | 0 | 1008 | 256 | 267 | 302 | 3605 |
| 71 NED | 0 | 664 | 0 | 119 | 16416 | 133 | 0 | 585 |
| 72 NED | 0 | 1565 | 118 | 518 | 1814 | 261 | 0 | 814 |
| 73 NED | 437 | 1214 | 238 | 1175 | 0 | 149 | 2112 | 938 |
| 74 NED | 0 | 335449 | 26985 | 826 | 6776 | 0 | 5319 | 1234 |
| 75 NED | 367 | 3875 | 400 | 878 | 1221 | 0 | 0 | 2825 |
| 76 NED | 0 | 1321 | 185 | 711 | 370 | 131 | 2829 | 1075 |
| 77 NED | 0 | 1052 | 1032 | 226 | 0 | 113 | 305 | 76366 |
| 78 NED | 111 | 493 | 0 | 265 | 15662 | 0 | 358 | 52604 |
| 79 NED | 0 | 952364 | 362 | 265 | 0 | 0 | 0 | 608 |
| 80 NED | 96 | 4257 | 313 | 0 | 0 | 0 | 0 | 0 |
| 81 NED | 513 | 315069 | 0 | 307 | 0 | 142 | 0 | 0 |
| 82 NED | 414 | 1110300 | 563 | 989 | 18050 | 380 | 4997 | 316804 |
| 83 NED | 0 | 1503 | 29607 | 249 | 0 | 175 | 0 | 19881 |
| 84 CRC | 0 | 43927 | 1791 | 22627 | 12105 | 616791 | 5920 | 8562 |
| 85 CRC | 0 | 3046269 | 3510 | 1513176 | 326293 | 131237 | 339883 | 449033 |
| 86 CRC | 0 | 0 | 635 | 464 | 0 | 2635 | 156 | 667 |
| 87 CRC | 2517 | 4574 | 249 | 820 | 15127 | 534748 | 0 | 112846 |
| 88 CRC | 1905 | 8249 | 0 | 910 | 2504 | 217 | 409 | 0 |
| 89 CRC | 0 | 5506 | 705 | 515 | 2856 | 2389 | 42586 | 0 |
| 90 CRC | 804 | 833115 | 0 | 8643 | 22976 | 0 | 277601 | 192857 |
| 91 CRC | 0 | 1170337 | 985703 | 2186089 | 67158 | 296712 | 596064 | 601603 |
| 92 CRC | 165902 | 1101960 | 1702 | 3423 | 193146 | 2173 | 5593 | 35777 |
| 93 CRC | 361220 | 5129806 | 1132486 | 1678913 | 44012 | 327563 | 602144 | 665561 |
| 94 CRC | 288 | 2722 | 157 | 1034 | 2869 | 162 | 310 | 8518 |
| 95 CRC | 166812 | 4857699 | 108274 | 13684 | 207713 | 847761 | 392766 | 463544 |
| 96 CRC | 0 | 8244 | 1941 | 4354 | 252258 | 1033 | 1913 | 3671 |
| 97 CRC | 815 | 2346238 | 343436 | 1337415 | 3392 | 395402 | 722510 | 896673 |
| 98 CRC | 183 | 2241 | 799 | 0 | 726 | 1009 | 0 | 0 |
| 99 CRC | 0 | 8829 | 1333 | 783 | 0 | 782277 | 657 | 2520 |
| 100 CRC | 592 | 1706852 | 95928 | 1856 | 46465 | 971973 | 85225 | 353695 |
| 101 CRC | 3068 | 1169911 | 0 | 125479 | 30665 | 231858 | 49403 | 39723 |
| 102 CRC | 362633 | 3990769 | 322591 | 1665 | 45428 | 290033 | 118564 | 64601 |
| 103 CRC | 0 | 29762 | 332 | 1819 | 44751 | 0 | 1635 | 0 |
| 104 CRC | 3540 | 2524110 | 2298340 | 1625458 | 351874 | 97251 | 529393 | 1094377 |
| 105 CRC | 813 | 1643831 | 3545 | 2333 | 0 | 226 | 1572 | 2905 |
| 106 CRC | 10866 | 131147 | 13304 | 83357 | 20478 | 486231 | 94253 | 157118 |
| 107 CRC | 430610 | 5606905 | 447770 | 1774381 | 1200 | 252042 | 221119 | 295499 |
| 108 CRC | 1007 | 3171059 | 266313 | 3610 | 73754 | 508140 | 94693 | 287534 |
| 109 CRC | 409837 | 4703778 | 848872 | 1339282 | 149222 | 66567 | 320594 | 152548 |
| 110 CRC | 0 | 673593 | 166 | 0 | 0 | 5646 | 491 | 0 |
| 111 CRC | 250555 | 1657131 | 0 | 458 | 0 | 25423 | 0 | 45856 |
| 112 CRC | 752 | 2161864 | 6147 | 1798 | 18431 | 1537884 | 62199 | 0 |
| 113 CRC | 3982 | 34885 | 8194 | 6872 | 4649 | 553 | 70306 | 172608 |
| 114 CRC | 1023 | 15723 | 115782 | 1515 | 933 | 1007585 | 0 | 652365 |
| 115 CRC | 604 | 2194955 | 17100 | 361 | 2637 | 6030 | 23009 | 6219 |
| 116 CRC | 50895 | 5133770 | 1205362 | 1084494 | 72232 | 501142 | 842834 | 372295 |
| 117 CRC | 0 | 42982 | 620 | 0 | 105897 | 99686 | 156140 | 3127 |
| 118 CRC | 407781 | 2746395 | 1284301 | 1516994 | 87742 | 229959 | 681791 | 299125 |
| 119 CRC | 50993 | 401356 | 251 | 4227 | 56364 | 280 | 120896 | 56304 |
| 120 CRC | 659196 | 4007776 | 2308726 | 1965882 | 66126 | 484737 | 913454 | 135148 |
| 121 CRC | 1725 | 5364 | 0 | 618 | 1286 | 1098 | 185 | 25908 |
| 122 CRC | 207191 | 1899708 | 826144 | 1125231 | 789757 | 109735 | 466370 | 2373714 |
| 123 CRC | 646 | 160285 | 0 | 412051 | 147643 | 352103 | 168979 | 937374 |
| 124 CRC | 279260 | 709 | 232 | 0 | 7579 | 1168 | 0 | 0 |
| 125 NED | 0 | 4878 | 399 | 1153 | 1092 | 1645 | 726 | 0 |
| 126 NED | 0 | 1544 | 2272 | 0 | 1096 | 28206 | 0 | 9547 |
| 127 NED | 576 | 5333 | 0 | 0 | 12287 | 671 | 1031 | 4222 |
| 128 NED | 0 | 3846 | 269 | 0 | 1066 | 453 | 0 | 16786 |
| 129 NED | 577 | 1341 | 1635 | 414 | 0 | 0 | 0 | 317 |
| 130 NED | 31933 | 4201 | 58407 | 1462 | 687 | 882 | 597 | 125026 |
| 131 NED | 1120 | 876263 | 1239 | 2343 | 1153 | 6968 | 72629 | 13403 |
| 132 NED | 79795 | 4166 | 0 | 172 | 0 | 322 | 1084 | 2973 |
| 133 NED | 753 | 11044 | 1477 | 2160 | 0 | 495 | 116482 | 414 |
| 134 NED | 0 | 2273 | 117 | 257 | 0 | 135 | 1627 | 0 |
| 135 NED | 0 | 1116018 | 1277 | 1400 | 486 | 69465 | 66014 | 151 |
| 136 NED | 352 | 693436 | 256 | 561 | 10609 | 0 | 0 | 180 |
| 137 NED | 0 | 22881 | 0 | 0 | 77790 | 1927 | 119565 | 0 |
| 138 NED | 0 | 298 | 439 | 0 | 1304 | 899 | 0 | 738 |
| 139 NED | 591 | 1219 | 92 | 504 | 0 | 108 | 453 | 2237 |
| 140 NED | 0 | 1589 | 0 | 414 | 1611 | 643 | 6145 | 0 |
| 141 NED | 244 | 2031 | 398 | 291 | 813 | 0 | 0 | 0 |
| 142 NED | 141 | 1253 | 615 | 245 | 4019 | 1002 | 151 | 1356 |
| 143 NED | 781 | 4009 | 744 | 1749 | 1987 | 619 | 419 | 87491 |
| 144 NED | 315 | 668308 | 49236 | 0 | 306 | 718 | 113 | 0 |
| 145 NED | 492 | 5466 | 77616 | 588 | 12110 | 274 | 264 | 473 |
| 146 NED | 340 | 7667 | 1480 | 1116 | 465 | 2112 | 4131 | 21053 |
| 147 NED | 0 | 2685 | 439 | 642 | 273 | 315 | 288 | 2029 |
| 148 NED | 983 | 14600 | 2543 | 3376 | 0 | 140 | 1715 | 1688 |
| 149 NED | 989 | 2884 | 674 | 887 | 22934 | 255 | 266 | 510 |
| 150 NED | 0 | 1363 | 594 | 0 | 3695 | 537712 | 0 | 73161 |
| 151 NED | 776 | 5298 | 2409 | 1390 | 0 | 9647 | 555 | 252006 |
| 152 NED | 101 | 1693 | 220 | 602 | 0 | 221 | 216 | 5417005 |
| 153 NED | 183391 | 3886 | 0 | 1858 | 0 | 1105 | 974 | 4985 |
| 154 NED | 491 | 5234 | 68889 | 1056 | 781 | 755 | 34051 | 24952 |
| 155 NED | 0 | 3074 | 476 | 870 | 826 | 165 | 1095 | 0 |
| 156 NED | 129 | 1228 | 0 | 1079 | 13937 | 160 | 291247 | 0 |
| 157 NED | 17050 | 565 | 0 | 228 | 35936 | 1789 | 0 | 13050 |
| 158 NED | 3868 | 15937 | 1806 | 2189 | 9222 | 1732 | 1311 | 2656 |
| 159 NED | 0 | 12347 | 0 | 0 | 3480 | 722246 | 21487 | 0 |
| 160 NED | 1555 | 2198 | 462 | 0 | 287 | 3430 | 911 | 1088 |
| 161 NED | 555 | 240332 | 127011 | 4569 | 125677 | 1232491 | 4620 | 272112 |
| 162 NED | 0 | 3505 | 123 | 808 | 4453 | 124 | 484 | 17653 |
| 163 NED | 4057 | 50681 | 8563 | 16509 | 0 | 1200 | 3947 | 2709 |
| 164 NED | 947 | 11867 | 589 | 970 | 24552 | 1014 | 1452 | 37533 |
| 165 NED | 1503 | 358 | 819 | 160 | 1744 | 263 | 230 | 94661 |
| 166 NED | 198 | 769 | 0 | 236 | 0 | 7245 | 74275 | 0 |
| 167 NED | 362470 | 2744 | 245 | 268 | 7514 | 151 | 241 | 463 |
| 168 NED | 438061 | 1244 | 0 | 365 | 1425 | 17037 | 7872 | 599 |
| 169 NED | 0 | 3390 | 128 | 701 | 183 | 0 | 0 | 340 |
| 170 NED | 0 | 1707565 | 971 | 399 | 0 | 369 | 598 | 84513 |

1. A method of detecting DNA methylation, comprising the step of detecting DNA methylation within at least one genomic DNA polynucleotide selected from the group consisting of polynucleotides having a sequence comprised in SEQ ID NO: 16 (mADCYAP1), SEQ ID NO: 56 and/or SEQ ID NO: 61 (mANKRD13B), SEQ ID NO: 41 and/or SEQ ID NO: 46 (mCLEC14A), SEQ ID NO: 71 (mCRMP1), SEQ ID NO: 81 and/or SEQ ID NO: 86 (mEYA4), SEQ ID NO: 31 (mKHDRBS2), SEQ ID NO: 96 and/or SEQ ID NO: 101 (mMSC), SEQ ID NO: 111 and/or SEQ ID NO: 116 (mNGFR), SEQ ID NO: 126 (mNKX2), SEQ ID NO: 141 and/or SEQ ID NO: 146 (mRASSF2), SEQ ID NO: 1 (mSEPT9), SEQ ID NO: 161 (mSND1), SEQ ID NO: 171 (mTBX18), SEQ ID NO: 186 and/or SEQ ID NO: 191 (mTFAP2E), SEQ ID NO: 201 and/or SEQ ID NO: 206 (mTMEFF2), or SEQ ID NO: 216 (mVAX1) in a subject's biological sample comprising genomic DNA, wherein the genomic DNA may comprise DNA derived from colorectal cancer (CRC) cells.

2. The method of claim 1, wherein DNA methylation is detected within at least two, preferably at least three, genomic DNA polynucleotides selected from said group.

3. The method of claim 1 or 2, comprising the steps of
(a) converting cytosine unmethylated in the 5-position to uracil or another base that does not hybridize to guanine in the genomic DNA of the biological sample; and
(b) detecting DNA methylation within the genomic DNA by detecting unconverted cytosine in the converted DNA of step (a).

4. The method of any one of claims 1 to 3, wherein the detecting of the DNA methylation comprises determining the amount of methylated genomic DNA.

5. The method of any one of claims 1 to 4, wherein the biological sample is a colon or rectum tissue sample or a liquid biopsy, preferably a blood sample, a sample comprising cell-free DNA from blood, a blood-derived sample or a saliva sample.

6. The method of any one of claims 1 to 5, wherein the genomic DNA is cell-free DNA.

7. The method of any one of claims 1 to 6, wherein the subject is suspected of having CRC, has an increased risk of developing CRC, has had CRC, or has CRC.

8. A method for detecting the presence or absence of colorectal cancer (CRC) in a subject, comprising detecting DNA methylation according to any one of claims 1 to 7, wherein the presence of detected methylated genomic DNA indicates the presence of CRC and the absence of detected methylated genomic DNA indicates the absence of CRC.

9. A method for monitoring a subject suspected of having CRC, having an increased risk of developing colorectal cancer (CRC), or who has had CRC, comprising detecting DNA methylation according to claim 8 repeatedly, wherein the presence of detected methylated genomic DNA indicates the presence of CRC and the absence of detected methylated genomic DNA indicates the absence of CRC.

10. An oligonucleotide selected from the group consisting of a primer and probe, comprising a sequence that is substantially identical to a stretch of contiguous nucleotides of one of SEQ ID NOs 17-20 (mADCYAP1), one of SEQ ID NOs 57-60 and/or one of SEQ ID NOs 62-65 (mANKRD13B), one of SEQ ID NOs 42-45 and/or one of SEQ ID NOs 47-50 (mCLEC14A), one of SEQ ID NOs 72-75 (mCRMP1), one of SEQ ID NOs 82-85 and/or one of SEQ ID NOs 87-90 (mEYA4), one of SEQ ID NOs 32-35 (mKHDRBS2), one of SEQ ID NOs 97-100 and/or one of SEQ ID NOs 102-105 (mMSC), one of SEQ ID NOs 112-115 and/or one of SEQ ID NOs 117-120 (mNGFR), one of SEQ ID NOs 127-130 (mNKX2), one of SEQ ID NOs 142-145 and/or one of SEQ ID NOs 147-150 (mRASSF2), one of SEQ ID NOs 2-5 (mSEPT9), one of SEQ ID NOs 162-165 (mSND1), one of SEQ ID NOs 172-175 (mTBX18), one of SEQ ID NOs 187-190 and/or one of SEQ ID NOs 192-195 (mTFAP2E), one of SEQ ID NOs 202-205 and/or one of SEQ ID NOs 207-210 (mTMEFF2), one of SEQ ID NOs 217-220 (mVAX1).

11. The oligonucleotide of claim 10, wherein the oligonucleotide is methylation-specific.

12. A kit comprising at least a first and a second oligonucleotide of claim 10 or 11.

13. The kit of claim 12, wherein the first and second oligonucleotides are primers forming a primer pair suitable for amplification of DNA having a sequence comprised in one of SEQ ID NOs 17-20 (mADCYAP1), one of SEQ ID NOs 57-60 and/or one of SEQ ID NOs 62-65 (mANKRD13B), one of SEQ ID NOs 42-45 and/or one of SEQ ID NOs 47-50 (mCLEC14A), one of SEQ ID NOs 72-75 (mCRMP1), one of SEQ ID NOs 82-85 and/or one of SEQ ID NOs 87-90 (mEYA4), one of SEQ ID NOs 32-35 (mKHDRBS2), one of SEQ ID NOs 97-100 and/or one of SEQ ID NOs 102-105 (mMSC), one of SEQ ID NOs 112-115 and/or one of SEQ ID NOs 117-120 (mNGFR), one of SEQ ID NOs 127-130 (mNKX2), one of SEQ ID NOs 142-145 and/or one of SEQ ID NOs 147-150 (mRASSF2), one of SEQ ID NOs 2-5 (mSEPT9), one of SEQ ID NOs 162-165 (mSND1), one of SEQ ID NOs 172-175 (mTBX18), one of SEQ ID NOs 187-190 and/or one of SEQ ID NOs 192-195 (mTFAP2E), one of SEQ ID NOs 202-205 and/or one of SEQ ID NOs 207-210 (mTMEFF2), one of SEQ ID NOs 217-220 (mVAX1).

14. The kit of claim 12 or 13, wherein the kit comprises polynucleotides forming at least two, preferably at least three primer pairs, and wherein each primer pair is suitable for amplification of DNA having a sequence of a different marker mADCYAP1, mANKRD13B, mCLEC14A, mCRMP1, mEYA4, mKHDRBS2, mMSC, mNGFR, mNKX2, mRASSF2, mSEPT9, mSND1, mTBX18, mTFAP2E, mTMEFF2 and mVAX1.

15. Use of the method of any one of claims 1 to 7, of the oligonucleotide of claim 10 or 11, or of the kit of any one of claims 12 to 14 for the detection of colorectal cancer (CRC), or for monitoring a subject having an increased risk of developing CRC, suspected of having CRC or that has had CRC.


SEQUENCE LISTING (255 sequences)

SEQ ID NO: 1 - DNA, 1819 bp, Homo sapiens
gcgtcgcccgtccctggcttctctgacagccgtgttccatccccgccctgtgccccttctcccggacagtgccttctccagggctcacccaggagggtgcagcggtggcccccggggcggtggtcgtggtgggggtgttagctgcaggggtgccctcggtgggtgggagttggtggcctctcgctggtgccatgggactcgcatgttcgccctgcgcccctcggctcttgagcccacaggccgggatcctgcctgccagccgcgtgcgctgccgtttaacccttgcaggcgcagagcgcgcggcggcggtgacagagaactttgtttggctgcccaaatacagcctcctgcagaaggaccctgcgcccggggaaggggaggaatctcttcccctctgggcgcccgccctcctcgccatggcccggcctccacatccgcccacatctggccgcagcggggcgcccggggggaggggctgaggccgcgtctctcgccgtcccctgggcgcgggccaggcggggaggaggggggcgctccggtcgtgtgcccaggactgtcccccagcggccactcgggccccagccccccaggcctggccttgacaggcgggcggagcagccagtgcgagacagggaggccggtgcgggtgcgggaacctgatccgcccgggaggcgggggcggggcgggggcgcagcgcgcggggaggggccggcgcccgccttcctcccccattcattcagctgagccagggggcctaggggctcctccggcggctagctctgcactgcaggagcgcgggcgcggcgccccagccagcgcgcagggcccgggccccgccgggggcgcttcctcgccgctgccctccgcgcgacccgctgcccaccagccatcatgtcggaccccgcggtcaacgcgcagctggatgggatcatttcggacttcgaaggtgggtgctgggctggctgctgcggccgcggacgtgctggagaggaccctgcgggtgggcctggcgcgggacgggggtgcgctgaggggagacgggagtgcgctgaggggagacgggacccctaatccaggcgccctcccgctgagagcgccgcgcgcccccggccccgtgcccgcgccgcctacgtgggggaccctgttaggggcacccgcgtagaccctgcgcgccctcacaggaccctgtgctcgttctgcgcactgccgcctgggtttccttccttttattgttgtttgtgtttgccaagcgacagcgacctcctcgagggctcgcgaggctgcctcggaactctccaggacgcacagtttcactctgggaaatccatcggtcccctccctttggctctccccggcggctctcgggccccgcttggacccggcaacgggatagggaggtcgttcctcacctccgactgagtggacagccgcgtcctgctcgggtggacagccctcccctcccccacgccagtttcggggccgccaagttgtgcagcccgtgggccgggagcaccgaacggacacagcccaggtcgtggcagggtctagagtgggatgtcccatggcccccatccaggcctggggatatcctcatccgcctcccagaatcgggccgtgggggacagaaggggcctgcgtgcgggcagggagagtattttggctctctcctgtcttcggggtttacaaagtgtgttgggacttgcggggctgctctgtccaagcctgggtctggcgtccgcgtctctgagcctgtgagtgcgtgcgctttcctgcgtcctcttgactgccggtgctggggctctgcgtcctgcgtccgcg

SEQ ID NO: 2 - DNA, 1819 bp, Artificial Sequence
  converted
gcgtcgttcgtttttggtttttttgatagtcgtgttttattttcgttttgtgttttttttttcggatagtgttttttttagggtttatttaggagggtgtagcggtggttttcggggcggtggtcgtggtgggggtgttagttgtaggggtgttttcggtgggtgggagttggtggtttttcgttggtgttatgggattcgtatgttcgttttgcgtttttcggtttttgagtttataggtcgggattttgtttgttagtcgcgtgcgttgtcgtttaatttttgtaggcgtagagcgcgcggcggcggtgatagagaattttgtttggttgtttaaatatagttttttgtagaaggattttgcgttcggggaaggggaggaatttttttttttttgggcgttcgtttttttcgttatggttcggtttttatattcgtttatatttggtcgtagcggggcgttcggggggaggggttgaggtcgcgtttttcgtcgttttttgggcgcgggttaggcggggaggaggggggcgtttcggtcgtgtgtttaggattgttttttagcggttattcgggttttagttttttaggtttggttttgataggcgggcggagtagttagtgcgagatagggaggtcggtgcgggtgcgggaatttgattcgttcgggaggcgggggcggggcgggggcgtagcgcgcggggaggggtcggcgttcgttttttttttttatttatttagttgagttagggggtttaggggttttttcggcggttagttttgtattgtaggagcgcgggcgcggcgttttagttagcgcgtagggttcgggtttcgtcgggggcgttttttcgtcgttgtttttcgcgcgattcgttgtttattagttattatgtcggatttcgcggttaacgcgtagttggatgggattatttcggatttcgaaggtgggtgttgggttggttgttgcggtcgcggacgtgttggagaggattttgcgggtgggtttggcgcgggacgggggtgcgttgaggggagacgggagtgcgttgaggggagacgggatttttaatttaggcgttttttcgttgagagcgtcgcgcgttttcggtttcgtgttcgcgtcgtttacgtgggggattttgttaggggtattcgcgtagattttgcgcgtttttataggattttgtgttcgttttgcgtattgtcgtttgggtttttttttttttattgttgtttgtgtttgttaagcgatagcgattttttcgagggttcgcgaggttgtttcggaattttttaggacgtatagttttattttgggaaatttatcggttttttttttttggtttttttcggcggttttcgggtttcgtttggattcggtaacgggatagggaggtcgttttttattttcgattgagtggatagtcgcgttttgttcgggtggatagttttttttttttttacgttagtttcggggtcgttaagttgtgtagttcgtgggtcgggagtatcgaacggatatagtttaggtcgtggtagggtttagagtgggatgttttatggtttttatttaggtttggggatatttttattcgttttttagaatcgggtcgtgggggatagaaggggtttgcgtgcgggtagggagagtattttggtttttttttgttttcggggtttataaagtgtgttgggatttgcggggttgttttgtttaagtttgggtttggcgttcgcgtttttgagtttgtgagtgcgtgcgtttttttgcgtttttttgattgtcggtgttggggttttgcgttttgcgttcgcg

SEQ ID NO: 3 - DNA, 1819 bp, Artificial Sequence
  converted
cgcgaacgcaaaacgcaaaaccccaacaccgacaatcaaaaaaacgcaaaaaaacgcacgcactcacaaactcaaaaacgcgaacgccaaacccaaacttaaacaaaacaaccccgcaaatcccaacacactttataaaccccgaaaacaaaaaaaaaccaaaatactctccctacccgcacgcaaaccccttctatcccccacgacccgattctaaaaaacgaataaaaatatccccaaacctaaataaaaaccataaaacatcccactctaaaccctaccacgacctaaactatatccgttcgatactcccgacccacgaactacacaacttaacgaccccgaaactaacgtaaaaaaaaaaaaaactatccacccgaacaaaacgcgactatccactcaatcgaaaataaaaaacgacctccctatcccgttaccgaatccaaacgaaacccgaaaaccgccgaaaaaaaccaaaaaaaaaaaaccgataaatttcccaaaataaaactatacgtcctaaaaaattccgaaacaacctcgcgaaccctcgaaaaaatcgctatcgcttaacaaacacaaacaacaataaaaaaaaaaaaacccaaacgacaatacgcaaaacgaacacaaaatcctataaaaacgcgcaaaatctacgcgaatacccctaacaaaatcccccacgtaaacgacgcgaacacgaaaccgaaaacgcgcgacgctctcaacgaaaaaacgcctaaattaaaaatcccgtctcccctcaacgcactcccgtctcccctcaacgcacccccgtcccgcgccaaacccacccgcaaaatcctctccaacacgtccgcgaccgcaacaaccaacccaacacccaccttcgaaatccgaaataatcccatccaactacgcgttaaccgcgaaatccgacataataactaataaacaacgaatcgcgcgaaaaacaacgacgaaaaaacgcccccgacgaaacccgaaccctacgcgctaactaaaacgccgcgcccgcgctcctacaatacaaaactaaccgccgaaaaaacccctaaaccccctaactcaactaaataaataaaaaaaaaaaacgaacgccgacccctccccgcgcgctacgcccccgccccgcccccgcctcccgaacgaatcaaattcccgcacccgcaccgacctccctatctcgcactaactactccgcccgcctatcaaaaccaaacctaaaaaactaaaacccgaataaccgctaaaaaacaatcctaaacacacgaccgaaacgcccccctcctccccgcctaacccgcgcccaaaaaacgacgaaaaacgcgacctcaacccctccccccgaacgccccgctacgaccaaatataaacgaatataaaaaccgaaccataacgaaaaaaacgaacgcccaaaaaaaaaaaaattcctccccttccccgaacgcaaaatccttctacaaaaaactatatttaaacaaccaaacaaaattctctatcaccgccgccgcgcgctctacgcctacaaaaattaaacgacaacgcacgcgactaacaaacaaaatcccgacctataaactcaaaaaccgaaaaacgcaaaacgaacatacgaatcccataacaccaacgaaaaaccaccaactcccacccaccgaaaacacccctacaactaacacccccaccacgaccaccgccccgaaaaccaccgctacaccctcctaaataaaccctaaaaaaaacactatccgaaaaaaaaaacacaaaacgaaaataaaacacgactatcaaaaaaaccaaaaacgaacgacgc

SEQ ID NO: 4 - DNA, 1819 bp, Artificial Sequence
  converted
acgtcgcccgtccctaacttctctaacaaccgtattccatccccgccctataccccttctcccgaacaataccttctccaaaactcacccaaaaaaatacaacgataacccccgaaacgataatcgtaataaaaatattaactacaaaaataccctcgataaataaaaattaataacctctcgctaataccataaaactcgcatattcgccctacgcccctcgactcttaaacccacaaaccgaaatcctacctaccaaccgcgtacgctaccgtttaacccttacaaacgcaaaacgcgcgacgacgataacaaaaaactttatttaactacccaaatacaacctcctacaaaaaaaccctacgcccgaaaaaaaaaaaaaatctcttcccctctaaacgcccgccctcctcgccataacccgacctccacatccgcccacatctaaccgcaacgaaacgcccgaaaaaaaaaactaaaaccgcgtctctcgccgtcccctaaacgcgaaccaaacgaaaaaaaaaaaaacgctccgatcgtatacccaaaactatcccccaacgaccactcgaaccccaaccccccaaacctaaccttaacaaacgaacgaaacaaccaatacgaaacaaaaaaaccgatacgaatacgaaaacctaatccgcccgaaaaacgaaaacgaaacgaaaacgcaacgcgcgaaaaaaaaccgacgcccgccttcctcccccattcattcaactaaaccaaaaaacctaaaaactcctccgacgactaactctacactacaaaaacgcgaacgcgacgccccaaccaacgcgcaaaacccgaaccccgccgaaaacgcttcctcgccgctaccctccgcgcgacccgctacccaccaaccatcatatcgaaccccgcgatcaacgcgcaactaaataaaatcatttcgaacttcgaaaataaatactaaactaactactacgaccgcgaacgtactaaaaaaaaccctacgaataaacctaacgcgaaacgaaaatacgctaaaaaaaaacgaaaatacgctaaaaaaaaacgaaacccctaatccaaacgccctcccgctaaaaacgccgcgcgcccccgaccccgtacccgcgccgcctacgtaaaaaaccctattaaaaacacccgcgtaaaccctacgcgccctcacaaaaccctatactcgttctacgcactaccgcctaaatttccttccttttattattatttatatttaccaaacgacaacgacctcctcgaaaactcgcgaaactacctcgaaactctccaaaacgcacaatttcactctaaaaaatccatcgatcccctccctttaactctccccgacgactctcgaaccccgcttaaacccgacaacgaaataaaaaaatcgttcctcacctccgactaaataaacaaccgcgtcctactcgaataaacaaccctcccctcccccacgccaatttcgaaaccgccaaattatacaacccgtaaaccgaaaacaccgaacgaacacaacccaaatcgtaacaaaatctaaaataaaatatcccataacccccatccaaacctaaaaatatcctcatccgcctcccaaaatcgaaccgtaaaaaacaaaaaaaacctacgtacgaacaaaaaaaatattttaactctctcctatcttcgaaatttacaaaatatattaaaacttacgaaactactctatccaaacctaaatctaacgtccgcgtctctaaacctataaatacgtacgctttcctacgtcctcttaactaccgatactaaaactctacgtcctacgtccgcg

SEQ ID NO: 5 - DNA, 1819 bp, Artificial Sequence
  converted
cgcggacgtaggacgtagagttttagtatcggtagttaagaggacgtaggaaagcgtacgtatttataggtttagagacgcggacgttagatttaggtttggatagagtagtttcgtaagttttaatatattttgtaaatttcgaagataggagagagttaaaatattttttttgttcgtacgtaggttttttttgttttttacggttcgattttgggaggcggatgaggatatttttaggtttggatgggggttatgggatattttattttagattttgttacgatttgggttgtgttcgttcggtgttttcggtttacgggttgtataatttggcggtttcgaaattggcgtgggggaggggagggttgtttattcgagtaggacgcggttgtttatttagtcggaggtgaggaacgatttttttatttcgttgtcgggtttaagcggggttcgagagtcgtcggggagagttaaagggaggggatcgatggattttttagagtgaaattgtgcgttttggagagtttcgaggtagtttcgcgagttttcgaggaggtcgttgtcgtttggtaaatataaataataataaaaggaaggaaatttaggcggtagtgcgtagaacgagtatagggttttgtgagggcgcgtagggtttacgcgggtgtttttaatagggttttttacgtaggcggcgcgggtacggggtcgggggcgcgcggcgtttttagcgggagggcgtttggattaggggtttcgtttttttttagcgtattttcgtttttttttagcgtattttcgtttcgcgttaggtttattcgtagggttttttttagtacgttcgcggtcgtagtagttagtttagtatttattttcgaagttcgaaatgattttatttagttgcgcgttgatcgcggggttcgatatgatggttggtgggtagcgggtcgcgcggagggtagcggcgaggaagcgttttcggcggggttcgggttttgcgcgttggttggggcgtcgcgttcgcgtttttgtagtgtagagttagtcgtcggaggagtttttaggttttttggtttagttgaatgaatgggggaggaaggcgggcgtcggttttttttcgcgcgttgcgttttcgtttcgttttcgtttttcgggcggattaggttttcgtattcgtatcggtttttttgtttcgtattggttgtttcgttcgtttgttaaggttaggtttggggggttggggttcgagtggtcgttgggggatagttttgggtatacgatcggagcgtttttttttttttcgtttggttcgcgtttaggggacggcgagagacgcggttttagttttttttttcgggcgtttcgttgcggttagatgtgggcggatgtggaggtcgggttatggcgaggagggcgggcgtttagaggggaagagattttttttttttttcgggcgtagggtttttttgtaggaggttgtatttgggtagttaaataaagttttttgttatcgtcgtcgcgcgttttgcgtttgtaagggttaaacggtagcgtacgcggttggtaggtaggatttcggtttgtgggtttaagagtcgaggggcgtagggcgaatatgcgagttttatggtattagcgagaggttattaatttttatttatcgagggtatttttgtagttaatatttttattacgattatcgtttcgggggttatcgttgtatttttttgggtgagttttggagaaggtattgttcgggagaaggggtatagggcggggatggaatacggttgttagagaagttagggacgggcgacgt

SEQ ID NO: 6 - DNA, 1062 bp, Homo sapiens
aggggaggaatctcttcccctctgggcgcccgccctcctcgccatggcccggcctccacatccgcccacatctggccgcagcggggcgcccggggggaggggctgaggccgcgtctctcgccgtcccctgggcgcgggccaggcggggaggaggggggcgctccggtcgtgtgcccaggactgtcccccagcggccactcgggccccagccccccaggcctggccttgacaggcgggcggagcagccagtgcgagacagggaggccggtgcgggtgcgggaacctgatccgcccgggaggcgggggcggggcgggggcgcagcgcgcggggaggggccggcgcccgccttcctcccccattcattcagctgagccagggggcctaggggctcctccggcggctagctctgcactgcaggagcgcgggcgcggcgccccagccagcgcgcagggcccgggccccgccgggggcgcttcctcgccgctgccctccgcgcgacccgctgcccaccagccatcatgtcggaccccgcggtcaacgcgcagctggatgggatcatttcggacttcgaaggtgggtgctgggctggctgctgcggccgcggacgtgctggagaggaccctgcgggtgggcctggcgcgggacgggggtgcgctgaggggagacgggagtgcgctgaggggagacgggacccctaatccaggcgccctcccgctgagagcgccgcgcgcccccggccccgtgcccgcgccgcctacgtgggggaccctgttaggggcacccgcgtagaccctgcgcgccctcacaggaccctgtgctcgttctgcgcactgccgcctgggtttccttccttttattgttgtttgtgtttgccaagcgacagcgacctcctcgagggctcgcgaggctgcctcggaactctccaggacgcacagtttcactctgggaaatccatcggtcccctccctttggctctccccggcggctctcgggccccgcttggacccggcaacgggatagggaggtcgttcctcacctccgactgagtggacagccgcgtc

SEQ ID NO: 7 - DNA, 1062 bp, Artificial Sequence
  converted
aggggaggaatttttttttttttgggcgttcgtttttttcgttatggttcggtttttatattcgtttatatttggtcgtagcggggcgttcggggggaggggttgaggtcgcgtttttcgtcgttttttgggcgcgggttaggcggggaggaggggggcgtttcggtcgtgtgtttaggattgttttttagcggttattcgggttttagttttttaggtttggttttgataggcgggcggagtagttagtgcgagatagggaggtcggtgcgggtgcgggaatttgattcgttcgggaggcgggggcggggcgggggcgtagcgcgcggggaggggtcggcgttcgttttttttttttatttatttagttgagttagggggtttaggggttttttcggcggttagttttgtattgtaggagcgcgggcgcggcgttttagttagcgcgtagggttcgggtttcgtcgggggcgttttttcgtcgttgtttttcgcgcgattcgttgtttattagttattatgtcggatttcgcggttaacgcgtagttggatgggattatttcggatttcgaaggtgggtgttgggttggttgttgcggtcgcggacgtgttggagaggattttgcgggtgggtttggcgcgggacgggggtgcgttgaggggagacgggagtgcgttgaggggagacgggatttttaatttaggcgttttttcgttgagagcgtcgcgcgttttcggtttcgtgttcgcgtcgtttacgtgggggattttgttaggggtattcgcgtagattttgcgcgtttttataggattttgtgttcgttttgcgtattgtcgtttgggtttttttttttttattgttgtttgtgtttgttaagcgatagcgattttttcgagggttcgcgaggttgtttcggaattttttaggacgtatagttttattttgggaaatttatcggttttttttttttggtttttttcggcggttttcgggtttcgtttggattcggtaacgggatagggaggtcgttttttattttcgattgagtggatagtcgcgtt

SEQ ID NO: 8 - DNA, 1062 bp, Artificial Sequence
  converted
aacgcgactatccactcaatcgaaaataaaaaacgacctccctatcccgttaccgaatccaaacgaaacccgaaaaccgccgaaaaaaaccaaaaaaaaaaaaccgataaatttcccaaaataaaactatacgtcctaaaaaattccgaaacaacctcgcgaaccctcgaaaaaatcgctatcgcttaacaaacacaaacaacaataaaaaaaaaaaaacccaaacgacaatacgcaaaacgaacacaaaatcctataaaaacgcgcaaaatctacgcgaatacccctaacaaaatcccccacgtaaacgacgcgaacacgaaaccgaaaacgcgcgacgctctcaacgaaaaaacgcctaaattaaaaatcccgtctcccctcaacgcactcccgtctcccctcaacgcacccccgtcccgcgccaaacccacccgcaaaatcctctccaacacgtccgcgaccgcaacaaccaacccaacacccaccttcgaaatccgaaataatcccatccaactacgcgttaaccgcgaaatccgacataataactaataaacaacgaatcgcgcgaaaaacaacgacgaaaaaacgcccccgacgaaacccgaaccctacgcgctaactaaaacgccgcgcccgcgctcctacaatacaaaactaaccgccgaaaaaacccctaaaccccctaactcaactaaataaataaaaaaaaaaaacgaacgccgacccctccccgcgcgctacgcccccgccccgcccccgcctcccgaacgaatcaaattcccgcacccgcaccgacctccctatctcgcactaactactccgcccgcctatcaaaaccaaacctaaaaaactaaaacccgaataaccgctaaaaaacaatcctaaacacacgaccgaaacgcccccctcctccccgcctaacccgcgcccaaaaaacgacgaaaaacgcgacctcaacccctccccccgaacgccccgctacgaccaaatataaacgaatataaaaaccgaaccataacgaaaaaaacgaacgcccaaaaaaaaaaaaattcctcccct

SEQ ID NO: 9 - DNA, 1062 bp, Artificial Sequence
  converted
aaaaaaaaaatctcttcccctctaaacgcccgccctcctcgccataacccgacctccacatccgcccacatctaaccgcaacgaaacgcccgaaaaaaaaaactaaaaccgcgtctctcgccgtcccctaaacgcgaaccaaacgaaaaaaaaaaaaacgctccgatcgtatacccaaaactatcccccaacgaccactcgaaccccaaccccccaaacctaaccttaacaaacgaacgaaacaaccaatacgaaacaaaaaaaccgatacgaatacgaaaacctaatccgcccgaaaaacgaaaacgaaacgaaaacgcaacgcgcgaaaaaaaaccgacgcccgccttcctcccccattcattcaactaaaccaaaaaacctaaaaactcctccgacgactaactctacactacaaaaacgcgaacgcgacgccccaaccaacgcgcaaaacccgaaccccgccgaaaacgcttcctcgccgctaccctccgcgcgacccgctacccaccaaccatcatatcgaaccccgcgatcaacgcgcaactaaataaaatcatttcgaacttcgaaaataaatactaaactaactactacgaccgcgaacgtactaaaaaaaaccctacgaataaacctaacgcgaaacgaaaatacgctaaaaaaaaacgaaaatacgctaaaaaaaaacgaaacccctaatccaaacgccctcccgctaaaaacgccgcgcgcccccgaccccgtacccgcgccgcctacgtaaaaaaccctattaaaaacacccgcgtaaaccctacgcgccctcacaaaaccctatactcgttctacgcactaccgcctaaatttccttccttttattattatttatatttaccaaacgacaacgacctcctcgaaaactcgcgaaactacctcgaaactctccaaaacgcacaatttcactctaaaaaatccatcgatcccctccctttaactctccccgacgactctcgaaccccgcttaaacccgacaacgaaataaaaaaatcgttcctcacctccgactaaataaacaaccgcgtc

SEQ ID NO: 10 - DNA, 1062 bp, Artificial Sequence
  converted
gacgcggttgtttatttagtcggaggtgaggaacgatttttttatttcgttgtcgggtttaagcggggttcgagagtcgtcggggagagttaaagggaggggatcgatggattttttagagtgaaattgtgcgttttggagagtttcgaggtagtttcgcgagttttcgaggaggtcgttgtcgtttggtaaatataaataataataaaaggaaggaaatttaggcggtagtgcgtagaacgagtatagggttttgtgagggcgcgtagggtttacgcgggtgtttttaatagggttttttacgtaggcggcgcgggtacggggtcgggggcgcgcggcgtttttagcgggagggcgtttggattaggggtttcgtttttttttagcgtattttcgtttttttttagcgtattttcgtttcgcgttaggtttattcgtagggttttttttagtacgttcgcggtcgtagtagttagtttagtatttattttcgaagttcgaaatgattttatttagttgcgcgttgatcgcggggttcgatatgatggttggtgggtagcgggtcgcgcggagggtagcggcgaggaagcgttttcggcggggttcgggttttgcgcgttggttggggcgtcgcgttcgcgtttttgtagtgtagagttagtcgtcggaggagtttttaggttttttggtttagttgaatgaatgggggaggaaggcgggcgtcggttttttttcgcgcgttgcgttttcgtttcgttttcgtttttcgggcggattaggttttcgtattcgtatcggtttttttgtttcgtattggttgtttcgttcgtttgttaaggttaggtttggggggttggggttcgagtggtcgttgggggatagttttgggtatacgatcggagcgtttttttttttttcgtttggttcgcgtttaggggacggcgagagacgcggttttagttttttttttcgggcgtttcgttgcggttagatgtgggcggatgtggaggtcgggttatggcgaggagggcgggcgtttagaggggaagagatttttttttt

SEQ ID NO: 11 - DNA, 62 bp, Homo sapiens
ccgctgcccaccagccatcatgtcggaccccgcggtcaacgcgcagctggatgggatcattt

SEQ ID NO: 12 - DNA, 62 bp, Artificial Sequence
  converted
tcgttgtttattagttattatgtcggatttcgcggttaacgcgtagttggatgggattattt

SEQ ID NO: 13 - DNA, 62 bp, Artificial Sequence
  converted
aaataatcccatccaactacgcgttaaccgcgaaatccgacataataactaataaacaacga

SEQ ID NO: 14 - DNA, 62 bp, Artificial Sequence
  converted
ccgctacccaccaaccatcatatcgaaccccgcgatcaacgcgcaactaaataaaatcattt

SEQ ID NO: 15 - DNA, 62 bp, Artificial Sequence
  converted
aaatgattttatttagttgcgcgttgatcgcggggttcgatatgatggttggtgggtagcgg

SEQ ID NO: 16 - DNA, 3318 bp, Homo sapiens
ccgggtttctaaccggcaggcgcggcccaggttcccggaggcagccccagagtcgcggaccgatgtgccaggctgtggatgagccctgggtaggggagggttcgtaccagcggcgcctggggcagcgaggagcgcgcgttctgcctgcgaagctgccttctccgagccccgcccaggaacattagctctggggggccgctgatcattgatttggacggagagatgggttctgggttctgtattaggattccagcatctgggctcgaggcagggcaatatccagaaagaccccagggttcggggtacccgggccagggctgaggcgcatcgccgagcaaaggctgggtgcgaggcgtgcggaatgatgcgcttgccttgcccgggcctctccaaggatggagaaaaggcgagtgaagcagcgaagtacgactccaaccccgcccagagagtgctactagcgctggctgcacgccaagtctctccaggggtccaaagcgagagggatttgttttaacccatctctacccgtcctgtgtcaagaacggaggctgtagagggcgactgcgaagtcgccaggcactcgctggatctcggtccccctcctcgtgctctggggttgagatggggcaccgccatcgataacagatcagcgcgaactattcgtttagtggccttaaaacaccctggtttcaccctcagctattttcaagttcccgtgtgcctggcactttctccgtgcgagaagcaccggagggtgcggacgcgccacagtctgagccgccgccgaactggctaagtttaggggcatttattattcatgttcctgccagatcctcgcctgcccaaaatagaaaccgaggttctccgtgacctacatctgctcggagaagggctcccctgggctcggaggctggggtgggggtggctgaggagttggcccccgcacgccccacgcatcctctcctttgctttctgggcctccccattcgggtcttcgcgtgggtcagcgcccggtctcccagggcctttctcgtccccgcccgttgctgctttggggaggctcgggagccaggcggggaggggggcggtccttttccgtagacaggtgtgcgcgatcggcggagacgcctcggtttcccagcgcttgttgaggccgtggcccgcaggacgaccctttacccgcgaagggggggtgggcgggccgcccggcggggtaggagtggttgggtgtcgttgcctcctccttacctctgctcccacccccagtcctgggagaagagacaattctcagcggaggacttttatcacctgtgaaaatccgcgcgagccccttactttggatcctcgccgagctggggaggaacttgcactgaccacaccttctgtccccggccaccccgcaggccagaggaagaggcgtacggcgaggacggaaacccgctgccagacttcgatggctcggagccgccgggcgcagggagccccgcctccgcgccgcgcgccgccgccgcctggtaccgcccggccgggagaaggtgagattcgcgcggcctcgcgcacacccgcggctgggagctcgggactgcggtgacgggaggggcagtgtggtgacccacccaggatttttttttttttcccgtgaaagtcctcaagcctgtcctctccctggcccgatcctattgcagcgacagaaaatcagcagcgggcgggtctgtgtggacctgagggccgcgtggggaccgaggggggctgtggcccaaagagtggcagtgagtggcgtcaaggaacccacactccgcatctgccactcctagagccgggactagctcccgatcctagcagttgctctcgagatcatcccgggagttattggcgagttctgggcctctggaggtttccctgtcagcctccccggccgccgagggggcgcgcgcccaacaagggggtctctagcggccacctggggacagaaacagtgaccctgggcgcgcactttgcctccccgttagagatgtcgcccacgggatccttaacgaggcctaccgcaaagtgctggaccagctgtccgccgggaagcacctgcagtcgctcgtggcccggggcgtggggtaagagtttgtggaaggattaacctgcgcgcgccggggtgggtgcctgtgcggggcgcgcggggcgggcggcggtgggtgcccgtgggggccagggtgagtctgcgcccctgggtctggggtgggcatccgccacgggtcgcagttggagattttgaagtggcactttaaatttgcccagagagctctggaagaggcaaaaagggaacgcgagccagggagtttgatccgttttgaatgaaaagaaagagaaaccaaaccaaacctctcagtcatccaaaaccttcaggcttccagggaggttttgctataattttctctaagcatgactgtttctgggggaggggaaaggggtggttgtatttactgaaaattcaaatcgaaataataaatggccaaatgtggacacttatggacccaaacagttttgctcacgccagagaaactgagagcacagggcttgtgtgaagcctatctcggcagaaggcaacattctaataaagcccgtgggaaaacagattacattttcgccatgaataagtcatgcagtgaaaaatattgcctacagcctgtcgacttatattattatcacgtttttcaactcggcgtgaggagggagaggagtgttcatatttgactaggaattgcaggatcgatgcaaactccagggcagcagccagactggcatatgtagggctctccggttactttctctgtatgtcgcgggtgagaggaacagcgaggacaatttagcgcaaacacacgaagggtcggatctcaagggggcagcgctgggagaaaggttaggcttgaagcgcgcgtcgcctgcccggatcttatcccgggccccctccgcagggtttggtgccaggagatcctgcgtggggaggggggcatcgaggggctgccgtctcggccctccccacggctgcttccaggcagaggcgggcgacgcggtgggcagtgcgagccccgggccctccccgaaggctcccgcgtggggtggggcccgcctgctccccgcggcgattgaacctgtgtctcccgccccgccaccctcttcccgacccctttgcttgcagtgggagcctcggcggcggcgcgggggacgacgcggagccgctctccaagcgccactcggacgggatcttcacggacagctacagccgctaccggaaacaaatggctgtcaagaaatacttggcggccg

SEQ ID NO: 17 - DNA, 3318 bp, Artificial Sequence
  converted
tcgggtttttaatcggtaggcgcggtttaggttttcggaggtagttttagagtcgcggatcgatgtgttaggttgtggatgagttttgggtaggggagggttcgtattagcggcgtttggggtagcgaggagcgcgcgttttgtttgcgaagttgtttttttcgagtttcgtttaggaatattagttttggggggtcgttgattattgatttggacggagagatgggttttgggttttgtattaggattttagtatttgggttcgaggtagggtaatatttagaaagattttagggttcggggtattcgggttagggttgaggcgtatcgtcgagtaaaggttgggtgcgaggcgtgcggaatgatgcgtttgttttgttcgggtttttttaaggatggagaaaaggcgagtgaagtagcgaagtacgattttaatttcgtttagagagtgttattagcgttggttgtacgttaagtttttttaggggtttaaagcgagagggatttgttttaatttatttttattcgttttgtgttaagaacggaggttgtagagggcgattgcgaagtcgttaggtattcgttggatttcggttttttttttcgtgttttggggttgagatggggtatcgttatcgataatagattagcgcgaattattcgtttagtggttttaaaatattttggttttatttttagttatttttaagttttcgtgtgtttggtatttttttcgtgcgagaagtatcggagggtgcggacgcgttatagtttgagtcgtcgtcgaattggttaagtttaggggtatttattatttatgtttttgttagattttcgtttgtttaaaatagaaatcgaggtttttcgtgatttatatttgttcggagaagggtttttttgggttcggaggttggggtgggggtggttgaggagttggttttcgtacgttttacgtatttttttttttgttttttgggtttttttattcgggttttcgcgtgggttagcgttcggttttttagggtttttttcgttttcgttcgttgttgttttggggaggttcgggagttaggcggggaggggggcggttttttttcgtagataggtgtgcgcgatcggcggagacgtttcggttttttagcgtttgttgaggtcgtggttcgtaggacgattttttattcgcgaagggggggtgggcgggtcgttcggcggggtaggagtggttgggtgtcgttgttttttttttatttttgtttttatttttagttttgggagaagagataatttttagcggaggatttttattatttgtgaaaattcgcgcgagttttttattttggattttcgtcgagttggggaggaatttgtattgattatattttttgttttcggttatttcgtaggttagaggaagaggcgtacggcgaggacggaaattcgttgttagatttcgatggttcggagtcgtcgggcgtagggagtttcgttttcgcgtcgcgcgtcgtcgtcgtttggtatcgttcggtcgggagaaggtgagattcgcgcggtttcgcgtatattcgcggttgggagttcgggattgcggtgacgggaggggtagtgtggtgatttatttaggatttttttttttttttcgtgaaagtttttaagtttgttttttttttggttcgattttattgtagcgatagaaaattagtagcgggcgggtttgtgtggatttgagggtcgcgtggggatcgaggggggttgtggtttaaagagtggtagtgagtggcgttaaggaatttatatttcgtatttgttatttttagagtcgggattagttttcgattttagtagttgttttcgagattatttcgggagttattggcgagttttgggtttttggaggtttttttgttagttttttcggtcgtcgagggggcgcgcgtttaataagggggtttttagcggttatttggggatagaaatagtgattttgggcgcgtattttgttttttcgttagagatgtcgtttacgggatttttaacgaggtttatcgtaaagtgttggattagttgttcgtcgggaagtatttgtagtcgttcgtggttcggggcgtggggtaagagtttgtggaaggattaatttgcgcgcgtcggggtgggtgtttgtgcggggcgcgcggggcgggcggcggtgggtgttcgtgggggttagggtgagtttgcgtttttgggtttggggtgggtattcgttacgggtcgtagttggagattttgaagtggtattttaaatttgtttagagagttttggaagaggtaaaaagggaacgcgagttagggagtttgattcgttttgaatgaaaagaaagagaaattaaattaaattttttagttatttaaaatttttaggtttttagggaggttttgttataattttttttaagtatgattgtttttgggggaggggaaaggggtggttgtatttattgaaaatttaaatcgaaataataaatggttaaatgtggatatttatggatttaaatagttttgtttacgttagagaaattgagagtatagggtttgtgtgaagtttatttcggtagaaggtaatattttaataaagttcgtgggaaaatagattatattttcgttatgaataagttatgtagtgaaaaatattgtttatagtttgtcgatttatattattattacgttttttaattcggcgtgaggagggagaggagtgtttatatttgattaggaattgtaggatcgatgtaaattttagggtagtagttagattggtatatgtagggtttttcggttattttttttgtatgtcgcgggtgagaggaatagcgaggataatttagcgtaaatatacgaagggtcggattttaagggggtagcgttgggagaaaggttaggtttgaagcgcgcgtcgtttgttcggattttatttcgggtttttttcgtagggtttggtgttaggagattttgcgtggggaggggggtatcgaggggttgtcgtttcggttttttttacggttgtttttaggtagaggcgggcgacgcggtgggtagtgcgagtttcgggtttttttcgaaggttttcgcgtggggtggggttcgtttgtttttcgcggcgattgaatttgtgtttttcgtttcgttatttttttttcgatttttttgtttgtagtgggagtttcggcggcggcgcgggggacgacgcggagtcgttttttaagcgttattcggacgggatttttacggatagttatagtcgttatcggaaataaatggttgttaagaaatatttggcggtcg

SEQ ID NO: 18 - DNA, 3318 bp, Artificial Sequence
  converted
cgaccgccaaatatttcttaacaaccatttatttccgataacgactataactatccgtaaaaatcccgtccgaataacgcttaaaaaacgactccgcgtcgtcccccgcgccgccgccgaaactcccactacaaacaaaaaaatcgaaaaaaaaataacgaaacgaaaaacacaaattcaatcgccgcgaaaaacaaacgaaccccaccccacgcgaaaaccttcgaaaaaaacccgaaactcgcactacccaccgcgtcgcccgcctctacctaaaaacaaccgtaaaaaaaaccgaaacgacaacccctcgatacccccctccccacgcaaaatctcctaacaccaaaccctacgaaaaaaacccgaaataaaatccgaacaaacgacgcgcgcttcaaacctaacctttctcccaacgctacccccttaaaatccgacccttcgtatatttacgctaaattatcctcgctattcctctcacccgcgacatacaaaaaaaataaccgaaaaaccctacatataccaatctaactactaccctaaaatttacatcgatcctacaattcctaatcaaatataaacactcctctccctcctcacgccgaattaaaaaacgtaataataatataaatcgacaaactataaacaatatttttcactacataacttattcataacgaaaatataatctattttcccacgaactttattaaaatattaccttctaccgaaataaacttcacacaaaccctatactctcaatttctctaacgtaaacaaaactatttaaatccataaatatccacatttaaccatttattatttcgatttaaattttcaataaatacaaccacccctttcccctcccccaaaaacaatcatacttaaaaaaaattataacaaaacctccctaaaaacctaaaaattttaaataactaaaaaatttaatttaatttctctttcttttcattcaaaacgaatcaaactccctaactcgcgttccctttttacctcttccaaaactctctaaacaaatttaaaataccacttcaaaatctccaactacgacccgtaacgaatacccaccccaaacccaaaaacgcaaactcaccctaacccccacgaacacccaccgccgcccgccccgcgcgccccgcacaaacacccaccccgacgcgcgcaaattaatccttccacaaactcttaccccacgccccgaaccacgaacgactacaaatacttcccgacgaacaactaatccaacactttacgataaacctcgttaaaaatcccgtaaacgacatctctaacgaaaaaacaaaatacgcgcccaaaatcactatttctatccccaaataaccgctaaaaacccccttattaaacgcgcgccccctcgacgaccgaaaaaactaacaaaaaaacctccaaaaacccaaaactcgccaataactcccgaaataatctcgaaaacaactactaaaatcgaaaactaatcccgactctaaaaataacaaatacgaaatataaattccttaacgccactcactaccactctttaaaccacaacccccctcgatccccacgcgaccctcaaatccacacaaacccgcccgctactaattttctatcgctacaataaaatcgaaccaaaaaaaaaacaaacttaaaaactttcacgaaaaaaaaaaaaaaatcctaaataaatcaccacactacccctcccgtcaccgcaatcccgaactcccaaccgcgaatatacgcgaaaccgcgcgaatctcaccttctcccgaccgaacgataccaaacgacgacgacgcgcgacgcgaaaacgaaactccctacgcccgacgactccgaaccatcgaaatctaacaacgaatttccgtcctcgccgtacgcctcttcctctaacctacgaaataaccgaaaacaaaaaatataatcaatacaaattcctccccaactcgacgaaaatccaaaataaaaaactcgcgcgaattttcacaaataataaaaatcctccgctaaaaattatctcttctcccaaaactaaaaataaaaacaaaaataaaaaaaaaacaacgacacccaaccactcctaccccgccgaacgacccgcccacccccccttcgcgaataaaaaatcgtcctacgaaccacgacctcaacaaacgctaaaaaaccgaaacgtctccgccgatcgcgcacacctatctacgaaaaaaaaccgcccccctccccgcctaactcccgaacctccccaaaacaacaacgaacgaaaacgaaaaaaaccctaaaaaaccgaacgctaacccacgcgaaaacccgaataaaaaaacccaaaaaacaaaaaaaaaaatacgtaaaacgtacgaaaaccaactcctcaaccacccccaccccaacctccgaacccaaaaaaacccttctccgaacaaatataaatcacgaaaaacctcgatttctattttaaacaaacgaaaatctaacaaaaacataaataataaatacccctaaacttaaccaattcgacgacgactcaaactataacgcgtccgcaccctccgatacttctcgcacgaaaaaaataccaaacacacgaaaacttaaaaataactaaaaataaaaccaaaatattttaaaaccactaaacgaataattcgcgctaatctattatcgataacgataccccatctcaaccccaaaacacgaaaaaaaaaaccgaaatccaacgaatacctaacgacttcgcaatcgccctctacaacctccgttcttaacacaaaacgaataaaaataaattaaaacaaatccctctcgctttaaacccctaaaaaaacttaacgtacaaccaacgctaataacactctctaaacgaaattaaaatcgtacttcgctacttcactcgccttttctccatccttaaaaaaacccgaacaaaacaaacgcatcattccgcacgcctcgcacccaacctttactcgacgatacgcctcaaccctaacccgaataccccgaaccctaaaatctttctaaatattaccctacctcgaacccaaatactaaaatcctaatacaaaacccaaaacccatctctccgtccaaatcaataatcaacgaccccccaaaactaatattcctaaacgaaactcgaaaaaaacaacttcgcaaacaaaacgcgcgctcctcgctaccccaaacgccgctaatacgaaccctcccctacccaaaactcatccacaacctaacacatcgatccgcgactctaaaactacctccgaaaacctaaaccgcgcctaccgattaaaaacccga

SEQ ID NO: 19 - DNA, 3318 bp, Artificial Sequence
  converted
ccgaatttctaaccgacaaacgcgacccaaattcccgaaaacaaccccaaaatcgcgaaccgatataccaaactataaataaaccctaaataaaaaaaaattcgtaccaacgacgcctaaaacaacgaaaaacgcgcgttctacctacgaaactaccttctccgaaccccgcccaaaaacattaactctaaaaaaccgctaatcattaatttaaacgaaaaaataaattctaaattctatattaaaattccaacatctaaactcgaaacaaaacaatatccaaaaaaaccccaaaattcgaaatacccgaaccaaaactaaaacgcatcgccgaacaaaaactaaatacgaaacgtacgaaataatacgcttaccttacccgaacctctccaaaaataaaaaaaaaacgaataaaacaacgaaatacgactccaaccccgcccaaaaaatactactaacgctaactacacgccaaatctctccaaaaatccaaaacgaaaaaaatttattttaacccatctctacccgtcctatatcaaaaacgaaaactataaaaaacgactacgaaatcgccaaacactcgctaaatctcgatccccctcctcgtactctaaaattaaaataaaacaccgccatcgataacaaatcaacgcgaactattcgtttaataaccttaaaacaccctaatttcaccctcaactattttcaaattcccgtatacctaacactttctccgtacgaaaaacaccgaaaaatacgaacgcgccacaatctaaaccgccgccgaactaactaaatttaaaaacatttattattcatattcctaccaaatcctcgcctacccaaaataaaaaccgaaattctccgtaacctacatctactcgaaaaaaaactcccctaaactcgaaaactaaaataaaaataactaaaaaattaacccccgcacgccccacgcatcctctcctttactttctaaacctccccattcgaatcttcgcgtaaatcaacgcccgatctcccaaaacctttctcgtccccgcccgttactactttaaaaaaactcgaaaaccaaacgaaaaaaaaaacgatccttttccgtaaacaaatatacgcgatcgacgaaaacgcctcgatttcccaacgcttattaaaaccgtaacccgcaaaacgaccctttacccgcgaaaaaaaaataaacgaaccgcccgacgaaataaaaataattaaatatcgttacctcctccttacctctactcccacccccaatcctaaaaaaaaaaacaattctcaacgaaaaacttttatcacctataaaaatccgcgcgaaccccttactttaaatcctcgccgaactaaaaaaaaacttacactaaccacaccttctatccccgaccaccccgcaaaccaaaaaaaaaaacgtacgacgaaaacgaaaacccgctaccaaacttcgataactcgaaaccgccgaacgcaaaaaaccccgcctccgcgccgcgcgccgccgccgcctaataccgcccgaccgaaaaaaaataaaattcgcgcgacctcgcgcacacccgcgactaaaaactcgaaactacgataacgaaaaaaacaatataataacccacccaaaatttttttttttttcccgtaaaaatcctcaaacctatcctctccctaacccgatcctattacaacgacaaaaaatcaacaacgaacgaatctatataaacctaaaaaccgcgtaaaaaccgaaaaaaactataacccaaaaaataacaataaataacgtcaaaaaacccacactccgcatctaccactcctaaaaccgaaactaactcccgatcctaacaattactctcgaaatcatcccgaaaattattaacgaattctaaacctctaaaaatttccctatcaacctccccgaccgccgaaaaaacgcgcgcccaacaaaaaaatctctaacgaccacctaaaaacaaaaacaataaccctaaacgcgcactttacctccccgttaaaaatatcgcccacgaaatccttaacgaaacctaccgcaaaatactaaaccaactatccgccgaaaaacacctacaatcgctcgtaacccgaaacgtaaaataaaaatttataaaaaaattaacctacgcgcgccgaaataaatacctatacgaaacgcgcgaaacgaacgacgataaatacccgtaaaaaccaaaataaatctacgcccctaaatctaaaataaacatccgccacgaatcgcaattaaaaattttaaaataacactttaaatttacccaaaaaactctaaaaaaaacaaaaaaaaaacgcgaaccaaaaaatttaatccgttttaaataaaaaaaaaaaaaaaccaaaccaaacctctcaatcatccaaaaccttcaaacttccaaaaaaattttactataattttctctaaacataactatttctaaaaaaaaaaaaaaaaataattatatttactaaaaattcaaatcgaaataataaataaccaaatataaacacttataaacccaaacaattttactcacgccaaaaaaactaaaaacacaaaacttatataaaacctatctcgacaaaaaacaacattctaataaaacccgtaaaaaaacaaattacattttcgccataaataaatcatacaataaaaaatattacctacaacctatcgacttatattattatcacgtttttcaactcgacgtaaaaaaaaaaaaaaatattcatatttaactaaaaattacaaaatcgatacaaactccaaaacaacaaccaaactaacatatataaaactctccgattactttctctatatatcgcgaataaaaaaaacaacgaaaacaatttaacgcaaacacacgaaaaatcgaatctcaaaaaaacaacgctaaaaaaaaaattaaacttaaaacgcgcgtcgcctacccgaatcttatcccgaaccccctccgcaaaatttaataccaaaaaatcctacgtaaaaaaaaaaacatcgaaaaactaccgtctcgaccctccccacgactacttccaaacaaaaacgaacgacgcgataaacaatacgaaccccgaaccctccccgaaaactcccgcgtaaaataaaacccgcctactccccgcgacgattaaacctatatctcccgccccgccaccctcttcccgacccctttacttacaataaaaacctcgacgacgacgcgaaaaacgacgcgaaaccgctctccaaacgccactcgaacgaaatcttcacgaacaactacaaccgctaccgaaaacaaataactatcaaaaaatacttaacgaccg

SEQ ID NO: 20 - DNA, 3318 bp, Artificial Sequence
  converted
cggtcgttaagtattttttgatagttatttgttttcggtagcggttgtagttgttcgtgaagatttcgttcgagtggcgtttggagagcggtttcgcgtcgtttttcgcgtcgtcgtcgaggtttttattgtaagtaaaggggtcgggaagagggtggcggggcgggagatataggtttaatcgtcgcggggagtaggcgggttttattttacgcgggagttttcggggagggttcggggttcgtattgtttatcgcgtcgttcgtttttgtttggaagtagtcgtggggagggtcgagacggtagtttttcgatgtttttttttttacgtaggattttttggtattaaattttgcggagggggttcgggataagattcgggtaggcgacgcgcgttttaagtttaattttttttttagcgttgtttttttgagattcgatttttcgtgtgtttgcgttaaattgttttcgttgttttttttattcgcgatatatagagaaagtaatcggagagttttatatatgttagtttggttgttgttttggagtttgtatcgattttgtaatttttagttaaatatgaatatttttttttttttttacgtcgagttgaaaaacgtgataataatataagtcgataggttgtaggtaatattttttattgtatgatttatttatggcgaaaatgtaatttgtttttttacgggttttattagaatgttgttttttgtcgagataggttttatataagttttgtgtttttagtttttttggcgtgagtaaaattgtttgggtttataagtgtttatatttggttatttattatttcgatttgaatttttagtaaatataattatttttttttttttttttagaaatagttatgtttagagaaaattatagtaaaatttttttggaagtttgaaggttttggatgattgagaggtttggtttggtttttttttttttttatttaaaacggattaaattttttggttcgcgttttttttttgttttttttagagttttttgggtaaatttaaagtgttattttaaaatttttaattgcgattcgtggcggatgtttattttagatttaggggcgtagatttattttggtttttacgggtatttatcgtcgttcgtttcgcgcgtttcgtataggtatttatttcggcgcgcgtaggttaatttttttataaatttttattttacgtttcgggttacgagcgattgtaggtgtttttcggcggatagttggtttagtattttgcggtaggtttcgttaaggatttcgtgggcgatatttttaacggggaggtaaagtgcgcgtttagggttattgtttttgtttttaggtggtcgttagagatttttttgttgggcgcgcgttttttcggcggtcggggaggttgatagggaaatttttagaggtttagaattcgttaataattttcgggatgatttcgagagtaattgttaggatcgggagttagtttcggttttaggagtggtagatgcggagtgtgggttttttgacgttatttattgttattttttgggttatagtttttttcggtttttacgcggtttttaggtttatatagattcgttcgttgttgattttttgtcgttgtaataggatcgggttagggagaggataggtttgaggatttttacgggaaaaaaaaaaaaattttgggtgggttattatattgtttttttcgttatcgtagtttcgagtttttagtcgcgggtgtgcgcgaggtcgcgcgaattttattttttttcggtcgggcggtattaggcggcggcggcgcgcggcgcggaggcggggttttttgcgttcggcggtttcgagttatcgaagtttggtagcgggttttcgttttcgtcgtacgtttttttttttggtttgcggggtggtcggggatagaaggtgtggttagtgtaagtttttttttagttcggcgaggatttaaagtaaggggttcgcgcggatttttataggtgataaaagtttttcgttgagaattgtttttttttttaggattgggggtgggagtagaggtaaggaggaggtaacgatatttaattatttttatttcgtcgggcggttcgtttatttttttttcgcgggtaaagggtcgttttgcgggttacggttttaataagcgttgggaaatcgaggcgttttcgtcgatcgcgtatatttgtttacggaaaaggatcgttttttttttcgtttggttttcgagtttttttaaagtagtaacgggcggggacgagaaaggttttgggagatcgggcgttgatttacgcgaagattcgaatggggaggtttagaaagtaaaggagaggatgcgtggggcgtgcgggggttaattttttagttatttttattttagttttcgagtttaggggagttttttttcgagtagatgtaggttacggagaatttcggtttttattttgggtaggcgaggatttggtaggaatatgaataataaatgtttttaaatttagttagttcggcggcggtttagattgtggcgcgttcgtatttttcggtgtttttcgtacggagaaagtgttaggtatacgggaatttgaaaatagttgagggtgaaattagggtgttttaaggttattaaacgaatagttcgcgttgatttgttatcgatggcggtgttttattttaattttagagtacgaggagggggatcgagatttagcgagtgtttggcgatttcgtagtcgttttttatagttttcgtttttgatataggacgggtagagatgggttaaaataaatttttttcgttttggatttttggagagatttggcgtgtagttagcgttagtagtattttttgggcggggttggagtcgtatttcgttgttttattcgttttttttttatttttggagaggttcgggtaaggtaagcgtattatttcgtacgtttcgtatttagtttttgttcggcgatgcgttttagttttggttcgggtatttcgaattttggggtttttttggatattgttttgtttcgagtttagatgttggaattttaatatagaatttagaatttattttttcgtttaaattaatgattagcggttttttagagttaatgtttttgggcggggttcggagaaggtagtttcgtaggtagaacgcgcgtttttcgttgttttaggcgtcgttggtacgaatttttttttatttagggtttatttatagtttggtatatcggttcgcgattttggggttgttttcgggaatttgggtcgcgtttgtcggttagaaattcgg

SEQ ID NO: 21 - DNA, 1094 bp, Homo sapiens
gtaggggagggttcgtaccagcggcgcctggggcagcgaggagcgcgcgttctgcctgcgaagctgccttctccgagccccgcccaggaacattagctctggggggccgctgatcattgatttggacggagagatgggttctgggttctgtattaggattccagcatctgggctcgaggcagggcaatatccagaaagaccccagggttcggggtacccgggccagggctgaggcgcatcgccgagcaaaggctgggtgcgaggcgtgcggaatgatgcgcttgccttgcccgggcctctccaaggatggagaaaaggcgagtgaagcagcgaagtacgactccaaccccgcccagagagtgctactagcgctggctgcacgccaagtctctccaggggtccaaagcgagagggatttgttttaacccatctctacccgtcctgtgtcaagaacggaggctgtagagggcgactgcgaagtcgccaggcactcgctggatctcggtccccctcctcgtgctctggggttgagatggggcaccgccatcgataacagatcagcgcgaactattcgtttagtggccttaaaacaccctggtttcaccctcagctattttcaagttcccgtgtgcctggcactttctccgtgcgagaagcaccggagggtgcggacgcgccacagtctgagccgccgccgaactggctaagtttaggggcatttattattcatgttcctgccagatcctcgcctgcccaaaatagaaaccgaggttctccgtgacctacatctgctcggagaagggctcccctgggctcggaggctggggtgggggtggctgaggagttggcccccgcacgccccacgcatcctctcctttgctttctgggcctccccattcgggtcttcgcgtgggtcagcgcccggtctcccagggcctttctcgtccccgcccgttgctgctttggggaggctcgggagccaggcggggaggggggcggtccttttccgtagacaggtgtgcgcgatcggcggagacgcctcggtttcccagcgcttgttgaggccgtggcccgcaggacgaccctttacccgc

SEQ ID NO: 22 - DNA, 1094 bp, Artificial Sequence
  converted
gtaggggagggttcgtattagcggcgtttggggtagcgaggagcgcgcgttttgtttgcgaagttgtttttttcgagtttcgtttaggaatattagttttggggggtcgttgattattgatttggacggagagatgggttttgggttttgtattaggattttagtatttgggttcgaggtagggtaatatttagaaagattttagggttcggggtattcgggttagggttgaggcgtatcgtcgagtaaaggttgggtgcgaggcgtgcggaatgatgcgtttgttttgttcgggtttttttaaggatggagaaaaggcgagtgaagtagcgaagtacgattttaatttcgtttagagagtgttattagcgttggttgtacgttaagtttttttaggggtttaaagcgagagggatttgttttaatttatttttattcgttttgtgttaagaacggaggttgtagagggcgattgcgaagtcgttaggtattcgttggatttcggttttttttttcgtgttttggggttgagatggggtatcgttatcgataatagattagcgcgaattattcgtttagtggttttaaaatattttggttttatttttagttatttttaagttttcgtgtgtttggtatttttttcgtgcgagaagtatcggagggtgcggacgcgttatagtttgagtcgtcgtcgaattggttaagtttaggggtatttattatttatgtttttgttagattttcgtttgtttaaaatagaaatcgaggtttttcgtgatttatatttgttcggagaagggtttttttgggttcggaggttggggtgggggtggttgaggagttggttttcgtacgttttacgtatttttttttttgttttttgggtttttttattcgggttttcgcgtgggttagcgttcggttttttagggtttttttcgttttcgttcgttgttgttttggggaggttcgggagttaggcggggaggggggcggttttttttcgtagataggtgtgcgcgatcggcggagacgtttcggttttttagcgtttgttgaggtcgtggttcgtaggacgattttttattcgt

SEQ ID NO: 23 - DNA, 1094 bp, Artificial Sequence
  converted
acgaataaaaaatcgtcctacgaaccacgacctcaacaaacgctaaaaaaccgaaacgtctccgccgatcgcgcacacctatctacgaaaaaaaaccgcccccctccccgcctaactcccgaacctccccaaaacaacaacgaacgaaaacgaaaaaaaccctaaaaaaccgaacgctaacccacgcgaaaacccgaataaaaaaacccaaaaaacaaaaaaaaaaatacgtaaaacgtacgaaaaccaactcctcaaccacccccaccccaacctccgaacccaaaaaaacccttctccgaacaaatataaatcacgaaaaacctcgatttctattttaaacaaacgaaaatctaacaaaaacataaataataaatacccctaaacttaaccaattcgacgacgactcaaactataacgcgtccgcaccctccgatacttctcgcacgaaaaaaataccaaacacacgaaaacttaaaaataactaaaaataaaaccaaaatattttaaaaccactaaacgaataattcgcgctaatctattatcgataacgataccccatctcaaccccaaaacacgaaaaaaaaaaccgaaatccaacgaatacctaacgacttcgcaatcgccctctacaacctccgttcttaacacaaaacgaataaaaataaattaaaacaaatccctctcgctttaaacccctaaaaaaacttaacgtacaaccaacgctaataacactctctaaacgaaattaaaatcgtacttcgctacttcactcgccttttctccatccttaaaaaaacccgaacaaaacaaacgcatcattccgcacgcctcgcacccaacctttactcgacgatacgcctcaaccctaacccgaataccccgaaccctaaaatctttctaaatattaccctacctcgaacccaaatactaaaatcctaatacaaaacccaaaacccatctctccgtccaaatcaataatcaacgaccccccaaaactaatattcctaaacgaaactcgaaaaaaacaacttcgcaaacaaaacgcgcgctcctcgctaccccaaacgccgctaatacgaaccctcccctac

SEQ ID NO: 24 - DNA, 1094 bp, Artificial Sequence
  converted
ataaaaaaaaattcgtaccaacgacgcctaaaacaacgaaaaacgcgcgttctacctacgaaactaccttctccgaaccccgcccaaaaacattaactctaaaaaaccgctaatcattaatttaaacgaaaaaataaattctaaattctatattaaaattccaacatctaaactcgaaacaaaacaatatccaaaaaaaccccaaaattcgaaatacccgaaccaaaactaaaacgcatcgccgaacaaaaactaaatacgaaacgtacgaaataatacgcttaccttacccgaacctctccaaaaataaaaaaaaaacgaataaaacaacgaaatacgactccaaccccgcccaaaaaatactactaacgctaactacacgccaaatctctccaaaaatccaaaacgaaaaaaatttattttaacccatctctacccgtcctatatcaaaaacgaaaactataaaaaacgactacgaaatcgccaaacactcgctaaatctcgatccccctcctcgtactctaaaattaaaataaaacaccgccatcgataacaaatcaacgcgaactattcgtttaataaccttaaaacaccctaatttcaccctcaactattttcaaattcccgtatacctaacactttctccgtacgaaaaacaccgaaaaatacgaacgcgccacaatctaaaccgccgccgaactaactaaatttaaaaacatttattattcatattcctaccaaatcctcgcctacccaaaataaaaaccgaaattctccgtaacctacatctactcgaaaaaaaactcccctaaactcgaaaactaaaataaaaataactaaaaaattaacccccgcacgccccacgcatcctctcctttactttctaaacctccccattcgaatcttcgcgtaaatcaacgcccgatctcccaaaacctttctcgtccccgcccgttactactttaaaaaaactcgaaaaccaaacgaaaaaaaaaacgatccttttccgtaaacaaatatacgcgatcgacgaaaacgcctcgatttcccaacgcttattaaaaccgtaacccgcaaaacgaccctttacccgc

SEQ ID NO: 25 - DNA, 1094 bp, Artificial Sequence
  converted
gcgggtaaagggtcgttttgcgggttacggttttaataagcgttgggaaatcgaggcgttttcgtcgatcgcgtatatttgtttacggaaaaggatcgttttttttttcgtttggttttcgagtttttttaaagtagtaacgggcggggacgagaaaggttttgggagatcgggcgttgatttacgcgaagattcgaatggggaggtttagaaagtaaaggagaggatgcgtggggcgtgcgggggttaattttttagttatttttattttagttttcgagtttaggggagttttttttcgagtagatgtaggttacggagaatttcggtttttattttgggtaggcgaggatttggtaggaatatgaataataaatgtttttaaatttagttagttcggcggcggtttagattgtggcgcgttcgtatttttcggtgtttttcgtacggagaaagtgttaggtatacgggaatttgaaaatagttgagggtgaaattagggtgttttaaggttattaaacgaatagttcgcgttgatttgttatcgatggcggtgttttattttaattttagagtacgaggagggggatcgagatttagcgagtgtttggcgatttcgtagtcgttttttatagttttcgtttttgatataggacgggtagagatgggttaaaataaatttttttcgttttggatttttggagagatttggcgtgtagttagcgttagtagtattttttgggcggggttggagtcgtatttcgttgttttattcgttttttttttatttttggagaggttcgggtaaggtaagcgtattatttcgtacgtttcgtatttagtttttgttcggcgatgcgttttagttttggttcgggtatttcgaattttggggtttttttggatattgttttgtttcgagtttagatgttggaattttaatatagaatttagaatttattttttcgtttaaattaatgattagcggttttttagagttaatgtttttgggcggggttcggagaaggtagtttcgtaggtagaacgcgcgtttttcgttgttttaggcgtcgttggtacgaatttttttttat

SEQ ID NO: 26 - DNA, 94 bp, Homo sapiens
ctcggtccccctcctcgtgctctggggttgagatggggcaccgccatcgataacagatcagcgcgaactattcgtttagtggccttaaaacacc

SEQ ID NO: 27 - DNA, 94 bp, Artificial Sequence
  converted
ttcggttttttttttcgtgttttggggttgagatggggtatcgttatcgataatagattagcgcgaattattcgtttagtggttttaaaatatt

SEQ ID NO: 28 - DNA, 94 bp, Artificial Sequence
  converted
aatattttaaaaccactaaacgaataattcgcgctaatctattatcgataacgataccccatctcaaccccaaaacacgaaaaaaaaaaccgaa

SEQ ID NO: 29 - DNA, 94 bp, Artificial Sequence
  converted
ctcgatccccctcctcgtactctaaaattaaaataaaacaccgccatcgataacaaatcaacgcgaactattcgtttaataaccttaaaacacc

SEQ ID NO: 30 - DNA, 94 bp, Artificial Sequence
  converted
ggtgttttaaggttattaaacgaatagttcgcgttgatttgttatcgatggcggtgttttattttaattttagagtacgaggagggggatcgag

SEQ ID NO: 31 - DNA, 1079 bp, Homo sapiens
ctctaggaatgaaatcccctttaacattacaggtacacaaaagaaaatgcacacttggtaaaacagttgatcaattatagagtgttcccagggaccaatatctcacagagtacccaatcatcacacttcaaataaatgtataagcactttagcctttacttaccacattaacatacatataagaacatgcatagtttaatgcataggttaattctaacactttccataggatagactttgattaaagggtatcaaaaatcacttcctaaatgcagcaaaaaataaaaataaaaataccgatctgtagctacgaagctcttagaacactacattgatcaccatttacttccaaactgctttcccttccctctgcgaagtgaaaatgtccctgtagaccgcgttacatacactttatggtttctctatcaatgatcctaacaggtcacatcgccactgccattagatctccgtagctttggtcttctccaaattaagacgtggagaaggagcactgctgtccgccccaagaacggggtctgtccgcccgagctctaaggcgagcatcttcagggggacagtttctgcgaaggcggggagaggagtccctccccaacttcactcccctaatcaagccgcgggtgagataggcagccggcggtttgtgcccatctgtgggggcaagtcctaccttctgccaaaaggcgcgacgcatgcacaaaagatggatccaggctatctttctctgccatcagctcaggcaaatatttctcctcttccatagcgcggacttcggattgtccccgggcgaagcgcgaggttccgctcgctcggacgcaggcagggtcttggggcagcgcctggctcccgcgctgctcctcctccgcgcggcgagggatctctgtgcgtcctcactggcccatgcacccagcacctgcgactcccgccgtcgggctgcgtggccccgcgcccacacctgcccgtcccttccgtcgtccctcgctcgcgcagagccccggctcacaccagcggccttaactggagaggcgggaacaggacgcggcccacctccgccaaccact

SEQ ID NO: 32 - DNA, 1079 bp, Artificial Sequence
  converted
ttttaggaatgaaattttttttaatattataggtatataaaagaaaatgtatatttggtaaaatagttgattaattatagagtgtttttagggattaatattttatagagtatttaattattatattttaaataaatgtataagtattttagtttttatttattatattaatatatatataagaatatgtatagtttaatgtataggttaattttaatattttttataggatagattttgattaaagggtattaaaaattattttttaaatgtagtaaaaaataaaaataaaaatatcgatttgtagttacgaagtttttagaatattatattgattattatttatttttaaattgtttttttttttttttgcgaagtgaaaatgtttttgtagatcgcgttatatatattttatggtttttttattaatgattttaataggttatatcgttattgttattagattttcgtagttttggttttttttaaattaagacgtggagaaggagtattgttgttcgttttaagaacggggtttgttcgttcgagttttaaggcgagtatttttagggggatagtttttgcgaaggcggggagaggagtttttttttaattttatttttttaattaagtcgcgggtgagataggtagtcggcggtttgtgtttatttgtgggggtaagttttattttttgttaaaaggcgcgacgtatgtataaaagatggatttaggttatttttttttgttattagtttaggtaaatatttttttttttttatagcgcggatttcggattgttttcgggcgaagcgcgaggtttcgttcgttcggacgtaggtagggttttggggtagcgtttggttttcgcgttgtttttttttcgcgcggcgagggatttttgtgcgtttttattggtttatgtatttagtatttgcgattttcgtcgtcgggttgcgtggtttcgcgtttatatttgttcgtttttttcgtcgtttttcgttcgcgtagagtttcggtttatattagcggttttaattggagaggcgggaataggacgcggtttattttcgttaattatt

SEQ ID NO: 33 - DNA, 1079 bp, Artificial Sequence
  converted
aataattaacgaaaataaaccgcgtcctattcccgcctctccaattaaaaccgctaatataaaccgaaactctacgcgaacgaaaaacgacgaaaaaaacgaacaaatataaacgcgaaaccacgcaacccgacgacgaaaatcgcaaatactaaatacataaaccaataaaaacgcacaaaaatccctcgccgcgcgaaaaaaaaacaacgcgaaaaccaaacgctaccccaaaaccctacctacgtccgaacgaacgaaacctcgcgcttcgcccgaaaacaatccgaaatccgcgctataaaaaaaaaaaaatatttacctaaactaataacaaaaaaaaataacctaaatccatcttttatacatacgtcgcgccttttaacaaaaaataaaacttacccccacaaataaacacaaaccgccgactacctatctcacccgcgacttaattaaaaaaataaaattaaaaaaaaactcctctccccgccttcgcaaaaactatccccctaaaaatactcgccttaaaactcgaacgaacaaaccccgttcttaaaacgaacaacaatactccttctccacgtcttaatttaaaaaaaaccaaaactacgaaaatctaataacaataacgatataacctattaaaatcattaataaaaaaaccataaaatatatataacgcgatctacaaaaacattttcacttcgcaaaaaaaaaaaaaaacaatttaaaaataaataataatcaatataatattctaaaaacttcgtaactacaaatcgatatttttatttttattttttactacatttaaaaaataatttttaataccctttaatcaaaatctatcctataaaaaatattaaaattaacctatacattaaactatacatattcttatatatatattaatataataaataaaaactaaaatacttatacatttatttaaaatataataattaaatactctataaaatattaatccctaaaaacactctataattaatcaactattttaccaaatatacattttcttttatatacctataatattaaaaaaaatttcattcctaaaa

SEQ ID NO: 34 - DNA, 1079 bp, Artificial Sequence
  converted
ctctaaaaataaaatcccctttaacattacaaatacacaaaaaaaaatacacacttaataaaacaattaatcaattataaaatattcccaaaaaccaatatctcacaaaatacccaatcatcacacttcaaataaatatataaacactttaacctttacttaccacattaacatacatataaaaacatacataatttaatacataaattaattctaacactttccataaaataaactttaattaaaaaatatcaaaaatcacttcctaaatacaacaaaaaataaaaataaaaataccgatctataactacgaaactcttaaaacactacattaatcaccatttacttccaaactactttcccttccctctacgaaataaaaatatccctataaaccgcgttacatacactttataatttctctatcaataatcctaacaaatcacatcgccactaccattaaatctccgtaactttaatcttctccaaattaaaacgtaaaaaaaaaacactactatccgccccaaaaacgaaatctatccgcccgaactctaaaacgaacatcttcaaaaaaacaatttctacgaaaacgaaaaaaaaaatccctccccaacttcactcccctaatcaaaccgcgaataaaataaacaaccgacgatttatacccatctataaaaacaaatcctaccttctaccaaaaaacgcgacgcatacacaaaaaataaatccaaactatctttctctaccatcaactcaaacaaatatttctcctcttccataacgcgaacttcgaattatccccgaacgaaacgcgaaattccgctcgctcgaacgcaaacaaaatcttaaaacaacgcctaactcccgcgctactcctcctccgcgcgacgaaaaatctctatacgtcctcactaacccatacacccaacacctacgactcccgccgtcgaactacgtaaccccgcgcccacacctacccgtcccttccgtcgtccctcgctcgcgcaaaaccccgactcacaccaacgaccttaactaaaaaaacgaaaacaaaacgcgacccacctccgccaaccact

SEQ ID NO: 35 - DNA, 1079 bp, Artificial Sequence
  converted
agtggttggcggaggtgggtcgcgttttgttttcgtttttttagttaaggtcgttggtgtgagtcggggttttgcgcgagcgagggacgacggaagggacgggtaggtgtgggcgcggggttacgtagttcgacggcgggagtcgtaggtgttgggtgtatgggttagtgaggacgtatagagatttttcgtcgcgcggaggaggagtagcgcgggagttaggcgttgttttaagattttgtttgcgttcgagcgagcggaatttcgcgtttcgttcggggataattcgaagttcgcgttatggaagaggagaaatatttgtttgagttgatggtagagaaagatagtttggatttattttttgtgtatgcgtcgcgttttttggtagaaggtaggatttgtttttatagatgggtataaatcgtcggttgtttattttattcgcggtttgattaggggagtgaagttggggagggattttttttttcgttttcgtagaaattgtttttttgaagatgttcgttttagagttcgggcggatagatttcgtttttggggcggatagtagtgttttttttttacgttttaatttggagaagattaaagttacggagatttaatggtagtggcgatgtgatttgttaggattattgatagagaaattataaagtgtatgtaacgcggtttatagggatatttttatttcgtagagggaagggaaagtagtttggaagtaaatggtgattaatgtagtgttttaagagtttcgtagttatagatcggtatttttatttttattttttgttgtatttaggaagtgatttttgatattttttaattaaagtttattttatggaaagtgttagaattaatttatgtattaaattatgtatgtttttatatgtatgttaatgtggtaagtaaaggttaaagtgtttatatatttatttgaagtgtgatgattgggtattttgtgagatattggtttttgggaatattttataattgattaattgttttattaagtgtgtatttttttttgtgtatttgtaatgttaaaggggattttatttttagag

SEQ ID NO: 36 - DNA, 79 bp, Homo sapiens
gagaaggagcactgctgtccgccccaagaacggggtctgtccgcccgagctctaaggcgagcatcttcagggggacagt

SEQ ID NO: 37 - DNA, 79 bp, Artificial Sequence
  converted
gagaaggagtattgttgttcgttttaagaacggggtttgttcgttcgagttttaaggcgagtatttttagggggatagt

SEQ ID NO: 38 - DNA, 79 bp, Artificial Sequence
  converted
actatccccctaaaaatactcgccttaaaactcgaacgaacaaaccccgttcttaaaacgaacaacaatactccttctc

SEQ ID NO: 39 - DNA, 79 bp, Artificial Sequence
  converted
aaaaaaaaacactactatccgccccaaaaacgaaatctatccgcccgaactctaaaacgaacatcttcaaaaaaacaat

SEQ ID NO: 40 - DNA, 79 bp, Artificial Sequence
  converted
attgtttttttgaagatgttcgttttagagttcgggcggatagatttcgtttttggggcggatagtagtgttttttttt

SEQ ID NO: 41 - DNA, 1284 bp, Homo sapiens
tcgtcgaccctgattggccatgttctctgcggcacggggctggttgcagtggccggcgggcgcctggtgggcaccccggtccccccaagggtcggctgtccttccccactggtcacacaagagcggccgtccttccccagctcgaagcccgtagcacattcgcaggcaaagcctcccaagtcgtctaggcagttagggagctctgcgcatttgccagcacggaggtacctcccggggcagggacacaacacatcgcccgagagtttgtcccagcgagcgccgatttcgtccgcgatgcaagtaactgagatcgggagctgtccccggcagagcgcactcacctcggtcccaggtggactgaagtccagagcggcgctgtgcagctggaagggcgcgcgatagctcaagttagaggcggccccggggcgcggcgcaggacacaagacctcaaactggtacttgcacaggtagccgttggcgcgcaggtggcatcgcatctccttccagcctgcgggctcgaccccaccggtggcctggagtaccgcgcatctccgcgcggtgcaggagcgttggggctcctccacccactgcagcgtgtcgctttcgagaccgccggggtcggaggacagccaggagaaaccccgcaaaggctcgttctccagggtgcagtgggaacgcctgcgctccagtgcgacccagaacagcaggtctttggagccccctccgggccctgggcctgcccgcaggagcgcgagcacagcgcgcagctcggcgcccgcacgcacggtgctgagcgccccacctcgcaggatgcaggcctcctcggccgcctgccgcttcatggtagcgtggtgcaggctgtagcaggcccccgaggccgagcagccagcacggtcggcagtggggtgttcgccgccgcccggcccgggccagagcgcctgccagaggaggcacagggcgaacgccggcctcattctctgaggccccgcacgcagagctgctcccaacttggatctgtcccgctcgaggacgcagagctgtgtctggagcgcggtcaccgccctctacagctggcttccccaacccggttccccctcccttcccctccgccctcgctcccgacggaccccccaactgccaagaatccccccgcgggcccagcggccaacagtttcccttaggccgcgacaggaaatgtgtgcggagcgctgccaggcccggtgcagccctgagttattctggggtcaggaacacagctggcgccgggtacaggggacgcagtcg

SEQ ID NO: 42 - DNA, 1284 bp, Artificial Sequence
  converted
tcgtcgattttgattggttatgttttttgcggtacggggttggttgtagtggtcggcgggcgtttggtgggtatttcggtttttttaagggtcggttgtttttttttattggttatataagagcggtcgtttttttttagttcgaagttcgtagtatattcgtaggtaaagttttttaagtcgtttaggtagttagggagttttgcgtatttgttagtacggaggtatttttcggggtagggatataatatatcgttcgagagtttgttttagcgagcgtcgatttcgttcgcgatgtaagtaattgagatcgggagttgttttcggtagagcgtatttatttcggttttaggtggattgaagtttagagcggcgttgtgtagttggaagggcgcgcgatagtttaagttagaggcggtttcggggcgcggcgtaggatataagattttaaattggtatttgtataggtagtcgttggcgcgtaggtggtatcgtatttttttttagtttgcgggttcgattttatcggtggtttggagtatcgcgtattttcgcgcggtgtaggagcgttggggtttttttatttattgtagcgtgtcgttttcgagatcgtcggggtcggaggatagttaggagaaatttcgtaaaggttcgttttttagggtgtagtgggaacgtttgcgttttagtgcgatttagaatagtaggtttttggagtttttttcgggttttgggtttgttcgtaggagcgcgagtatagcgcgtagttcggcgttcgtacgtacggtgttgagcgttttatttcgtaggatgtaggttttttcggtcgtttgtcgttttatggtagcgtggtgtaggttgtagtaggttttcgaggtcgagtagttagtacggtcggtagtggggtgttcgtcgtcgttcggttcgggttagagcgtttgttagaggaggtatagggcgaacgtcggttttattttttgaggtttcgtacgtagagttgtttttaatttggatttgtttcgttcgaggacgtagagttgtgtttggagcgcggttatcgttttttatagttggtttttttaattcggtttttttttttttttttttcgttttcgttttcgacggattttttaattgttaagaattttttcgcgggtttagcggttaatagttttttttaggtcgcgataggaaatgtgtgcggagcgttgttaggttcggtgtagttttgagttattttggggttaggaatatagttggcgtcgggtataggggacgtagtcg

SEQ ID NO: 43 - DNA, 1284 bp, Artificial Sequence
  converted
cgactacgtcccctatacccgacgccaactatattcctaaccccaaaataactcaaaactacaccgaacctaacaacgctccgcacacatttcctatcgcgacctaaaaaaaactattaaccgctaaacccgcgaaaaaattcttaacaattaaaaaatccgtcgaaaacgaaaacgaaaaaaaaaaaaaaaaaaaccgaattaaaaaaaccaactataaaaaacgataaccgcgctccaaacacaactctacgtcctcgaacgaaacaaatccaaattaaaaacaactctacgtacgaaacctcaaaaaataaaaccgacgttcgccctatacctcctctaacaaacgctctaacccgaaccgaacgacgacgaacaccccactaccgaccgtactaactactcgacctcgaaaacctactacaacctacaccacgctaccataaaacgacaaacgaccgaaaaaacctacatcctacgaaataaaacgctcaacaccgtacgtacgaacgccgaactacgcgctatactcgcgctcctacgaacaaacccaaaacccgaaaaaaactccaaaaacctactattctaaatcgcactaaaacgcaaacgttcccactacaccctaaaaaacgaacctttacgaaatttctcctaactatcctccgaccccgacgatctcgaaaacgacacgctacaataaataaaaaaaccccaacgctcctacaccgcgcgaaaatacgcgatactccaaaccaccgataaaatcgaacccgcaaactaaaaaaaaatacgataccacctacgcgccaacgactacctatacaaataccaatttaaaatcttatatcctacgccgcgccccgaaaccgcctctaacttaaactatcgcgcgcccttccaactacacaacgccgctctaaacttcaatccacctaaaaccgaaataaatacgctctaccgaaaacaactcccgatctcaattacttacatcgcgaacgaaatcgacgctcgctaaaacaaactctcgaacgatatattatatccctaccccgaaaaatacctccgtactaacaaatacgcaaaactccctaactacctaaacgacttaaaaaactttacctacgaatatactacgaacttcgaactaaaaaaaaacgaccgctcttatataaccaataaaaaaaaacaaccgacccttaaaaaaaccgaaatacccaccaaacgcccgccgaccactacaaccaaccccgtaccgcaaaaaacataaccaatcaaaatcgacga

SEQ ID NO: 44 - DNA, 1284 bp, Artificial Sequence
  converted
tcgtcgaccctaattaaccatattctctacgacacgaaactaattacaataaccgacgaacgcctaataaacaccccgatccccccaaaaatcgactatccttccccactaatcacacaaaaacgaccgtccttccccaactcgaaacccgtaacacattcgcaaacaaaacctcccaaatcgtctaaacaattaaaaaactctacgcatttaccaacacgaaaatacctcccgaaacaaaaacacaacacatcgcccgaaaatttatcccaacgaacgccgatttcgtccgcgatacaaataactaaaatcgaaaactatccccgacaaaacgcactcacctcgatcccaaataaactaaaatccaaaacgacgctatacaactaaaaaaacgcgcgataactcaaattaaaaacgaccccgaaacgcgacgcaaaacacaaaacctcaaactaatacttacacaaataaccgttaacgcgcaaataacatcgcatctccttccaacctacgaactcgaccccaccgataacctaaaataccgcgcatctccgcgcgatacaaaaacgttaaaactcctccacccactacaacgtatcgctttcgaaaccgccgaaatcgaaaaacaaccaaaaaaaaccccgcaaaaactcgttctccaaaatacaataaaaacgcctacgctccaatacgacccaaaacaacaaatctttaaaaccccctccgaaccctaaacctacccgcaaaaacgcgaacacaacgcgcaactcgacgcccgcacgcacgatactaaacgccccacctcgcaaaatacaaacctcctcgaccgcctaccgcttcataataacgtaatacaaactataacaaacccccgaaaccgaacaaccaacacgatcgacaataaaatattcgccgccgcccgacccgaaccaaaacgcctaccaaaaaaaacacaaaacgaacgccgacctcattctctaaaaccccgcacgcaaaactactcccaacttaaatctatcccgctcgaaaacgcaaaactatatctaaaacgcgatcaccgccctctacaactaacttccccaacccgattccccctcccttcccctccgccctcgctcccgacgaaccccccaactaccaaaaatccccccgcgaacccaacgaccaacaatttcccttaaaccgcgacaaaaaatatatacgaaacgctaccaaacccgatacaaccctaaattattctaaaatcaaaaacacaactaacgccgaatacaaaaaacgcaatcg

SEQ ID NO: 45 - DNA, 1284 bp, Artificial Sequence
  converted
cgattgcgttttttgtattcggcgttagttgtgtttttgattttagaataatttagggttgtatcgggtttggtagcgtttcgtatatattttttgtcgcggtttaagggaaattgttggtcgttgggttcgcggggggatttttggtagttggggggttcgtcgggagcgagggcggaggggaagggagggggaatcgggttggggaagttagttgtagagggcggtgatcgcgttttagatatagttttgcgttttcgagcgggatagatttaagttgggagtagttttgcgtgcggggttttagagaatgaggtcggcgttcgttttgtgttttttttggtaggcgttttggttcgggtcgggcggcggcgaatattttattgtcgatcgtgttggttgttcggtttcgggggtttgttatagtttgtattacgttattatgaagcggtaggcggtcgaggaggtttgtattttgcgaggtggggcgtttagtatcgtgcgtgcgggcgtcgagttgcgcgttgtgttcgcgtttttgcgggtaggtttagggttcggagggggttttaaagatttgttgttttgggtcgtattggagcgtaggcgtttttattgtattttggagaacgagtttttgcggggttttttttggttgtttttcgatttcggcggtttcgaaagcgatacgttgtagtgggtggaggagttttaacgtttttgtatcgcgcggagatgcgcggtattttaggttatcggtggggtcgagttcgtaggttggaaggagatgcgatgttatttgcgcgttaacggttatttgtgtaagtattagtttgaggttttgtgttttgcgtcgcgtttcggggtcgtttttaatttgagttatcgcgcgtttttttagttgtatagcgtcgttttggattttagtttatttgggatcgaggtgagtgcgttttgtcggggatagttttcgattttagttatttgtatcgcggacgaaatcggcgttcgttgggataaattttcgggcgatgtgttgtgtttttgtttcgggaggtattttcgtgttggtaaatgcgtagagttttttaattgtttagacgatttgggaggttttgtttgcgaatgtgttacgggtttcgagttggggaaggacggtcgtttttgtgtgattagtggggaaggatagtcgatttttggggggatcggggtgtttattaggcgttcgtcggttattgtaattagtttcgtgtcgtagagaatatggttaattagggtcgacga

SEQ ID NO: 46 - DNA, 1102 bp, Homo sapiens
gtggactgaagtccagagcggcgctgtgcagctggaagggcgcgcgatagctcaagttagaggcggccccggggcgcggcgcaggacacaagacctcaaactggtacttgcacaggtagccgttggcgcgcaggtggcatcgcatctccttccagcctgcgggctcgaccccaccggtggcctggagtaccgcgcatctccgcgcggtgcaggagcgttggggctcctccacccactgcagcgtgtcgctttcgagaccgccggggtcggaggacagccaggagaaaccccgcaaaggctcgttctccagggtgcagtgggaacgcctgcgctccagtgcgacccagaacagcaggtctttggagccccctccgggccctgggcctgcccgcaggagcgcgagcacagcgcgcagctcggcgcccgcacgcacggtgctgagcgccccacctcgcaggatgcaggcctcctcggccgcctgccgcttcatggtagcgtggtgcaggctgtagcaggcccccgaggccgagcagccagcacggtcggcagtggggtgttcgccgccgcccggcccgggccagagcgcctgccagaggaggcacagggcgaacgccggcctcattctctgaggccccgcacgcagagctgctcccaacttggatctgtcccgctcgaggacgcagagctgtgtctggagcgcggtcaccgccctctacagctggcttccccaacccggttccccctcccttcccctccgccctcgctcccgacggaccccccaactgccaagaatccccccgcgggcccagcggccaacagtttcccttaggccgcgacaggaaatgtgtgcggagcgctgccaggcccggtgcagccctgagttattctggggtcaggaacacagctggcgccgggtacaggggacgcagtcgactagaaggtcaaacaccctgcttagagcaagaggagaaattcgccactgggatgaagtgtgtggtttttctccgttcagggagtttcatcaggaccgcagaacccataacacgtaagcgaacataggcaggaaggacttgagaaaggaactggcttatgattataagac

SEQ ID NO: 47 - DNA, 1102 bp, Artificial Sequence
  converted
gtggattgaagtttagagcggcgttgtgtagttggaagggcgcgcgatagtttaagttagaggcggtttcggggcgcggcgtaggatataagattttaaattggtatttgtataggtagtcgttggcgcgtaggtggtatcgtatttttttttagtttgcgggttcgattttatcggtggtttggagtatcgcgtattttcgcgcggtgtaggagcgttggggtttttttatttattgtagcgtgtcgttttcgagatcgtcggggtcggaggatagttaggagaaatttcgtaaaggttcgttttttagggtgtagtgggaacgtttgcgttttagtgcgatttagaatagtaggtttttggagtttttttcgggttttgggtttgttcgtaggagcgcgagtatagcgcgtagttcggcgttcgtacgtacggtgttgagcgttttatttcgtaggatgtaggttttttcggtcgtttgtcgttttatggtagcgtggtgtaggttgtagtaggttttcgaggtcgagtagttagtacggtcggtagtggggtgttcgtcgtcgttcggttcgggttagagcgtttgttagaggaggtatagggcgaacgtcggttttattttttgaggtttcgtacgtagagttgtttttaatttggatttgtttcgttcgaggacgtagagttgtgtttggagcgcggttatcgttttttatagttggtttttttaattcggtttttttttttttttttttcgttttcgttttcgacggattttttaattgttaagaattttttcgcgggtttagcggttaatagttttttttaggtcgcgataggaaatgtgtgcggagcgttgttaggttcggtgtagttttgagttattttggggttaggaatatagttggcgtcgggtataggggacgtagtcgattagaaggttaaatattttgtttagagtaagaggagaaattcgttattgggatgaagtgtgtggttttttttcgtttagggagttttattaggatcgtagaatttataatacgtaagcgaatataggtaggaaggatttgagaaaggaattggtttatgattataagat

SEQ ID NO: 48 - DNA, 1102 bp, Artificial Sequence
  converted
atcttataatcataaaccaattcctttctcaaatccttcctacctatattcgcttacgtattataaattctacgatcctaataaaactccctaaacgaaaaaaaaccacacacttcatcccaataacgaatttctcctcttactctaaacaaaatatttaaccttctaatcgactacgtcccctatacccgacgccaactatattcctaaccccaaaataactcaaaactacaccgaacctaacaacgctccgcacacatttcctatcgcgacctaaaaaaaactattaaccgctaaacccgcgaaaaaattcttaacaattaaaaaatccgtcgaaaacgaaaacgaaaaaaaaaaaaaaaaaaaccgaattaaaaaaaccaactataaaaaacgataaccgcgctccaaacacaactctacgtcctcgaacgaaacaaatccaaattaaaaacaactctacgtacgaaacctcaaaaaataaaaccgacgttcgccctatacctcctctaacaaacgctctaacccgaaccgaacgacgacgaacaccccactaccgaccgtactaactactcgacctcgaaaacctactacaacctacaccacgctaccataaaacgacaaacgaccgaaaaaacctacatcctacgaaataaaacgctcaacaccgtacgtacgaacgccgaactacgcgctatactcgcgctcctacgaacaaacccaaaacccgaaaaaaactccaaaaacctactattctaaatcgcactaaaacgcaaacgttcccactacaccctaaaaaacgaacctttacgaaatttctcctaactatcctccgaccccgacgatctcgaaaacgacacgctacaataaataaaaaaaccccaacgctcctacaccgcgcgaaaatacgcgatactccaaaccaccgataaaatcgaacccgcaaactaaaaaaaaatacgataccacctacgcgccaacgactacctatacaaataccaatttaaaatcttatatcctacgccgcgccccgaaaccgcctctaacttaaactatcgcgcgcccttccaactacacaacgccgctctaaacttcaatccac

SEQ ID NO: 49 - DNA, 1102 bp, Artificial Sequence
  converted
ataaactaaaatccaaaacgacgctatacaactaaaaaaacgcgcgataactcaaattaaaaacgaccccgaaacgcgacgcaaaacacaaaacctcaaactaatacttacacaaataaccgttaacgcgcaaataacatcgcatctccttccaacctacgaactcgaccccaccgataacctaaaataccgcgcatctccgcgcgatacaaaaacgttaaaactcctccacccactacaacgtatcgctttcgaaaccgccgaaatcgaaaaacaaccaaaaaaaaccccgcaaaaactcgttctccaaaatacaataaaaacgcctacgctccaatacgacccaaaacaacaaatctttaaaaccccctccgaaccctaaacctacccgcaaaaacgcgaacacaacgcgcaactcgacgcccgcacgcacgatactaaacgccccacctcgcaaaatacaaacctcctcgaccgcctaccgcttcataataacgtaatacaaactataacaaacccccgaaaccgaacaaccaacacgatcgacaataaaatattcgccgccgcccgacccgaaccaaaacgcctaccaaaaaaaacacaaaacgaacgccgacctcattctctaaaaccccgcacgcaaaactactcccaacttaaatctatcccgctcgaaaacgcaaaactatatctaaaacgcgatcaccgccctctacaactaacttccccaacccgattccccctcccttcccctccgccctcgctcccgacgaaccccccaactaccaaaaatccccccgcgaacccaacgaccaacaatttcccttaaaccgcgacaaaaaatatatacgaaacgctaccaaacccgatacaaccctaaattattctaaaatcaaaaacacaactaacgccgaatacaaaaaacgcaatcgactaaaaaatcaaacaccctacttaaaacaaaaaaaaaaattcgccactaaaataaaatatataatttttctccgttcaaaaaatttcatcaaaaccgcaaaacccataacacgtaaacgaacataaacaaaaaaaacttaaaaaaaaaactaacttataattataaaac

SEQ ID NO: 50 - DNA, 1102 bp, Artificial Sequence
  converted
gttttataattataagttagttttttttttaagttttttttgtttatgttcgtttacgtgttatgggttttgcggttttgatgaaattttttgaacggagaaaaattatatattttattttagtggcgaatttttttttttgttttaagtagggtgtttgattttttagtcgattgcgttttttgtattcggcgttagttgtgtttttgattttagaataatttagggttgtatcgggtttggtagcgtttcgtatatattttttgtcgcggtttaagggaaattgttggtcgttgggttcgcggggggatttttggtagttggggggttcgtcgggagcgagggcggaggggaagggagggggaatcgggttggggaagttagttgtagagggcggtgatcgcgttttagatatagttttgcgttttcgagcgggatagatttaagttgggagtagttttgcgtgcggggttttagagaatgaggtcggcgttcgttttgtgttttttttggtaggcgttttggttcgggtcgggcggcggcgaatattttattgtcgatcgtgttggttgttcggtttcgggggtttgttatagtttgtattacgttattatgaagcggtaggcggtcgaggaggtttgtattttgcgaggtggggcgtttagtatcgtgcgtgcgggcgtcgagttgcgcgttgtgttcgcgtttttgcgggtaggtttagggttcggagggggttttaaagatttgttgttttgggtcgtattggagcgtaggcgtttttattgtattttggagaacgagtttttgcggggttttttttggttgtttttcgatttcggcggtttcgaaagcgatacgttgtagtgggtggaggagttttaacgtttttgtatcgcgcggagatgcgcggtattttaggttatcggtggggtcgagttcgtaggttggaaggagatgcgatgttatttgcgcgttaacggttatttgtgtaagtattagtttgaggttttgtgttttgcgtcgcgtttcggggtcgtttttaatttgagttatcgcgcgtttttttagttgtatagcgtcgttttggattttagtttat

SEQ ID NO: 51 - DNA, 102 bp, Homo sapiens
tgcaggctgtagcaggcccccgaggccgagcagccagcacggtcggcagtggggtgttcgccgccgcccggcccgggccagagcgcctgccagaggaggcac

SEQ ID NO: 52 - DNA, 102 bp, Artificial Sequence
  converted
tgtaggttgtagtaggttttcgaggtcgagtagttagtacggtcggtagtggggtgttcgtcgtcgttcggttcgggttagagcgtttgttagaggaggtat

SEQ ID NO: 53 - DNA, 102 bp, Artificial Sequence
  converted
atacctcctctaacaaacgctctaacccgaaccgaacgacgacgaacaccccactaccgaccgtactaactactcgacctcgaaaacctactacaacctaca

SEQ ID NO: 54 - DNA, 102 bp, Artificial Sequence
  converted
tacaaactataacaaacccccgaaaccgaacaaccaacacgatcgacaataaaatattcgccgccgcccgacccgaaccaaaacgcctaccaaaaaaaacac

SEQ ID NO: 55 - DNA, 102 bp, Artificial Sequence
  converted
gtgttttttttggtaggcgttttggttcgggtcgggcggcggcgaatattttattgtcgatcgtgttggttgttcggtttcgggggtttgttatagtttgta

SEQ ID NO: 56 - DNA, 1327 bp, Homo sapiens
acgcccgcatcaccttcgggaacctcaacggctgcgacgaaccggtgccatcggtgcgaggcagccccagcagcgagacgccttccccaggcagcgactcctccagcgtcagcagctccagctccacgagtgaggccccccgcgagaacgcctgcccctcggctctccccggggtgggtgggaggggcgcgcccggccgtgcctgacccagcccccgcgcccccagcctcctgccgcggctgcgagatctccccagcgttgttcgaggccccgcgcggctacagcatgatgggcggccagcgggaggcggcgacccgggacgacgacgacgacctgctgcaattcgccatccagcagagcctgcttgaggcgggcagtgagtatgaccaggtgcgtctccgcgggcgcgcggggccacgggactccgcgccgccacggctaacgcccacgcctccccgcaggtcaccatctgggaggcgctaaccaacagcaagccaggcacccaccccatgtcctacgagggtcgccgacaggacaggtcagtgcccgctgggccggagagaatcctccggagaggatcctgtctcccctaagccaggacacagccggagaacccgcggggccctcggcagggaccctcctggtatcgggatggcttctgccaccactcctgcctccgaggcggcaggcaggggtcagggatccaggcttcaccgcggagttcagactctccccaggcattgacggggagaacttggtagggtctgggctccgccacgaccaggtgggggccaggacgccgcgggactgaccgcctccactagagggtggtggccgcgggccgccctggagcctccacgccgtgtcccggccccggccggtgggtgggtgcgcccgggagcccgcgacattccttccccacccccaggagcgccccgcccacgccgcagcgccagcctgcgcccccggcgtcagtgcccagccctcggcccagctcagggccaggttccggcggccacgtgttccggagctacgacgagcagctgcggctggcgatggaactgtcggcgcaggagcaggaggagaggcggcggcgcgcgcgccaggaggaggaggagctggagcgcatcctgaggctctcactgaccgagcagtagcgccccctgccgggaccctcgccagcgccacgcgcgccacgcccagggccaggagccagacaaaccccggcctgcgcgcctgcagagcggcggctggagactggagccaccgcctcgcgggtgcagcagcacagcaggcacggttcggggagggattcggcatggccgcg

SEQ ID NO: 57 - DNA, 1327 bp, Artificial Sequence
  converted
acgttcgtattattttcgggaattttaacggttgcgacgaatcggtgttatcggtgcgaggtagttttagtagcgagacgttttttttaggtagcgatttttttagcgttagtagttttagttttacgagtgaggtttttcgcgagaacgtttgtttttcggtttttttcggggtgggtgggaggggcgcgttcggtcgtgtttgatttagttttcgcgtttttagttttttgtcgcggttgcgagatttttttagcgttgttcgaggtttcgcgcggttatagtatgatgggcggttagcgggaggcggcgattcgggacgacgacgacgatttgttgtaattcgttatttagtagagtttgtttgaggcgggtagtgagtatgattaggtgcgttttcgcgggcgcgcggggttacgggatttcgcgtcgttacggttaacgtttacgttttttcgtaggttattatttgggaggcgttaattaatagtaagttaggtatttattttatgttttacgagggtcgtcgataggataggttagtgttcgttgggtcggagagaatttttcggagaggattttgttttttttaagttaggatatagtcggagaattcgcggggttttcggtagggatttttttggtatcgggatggtttttgttattatttttgttttcgaggcggtaggtaggggttagggatttaggttttatcgcggagtttagattttttttaggtattgacggggagaatttggtagggtttgggtttcgttacgattaggtgggggttaggacgtcgcgggattgatcgtttttattagagggtggtggtcgcgggtcgttttggagtttttacgtcgtgtttcggtttcggtcggtgggtgggtgcgttcgggagttcgcgatattttttttttatttttaggagcgtttcgtttacgtcgtagcgttagtttgcgttttcggcgttagtgtttagttttcggtttagtttagggttaggtttcggcggttacgtgtttcggagttacgacgagtagttgcggttggcgatggaattgtcggcgtaggagtaggaggagaggcggcggcgcgcgcgttaggaggaggaggagttggagcgtattttgaggtttttattgatcgagtagtagcgttttttgtcgggattttcgttagcgttacgcgcgttacgtttagggttaggagttagataaatttcggtttgcgcgtttgtagagcggcggttggagattggagttatcgtttcgcgggtgtagtagtatagtaggtacggttcggggagggattcggtatggtcgcg

SEQ ID NO: 58 - DNA, 1327 bp, Artificial Sequence
  converted
cgcgaccataccgaatccctccccgaaccgtacctactatactactacacccgcgaaacgataactccaatctccaaccgccgctctacaaacgcgcaaaccgaaatttatctaactcctaaccctaaacgtaacgcgcgtaacgctaacgaaaatcccgacaaaaaacgctactactcgatcaataaaaacctcaaaatacgctccaactcctcctcctcctaacgcgcgcgccgccgcctctcctcctactcctacgccgacaattccatcgccaaccgcaactactcgtcgtaactccgaaacacgtaaccgccgaaacctaaccctaaactaaaccgaaaactaaacactaacgccgaaaacgcaaactaacgctacgacgtaaacgaaacgctcctaaaaataaaaaaaaaatatcgcgaactcccgaacgcacccacccaccgaccgaaaccgaaacacgacgtaaaaactccaaaacgacccgcgaccaccaccctctaataaaaacgatcaatcccgcgacgtcctaacccccacctaatcgtaacgaaacccaaaccctaccaaattctccccgtcaatacctaaaaaaaatctaaactccgcgataaaacctaaatccctaacccctacctaccgcctcgaaaacaaaaataataacaaaaaccatcccgataccaaaaaaatccctaccgaaaaccccgcgaattctccgactatatcctaacttaaaaaaaacaaaatcctctccgaaaaattctctccgacccaacgaacactaacctatcctatcgacgaccctcgtaaaacataaaataaatacctaacttactattaattaacgcctcccaaataataacctacgaaaaaacgtaaacgttaaccgtaacgacgcgaaatcccgtaaccccgcgcgcccgcgaaaacgcacctaatcatactcactacccgcctcaaacaaactctactaaataacgaattacaacaaatcgtcgtcgtcgtcccgaatcgccgcctcccgctaaccgcccatcatactataaccgcgcgaaacctcgaacaacgctaaaaaaatctcgcaaccgcgacaaaaaactaaaaacgcgaaaactaaatcaaacacgaccgaacgcgcccctcccacccaccccgaaaaaaaccgaaaaacaaacgttctcgcgaaaaacctcactcgtaaaactaaaactactaacgctaaaaaaatcgctacctaaaaaaaacgtctcgctactaaaactacctcgcaccgataacaccgattcgtcgcaaccgttaaaattcccgaaaataatacgaacgt

SEQ ID NO: 59 - DNA, 1327 bp, Artificial Sequence
  converted
acgcccgcatcaccttcgaaaacctcaacgactacgacgaaccgataccatcgatacgaaacaaccccaacaacgaaacgccttccccaaacaacgactcctccaacgtcaacaactccaactccacgaataaaaccccccgcgaaaacgcctacccctcgactctccccgaaataaataaaaaaaacgcgcccgaccgtacctaacccaacccccgcgcccccaacctcctaccgcgactacgaaatctccccaacgttattcgaaaccccgcgcgactacaacataataaacgaccaacgaaaaacgacgacccgaaacgacgacgacgacctactacaattcgccatccaacaaaacctacttaaaacgaacaataaatataaccaaatacgtctccgcgaacgcgcgaaaccacgaaactccgcgccgccacgactaacgcccacgcctccccgcaaatcaccatctaaaaaacgctaaccaacaacaaaccaaacacccaccccatatcctacgaaaatcgccgacaaaacaaatcaatacccgctaaaccgaaaaaaatcctccgaaaaaaatcctatctcccctaaaccaaaacacaaccgaaaaacccgcgaaaccctcgacaaaaaccctcctaatatcgaaataacttctaccaccactcctacctccgaaacgacaaacaaaaatcaaaaatccaaacttcaccgcgaaattcaaactctccccaaacattaacgaaaaaaacttaataaaatctaaactccgccacgaccaaataaaaaccaaaacgccgcgaaactaaccgcctccactaaaaaataataaccgcgaaccgccctaaaacctccacgccgtatcccgaccccgaccgataaataaatacgcccgaaaacccgcgacattccttccccacccccaaaaacgccccgcccacgccgcaacgccaacctacgcccccgacgtcaatacccaaccctcgacccaactcaaaaccaaattccgacgaccacgtattccgaaactacgacgaacaactacgactaacgataaaactatcgacgcaaaaacaaaaaaaaaaacgacgacgcgcgcgccaaaaaaaaaaaaaactaaaacgcatcctaaaactctcactaaccgaacaataacgccccctaccgaaaccctcgccaacgccacgcgcgccacgcccaaaaccaaaaaccaaacaaaccccgacctacgcgcctacaaaacgacgactaaaaactaaaaccaccgcctcgcgaatacaacaacacaacaaacacgattcgaaaaaaaattcgacataaccgcg

SEQ ID NO: 60 - DNA, 1327 bp, Artificial Sequence
  converted
cgcggttatgtcgaattttttttcgaatcgtgtttgttgtgttgttgtattcgcgaggcggtggttttagtttttagtcgtcgttttgtaggcgcgtaggtcggggtttgtttggtttttggttttgggcgtggcgcgcgtggcgttggcgagggtttcggtagggggcgttattgttcggttagtgagagttttaggatgcgttttagttttttttttttttggcgcgcgcgtcgtcgtttttttttttgtttttgcgtcgatagttttatcgttagtcgtagttgttcgtcgtagtttcggaatacgtggtcgtcggaatttggttttgagttgggtcgagggttgggtattgacgtcgggggcgtaggttggcgttgcggcgtgggcggggcgtttttgggggtggggaaggaatgtcgcgggttttcgggcgtatttatttatcggtcggggtcgggatacggcgtggaggttttagggcggttcgcggttattattttttagtggaggcggttagtttcgcggcgttttggtttttatttggtcgtggcggagtttagattttattaagtttttttcgttaatgtttggggagagtttgaatttcgcggtgaagtttggatttttgatttttgtttgtcgtttcggaggtaggagtggtggtagaagttatttcgatattaggagggtttttgtcgagggtttcgcgggtttttcggttgtgttttggtttaggggagataggatttttttcggaggatttttttcggtttagcgggtattgatttgttttgtcggcgattttcgtaggatatggggtgggtgtttggtttgttgttggttagcgttttttagatggtgatttgcggggaggcgtgggcgttagtcgtggcggcgcggagtttcgtggtttcgcgcgttcgcggagacgtatttggttatatttattgttcgttttaagtaggttttgttggatggcgaattgtagtaggtcgtcgtcgtcgtttcgggtcgtcgtttttcgttggtcgtttattatgttgtagtcgcgcggggtttcgaataacgttggggagatttcgtagtcgcggtaggaggttgggggcgcgggggttgggttaggtacggtcgggcgcgttttttttatttatttcggggagagtcgaggggtaggcgttttcgcggggggttttattcgtggagttggagttgttgacgttggaggagtcgttgtttggggaaggcgtttcgttgttggggttgtttcgtatcgatggtatcggttcgtcgtagtcgttgaggttttcgaaggtgatgcgggcgt

SEQ ID NO: 61 - DNA, 1103 bp, Homo sapiens
tgccaccactcctgcctccgaggcggcaggcaggggtcagggatccaggcttcaccgcggagttcagactctccccaggcattgacggggagaacttggtagggtctgggctccgccacgaccaggtgggggccaggacgccgcgggactgaccgcctccactagagggtggtggccgcgggccgccctggagcctccacgccgtgtcccggccccggccggtgggtgggtgcgcccgggagcccgcgacattccttccccacccccaggagcgccccgcccacgccgcagcgccagcctgcgcccccggcgtcagtgcccagccctcggcccagctcagggccaggttccggcggccacgtgttccggagctacgacgagcagctgcggctggcgatggaactgtcggcgcaggagcaggaggagaggcggcggcgcgcgcgccaggaggaggaggagctggagcgcatcctgaggctctcactgaccgagcagtagcgccccctgccgggaccctcgccagcgccacgcgcgccacgcccagggccaggagccagacaaaccccggcctgcgcgcctgcagagcggcggctggagactggagccaccgcctcgcgggtgcagcagcacagcaggcacggttcggggagggattcggcatggccgcggggtaccttcccaggccagggccctggaggcaactggcacggcctggtccccctgctttgctgtattctgattccccaacccgctcccctgggctcagatctgtcctgtcctagggcggagccaggcggtcctgagggggagatgaatccttagaggagcgctgtccctatcccttgctccttctggccggtccgcatttctgttcactaaccccactccaggacactgcccctgaagcctttgcatctctgctcttcactcctggggggcagctgagctccccgcgtgctgtgtcactgctttgttccagacacaaaccagcacgtctagggcccagcccctcccccaccccggcatttcagcgtcaagtgcacttagcggggtacccggctcccccagcccccacacccgtcctgggtctcagggtggttcca

SEQ ID NO: 62 - DNA, 1103 bp, Artificial Sequence
  converted
tgttattatttttgttttcgaggcggtaggtaggggttagggatttaggttttatcgcggagtttagattttttttaggtattgacggggagaatttggtagggtttgggtttcgttacgattaggtgggggttaggacgtcgcgggattgatcgtttttattagagggtggtggtcgcgggtcgttttggagtttttacgtcgtgtttcggtttcggtcggtgggtgggtgcgttcgggagttcgcgatattttttttttatttttaggagcgtttcgtttacgtcgtagcgttagtttgcgttttcggcgttagtgtttagttttcggtttagtttagggttaggtttcggcggttacgtgtttcggagttacgacgagtagttgcggttggcgatggaattgtcggcgtaggagtaggaggagaggcggcggcgcgcgcgttaggaggaggaggagttggagcgtattttgaggtttttattgatcgagtagtagcgttttttgtcgggattttcgttagcgttacgcgcgttacgtttagggttaggagttagataaatttcggtttgcgcgtttgtagagcggcggttggagattggagttatcgtttcgcgggtgtagtagtatagtaggtacggttcggggagggattcggtatggtcgcggggtatttttttaggttagggttttggaggtaattggtacggtttggtttttttgttttgttgtattttgattttttaattcgtttttttgggtttagatttgttttgttttagggcggagttaggcggttttgagggggagatgaatttttagaggagcgttgtttttattttttgttttttttggtcggttcgtatttttgtttattaattttattttaggatattgtttttgaagtttttgtatttttgttttttatttttggggggtagttgagtttttcgcgtgttgtgttattgttttgttttagatataaattagtacgtttagggtttagttttttttttatttcggtattttagcgttaagtgtatttagcggggtattcggtttttttagtttttatattcgttttgggttttagggtggtttta

SEQ ID NO: 63 - DNA, 1103 bp, Artificial Sequence
  converted
taaaaccaccctaaaacccaaaacgaatataaaaactaaaaaaaccgaataccccgctaaatacacttaacgctaaaataccgaaataaaaaaaaaactaaaccctaaacgtactaatttatatctaaaacaaaacaataacacaacacgcgaaaaactcaactaccccccaaaaataaaaaacaaaaatacaaaaacttcaaaaacaatatcctaaaataaaattaataaacaaaaatacgaaccgaccaaaaaaaacaaaaaataaaaacaacgctcctctaaaaattcatctccccctcaaaaccgcctaactccgccctaaaacaaaacaaatctaaacccaaaaaaacgaattaaaaaatcaaaatacaacaaaacaaaaaaaccaaaccgtaccaattacctccaaaaccctaacctaaaaaaataccccgcgaccataccgaatccctccccgaaccgtacctactatactactacacccgcgaaacgataactccaatctccaaccgccgctctacaaacgcgcaaaccgaaatttatctaactcctaaccctaaacgtaacgcgcgtaacgctaacgaaaatcccgacaaaaaacgctactactcgatcaataaaaacctcaaaatacgctccaactcctcctcctcctaacgcgcgcgccgccgcctctcctcctactcctacgccgacaattccatcgccaaccgcaactactcgtcgtaactccgaaacacgtaaccgccgaaacctaaccctaaactaaaccgaaaactaaacactaacgccgaaaacgcaaactaacgctacgacgtaaacgaaacgctcctaaaaataaaaaaaaaatatcgcgaactcccgaacgcacccacccaccgaccgaaaccgaaacacgacgtaaaaactccaaaacgacccgcgaccaccaccctctaataaaaacgatcaatcccgcgacgtcctaacccccacctaatcgtaacgaaacccaaaccctaccaaattctccccgtcaatacctaaaaaaaatctaaactccgcgataaaacctaaatccctaacccctacctaccgcctcgaaaacaaaaataataaca

SEQ ID NO: 64 - DNA, 1103 bp, Artificial Sequence
  converted
taccaccactcctacctccgaaacgacaaacaaaaatcaaaaatccaaacttcaccgcgaaattcaaactctccccaaacattaacgaaaaaaacttaataaaatctaaactccgccacgaccaaataaaaaccaaaacgccgcgaaactaaccgcctccactaaaaaataataaccgcgaaccgccctaaaacctccacgccgtatcccgaccccgaccgataaataaatacgcccgaaaacccgcgacattccttccccacccccaaaaacgccccgcccacgccgcaacgccaacctacgcccccgacgtcaatacccaaccctcgacccaactcaaaaccaaattccgacgaccacgtattccgaaactacgacgaacaactacgactaacgataaaactatcgacgcaaaaacaaaaaaaaaaacgacgacgcgcgcgccaaaaaaaaaaaaaactaaaacgcatcctaaaactctcactaaccgaacaataacgccccctaccgaaaccctcgccaacgccacgcgcgccacgcccaaaaccaaaaaccaaacaaaccccgacctacgcgcctacaaaacgacgactaaaaactaaaaccaccgcctcgcgaatacaacaacacaacaaacacgattcgaaaaaaaattcgacataaccgcgaaataccttcccaaaccaaaaccctaaaaacaactaacacgacctaatccccctactttactatattctaattccccaacccgctcccctaaactcaaatctatcctatcctaaaacgaaaccaaacgatcctaaaaaaaaaataaatccttaaaaaaacgctatccctatcccttactccttctaaccgatccgcatttctattcactaaccccactccaaaacactacccctaaaacctttacatctctactcttcactcctaaaaaacaactaaactccccgcgtactatatcactactttattccaaacacaaaccaacacgtctaaaacccaacccctcccccaccccgacatttcaacgtcaaatacacttaacgaaatacccgactcccccaacccccacacccgtcctaaatctcaaaataattcca

SEQ ID NO: 65 - DNA, 1103 bp, Artificial Sequence
  converted
tggaattattttgagatttaggacgggtgtgggggttgggggagtcgggtatttcgttaagtgtatttgacgttgaaatgtcggggtgggggaggggttgggttttagacgtgttggtttgtgtttggaataaagtagtgatatagtacgcggggagtttagttgttttttaggagtgaagagtagagatgtaaaggttttaggggtagtgttttggagtggggttagtgaatagaaatgcggatcggttagaaggagtaagggatagggatagcgtttttttaaggatttattttttttttaggatcgtttggtttcgttttaggataggatagatttgagtttaggggagcgggttggggaattagaatatagtaaagtagggggattaggtcgtgttagttgtttttagggttttggtttgggaaggtatttcgcggttatgtcgaattttttttcgaatcgtgtttgttgtgttgttgtattcgcgaggcggtggttttagtttttagtcgtcgttttgtaggcgcgtaggtcggggtttgtttggtttttggttttgggcgtggcgcgcgtggcgttggcgagggtttcggtagggggcgttattgttcggttagtgagagttttaggatgcgttttagttttttttttttttggcgcgcgcgtcgtcgtttttttttttgtttttgcgtcgatagttttatcgttagtcgtagttgttcgtcgtagtttcggaatacgtggtcgtcggaatttggttttgagttgggtcgagggttgggtattgacgtcgggggcgtaggttggcgttgcggcgtgggcggggcgtttttgggggtggggaaggaatgtcgcgggttttcgggcgtatttatttatcggtcggggtcgggatacggcgtggaggttttagggcggttcgcggttattattttttagtggaggcggttagtttcgcggcgttttggtttttatttggtcgtggcggagtttagattttattaagtttttttcgttaatgtttggggagagtttgaatttcgcggtgaagtttggatttttgatttttgtttgtcgtttcggaggtaggagtggtggta

SEQ ID NO: 66 - DNA, 103 bp, Homo sapiens
ccccctgccgggaccctcgccagcgccacgcgcgccacgcccagggccaggagccagacaaaccccggcctgcgcgcctgcagagcggcggctggagactgga

SEQ ID NO: 67 - DNA, 103 bp, Artificial Sequence
  converted
ttttttgtcgggattttcgttagcgttacgcgcgttacgtttagggttaggagttagataaatttcggtttgcgcgtttgtagagcggcggttggagattgga

SEQ ID NO: 68 - DNA, 103 bp, Artificial Sequence
  converted
tccaatctccaaccgccgctctacaaacgcgcaaaccgaaatttatctaactcctaaccctaaacgtaacgcgcgtaacgctaacgaaaatcccgacaaaaaa

SEQ ID NO: 69 - DNA, 103 bp, Artificial Sequence
  converted
ccccctaccgaaaccctcgccaacgccacgcgcgccacgcccaaaaccaaaaaccaaacaaaccccgacctacgcgcctacaaaacgacgactaaaaactaaa

SEQ ID NO: 70 - DNA, 103 bp, Artificial Sequence
  converted
tttagtttttagtcgtcgttttgtaggcgcgtaggtcggggtttgtttggtttttggttttgggcgtggcgcgcgtggcgttggcgagggtttcggtaggggg

SEQ ID NO: 71 - DNA, 1071 bp, Homo sapiens
cggaacccgagcccactgtaacccaagcctcaaggccgctctgccggcagaaagtaaaaccctccctccgtggagatcgcgagcccctgagttccacagtcgcgaagctcgagtgctttcggagccccgggagcgccagaggcgctgccttttgtccggtgcctgagaagccatggagaaaggcagaggggaccacagggagaagaggaaggagtaggggagaggaacgggagaggtgaagcgaccagcgtccccaagggtcagggcgcagctgcggggctggcccaggctgcgccctgggggagatgctggcgttggtgacattgaccgcagttccagaggaactctccttgggacagctacgggccgtgcacaaagcgccctcgcctccctagtactccaccacgctttgaggaaggccggaagaagacggccacccccatctgacagatggagaagctgaggcccaagagagcaaagcgccttggctggaaacccggattccctggtgctggtcccgcttctcggcccgcggcccagaagcctccccatcgggctcgggagtttggatcccaagagaggtcggaccaccagggaaggtagtggacagggggagatacagaaggggtgggggaagaggaagctggactctcgcaccatctccctttctgatcaccccaccaccacacacacatacacacacacacacacacacacacacacacacacacacccttgctgttggacctctccctcgcgaggctccggcttcaggaccacggagagctctggagcaacagcccgcccgcgaagggatggggcccgaagaggcccaccaccgtgtttaccagttccctgctcctattcctccaggaagccctcccagatatctgccccgatcaagctctgcttccctccaggctcactttgggtcactcatagcatttaagaggggaaggtttaattaaaatgataatgattgtaataaaagtcaacaacaaacatttattgaatgctcactaccgaccggcattaactgatttaattctcacaatgaatccgtcgctcacc

SEQ ID NO: 72 - DNA, 1071 bp, Artificial Sequence
  converted
cggaattcgagtttattgtaatttaagttttaaggtcgttttgtcggtagaaagtaaaatttttttttcgtggagatcgcgagtttttgagttttatagtcgcgaagttcgagtgttttcggagtttcgggagcgttagaggcgttgttttttgttcggtgtttgagaagttatggagaaaggtagaggggattatagggagaagaggaaggagtaggggagaggaacgggagaggtgaagcgattagcgtttttaagggttagggcgtagttgcggggttggtttaggttgcgttttgggggagatgttggcgttggtgatattgatcgtagttttagaggaattttttttgggatagttacgggtcgtgtataaagcgttttcgtttttttagtattttattacgttttgaggaaggtcggaagaagacggttatttttatttgatagatggagaagttgaggtttaagagagtaaagcgttttggttggaaattcggattttttggtgttggtttcgtttttcggttcgcggtttagaagtttttttatcgggttcgggagtttggattttaagagaggtcggattattagggaaggtagtggatagggggagatatagaaggggtgggggaagaggaagttggattttcgtattatttttttttttgattattttattattatatatatatatatatatatatatatatatatatatatatatatatatttttgttgttggatttttttttcgcgaggtttcggttttaggattacggagagttttggagtaatagttcgttcgcgaagggatggggttcgaagaggtttattatcgtgtttattagttttttgtttttatttttttaggaagtttttttagatatttgtttcgattaagttttgtttttttttaggtttattttgggttatttatagtatttaagaggggaaggtttaattaaaatgataatgattgtaataaaagttaataataaatatttattgaatgtttattatcgatcggtattaattgatttaatttttataatgaattcgtcgtttatt

SEQ ID NO: 73 - DNA, 1071 bp, Artificial Sequence
  converted
aataaacgacgaattcattataaaaattaaatcaattaataccgatcgataataaacattcaataaatatttattattaacttttattacaatcattatcattttaattaaaccttcccctcttaaatactataaataacccaaaataaacctaaaaaaaaacaaaacttaatcgaaacaaatatctaaaaaaacttcctaaaaaaataaaaacaaaaaactaataaacacgataataaacctcttcgaaccccatcccttcgcgaacgaactattactccaaaactctccgtaatcctaaaaccgaaacctcgcgaaaaaaaaatccaacaacaaaaatatatatatatatatatatatatatatatatatatatatatatatataataataaaataatcaaaaaaaaaaataatacgaaaatccaacttcctcttcccccaccccttctatatctccccctatccactaccttccctaataatccgacctctcttaaaatccaaactcccgaacccgataaaaaaacttctaaaccgcgaaccgaaaaacgaaaccaacaccaaaaaatccgaatttccaaccaaaacgctttactctcttaaacctcaacttctccatctatcaaataaaaataaccgtcttcttccgaccttcctcaaaacgtaataaaatactaaaaaaacgaaaacgctttatacacgacccgtaactatcccaaaaaaaattcctctaaaactacgatcaatatcaccaacgccaacatctcccccaaaacgcaacctaaaccaaccccgcaactacgccctaacccttaaaaacgctaatcgcttcacctctcccgttcctctcccctactccttcctcttctccctataatcccctctacctttctccataacttctcaaacaccgaacaaaaaacaacgcctctaacgctcccgaaactccgaaaacactcgaacttcgcgactataaaactcaaaaactcgcgatctccacgaaaaaaaaattttactttctaccgacaaaacgaccttaaaacttaaattacaataaactcgaattccg

SEQ ID NO: 74 - DNA, 1071 bp, Artificial Sequence
  converted
cgaaacccgaacccactataacccaaacctcaaaaccgctctaccgacaaaaaataaaaccctccctccgtaaaaatcgcgaacccctaaattccacaatcgcgaaactcgaatactttcgaaaccccgaaaacgccaaaaacgctaccttttatccgatacctaaaaaaccataaaaaaaaacaaaaaaaaccacaaaaaaaaaaaaaaaaaataaaaaaaaaaaacgaaaaaaataaaacgaccaacgtccccaaaaatcaaaacgcaactacgaaactaacccaaactacgccctaaaaaaaatactaacgttaataacattaaccgcaattccaaaaaaactctccttaaaacaactacgaaccgtacacaaaacgccctcgcctccctaatactccaccacgctttaaaaaaaaccgaaaaaaaacgaccacccccatctaacaaataaaaaaactaaaacccaaaaaaacaaaacgccttaactaaaaacccgaattccctaatactaatcccgcttctcgacccgcgacccaaaaacctccccatcgaactcgaaaatttaaatcccaaaaaaaatcgaaccaccaaaaaaaataataaacaaaaaaaaatacaaaaaaaataaaaaaaaaaaaaactaaactctcgcaccatctccctttctaatcaccccaccaccacacacacatacacacacacacacacacacacacacacacacacacacccttactattaaacctctccctcgcgaaactccgacttcaaaaccacgaaaaactctaaaacaacaacccgcccgcgaaaaaataaaacccgaaaaaacccaccaccgtatttaccaattccctactcctattcctccaaaaaaccctcccaaatatctaccccgatcaaactctacttccctccaaactcactttaaatcactcataacatttaaaaaaaaaaaatttaattaaaataataataattataataaaaatcaacaacaaacatttattaaatactcactaccgaccgacattaactaatttaattctcacaataaatccgtcgctcacc

SEQ ID NO: 75 - DNA, 1071 bp, Artificial Sequence
  converted
ggtgagcgacggatttattgtgagaattaaattagttaatgtcggtcggtagtgagtatttaataaatgtttgttgttgatttttattataattattattattttaattaaattttttttttttaaatgttatgagtgatttaaagtgagtttggagggaagtagagtttgatcggggtagatatttgggagggttttttggaggaataggagtagggaattggtaaatacggtggtgggttttttcgggttttattttttcgcgggcgggttgttgttttagagtttttcgtggttttgaagtcggagtttcgcgagggagaggtttaatagtaagggtgtgtgtgtgtgtgtgtgtgtgtgtgtgtgtgtgtgtatgtgtgtgtggtggtggggtgattagaaagggagatggtgcgagagtttagttttttttttttttattttttttgtatttttttttgtttattattttttttggtggttcgattttttttgggatttaaattttcgagttcgatggggaggtttttgggtcgcgggtcgagaagcgggattagtattagggaattcgggtttttagttaaggcgttttgtttttttgggttttagtttttttatttgttagatgggggtggtcgttttttttcggttttttttaaagcgtggtggagtattagggaggcgagggcgttttgtgtacggttcgtagttgttttaaggagagtttttttggaattgcggttaatgttattaacgttagtattttttttagggcgtagtttgggttagtttcgtagttgcgttttgatttttggggacgttggtcgttttatttttttcgttttttttttttattttttttttttttttttgtggttttttttgtttttttttatggttttttaggtatcggataaaaggtagcgtttttggcgttttcggggtttcgaaagtattcgagtttcgcgattgtggaatttaggggttcgcgatttttacggagggagggttttattttttgtcggtagagcggttttgaggtttgggttatagtgggttcgggtttcg

SEQ ID NO: 76 - DNA, 71 bp, Homo sapiens
attccctggtgctggtcccgcttctcggcccgcggcccagaagcctccccatcgggctcgggagtttggat

SEQ ID NO: 77 - DNA, 71 bp, Artificial Sequence
  converted
attttttggtgttggtttcgtttttcggttcgcggtttagaagtttttttatcgggttcgggagtttggat

SEQ ID NO: 78 - DNA, 71 bp, Artificial Sequence
  converted
atccaaactcccgaacccgataaaaaaacttctaaaccgcgaaccgaaaaacgaaaccaacaccaaaaaat

SEQ ID NO: 79 - DNA, 71 bp, Artificial Sequence
  converted
attccctaatactaatcccgcttctcgacccgcgacccaaaaacctccccatcgaactcgaaaatttaaat

SEQ ID NO: 80 - DNA, 71 bp, Artificial Sequence
  converted
atttaaattttcgagttcgatggggaggtttttgggtcgcgggtcgagaagcgggattagtattagggaat

SEQ ID NO: 81 - DNA, 1501 bp, Homo sapiens
tcgacagtggctctccggcccagggaggagagcgcggtcgcgggtccctcccctcagcctggaggctgcagccgctcgagtcggcccgggtgggggcggggtgggggcggcgcggagggcacggagattacggcggcgccacccgggacatccagggccccgaggccctgggcggtccccacgcgagatcgcaaaccatgacaataggcagtcacccgaggtcaaataaaaacggagtgggtcccccgcgcgccgccgccccccgcgtccctggcggcctcccccgaggcccccggcggcctcacgagcccgcagtagccggtggcgacgtcgcccccgccccacctccctgcgcaagtgcgaggctgccggcagcgcggcgcacgctccggccgttcccggcttccgcgcaaaacttccatcctgtccacgtgaagttgtcgctgccttagagagggggaaagagctgcgggaaaagccggggagtgacgactgcggcggctgggcgcgctctctcattttcttttcttctcctttcccccctgtcgcagtccggagttttggctcctctcctttcctcctccccctcggagccggcttctccctccgccccgcttctcccccgcttgtgtacgctatttgttgtggggtggccgaaggggatgtcctgttttcaccagaggcacagcgcgaaggggaaacttcgacactggaaggaacgagaataaatacttaattacggacgcactgaaccgcggctgggacagacacttcgggaacccgaggcggaccgggcgacgaggtgagtgaccccttcttccaacccccgccccagggctcccgggggagcctgagttgagagaacccccaaactttccgggaaagtgcgcgaggctccgccggggacgccgagcgctgggtactgaggacgcgcagctggacggtgcgtgggcgcctgcgtccccggggggcgcttggaggccgggtgccccacgcctgagggcccgggccgctcggaccgcagcggtgctctctgccctagaagacgtccccaagccccaagggtcccttccgagcctgcctgtcccttccggggtcggcgcggagcctgcgcgtaacggagttcatccagcagtccagcgcgcggcttctacctgcaccccgcctccacctggcagaggcgcgagcatcggggtctcccccacatctttcttatgacgtgtattactttctgatgaccccctagatggtccaggcgcgaggatgctgacccagagtccttcggagggtcacaggcgcctgggctttcccggtgccgggtgcgtgtgtactttaaaggctcgcgttctaatctccaggcactgatcgggcttttcaactgcggcgatcccactttaatagtttttatgtggcgtggactgaatgtctcctgcagtttgccagggtcggtgaaattagaggcgccttgtcagagcagtcgcgttcattggctcgagtagcg

SEQ ID NO: 82 - DNA, 1501 bp, Artificial Sequence
  converted
tcgatagtggtttttcggtttagggaggagagcgcggtcgcgggtttttttttttagtttggaggttgtagtcgttcgagtcggttcgggtgggggcggggtgggggcggcgcggagggtacggagattacggcggcgttattcgggatatttagggtttcgaggttttgggcggtttttacgcgagatcgtaaattatgataataggtagttattcgaggttaaataaaaacggagtgggtttttcgcgcgtcgtcgtttttcgcgtttttggcggtttttttcgaggttttcggcggttttacgagttcgtagtagtcggtggcgacgtcgttttcgttttatttttttgcgtaagtgcgaggttgtcggtagcgcggcgtacgtttcggtcgttttcggttttcgcgtaaaatttttattttgtttacgtgaagttgtcgttgttttagagagggggaaagagttgcgggaaaagtcggggagtgacgattgcggcggttgggcgcgtttttttattttttttttttttttttttttttttgtcgtagttcggagttttggttttttttttttttttttttttttcggagtcggttttttttttcgtttcgtttttttttcgtttgtgtacgttatttgttgtggggtggtcgaaggggatgttttgtttttattagaggtatagcgcgaaggggaaatttcgatattggaaggaacgagaataaatatttaattacggacgtattgaatcgcggttgggatagatatttcgggaattcgaggcggatcgggcgacgaggtgagtgatttttttttttaattttcgttttagggttttcgggggagtttgagttgagagaatttttaaatttttcgggaaagtgcgcgaggtttcgtcggggacgtcgagcgttgggtattgaggacgcgtagttggacggtgcgtgggcgtttgcgttttcggggggcgtttggaggtcgggtgttttacgtttgagggttcgggtcgttcggatcgtagcggtgttttttgttttagaagacgtttttaagttttaagggtttttttcgagtttgtttgtttttttcggggtcggcgcggagtttgcgcgtaacggagtttatttagtagtttagcgcgcggtttttatttgtatttcgtttttatttggtagaggcgcgagtatcggggttttttttatattttttttatgacgtgtattattttttgatgattttttagatggtttaggcgcgaggatgttgatttagagtttttcggagggttataggcgtttgggttttttcggtgtcgggtgcgtgtgtattttaaaggttcgcgttttaatttttaggtattgatcgggttttttaattgcggcgattttattttaatagtttttatgtggcgtggattgaatgttttttgtagtttgttagggtcggtgaaattagaggcgttttgttagagtagtcgcgtttattggttcgagtagcg

SEQ ID NO: 83 - DNA, 1501 bp, Artificial Sequence
  converted
cgctactcgaaccaataaacgcgactactctaacaaaacgcctctaatttcaccgaccctaacaaactacaaaaaacattcaatccacgccacataaaaactattaaaataaaatcgccgcaattaaaaaacccgatcaatacctaaaaattaaaacgcgaacctttaaaatacacacgcacccgacaccgaaaaaacccaaacgcctataaccctccgaaaaactctaaatcaacatcctcgcgcctaaaccatctaaaaaatcatcaaaaaataatacacgtcataaaaaaaatataaaaaaaaccccgatactcgcgcctctaccaaataaaaacgaaatacaaataaaaaccgcgcgctaaactactaaataaactccgttacgcgcaaactccgcgccgaccccgaaaaaaacaaacaaactcgaaaaaaacccttaaaacttaaaaacgtcttctaaaacaaaaaacaccgctacgatccgaacgacccgaaccctcaaacgtaaaacacccgacctccaaacgccccccgaaaacgcaaacgcccacgcaccgtccaactacgcgtcctcaatacccaacgctcgacgtccccgacgaaacctcgcgcactttcccgaaaaatttaaaaattctctcaactcaaactcccccgaaaaccctaaaacgaaaattaaaaaaaaaaatcactcacctcgtcgcccgatccgcctcgaattcccgaaatatctatcccaaccgcgattcaatacgtccgtaattaaatatttattctcgttccttccaatatcgaaatttccccttcgcgctatacctctaataaaaacaaaacatccccttcgaccaccccacaacaaataacgtacacaaacgaaaaaaaaacgaaacgaaaaaaaaaaccgactccgaaaaaaaaaaaaaaaaaaaaaaaaccaaaactccgaactacgacaaaaaaaaaaaaaaaaaaaaaaaaaataaaaaaacgcgcccaaccgccgcaatcgtcactccccgacttttcccgcaactctttccccctctctaaaacaacgacaacttcacgtaaacaaaataaaaattttacgcgaaaaccgaaaacgaccgaaacgtacgccgcgctaccgacaacctcgcacttacgcaaaaaaataaaacgaaaacgacgtcgccaccgactactacgaactcgtaaaaccgccgaaaacctcgaaaaaaaccgccaaaaacgcgaaaaacgacgacgcgcgaaaaacccactccgtttttatttaacctcgaataactacctattatcataatttacgatctcgcgtaaaaaccgcccaaaacctcgaaaccctaaatatcccgaataacgccgccgtaatctccgtaccctccgcgccgcccccaccccgcccccacccgaaccgactcgaacgactacaacctccaaactaaaaaaaaaaacccgcgaccgcgctctcctccctaaaccgaaaaaccactatcga

SEQ ID NO: 84 - DNA, 1501 bp, Artificial Sequence
  converted
tcgacaataactctccgacccaaaaaaaaaaacgcgatcgcgaatccctcccctcaacctaaaaactacaaccgctcgaatcgacccgaataaaaacgaaataaaaacgacgcgaaaaacacgaaaattacgacgacgccacccgaaacatccaaaaccccgaaaccctaaacgatccccacgcgaaatcgcaaaccataacaataaacaatcacccgaaatcaaataaaaacgaaataaatcccccgcgcgccgccgccccccgcgtccctaacgacctcccccgaaacccccgacgacctcacgaacccgcaataaccgataacgacgtcgcccccgccccacctccctacgcaaatacgaaactaccgacaacgcgacgcacgctccgaccgttcccgacttccgcgcaaaacttccatcctatccacgtaaaattatcgctaccttaaaaaaaaaaaaaaaactacgaaaaaaaccgaaaaataacgactacgacgactaaacgcgctctctcattttcttttcttctcctttcccccctatcgcaatccgaaattttaactcctctcctttcctcctccccctcgaaaccgacttctccctccgccccgcttctcccccgcttatatacgctatttattataaaataaccgaaaaaaatatcctattttcaccaaaaacacaacgcgaaaaaaaaacttcgacactaaaaaaaacgaaaataaatacttaattacgaacgcactaaaccgcgactaaaacaaacacttcgaaaacccgaaacgaaccgaacgacgaaataaataaccccttcttccaacccccgccccaaaactcccgaaaaaacctaaattaaaaaaacccccaaactttccgaaaaaatacgcgaaactccgccgaaaacgccgaacgctaaatactaaaaacgcgcaactaaacgatacgtaaacgcctacgtccccgaaaaacgcttaaaaaccgaataccccacgcctaaaaacccgaaccgctcgaaccgcaacgatactctctaccctaaaaaacgtccccaaaccccaaaaatcccttccgaacctacctatcccttccgaaatcgacgcgaaacctacgcgtaacgaaattcatccaacaatccaacgcgcgacttctacctacaccccgcctccacctaacaaaaacgcgaacatcgaaatctcccccacatctttcttataacgtatattactttctaataaccccctaaataatccaaacgcgaaaatactaacccaaaatccttcgaaaaatcacaaacgcctaaactttcccgataccgaatacgtatatactttaaaaactcgcgttctaatctccaaacactaatcgaacttttcaactacgacgatcccactttaataatttttatataacgtaaactaaatatctcctacaatttaccaaaatcgataaaattaaaaacgccttatcaaaacaatcgcgttcattaactcgaataacg

SEQ ID NO: 85 - DNA, 1501 bp, Artificial Sequence
  converted
cgttattcgagttaatgaacgcgattgttttgataaggcgtttttaattttatcgattttggtaaattgtaggagatatttagtttacgttatataaaaattattaaagtgggatcgtcgtagttgaaaagttcgattagtgtttggagattagaacgcgagtttttaaagtatatacgtattcggtatcgggaaagtttaggcgtttgtgatttttcgaaggattttgggttagtattttcgcgtttggattatttagggggttattagaaagtaatatacgttataagaaagatgtgggggagatttcgatgttcgcgtttttgttaggtggaggcggggtgtaggtagaagtcgcgcgttggattgttggatgaatttcgttacgcgtaggtttcgcgtcgatttcggaagggataggtaggttcggaagggatttttggggtttggggacgttttttagggtagagagtatcgttgcggttcgagcggttcgggtttttaggcgtggggtattcggtttttaagcgtttttcggggacgtaggcgtttacgtatcgtttagttgcgcgtttttagtatttagcgttcggcgttttcggcggagtttcgcgtattttttcggaaagtttgggggtttttttaatttaggttttttcgggagttttggggcgggggttggaagaaggggttatttatttcgtcgttcggttcgtttcgggttttcgaagtgtttgttttagtcgcggtttagtgcgttcgtaattaagtatttattttcgttttttttagtgtcgaagtttttttttcgcgttgtgtttttggtgaaaataggatatttttttcggttattttataataaatagcgtatataagcgggggagaagcggggcggagggagaagtcggtttcgagggggaggaggaaaggagaggagttaaaatttcggattgcgataggggggaaaggagaagaaaagaaaatgagagagcgcgtttagtcgtcgtagtcgttatttttcggtttttttcgtagttttttttttttttttaaggtagcgataattttacgtggataggatggaagttttgcgcggaagtcgggaacggtcggagcgtgcgtcgcgttgtcggtagtttcgtatttgcgtagggaggtggggcgggggcgacgtcgttatcggttattgcgggttcgtgaggtcgtcgggggtttcgggggaggtcgttagggacgcggggggcggcggcgcgcgggggatttatttcgtttttatttgatttcgggtgattgtttattgttatggtttgcgatttcgcgtggggatcgtttagggtttcggggttttggatgtttcgggtggcgtcgtcgtaattttcgtgtttttcgcgtcgtttttatttcgtttttattcgggtcgattcgagcggttgtagtttttaggttgaggggagggattcgcgatcgcgtttttttttttgggtcggagagttattgtcga

SEQ ID NO: 86 - DNA, 1194 bp, Homo sapiens
cgcaagtgcgaggctgccggcagcgcggcgcacgctccggccgttcccggcttccgcgcaaaacttccatcctgtccacgtgaagttgtcgctgccttagagagggggaaagagctgcgggaaaagccggggagtgacgactgcggcggctgggcgcgctctctcattttcttttcttctcctttcccccctgtcgcagtccggagttttggctcctctcctttcctcctccccctcggagccggcttctccctccgccccgcttctcccccgcttgtgtacgctatttgttgtggggtggccgaaggggatgtcctgttttcaccagaggcacagcgcgaaggggaaacttcgacactggaaggaacgagaataaatacttaattacggacgcactgaaccgcggctgggacagacacttcgggaacccgaggcggaccgggcgacgaggtgagtgaccccttcttccaacccccgccccagggctcccgggggagcctgagttgagagaacccccaaactttccgggaaagtgcgcgaggctccgccggggacgccgagcgctgggtactgaggacgcgcagctggacggtgcgtgggcgcctgcgtccccggggggcgcttggaggccgggtgccccacgcctgagggcccgggccgctcggaccgcagcggtgctctctgccctagaagacgtccccaagccccaagggtcccttccgagcctgcctgtcccttccggggtcggcgcggagcctgcgcgtaacggagttcatccagcagtccagcgcgcggcttctacctgcaccccgcctccacctggcagaggcgcgagcatcggggtctcccccacatctttcttatgacgtgtattactttctgatgaccccctagatggtccaggcgcgaggatgctgacccagagtccttcggagggtcacaggcgcctgggctttcccggtgccgggtgcgtgtgtactttaaaggctcgcgttctaatctccaggcactgatcgggcttttcaactgcggcgatcccactttaatagtttttatgtggcgtggactgaatgtctcctgcagtttgccagggtcggtgaaattagaggcgccttgtcagagcagtcgcgttcattggctcgagtagcgggtgccatggaaggcttataacttctccaaaggaagggacctggc

SEQ ID NO: 87 - DNA, 1194 bp, Artificial Sequence
  converted
cgtaagtgcgaggttgtcggtagcgcggcgtacgtttcggtcgttttcggttttcgcgtaaaatttttattttgtttacgtgaagttgtcgttgttttagagagggggaaagagttgcgggaaaagtcggggagtgacgattgcggcggttgggcgcgtttttttattttttttttttttttttttttttttgtcgtagttcggagttttggttttttttttttttttttttttttcggagtcggttttttttttcgtttcgtttttttttcgtttgtgtacgttatttgttgtggggtggtcgaaggggatgttttgtttttattagaggtatagcgcgaaggggaaatttcgatattggaaggaacgagaataaatatttaattacggacgtattgaatcgcggttgggatagatatttcgggaattcgaggcggatcgggcgacgaggtgagtgatttttttttttaattttcgttttagggttttcgggggagtttgagttgagagaatttttaaatttttcgggaaagtgcgcgaggtttcgtcggggacgtcgagcgttgggtattgaggacgcgtagttggacggtgcgtgggcgtttgcgttttcggggggcgtttggaggtcgggtgttttacgtttgagggttcgggtcgttcggatcgtagcggtgttttttgttttagaagacgtttttaagttttaagggtttttttcgagtttgtttgtttttttcggggtcggcgcggagtttgcgcgtaacggagtttatttagtagtttagcgcgcggtttttatttgtatttcgtttttatttggtagaggcgcgagtatcggggttttttttatattttttttatgacgtgtattattttttgatgattttttagatggtttaggcgcgaggatgttgatttagagtttttcggagggttataggcgtttgggttttttcggtgtcgggtgcgtgtgtattttaaaggttcgcgttttaatttttaggtattgatcgggttttttaattgcggcgattttattttaatagtttttatgtggcgtggattgaatgttttttgtagtttgttagggtcggtgaaattagaggcgttttgttagagtagtcgcgtttattggttcgagtagcgggtgttatggaaggtttataatttttttaaaggaagggatttggt

SEQ ID NO: 88 - DNA, 1194 bp, Artificial Sequence
  converted
accaaatcccttcctttaaaaaaattataaaccttccataacacccgctactcgaaccaataaacgcgactactctaacaaaacgcctctaatttcaccgaccctaacaaactacaaaaaacattcaatccacgccacataaaaactattaaaataaaatcgccgcaattaaaaaacccgatcaatacctaaaaattaaaacgcgaacctttaaaatacacacgcacccgacaccgaaaaaacccaaacgcctataaccctccgaaaaactctaaatcaacatcctcgcgcctaaaccatctaaaaaatcatcaaaaaataatacacgtcataaaaaaaatataaaaaaaaccccgatactcgcgcctctaccaaataaaaacgaaatacaaataaaaaccgcgcgctaaactactaaataaactccgttacgcgcaaactccgcgccgaccccgaaaaaaacaaacaaactcgaaaaaaacccttaaaacttaaaaacgtcttctaaaacaaaaaacaccgctacgatccgaacgacccgaaccctcaaacgtaaaacacccgacctccaaacgccccccgaaaacgcaaacgcccacgcaccgtccaactacgcgtcctcaatacccaacgctcgacgtccccgacgaaacctcgcgcactttcccgaaaaatttaaaaattctctcaactcaaactcccccgaaaaccctaaaacgaaaattaaaaaaaaaaatcactcacctcgtcgcccgatccgcctcgaattcccgaaatatctatcccaaccgcgattcaatacgtccgtaattaaatatttattctcgttccttccaatatcgaaatttccccttcgcgctatacctctaataaaaacaaaacatccccttcgaccaccccacaacaaataacgtacacaaacgaaaaaaaaacgaaacgaaaaaaaaaaccgactccgaaaaaaaaaaaaaaaaaaaaaaaaccaaaactccgaactacgacaaaaaaaaaaaaaaaaaaaaaaaaaataaaaaaacgcgcccaaccgccgcaatcgtcactccccgacttttcccgcaactctttccccctctctaaaacaacgacaacttcacgtaaacaaaataaaaattttacgcgaaaaccgaaaacgaccgaaacgtacgccgcgctaccgacaacctcgcacttacg

SEQ ID NO: 89 - DNA, 1194 bp, Artificial Sequence
  converted
cgcaaatacgaaactaccgacaacgcgacgcacgctccgaccgttcccgacttccgcgcaaaacttccatcctatccacgtaaaattatcgctaccttaaaaaaaaaaaaaaaactacgaaaaaaaccgaaaaataacgactacgacgactaaacgcgctctctcattttcttttcttctcctttcccccctatcgcaatccgaaattttaactcctctcctttcctcctccccctcgaaaccgacttctccctccgccccgcttctcccccgcttatatacgctatttattataaaataaccgaaaaaaatatcctattttcaccaaaaacacaacgcgaaaaaaaaacttcgacactaaaaaaaacgaaaataaatacttaattacgaacgcactaaaccgcgactaaaacaaacacttcgaaaacccgaaacgaaccgaacgacgaaataaataaccccttcttccaacccccgccccaaaactcccgaaaaaacctaaattaaaaaaacccccaaactttccgaaaaaatacgcgaaactccgccgaaaacgccgaacgctaaatactaaaaacgcgcaactaaacgatacgtaaacgcctacgtccccgaaaaacgcttaaaaaccgaataccccacgcctaaaaacccgaaccgctcgaaccgcaacgatactctctaccctaaaaaacgtccccaaaccccaaaaatcccttccgaacctacctatcccttccgaaatcgacgcgaaacctacgcgtaacgaaattcatccaacaatccaacgcgcgacttctacctacaccccgcctccacctaacaaaaacgcgaacatcgaaatctcccccacatctttcttataacgtatattactttctaataaccccctaaataatccaaacgcgaaaatactaacccaaaatccttcgaaaaatcacaaacgcctaaactttcccgataccgaatacgtatatactttaaaaactcgcgttctaatctccaaacactaatcgaacttttcaactacgacgatcccactttaataatttttatataacgtaaactaaatatctcctacaatttaccaaaatcgataaaattaaaaacgccttatcaaaacaatcgcgttcattaactcgaataacgaataccataaaaaacttataacttctccaaaaaaaaaaacctaac

SEQ ID NO: 90 - DNA, 1194 bp, Artificial Sequence
  converted
gttaggtttttttttttggagaagttataagttttttatggtattcgttattcgagttaatgaacgcgattgttttgataaggcgtttttaattttatcgattttggtaaattgtaggagatatttagtttacgttatataaaaattattaaagtgggatcgtcgtagttgaaaagttcgattagtgtttggagattagaacgcgagtttttaaagtatatacgtattcggtatcgggaaagtttaggcgtttgtgatttttcgaaggattttgggttagtattttcgcgtttggattatttagggggttattagaaagtaatatacgttataagaaagatgtgggggagatttcgatgttcgcgtttttgttaggtggaggcggggtgtaggtagaagtcgcgcgttggattgttggatgaatttcgttacgcgtaggtttcgcgtcgatttcggaagggataggtaggttcggaagggatttttggggtttggggacgttttttagggtagagagtatcgttgcggttcgagcggttcgggtttttaggcgtggggtattcggtttttaagcgtttttcggggacgtaggcgtttacgtatcgtttagttgcgcgtttttagtatttagcgttcggcgttttcggcggagtttcgcgtattttttcggaaagtttgggggtttttttaatttaggttttttcgggagttttggggcgggggttggaagaaggggttatttatttcgtcgttcggttcgtttcgggttttcgaagtgtttgttttagtcgcggtttagtgcgttcgtaattaagtatttattttcgttttttttagtgtcgaagtttttttttcgcgttgtgtttttggtgaaaataggatatttttttcggttattttataataaatagcgtatataagcgggggagaagcggggcggagggagaagtcggtttcgagggggaggaggaaaggagaggagttaaaatttcggattgcgataggggggaaaggagaagaaaagaaaatgagagagcgcgtttagtcgtcgtagtcgttatttttcggtttttttcgtagttttttttttttttttaaggtagcgataattttacgtggataggatggaagttttgcgcggaagtcgggaacggtcggagcgtgcgtcgcgttgtcggtagtttcgtatttgcg

SEQ ID NO: 91 - DNA, 194 bp, Homo sapiens
gagttgagagaacccccaaactttccgggaaagtgcgcgaggctccgccggggacgccgagcgctgggtactgaggacgcgcagctggacggtgcgtgggcgcctgcgtccccggggggcgcttggaggccgggtgccccacgcctgagggcccgggccgctcggaccgcagcggtgctctctgccctagaaga

SEQ ID NO: 92 - DNA, 194 bp, Artificial Sequence
  converted
gagttgagagaatttttaaatttttcgggaaagtgcgcgaggtttcgtcggggacgtcgagcgttgggtattgaggacgcgtagttggacggtgcgtgggcgtttgcgttttcggggggcgtttggaggtcgggtgttttacgtttgagggttcgggtcgttcggatcgtagcggtgttttttgttttagaaga

SEQ ID NO: 93 - DNA, 194 bp, Artificial Sequence
  converted
tcttctaaaacaaaaaacaccgctacgatccgaacgacccgaaccctcaaacgtaaaacacccgacctccaaacgccccccgaaaacgcaaacgcccacgcaccgtccaactacgcgtcctcaatacccaacgctcgacgtccccgacgaaacctcgcgcactttcccgaaaaatttaaaaattctctcaactc

SEQ ID NO: 94 - DNA, 194 bp, Artificial Sequence
  converted
aaattaaaaaaacccccaaactttccgaaaaaatacgcgaaactccgccgaaaacgccgaacgctaaatactaaaaacgcgcaactaaacgatacgtaaacgcctacgtccccgaaaaacgcttaaaaaccgaataccccacgcctaaaaacccgaaccgctcgaaccgcaacgatactctctaccctaaaaaa

SEQ ID NO: 95 - DNA, 194 bp, Artificial Sequence
  converted
ttttttagggtagagagtatcgttgcggttcgagcggttcgggtttttaggcgtggggtattcggtttttaagcgtttttcggggacgtaggcgtttacgtatcgtttagttgcgcgtttttagtatttagcgttcggcgttttcggcggagtttcgcgtattttttcggaaagtttgggggtttttttaattt

SEQ ID NO: 96 - DNA, 882 bp, Homo sapiens
tcggggcaagggcgcgttccctatcgcaggatcacttgctatggtaagccgcccaccctgcgcgctcctccgcgcggggaagaacctgcgcggcaggacgtggtgttggagttggggcgcccggagcgtggagtggggagacctgatgcagagagtctggagtcggagctgggggtgctgcaggtaggagcaggagcggggcggagagggaggcccgaagaagaccccacacaggttggcgcagcggggcttggggaggcttcagcccagaagtggagagggttgacagacgcctgcctgattagaaaaagccgggagcttgggaaggagacgggattgaagaagccacccggcagggaggccgaacggcccagagctctccgggtaaaacccgcctgcggtgatctgggaagtgtgtctccacctagccctgcgagcagcggccttcctcccgcccgttagaagggcgctgtgctggagtacgaacccggcccagagaagccactcgcccttctttgtcacttaaaaccctgtcccgacgcggatctcacgtctagacctctgtctttaaagcggatgtagagcgcgttctaaccgttccctaaccattgtgtcaccgcgaaaggccggggctgtgtggaaccgtcccgcacgtgtgcgatgaatctggctccgctgagaacggatccgtgggctgtctggcgcgggctggggcagcagccgagagttagtctacagagctagggcccgagggtggacctgcgtccgcgtctcgtcacgaaaggaagctctttttggagaggcaaaacgtggtcgcccagatccggcgcagctgtagccgtgggcgctgtgcagtgacagccacacccgccacctgtcacg

SEQ ID NO: 97 - DNA, 882 bp, Artificial Sequence
  converted
tcggggtaagggcgcgttttttatcgtaggattatttgttatggtaagtcgtttattttgcgcgttttttcgcgcggggaagaatttgcgcggtaggacgtggtgttggagttggggcgttcggagcgtggagtggggagatttgatgtagagagtttggagtcggagttgggggtgttgtaggtaggagtaggagcggggcggagagggaggttcgaagaagattttatataggttggcgtagcggggtttggggaggttttagtttagaagtggagagggttgatagacgtttgtttgattagaaaaagtcgggagtttgggaaggagacgggattgaagaagttattcggtagggaggtcgaacggtttagagtttttcgggtaaaattcgtttgcggtgatttgggaagtgtgtttttatttagttttgcgagtagcggtttttttttcgttcgttagaagggcgttgtgttggagtacgaattcggtttagagaagttattcgtttttttttgttatttaaaattttgtttcgacgcggattttacgtttagatttttgtttttaaagcggatgtagagcgcgttttaatcgttttttaattattgtgttatcgcgaaaggtcggggttgtgtggaatcgtttcgtacgtgtgcgatgaatttggtttcgttgagaacggattcgtgggttgtttggcgcgggttggggtagtagtcgagagttagtttatagagttagggttcgagggtggatttgcgttcgcgtttcgttacgaaaggaagttttttttggagaggtaaaacgtggtcgtttagattcggcgtagttgtagtcgtgggcgttgtgtagtgatagttatattcgttatttgttacg

SEQ ID NO: 98 - DNA, 882 bp, Artificial Sequence
  converted
cgtaacaaataacgaatataactatcactacacaacgcccacgactacaactacgccgaatctaaacgaccacgttttacctctccaaaaaaaacttcctttcgtaacgaaacgcgaacgcaaatccaccctcgaaccctaactctataaactaactctcgactactaccccaacccgcgccaaacaacccacgaatccgttctcaacgaaaccaaattcatcgcacacgtacgaaacgattccacacaaccccgacctttcgcgataacacaataattaaaaaacgattaaaacgcgctctacatccgctttaaaaacaaaaatctaaacgtaaaatccgcgtcgaaacaaaattttaaataacaaaaaaaaacgaataacttctctaaaccgaattcgtactccaacacaacgcccttctaacgaacgaaaaaaaaaccgctactcgcaaaactaaataaaaacacacttcccaaatcaccgcaaacgaattttacccgaaaaactctaaaccgttcgacctccctaccgaataacttcttcaatcccgtctccttcccaaactcccgactttttctaatcaaacaaacgtctatcaaccctctccacttctaaactaaaacctccccaaaccccgctacgccaacctatataaaatcttcttcgaacctccctctccgccccgctcctactcctacctacaacacccccaactccgactccaaactctctacatcaaatctccccactccacgctccgaacgccccaactccaacaccacgtcctaccgcgcaaattcttccccgcgcgaaaaaacgcgcaaaataaacgacttaccataacaaataatcctacgataaaaaacgcgcccttaccccga

SEQ ID NO: 99 - DNA, 882 bp, Artificial Sequence
  converted
tcgaaacaaaaacgcgttccctatcgcaaaatcacttactataataaaccgcccaccctacgcgctcctccgcgcgaaaaaaaacctacgcgacaaaacgtaatattaaaattaaaacgcccgaaacgtaaaataaaaaaacctaatacaaaaaatctaaaatcgaaactaaaaatactacaaataaaaacaaaaacgaaacgaaaaaaaaaacccgaaaaaaaccccacacaaattaacgcaacgaaacttaaaaaaacttcaacccaaaaataaaaaaaattaacaaacgcctacctaattaaaaaaaaccgaaaacttaaaaaaaaaacgaaattaaaaaaaccacccgacaaaaaaaccgaacgacccaaaactctccgaataaaacccgcctacgataatctaaaaaatatatctccacctaaccctacgaacaacgaccttcctcccgcccgttaaaaaaacgctatactaaaatacgaacccgacccaaaaaaaccactcgcccttctttatcacttaaaaccctatcccgacgcgaatctcacgtctaaacctctatctttaaaacgaatataaaacgcgttctaaccgttccctaaccattatatcaccgcgaaaaaccgaaactatataaaaccgtcccgcacgtatacgataaatctaactccgctaaaaacgaatccgtaaactatctaacgcgaactaaaacaacaaccgaaaattaatctacaaaactaaaacccgaaaataaacctacgtccgcgtctcgtcacgaaaaaaaactctttttaaaaaaacaaaacgtaatcgcccaaatccgacgcaactataaccgtaaacgctatacaataacaaccacacccgccacctatcacg

SEQ ID NO: 100 - DNA, 882 bp, Artificial Sequence
  converted
cgtgataggtggcgggtgtggttgttattgtatagcgtttacggttatagttgcgtcggatttgggcgattacgttttgtttttttaaaaagagttttttttcgtgacgagacgcggacgtaggtttattttcgggttttagttttgtagattaattttcggttgttgttttagttcgcgttagatagtttacggattcgtttttagcggagttagatttatcgtatacgtgcgggacggttttatatagtttcggtttttcgcggtgatataatggttagggaacggttagaacgcgttttatattcgttttaaagatagaggtttagacgtgagattcgcgtcgggatagggttttaagtgataaagaagggcgagtggtttttttgggtcgggttcgtattttagtatagcgtttttttaacgggcgggaggaaggtcgttgttcgtagggttaggtggagatatattttttagattatcgtaggcgggttttattcggagagttttgggtcgttcggtttttttgtcgggtggtttttttaatttcgttttttttttaagttttcggttttttttaattaggtaggcgtttgttaattttttttatttttgggttgaagtttttttaagtttcgttgcgttaatttgtgtggggtttttttcgggttttttttttcgtttcgtttttgtttttatttgtagtatttttagtttcgattttagattttttgtattaggtttttttattttacgtttcgggcgttttaattttaatattacgttttgtcgcgtaggttttttttcgcgcggaggagcgcgtagggtgggcggtttattatagtaagtgattttgcgatagggaacgcgtttttgtttcga

SEQ ID NO: 101 - DNA, 1070 bp, Homo sapiens
cacaggttggcgcagcggggcttggggaggcttcagcccagaagtggagagggttgacagacgcctgcctgattagaaaaagccgggagcttgggaaggagacgggattgaagaagccacccggcagggaggccgaacggcccagagctctccgggtaaaacccgcctgcggtgatctgggaagtgtgtctccacctagccctgcgagcagcggccttcctcccgcccgttagaagggcgctgtgctggagtacgaacccggcccagagaagccactcgcccttctttgtcacttaaaaccctgtcccgacgcggatctcacgtctagacctctgtctttaaagcggatgtagagcgcgttctaaccgttccctaaccattgtgtcaccgcgaaaggccggggctgtgtggaaccgtcccgcacgtgtgcgatgaatctggctccgctgagaacggatccgtgggctgtctggcgcgggctggggcagcagccgagagttagtctacagagctagggcccgagggtggacctgcgtccgcgtctcgtcacgaaaggaagctctttttggagaggcaaaacgtggtcgcccagatccggcgcagctgtagccgtgggcgctgtgcagtgacagccacacccgccacctgtcacgatcacagttccagggaaaggggaagggtcaaggagaatccagcggaaacttcttcccatctcacgagctctcccttctctccgtggcccccaaacactcgcatttaacgaataatcccatcaagtgagttccagtccgatttaagcggtggttccacatagtctgttggctgcggaaacttctttggtgtcagagtccggtcttcccgagaccacgaatggccatgtctgtaaatcaaaaagaacgtgagatattagagagaaacgattgtctcaaaccgaaacagctctcctacgcgaaccccagatattcctgacttggagtagctaagattttatcagcattctgggaatttgcatgtcctctctctgaactaagattttcattttactggaggttgataatccgagacttgcc

SEQ ID NO: 102 - DNA, 1070 bp, Artificial Sequence
  converted
tataggttggcgtagcggggtttggggaggttttagtttagaagtggagagggttgatagacgtttgtttgattagaaaaagtcgggagtttgggaaggagacgggattgaagaagttattcggtagggaggtcgaacggtttagagtttttcgggtaaaattcgtttgcggtgatttgggaagtgtgtttttatttagttttgcgagtagcggtttttttttcgttcgttagaagggcgttgtgttggagtacgaattcggtttagagaagttattcgtttttttttgttatttaaaattttgtttcgacgcggattttacgtttagatttttgtttttaaagcggatgtagagcgcgttttaatcgttttttaattattgtgttatcgcgaaaggtcggggttgtgtggaatcgtttcgtacgtgtgcgatgaatttggtttcgttgagaacggattcgtgggttgtttggcgcgggttggggtagtagtcgagagttagtttatagagttagggttcgagggtggatttgcgttcgcgtttcgttacgaaaggaagttttttttggagaggtaaaacgtggtcgtttagattcggcgtagttgtagtcgtgggcgttgtgtagtgatagttatattcgttatttgttacgattatagttttagggaaaggggaagggttaaggagaatttagcggaaatttttttttattttacgagttttttttttttttcgtggtttttaaatattcgtatttaacgaataattttattaagtgagttttagttcgatttaagcggtggttttatatagtttgttggttgcggaaatttttttggtgttagagttcggttttttcgagattacgaatggttatgtttgtaaattaaaaagaacgtgagatattagagagaaacgattgttttaaatcgaaatagtttttttacgcgaattttagatatttttgatttggagtagttaagattttattagtattttgggaatttgtatgttttttttttgaattaagatttttattttattggaggttgataattcgagatttgtt

SEQ ID NO: 103 - DNA, 1070 bp, Artificial Sequence
  converted
aacaaatctcgaattatcaacctccaataaaataaaaatcttaattcaaaaaaaaaacatacaaattcccaaaatactaataaaatcttaactactccaaatcaaaaatatctaaaattcgcgtaaaaaaactatttcgatttaaaacaatcgtttctctctaatatctcacgttctttttaatttacaaacataaccattcgtaatctcgaaaaaaccgaactctaacaccaaaaaaatttccgcaaccaacaaactatataaaaccaccgcttaaatcgaactaaaactcacttaataaaattattcgttaaatacgaatatttaaaaaccacgaaaaaaaaaaaaaactcgtaaaataaaaaaaaatttccgctaaattctccttaacccttcccctttccctaaaactataatcgtaacaaataacgaatataactatcactacacaacgcccacgactacaactacgccgaatctaaacgaccacgttttacctctccaaaaaaaacttcctttcgtaacgaaacgcgaacgcaaatccaccctcgaaccctaactctataaactaactctcgactactaccccaacccgcgccaaacaacccacgaatccgttctcaacgaaaccaaattcatcgcacacgtacgaaacgattccacacaaccccgacctttcgcgataacacaataattaaaaaacgattaaaacgcgctctacatccgctttaaaaacaaaaatctaaacgtaaaatccgcgtcgaaacaaaattttaaataacaaaaaaaaacgaataacttctctaaaccgaattcgtactccaacacaacgcccttctaacgaacgaaaaaaaaaccgctactcgcaaaactaaataaaaacacacttcccaaatcaccgcaaacgaattttacccgaaaaactctaaaccgttcgacctccctaccgaataacttcttcaatcccgtctccttcccaaactcccgactttttctaatcaaacaaacgtctatcaaccctctccacttctaaactaaaacctccccaaaccccgctacgccaacctata

SEQ ID NO: 104 - DNA, 1070 bp, Artificial Sequence
  converted
cacaaattaacgcaacgaaacttaaaaaaacttcaacccaaaaataaaaaaaattaacaaacgcctacctaattaaaaaaaaccgaaaacttaaaaaaaaaacgaaattaaaaaaaccacccgacaaaaaaaccgaacgacccaaaactctccgaataaaacccgcctacgataatctaaaaaatatatctccacctaaccctacgaacaacgaccttcctcccgcccgttaaaaaaacgctatactaaaatacgaacccgacccaaaaaaaccactcgcccttctttatcacttaaaaccctatcccgacgcgaatctcacgtctaaacctctatctttaaaacgaatataaaacgcgttctaaccgttccctaaccattatatcaccgcgaaaaaccgaaactatataaaaccgtcccgcacgtatacgataaatctaactccgctaaaaacgaatccgtaaactatctaacgcgaactaaaacaacaaccgaaaattaatctacaaaactaaaacccgaaaataaacctacgtccgcgtctcgtcacgaaaaaaaactctttttaaaaaaacaaaacgtaatcgcccaaatccgacgcaactataaccgtaaacgctatacaataacaaccacacccgccacctatcacgatcacaattccaaaaaaaaaaaaaaaatcaaaaaaaatccaacgaaaacttcttcccatctcacgaactctcccttctctccgtaacccccaaacactcgcatttaacgaataatcccatcaaataaattccaatccgatttaaacgataattccacataatctattaactacgaaaacttctttaatatcaaaatccgatcttcccgaaaccacgaataaccatatctataaatcaaaaaaaacgtaaaatattaaaaaaaaacgattatctcaaaccgaaacaactctcctacgcgaaccccaaatattcctaacttaaaataactaaaattttatcaacattctaaaaatttacatatcctctctctaaactaaaattttcattttactaaaaattaataatccgaaacttacc

SEQ ID NO: 105 - DNA, 1070 bp, Artificial Sequence
  converted
ggtaagtttcggattattaatttttagtaaaatgaaaattttagtttagagagaggatatgtaaatttttagaatgttgataaaattttagttattttaagttaggaatatttggggttcgcgtaggagagttgtttcggtttgagataatcgtttttttttaatattttacgttttttttgatttatagatatggttattcgtggtttcgggaagatcggattttgatattaaagaagttttcgtagttaatagattatgtggaattatcgtttaaatcggattggaatttatttgatgggattattcgttaaatgcgagtgtttgggggttacggagagaagggagagttcgtgagatgggaagaagttttcgttggattttttttgattttttttttttttttggaattgtgatcgtgataggtggcgggtgtggttgttattgtatagcgtttacggttatagttgcgtcggatttgggcgattacgttttgtttttttaaaaagagttttttttcgtgacgagacgcggacgtaggtttattttcgggttttagttttgtagattaattttcggttgttgttttagttcgcgttagatagtttacggattcgtttttagcggagttagatttatcgtatacgtgcgggacggttttatatagtttcggtttttcgcggtgatataatggttagggaacggttagaacgcgttttatattcgttttaaagatagaggtttagacgtgagattcgcgtcgggatagggttttaagtgataaagaagggcgagtggtttttttgggtcgggttcgtattttagtatagcgtttttttaacgggcgggaggaaggtcgttgttcgtagggttaggtggagatatattttttagattatcgtaggcgggttttattcggagagttttgggtcgttcggtttttttgtcgggtggtttttttaatttcgttttttttttaagttttcggttttttttaattaggtaggcgtttgttaattttttttatttttgggttgaagtttttttaagtttcgttgcgttaatttgtg

SEQ ID NO: 106 - DNA, 70 bp, Homo sapiens
agtctacagagctagggcccgagggtggacctgcgtccgcgtctcgtcacgaaaggaagctctttttgga

SEQ ID NO: 107 - DNA, 70 bp, Artificial Sequence
  converted
agtttatagagttagggttcgagggtggatttgcgttcgcgtttcgttacgaaaggaagttttttttgga

SEQ ID NO: 108 - DNA, 70 bp, Artificial Sequence
  converted
tccaaaaaaaacttcctttcgtaacgaaacgcgaacgcaaatccaccctcgaaccctaactctataaact

SEQ ID NO: 109 - DNA, 70 bp, Artificial Sequence
  converted
aatctacaaaactaaaacccgaaaataaacctacgtccgcgtctcgtcacgaaaaaaaactctttttaaa

SEQ ID NO: 110 - DNA, 70 bp, Artificial Sequence
  converted
tttaaaaagagttttttttcgtgacgagacgcggacgtaggtttattttcgggttttagttttgtagatt

SEQ ID NO: 111 - DNA, 2972 bp, Homo sapiens
gcgggccgcagccggagaggaacaggaaccgcagtggggacgccctggtccccgggcccacagcatccgggacggacgcgcagtagcgcgggccgggaactgggtaccagggcgggatgggtgagaggctctaagggacaaggcagggagaagcgcagcggggtgcggggaaccgcacgccctccctttgcctctgcttcccaccccgaggcggcagggcgggcgggcgcggttccgggggtgggcgggctgggcggggcggaggcggggccgcagcactggcttcacccagcctctcccgcccgcagccagagcgagccgagccgcggccagctccggcgggcagggggggcgctggagcgcagcgcagcgcagccccatcagtccgcaaagcggaccgagctggaagtcgagcgctgccgcgggaggcgggcgatgggggcaggtgccaccggccgcgccatggacgggccgcgcctgctgctgttgctgcttctgggggtgagtgttagccggagggggcccgctccctttcccgggatcagaactccgagaagagccgggcgccgccaccaaggaaacagaacagagcattggggtcccagatactgagggtgggtggtgggaaaggacctctgatgccgggaccacgaaggagggtctagggttcccggagcgcagaggcgactctccagggtggagatgagggcaagaccggagcacggatgccggtcctcaggtaccgcagggggcggtgggggagctgggaggggtctttcaagagggggcatggggctctccgatgcccaggttcttcggaagaggacactcgaatgccgggatcccgaagggactttcccctcagcatctcggtctctggagagtcgtgggacagaaccaaggcgagagaaggagggggaactggacggggactagagatgagaggggcgagctgggctggggcggggagcccgggacgacgggatggaacaatggaggaggtcggagggactggaggcagaggggacccggcaaggacaatggggaccggccagacaatgggggggaggggcgtcggagggagaggttggggagcgggagagaggttgagaagtgggttagggagggcagaagaggggcgacggcgagggcggcagagagatggaagcagaaggaaaggagacagagcggggcgcggaccctgaagtggtcggggtcccgcctgcccgctgagggacagtgagacgagaagagagagcagaggcttggacatttccagcgcctgtccccgcgctcctgctcgcggcctctcccagtcccggaattggggctgcggatctaaggctgggaggggagtgcccacttcgccggggcgaacccgctcccggggttcccccacggccggcgggctcctccgacgcgcgtgttctcacggcttgaagcagcgcagcggatcgcgagggacggagcgggtgtgcccctacccgggctggcggtggctacgtctcggggtgcagagcgccggggcgcactaggcggggacccttcctggccgcgcgcagctctggctacgcaggcgggaggcggcaggcagggggcgctgcggggcgaggcaagtgcagtccgccgctgcctgcctgcgagtctggggctgcgcgccgctccccacctagctgcgcgacccgagcgccgcgcccaccgcgctccgtgggctcccgcgcctcgaagggtcgggatccggtctgagcctctcctggctctcggtgcaagccgctgggcacctaacgagacgggggcgcccggacgcctcggattctgtagcgcggtatcccgggccaccgactcggcaaaggtcggcgggtcctccgggtggtccgacgagattgtcagatgtggcctgctttatggccatgaaccgaatgccctggaaggtctggcaacggaggaggcgtcttcactctccccgccagcatctttcatcagagcactgcgccccaaattccggctttacagatacggagactgaggctctgagagtttaaacgacttggccaagatctcacagcaagttgggccagagtcgtggctagactccccgacctgattcccagctcggtactcccccaccttttcagggggcagccgctgtcccagctcggggtcacggctgcgtttcccagcagggcggggggggttgacactgtccccagagtccgtcggcggtagagcctgcgggtgaggtgggcagagattctgtccgggtgcgggccccagcccgaaagctcagccccagttccctggagctgtcaggacctgtggctgcgtccggggatttgggagcgaccgcgggcacgtctgctgactaacgccgctggttagagacgctgctcagacgcgggtgggcgagcgtagaccaggagagcggggaggagggaccggatcccagaggggcaacgacttgggctccgggtctgggcaagagcgagggtccccgcgcgaagacccaaggaaggaggggagctgggcgtggaggacctgggcgcttgggaaggtgtaccccgacaggaacctgccgtctgcagcccctgcggctcccgcctccgcctcggcgcgcggctgctcggtctgggatggctgtgcgcggtccgagtttcttcctgggccgcggtgagagagagagggctgaaaccagagcgcgtcccggcgggtcggctggcgggccgcgccggtaatggaggcactttgtcattcagacgtctgtaaccagagccgccgggctggctaatgcgcctaatagggatggaacgagggcagcaaatgggcgtgcgtgagcggccgggctgagctgggtggatggtggatggggaggcagctccgcgggggacgggcccctaccgcactgttccagcccagtctggctcaagcgcctcgcttcttccctgggggaccgcgtggggaggcgggaataatcg

SEQ ID NO: 112 - DNA, 2972 bp, Artificial Sequence
  converted
gcgggtcgtagtcggagaggaataggaatcgtagtggggacgttttggttttcgggtttatagtattcgggacggacgcgtagtagcgcgggtcgggaattgggtattagggcgggatgggtgagaggttttaagggataaggtagggagaagcgtagcggggtgcggggaatcgtacgttttttttttgtttttgttttttatttcgaggcggtagggcgggcgggcgcggtttcgggggtgggcgggttgggcggggcggaggcggggtcgtagtattggttttatttagtttttttcgttcgtagttagagcgagtcgagtcgcggttagtttcggcgggtagggggggcgttggagcgtagcgtagcgtagttttattagttcgtaaagcggatcgagttggaagtcgagcgttgtcgcgggaggcgggcgatgggggtaggtgttatcggtcgcgttatggacgggtcgcgtttgttgttgttgttgtttttgggggtgagtgttagtcggagggggttcgttttttttttcgggattagaatttcgagaagagtcgggcgtcgttattaaggaaatagaatagagtattggggttttagatattgagggtgggtggtgggaaaggatttttgatgtcgggattacgaaggagggtttagggttttcggagcgtagaggcgattttttagggtggagatgagggtaagatcggagtacggatgtcggtttttaggtatcgtagggggcggtgggggagttgggaggggttttttaagagggggtatggggtttttcgatgtttaggtttttcggaagaggatattcgaatgtcgggatttcgaagggattttttttttagtatttcggtttttggagagtcgtgggatagaattaaggcgagagaaggagggggaattggacggggattagagatgagaggggcgagttgggttggggcggggagttcgggacgacgggatggaataatggaggaggtcggagggattggaggtagaggggattcggtaaggataatggggatcggttagataatgggggggaggggcgtcggagggagaggttggggagcgggagagaggttgagaagtgggttagggagggtagaagaggggcgacggcgagggcggtagagagatggaagtagaaggaaaggagatagagcggggcgcggattttgaagtggtcggggtttcgtttgttcgttgagggatagtgagacgagaagagagagtagaggtttggatatttttagcgtttgttttcgcgtttttgttcgcggttttttttagtttcggaattggggttgcggatttaaggttgggaggggagtgtttatttcgtcggggcgaattcgttttcggggtttttttacggtcggcgggttttttcgacgcgcgtgtttttacggtttgaagtagcgtagcggatcgcgagggacggagcgggtgtgtttttattcgggttggcggtggttacgtttcggggtgtagagcgtcggggcgtattaggcggggattttttttggtcgcgcgtagttttggttacgtaggcgggaggcggtaggtagggggcgttgcggggcgaggtaagtgtagttcgtcgttgtttgtttgcgagtttggggttgcgcgtcgttttttatttagttgcgcgattcgagcgtcgcgtttatcgcgtttcgtgggttttcgcgtttcgaagggtcgggattcggtttgagttttttttggttttcggtgtaagtcgttgggtatttaacgagacgggggcgttcggacgtttcggattttgtagcgcggtatttcgggttatcgattcggtaaaggtcggcgggtttttcgggtggttcgacgagattgttagatgtggtttgttttatggttatgaatcgaatgttttggaaggtttggtaacggaggaggcgtttttatttttttcgttagtattttttattagagtattgcgttttaaatttcggttttatagatacggagattgaggttttgagagtttaaacgatttggttaagattttatagtaagttgggttagagtcgtggttagatttttcgatttgatttttagttcggtatttttttatttttttagggggtagtcgttgttttagttcggggttacggttgcgttttttagtagggcggggggggttgatattgtttttagagttcgtcggcggtagagtttgcgggtgaggtgggtagagattttgttcgggtgcgggttttagttcgaaagtttagttttagttttttggagttgttaggatttgtggttgcgttcggggatttgggagcgatcgcgggtacgtttgttgattaacgtcgttggttagagacgttgtttagacgcgggtgggcgagcgtagattaggagagcggggaggagggatcggattttagaggggtaacgatttgggtttcgggtttgggtaagagcgagggttttcgcgcgaagatttaaggaaggaggggagttgggcgtggaggatttgggcgtttgggaaggtgtatttcgataggaatttgtcgtttgtagtttttgcggttttcgttttcgtttcggcgcgcggttgttcggtttgggatggttgtgcgcggttcgagtttttttttgggtcgcggtgagagagagagggttgaaattagagcgcgtttcggcgggtcggttggcgggtcgcgtcggtaatggaggtattttgttatttagacgtttgtaattagagtcgtcgggttggttaatgcgtttaatagggatggaacgagggtagtaaatgggcgtgcgtgagcggtcgggttgagttgggtggatggtggatggggaggtagtttcgcgggggacgggtttttatcgtattgttttagtttagtttggtttaagcgtttcgttttttttttgggggatcgcgtggggaggcgggaataatcg

SEQ ID NO: 113 - DNA, 2972 bp, Artificial Sequence
  converted
cgattattcccgcctccccacgcgatcccccaaaaaaaaaacgaaacgcttaaaccaaactaaactaaaacaatacgataaaaacccgtcccccgcgaaactacctccccatccaccatccacccaactcaacccgaccgctcacgcacgcccatttactaccctcgttccatccctattaaacgcattaaccaacccgacgactctaattacaaacgtctaaataacaaaatacctccattaccgacgcgacccgccaaccgacccgccgaaacgcgctctaatttcaaccctctctctctcaccgcgacccaaaaaaaaactcgaaccgcgcacaaccatcccaaaccgaacaaccgcgcgccgaaacgaaaacgaaaaccgcaaaaactacaaacgacaaattcctatcgaaatacaccttcccaaacgcccaaatcctccacgcccaactcccctccttccttaaatcttcgcgcgaaaaccctcgctcttacccaaacccgaaacccaaatcgttacccctctaaaatccgatccctcctccccgctctcctaatctacgctcgcccacccgcgtctaaacaacgtctctaaccaacgacgttaatcaacaaacgtacccgcgatcgctcccaaatccccgaacgcaaccacaaatcctaacaactccaaaaaactaaaactaaactttcgaactaaaacccgcacccgaacaaaatctctacccacctcacccgcaaactctaccgccgacgaactctaaaaacaatatcaaccccccccgccctactaaaaaacgcaaccgtaaccccgaactaaaacaacgactaccccctaaaaaaataaaaaaataccgaactaaaaatcaaatcgaaaaatctaaccacgactctaacccaacttactataaaatcttaaccaaatcgtttaaactctcaaaacctcaatctccgtatctataaaaccgaaatttaaaacgcaatactctaataaaaaatactaacgaaaaaaataaaaacgcctcctccgttaccaaaccttccaaaacattcgattcataaccataaaacaaaccacatctaacaatctcgtcgaaccacccgaaaaacccgccgacctttaccgaatcgataacccgaaataccgcgctacaaaatccgaaacgtccgaacgcccccgtctcgttaaatacccaacgacttacaccgaaaaccaaaaaaaactcaaaccgaatcccgacccttcgaaacgcgaaaacccacgaaacgcgataaacgcgacgctcgaatcgcgcaactaaataaaaaacgacgcgcaaccccaaactcgcaaacaaacaacgacgaactacacttacctcgccccgcaacgccccctacctaccgcctcccgcctacgtaaccaaaactacgcgcgaccaaaaaaaatccccgcctaatacgccccgacgctctacaccccgaaacgtaaccaccgccaacccgaataaaaacacacccgctccgtccctcgcgatccgctacgctacttcaaaccgtaaaaacacgcgcgtcgaaaaaacccgccgaccgtaaaaaaaccccgaaaacgaattcgccccgacgaaataaacactcccctcccaaccttaaatccgcaaccccaattccgaaactaaaaaaaaccgcgaacaaaaacgcgaaaacaaacgctaaaaatatccaaacctctactctctcttctcgtctcactatccctcaacgaacaaacgaaaccccgaccacttcaaaatccgcgccccgctctatctcctttccttctacttccatctctctaccgccctcgccgtcgcccctcttctaccctccctaacccacttctcaacctctctcccgctccccaacctctccctccgacgcccctcccccccattatctaaccgatccccattatccttaccgaatcccctctacctccaatccctccgacctcctccattattccatcccgtcgtcccgaactccccgccccaacccaactcgcccctctcatctctaatccccgtccaattccccctccttctctcgccttaattctatcccacgactctccaaaaaccgaaatactaaaaaaaaaatcccttcgaaatcccgacattcgaatatcctcttccgaaaaacctaaacatcgaaaaaccccataccccctcttaaaaaacccctcccaactcccccaccgccccctacgatacctaaaaaccgacatccgtactccgatcttaccctcatctccaccctaaaaaatcgcctctacgctccgaaaaccctaaaccctccttcgtaatcccgacatcaaaaatcctttcccaccacccaccctcaatatctaaaaccccaatactctattctatttccttaataacgacgcccgactcttctcgaaattctaatcccgaaaaaaaaaacgaaccccctccgactaacactcacccccaaaaacaacaacaacaacaaacgcgacccgtccataacgcgaccgataacacctacccccatcgcccgcctcccgcgacaacgctcgacttccaactcgatccgctttacgaactaataaaactacgctacgctacgctccaacgccccccctacccgccgaaactaaccgcgactcgactcgctctaactacgaacgaaaaaaactaaataaaaccaatactacgaccccgcctccgccccgcccaacccgcccacccccgaaaccgcgcccgcccgccctaccgcctcgaaataaaaaacaaaaacaaaaaaaaaacgtacgattccccgcaccccgctacgcttctccctaccttatcccttaaaacctctcacccatcccgccctaatacccaattcccgacccgcgctactacgcgtccgtcccgaatactataaacccgaaaaccaaaacgtccccactacgattcctattcctctccgactacgacccgc

SEQ ID NO: 114 - DNA, 2972 bp, Artificial Sequence
  converted
acgaaccgcaaccgaaaaaaaacaaaaaccgcaataaaaacgccctaatccccgaacccacaacatccgaaacgaacgcgcaataacgcgaaccgaaaactaaataccaaaacgaaataaataaaaaactctaaaaaacaaaacaaaaaaaaacgcaacgaaatacgaaaaaccgcacgccctccctttacctctacttcccaccccgaaacgacaaaacgaacgaacgcgattccgaaaataaacgaactaaacgaaacgaaaacgaaaccgcaacactaacttcacccaacctctcccgcccgcaaccaaaacgaaccgaaccgcgaccaactccgacgaacaaaaaaaacgctaaaacgcaacgcaacgcaaccccatcaatccgcaaaacgaaccgaactaaaaatcgaacgctaccgcgaaaaacgaacgataaaaacaaataccaccgaccgcgccataaacgaaccgcgcctactactattactacttctaaaaataaatattaaccgaaaaaaacccgctccctttcccgaaatcaaaactccgaaaaaaaccgaacgccgccaccaaaaaaacaaaacaaaacattaaaatcccaaatactaaaaataaataataaaaaaaaacctctaataccgaaaccacgaaaaaaaatctaaaattcccgaaacgcaaaaacgactctccaaaataaaaataaaaacaaaaccgaaacacgaataccgatcctcaaataccgcaaaaaacgataaaaaaactaaaaaaaatctttcaaaaaaaaacataaaactctccgatacccaaattcttcgaaaaaaaacactcgaataccgaaatcccgaaaaaactttcccctcaacatctcgatctctaaaaaatcgtaaaacaaaaccaaaacgaaaaaaaaaaaaaaaactaaacgaaaactaaaaataaaaaaaacgaactaaactaaaacgaaaaacccgaaacgacgaaataaaacaataaaaaaaatcgaaaaaactaaaaacaaaaaaaacccgacaaaaacaataaaaaccgaccaaacaataaaaaaaaaaaacgtcgaaaaaaaaaattaaaaaacgaaaaaaaaattaaaaaataaattaaaaaaaacaaaaaaaaaacgacgacgaaaacgacaaaaaaataaaaacaaaaaaaaaaaaaacaaaacgaaacgcgaaccctaaaataatcgaaatcccgcctacccgctaaaaaacaataaaacgaaaaaaaaaaacaaaaacttaaacatttccaacgcctatccccgcgctcctactcgcgacctctcccaatcccgaaattaaaactacgaatctaaaactaaaaaaaaaatacccacttcgccgaaacgaacccgctcccgaaattcccccacgaccgacgaactcctccgacgcgcgtattctcacgacttaaaacaacgcaacgaatcgcgaaaaacgaaacgaatatacccctacccgaactaacgataactacgtctcgaaatacaaaacgccgaaacgcactaaacgaaaacccttcctaaccgcgcgcaactctaactacgcaaacgaaaaacgacaaacaaaaaacgctacgaaacgaaacaaatacaatccgccgctacctacctacgaatctaaaactacgcgccgctccccacctaactacgcgacccgaacgccgcgcccaccgcgctccgtaaactcccgcgcctcgaaaaatcgaaatccgatctaaacctctcctaactctcgatacaaaccgctaaacacctaacgaaacgaaaacgcccgaacgcctcgaattctataacgcgatatcccgaaccaccgactcgacaaaaatcgacgaatcctccgaataatccgacgaaattatcaaatataacctactttataaccataaaccgaataccctaaaaaatctaacaacgaaaaaaacgtcttcactctccccgccaacatctttcatcaaaacactacgccccaaattccgactttacaaatacgaaaactaaaactctaaaaatttaaacgacttaaccaaaatctcacaacaaattaaaccaaaatcgtaactaaactccccgacctaattcccaactcgatactcccccaccttttcaaaaaacaaccgctatcccaactcgaaatcacgactacgtttcccaacaaaacgaaaaaaattaacactatccccaaaatccgtcgacgataaaacctacgaataaaataaacaaaaattctatccgaatacgaaccccaacccgaaaactcaaccccaattccctaaaactatcaaaacctataactacgtccgaaaatttaaaaacgaccgcgaacacgtctactaactaacgccgctaattaaaaacgctactcaaacgcgaataaacgaacgtaaaccaaaaaaacgaaaaaaaaaaaccgaatcccaaaaaaacaacgacttaaactccgaatctaaacaaaaacgaaaatccccgcgcgaaaacccaaaaaaaaaaaaaaactaaacgtaaaaaacctaaacgcttaaaaaaatataccccgacaaaaacctaccgtctacaacccctacgactcccgcctccgcctcgacgcgcgactactcgatctaaaataactatacgcgatccgaatttcttcctaaaccgcgataaaaaaaaaaaaactaaaaccaaaacgcgtcccgacgaatcgactaacgaaccgcgccgataataaaaacactttatcattcaaacgtctataaccaaaaccgccgaactaactaatacgcctaataaaaataaaacgaaaacaacaaataaacgtacgtaaacgaccgaactaaactaaataaataataaataaaaaaacaactccgcgaaaaacgaacccctaccgcactattccaacccaatctaactcaaacgcctcgcttcttccctaaaaaaccgcgtaaaaaaacgaaaataatcg

SEQ ID NO: 115 - DNA, 2972 bp, Artificial Sequence
  converted
cgattattttcgtttttttacgcggttttttagggaagaagcgaggcgtttgagttagattgggttggaatagtgcggtaggggttcgtttttcgcggagttgtttttttatttattatttatttagtttagttcggtcgtttacgtacgtttatttgttgttttcgttttatttttattaggcgtattagttagttcggcggttttggttatagacgtttgaatgataaagtgtttttattatcggcgcggttcgttagtcgattcgtcgggacgcgttttggttttagtttttttttttttatcgcggtttaggaagaaattcggatcgcgtatagttattttagatcgagtagtcgcgcgtcgaggcggaggcgggagtcgtaggggttgtagacggtaggtttttgtcggggtatatttttttaagcgtttaggttttttacgtttagtttttttttttttttgggttttcgcgcggggattttcgtttttgtttagattcggagtttaagtcgttgtttttttgggattcggtttttttttttcgtttttttggtttacgttcgtttattcgcgtttgagtagcgtttttaattagcggcgttagttagtagacgtgttcgcggtcgtttttaaattttcggacgtagttataggttttgatagttttagggaattggggttgagttttcgggttggggttcgtattcggatagaatttttgtttattttattcgtaggttttatcgtcgacggattttggggatagtgttaatttttttcgttttgttgggaaacgtagtcgtgatttcgagttgggatagcggttgttttttgaaaaggtgggggagtatcgagttgggaattaggtcggggagtttagttacgattttggtttaatttgttgtgagattttggttaagtcgtttaaatttttagagttttagttttcgtatttgtaaagtcggaatttggggcgtagtgttttgatgaaagatgttggcggggagagtgaagacgtttttttcgttgttagattttttagggtattcggtttatggttataaagtaggttatatttgataatttcgtcggattattcggaggattcgtcgatttttgtcgagtcggtggttcgggatatcgcgttatagaattcgaggcgttcgggcgttttcgtttcgttaggtgtttagcggtttgtatcgagagttaggagaggtttagatcggatttcgatttttcgaggcgcgggagtttacggagcgcggtgggcgcggcgttcgggtcgcgtagttaggtggggagcggcgcgtagttttagattcgtaggtaggtagcggcggattgtatttgtttcgtttcgtagcgttttttgtttgtcgtttttcgtttgcgtagttagagttgcgcgcggttaggaagggttttcgtttagtgcgtttcggcgttttgtatttcgagacgtagttatcgttagttcgggtaggggtatattcgtttcgtttttcgcgattcgttgcgttgttttaagtcgtgagaatacgcgcgtcggaggagttcgtcggtcgtgggggaatttcgggagcgggttcgtttcggcgaagtgggtattttttttttagttttagattcgtagttttaatttcgggattgggagaggtcgcgagtaggagcgcggggataggcgttggaaatgtttaagtttttgtttttttttttcgttttattgttttttagcgggtaggcgggatttcgattattttagggttcgcgtttcgttttgttttttttttttttgtttttatttttttgtcgttttcgtcgtcgttttttttttgttttttttaatttattttttaatttttttttcgttttttaattttttttttcgacgttttttttttttattgtttggtcggtttttattgtttttgtcgggttttttttgtttttagttttttcgattttttttattgttttatttcgtcgtttcgggtttttcgttttagtttagttcgttttttttatttttagttttcgtttagttttttttttttttttcgttttggttttgttttacgattttttagagatcgagatgttgaggggaaagttttttcgggatttcggtattcgagtgttttttttcgaagaatttgggtatcggagagttttatgtttttttttgaaagattttttttagtttttttatcgttttttgcggtatttgaggatcggtattcgtgtttcggttttgtttttatttttattttggagagtcgtttttgcgtttcgggaattttagattttttttcgtggtttcggtattagaggtttttttttattatttatttttagtatttgggattttaatgttttgttttgtttttttggtggcggcgttcggtttttttcggagttttgatttcgggaaagggagcgggtttttttcggttaatatttatttttagaagtagtaatagtagtaggcgcggttcgtttatggcgcggtcggtggtatttgtttttatcgttcgtttttcgcggtagcgttcgatttttagttcggttcgttttgcggattgatggggttgcgttgcgttgcgttttagcgttttttttgttcgtcggagttggtcgcggttcggttcgttttggttgcgggcgggagaggttgggtgaagttagtgttgcggtttcgttttcgtttcgtttagttcgtttattttcggaatcgcgttcgttcgttttgtcgtttcggggtgggaagtagaggtaaagggagggcgtgcggtttttcgtatttcgttgcgtttttttttgttttgttttttagagttttttatttatttcgttttggtatttagttttcggttcgcgttattgcgcgttcgtttcggatgttgtgggttcggggattagggcgtttttattgcggtttttgttttttttcggttgcggttcgt

SEQ ID NO: 116 - DNA, 1060 bp, Homo sapiens
cagggcggggggggttgacactgtccccagagtccgtcggcggtagagcctgcgggtgaggtgggcagagattctgtccgggtgcgggccccagcccgaaagctcagccccagttccctggagctgtcaggacctgtggctgcgtccggggatttgggagcgaccgcgggcacgtctgctgactaacgccgctggttagagacgctgctcagacgcgggtgggcgagcgtagaccaggagagcggggaggagggaccggatcccagaggggcaacgacttgggctccgggtctgggcaagagcgagggtccccgcgcgaagacccaaggaaggaggggagctgggcgtggaggacctgggcgcttgggaaggtgtaccccgacaggaacctgccgtctgcagcccctgcggctcccgcctccgcctcggcgcgcggctgctcggtctgggatggctgtgcgcggtccgagtttcttcctgggccgcggtgagagagagagggctgaaaccagagcgcgtcccggcgggtcggctggcgggccgcgccggtaatggaggcactttgtcattcagacgtctgtaaccagagccgccgggctggctaatgcgcctaatagggatggaacgagggcagcaaatgggcgtgcgtgagcggccgggctgagctgggtggatggtggatggggaggcagctccgcgggggacgggcccctaccgcactgttccagcccagtctggctcaagcgcctcgcttcttccctgggggaccgcgtggggaggcgggaataatcgcatgtgctgagttcctactaagtgccttgcacttccctggtagtttctcttgaattatttaatccttataaggtcctagcaaggtgaatttagttattcccacttcgcagttgaggaaacagaaccttctagactttcaggcccgagaccaggctcccccagtgagtcagtggtggcgtctttgttggaacccaggatgcttggcctgggatgttctctttcctacatatgccaggctccctcccgccccctaccttctagatcatgg

SEQ ID NO: 117 - DNA, 1060 bp, Artificial Sequence
  converted
tagggcggggggggttgatattgtttttagagttcgtcggcggtagagtttgcgggtgaggtgggtagagattttgttcgggtgcgggttttagttcgaaagtttagttttagttttttggagttgttaggatttgtggttgcgttcggggatttgggagcgatcgcgggtacgtttgttgattaacgtcgttggttagagacgttgtttagacgcgggtgggcgagcgtagattaggagagcggggaggagggatcggattttagaggggtaacgatttgggtttcgggtttgggtaagagcgagggttttcgcgcgaagatttaaggaaggaggggagttgggcgtggaggatttgggcgtttgggaaggtgtatttcgataggaatttgtcgtttgtagtttttgcggttttcgttttcgtttcggcgcgcggttgttcggtttgggatggttgtgcgcggttcgagtttttttttgggtcgcggtgagagagagagggttgaaattagagcgcgtttcggcgggtcggttggcgggtcgcgtcggtaatggaggtattttgttatttagacgtttgtaattagagtcgtcgggttggttaatgcgtttaatagggatggaacgagggtagtaaatgggcgtgcgtgagcggtcgggttgagttgggtggatggtggatggggaggtagtttcgcgggggacgggtttttatcgtattgttttagtttagtttggtttaagcgtttcgttttttttttgggggatcgcgtggggaggcgggaataatcgtatgtgttgagtttttattaagtgttttgtatttttttggtagttttttttgaattatttaatttttataaggttttagtaaggtgaatttagttatttttatttcgtagttgaggaaatagaattttttagatttttaggttcgagattaggtttttttagtgagttagtggtggcgtttttgttggaatttaggatgtttggtttgggatgtttttttttttatatatgttaggttttttttcgttttttattttttagattatgg

SEQ ID NO: 118 - DNA, 1060 bp, Artificial Sequence
  converted
ccataatctaaaaaataaaaaacgaaaaaaaacctaacatatataaaaaaaaaaacatcccaaaccaaacatcctaaattccaacaaaaacgccaccactaactcactaaaaaaacctaatctcgaacctaaaaatctaaaaaattctatttcctcaactacgaaataaaaataactaaattcaccttactaaaaccttataaaaattaaataattcaaaaaaaactaccaaaaaaatacaaaacacttaataaaaactcaacacatacgattattcccgcctccccacgcgatcccccaaaaaaaaaacgaaacgcttaaaccaaactaaactaaaacaatacgataaaaacccgtcccccgcgaaactacctccccatccaccatccacccaactcaacccgaccgctcacgcacgcccatttactaccctcgttccatccctattaaacgcattaaccaacccgacgactctaattacaaacgtctaaataacaaaatacctccattaccgacgcgacccgccaaccgacccgccgaaacgcgctctaatttcaaccctctctctctcaccgcgacccaaaaaaaaactcgaaccgcgcacaaccatcccaaaccgaacaaccgcgcgccgaaacgaaaacgaaaaccgcaaaaactacaaacgacaaattcctatcgaaatacaccttcccaaacgcccaaatcctccacgcccaactcccctccttccttaaatcttcgcgcgaaaaccctcgctcttacccaaacccgaaacccaaatcgttacccctctaaaatccgatccctcctccccgctctcctaatctacgctcgcccacccgcgtctaaacaacgtctctaaccaacgacgttaatcaacaaacgtacccgcgatcgctcccaaatccccgaacgcaaccacaaatcctaacaactccaaaaaactaaaactaaactttcgaactaaaacccgcacccgaacaaaatctctacccacctcacccgcaaactctaccgccgacgaactctaaaaacaatatcaaccccccccgcccta

SEQ ID NO: 119 - DNA, 1060 bp, Artificial Sequence
  converted
caaaacgaaaaaaattaacactatccccaaaatccgtcgacgataaaacctacgaataaaataaacaaaaattctatccgaatacgaaccccaacccgaaaactcaaccccaattccctaaaactatcaaaacctataactacgtccgaaaatttaaaaacgaccgcgaacacgtctactaactaacgccgctaattaaaaacgctactcaaacgcgaataaacgaacgtaaaccaaaaaaacgaaaaaaaaaaaccgaatcccaaaaaaacaacgacttaaactccgaatctaaacaaaaacgaaaatccccgcgcgaaaacccaaaaaaaaaaaaaaactaaacgtaaaaaacctaaacgcttaaaaaaatataccccgacaaaaacctaccgtctacaacccctacgactcccgcctccgcctcgacgcgcgactactcgatctaaaataactatacgcgatccgaatttcttcctaaaccgcgataaaaaaaaaaaaactaaaaccaaaacgcgtcccgacgaatcgactaacgaaccgcgccgataataaaaacactttatcattcaaacgtctataaccaaaaccgccgaactaactaatacgcctaataaaaataaaacgaaaacaacaaataaacgtacgtaaacgaccgaactaaactaaataaataataaataaaaaaacaactccgcgaaaaacgaacccctaccgcactattccaacccaatctaactcaaacgcctcgcttcttccctaaaaaaccgcgtaaaaaaacgaaaataatcgcatatactaaattcctactaaataccttacacttccctaataatttctcttaaattatttaatccttataaaatcctaacaaaataaatttaattattcccacttcgcaattaaaaaaacaaaaccttctaaactttcaaacccgaaaccaaactcccccaataaatcaataataacgtctttattaaaacccaaaatacttaacctaaaatattctctttcctacatataccaaactccctcccgccccctaccttctaaatcataa

SEQ ID NO: 120 - DNA, 1060 bp, Artificial Sequence
  converted
ttatgatttagaaggtagggggcgggagggagtttggtatatgtaggaaagagaatattttaggttaagtattttgggttttaataaagacgttattattgatttattgggggagtttggtttcgggtttgaaagtttagaaggttttgtttttttaattgcgaagtgggaataattaaatttattttgttaggattttataaggattaaataatttaagagaaattattagggaagtgtaaggtatttagtaggaatttagtatatgcgattattttcgtttttttacgcggttttttagggaagaagcgaggcgtttgagttagattgggttggaatagtgcggtaggggttcgtttttcgcggagttgtttttttatttattatttatttagtttagttcggtcgtttacgtacgtttatttgttgttttcgttttatttttattaggcgtattagttagttcggcggttttggttatagacgtttgaatgataaagtgtttttattatcggcgcggttcgttagtcgattcgtcgggacgcgttttggttttagtttttttttttttatcgcggtttaggaagaaattcggatcgcgtatagttattttagatcgagtagtcgcgcgtcgaggcggaggcgggagtcgtaggggttgtagacggtaggtttttgtcggggtatatttttttaagcgtttaggttttttacgtttagtttttttttttttttgggttttcgcgcggggattttcgtttttgtttagattcggagtttaagtcgttgtttttttgggattcggtttttttttttcgtttttttggtttacgttcgtttattcgcgtttgagtagcgtttttaattagcggcgttagttagtagacgtgttcgcggtcgtttttaaattttcggacgtagttataggttttgatagttttagggaattggggttgagttttcgggttggggttcgtattcggatagaatttttgtttattttattcgtaggttttatcgtcgacggattttggggatagtgttaatttttttcgttttg

SEQ ID NO: 121 - DNA, 60 bp, Homo sapiens
ggctgaaaccagagcgcgtcccggcgggtcggctggcgggccgcgccggtaatggaggca

SEQ ID NO: 122 - DNA, 60 bp, Artificial Sequence
  converted
ggttgaaattagagcgcgtttcggcgggtcggttggcgggtcgcgtcggtaatggaggta

SEQ ID NO: 123 - DNA, 60 bp, Artificial Sequence
  converted
tacctccattaccgacgcgacccgccaaccgacccgccgaaacgcgctctaatttcaacc

SEQ ID NO: 124 - DNA, 60 bp, Artificial Sequence
  converted
aactaaaaccaaaacgcgtcccgacgaatcgactaacgaaccgcgccgataataaaaaca

SEQ ID NO: 125 - DNA, 60 bp, Artificial Sequence
  converted
tgtttttattatcggcgcggttcgttagtcgattcgtcgggacgcgttttggttttagtt

SEQ ID NO: 126 - DNA, 3088 bp, Homo sapiens
tcgcctttgcggctcccgaagtcacttttcctcccgtagccggacgctgccgcccggggttcagacagcgcgcgagaccaggggagatgcgggggagggtgggcggccctgggcttccggccgaccgctcgcaggccggaggccgagcgttctcctccccgccccaatcctaggcgcggggtgcagcctgtggtcggaggggcctgcaccgggagccccgcccgccccagggccccggtacccccggcctccccgcccgagaggaaccgcctgtgggagccccgcgggcggccgggcccggctcccaggcccaggcctccggcccggcgcagtaggacaaacgggagggcagggattttttctttttttcttttttaaacacaggatttttattaaaattcttatttaaaaaatcgaaagctttctgcgcccggcgctgttggggaaccgcgccgaatagctgagctccaagttcggggctgaaggcccggaggacagggcagagggctccctgcctacagggttttcttttccatatttgagaaatgtttgcacaataaaataaaaagagaaaggaagaaagcaggggaaaacgcaaaaacaaaaacaaaacaaaaaacaaacccaaacaagccacaaagaaaggagttggacccagacgggaaacaggccggtcctgaaggtcattttggcaacaatcaccaccgatatttacaacgaaaagcgaaatctgccaccagttgtcagaacgtttacatggccataaatatttagcattttcttatttttttttaaaaaaaggaagaaattctctgtatttttaaagtgttctccacttgctttagaagacggctgacaatatcgctactcacacaaacatattttacaaaatgcacgaaagcaggggtggggggtcggtctttttctcgttttcaagtgacgacattaacgctgggacggtttggtccccccggggagaggcaaagaagcgaagctgcgcaaacattctgtaaacacggcgtagagttcagccctctcccaaggttcagaaggagaggcatgggcagagctgggtgggtggaatctgccactccaaggagacgcaggcaacctcccggcgcctccctagtggagccgagagtcaactcgactccataataataattataataataataataaccaccataaggaccgaggcctcctcgccgccaccgccgccggggtggggcctgggcctggggccgcgagtctcgttggggcggcgctcaccaagtccactgctgggcctggaccagggggtgtgctgtcgggtactggggggtgctggccgagctgtactgggcgttgtactgcatgtgctgcagcgactgcgcgctgtaggcagaaaagggaatgcccgcctggaaggtggcggctgccaggtcctgggctttgagcgcgtgacatggtttgccgtccctgaccaagacgggcacggccacccggcgcggcgagggcaggggcgtcacctccatacctttctcggcccgggcgcgcttcatcttgtagcggtggttctggaaccagatcttgacctgcgtgggcgtgaggcggatgaggctggccaggtgttcgcgctcgggcgccgacaggtaccgctgctgccgaaagcgccgctccagctcgtaggtctgcgccttggagaaaagcactcgccgctttcgcttcttgccggcgtcccccccgccgcccggggtctccttgtcattgtccggtgactcgtcggccgagggctccggggacttggagcttgagtcctgagggggcgccccggcagccagaccgtgcactggggggagggggagagagaagcgcgtcaggcgtctggaggccgcgcgcagcctgcaccagactcggagcaccctggatcctccgtgcgaccctggacctccgcgcctggcagcccagccccgctgcctttgccatgacccctgccttcctctacgcctgacgaagacgtcgggacctcccagggtaaagagggaaatccgtaggagtgggggctaggggctcgcctttcaaggggctcacatagtgtgggtgagaatttggggggggaagtggcactgaagatcatcgcgggttaagggctccggcgcccttaagcggtttcctctccggcgagacgtgggcagatttttaaaagaaataccccacaatcccagcattgatcggctcggccatttaaggcaggtttttgggggctttcgtttctgctttcgggccttctagacaccccctgcactgcacaaagcgtcctctatgatcgcgcagggggcagggtgagctatttatacccgggccacccgaagcctccccaccctccacccgtacccggacgaggctcgctggctacctccagggcgccaggccagtccctcccagcggccggagtccgggggctgcggctggagccatcggtccgggttgacatcatataccagcccctgggaggtatagcccttctccctctttctccttaacttctcgcattgaaaaaaattttggagaccaagttctttcccggaactaaggaggcttgaagaggagggcagaggacatcctatacaggtgttaaaaatctttctacggatccgagtgagggggtccgggcttacatggccccttcccctttcactcccagcgtccaacccgggctgcggctgcaggaatggaggggaccacggcctcggcagccaagttttgctacttacgggagtactgaaggccctcggtgctggccagccagcgcgtgtacgggttgtcgctgctgtcgtagaaggggttcttcaggggcaggctctgcaccgcgtccagggcgccctgccccagcggcccggccctcttggctggctcgggcccctcgttctcttcctccggaccttcggccacagagccctcctcatcgttggtgtccggcaggtctaagatgtccttgaccgaaaaccccgtctttgtgttggtcagcgacatggttcgagaccccaaaatttatgtcgcaaagttgtagcttcacttggtcaattcgtggcgctcccctgccccg

SEQ ID NO: 127 - DNA, 3088 bp, Artificial Sequence
  converted
tcgtttttgcggttttcgaagttattttttttttcgtagtcggacgttgtcgttcggggtttagatagcgcgcgagattaggggagatgcgggggagggtgggcggttttgggttttcggtcgatcgttcgtaggtcggaggtcgagcgttttttttttcgttttaattttaggcgcggggtgtagtttgtggtcggaggggtttgtatcgggagtttcgttcgttttagggtttcggtattttcggttttttcgttcgagaggaatcgtttgtgggagtttcgcgggcggtcgggttcggtttttaggtttaggttttcggttcggcgtagtaggataaacgggagggtagggatttttttttttttttttttttaaatataggatttttattaaaatttttatttaaaaaatcgaaagttttttgcgttcggcgttgttggggaatcgcgtcgaatagttgagttttaagttcggggttgaaggttcggaggatagggtagagggttttttgtttatagggtttttttttttatatttgagaaatgtttgtataataaaataaaaagagaaaggaagaaagtaggggaaaacgtaaaaataaaaataaaataaaaaataaatttaaataagttataaagaaaggagttggatttagacgggaaataggtcggttttgaaggttattttggtaataattattatcgatatttataacgaaaagcgaaatttgttattagttgttagaacgtttatatggttataaatatttagtatttttttatttttttttaaaaaaaggaagaaattttttgtatttttaaagtgttttttatttgttttagaagacggttgataatatcgttatttatataaatatattttataaaatgtacgaaagtaggggtggggggtcggtttttttttcgtttttaagtgacgatattaacgttgggacggtttggttttttcggggagaggtaaagaagcgaagttgcgtaaatattttgtaaatacggcgtagagtttagtttttttttaaggtttagaaggagaggtatgggtagagttgggtgggtggaatttgttattttaaggagacgtaggtaatttttcggcgtttttttagtggagtcgagagttaattcgattttataataataattataataataataataattattataaggatcgaggttttttcgtcgttatcgtcgtcggggtggggtttgggtttggggtcgcgagtttcgttggggcggcgtttattaagtttattgttgggtttggattagggggtgtgttgtcgggtattggggggtgttggtcgagttgtattgggcgttgtattgtatgtgttgtagcgattgcgcgttgtaggtagaaaagggaatgttcgtttggaaggtggcggttgttaggttttgggttttgagcgcgtgatatggtttgtcgtttttgattaagacgggtacggttattcggcgcggcgagggtaggggcgttatttttatatttttttcggttcgggcgcgttttattttgtagcggtggttttggaattagattttgatttgcgtgggcgtgaggcggatgaggttggttaggtgttcgcgttcgggcgtcgataggtatcgttgttgtcgaaagcgtcgttttagttcgtaggtttgcgttttggagaaaagtattcgtcgttttcgttttttgtcggcgtttttttcgtcgttcggggtttttttgttattgttcggtgattcgtcggtcgagggtttcggggatttggagtttgagttttgagggggcgtttcggtagttagatcgtgtattggggggagggggagagagaagcgcgttaggcgtttggaggtcgcgcgtagtttgtattagattcggagtattttggatttttcgtgcgattttggattttcgcgtttggtagtttagtttcgttgtttttgttatgatttttgtttttttttacgtttgacgaagacgtcgggattttttagggtaaagagggaaattcgtaggagtgggggttaggggttcgttttttaaggggtttatatagtgtgggtgagaatttggggggggaagtggtattgaagattatcgcgggttaagggtttcggcgtttttaagcggtttttttttcggcgagacgtgggtagatttttaaaagaaatattttataattttagtattgatcggttcggttatttaaggtaggtttttgggggttttcgtttttgttttcgggttttttagatattttttgtattgtataaagcgttttttatgatcgcgtagggggtagggtgagttatttatattcgggttattcgaagtttttttattttttattcgtattcggacgaggttcgttggttatttttagggcgttaggttagttttttttagcggtcggagttcgggggttgcggttggagttatcggttcgggttgatattatatattagtttttgggaggtatagtttttttttttttttttttttaatttttcgtattgaaaaaaattttggagattaagttttttttcggaattaaggaggtttgaagaggagggtagaggatattttatataggtgttaaaaatttttttacggattcgagtgagggggttcgggtttatatggttttttttttttttatttttagcgtttaattcgggttgcggttgtaggaatggaggggattacggtttcggtagttaagttttgttatttacgggagtattgaaggttttcggtgttggttagttagcgcgtgtacgggttgtcgttgttgtcgtagaaggggttttttaggggtaggttttgtatcgcgtttagggcgttttgttttagcggttcggtttttttggttggttcgggtttttcgtttttttttttcggattttcggttatagagttttttttatcgttggtgttcggtaggtttaagatgtttttgatcgaaaatttcgtttttgtgttggttagcgatatggttcgagattttaaaatttatgtcgtaaagttgtagttttatttggttaattcgtggcgtttttttgtttcg

SEQ ID NO: 128 - DNA, 3088 bp, Artificial Sequence
  converted
cgaaacaaaaaaacgccacgaattaaccaaataaaactacaactttacgacataaattttaaaatctcgaaccatatcgctaaccaacacaaaaacgaaattttcgatcaaaaacatcttaaacctaccgaacaccaacgataaaaaaaactctataaccgaaaatccgaaaaaaaaaaacgaaaaacccgaaccaaccaaaaaaaccgaaccgctaaaacaaaacgccctaaacgcgatacaaaacctacccctaaaaaaccccttctacgacaacaacgacaacccgtacacgcgctaactaaccaacaccgaaaaccttcaatactcccgtaaataacaaaacttaactaccgaaaccgtaatcccctccattcctacaaccgcaacccgaattaaacgctaaaaataaaaaaaaaaaaaaccatataaacccgaaccccctcactcgaatccgtaaaaaaatttttaacacctatataaaatatcctctaccctcctcttcaaacctccttaattccgaaaaaaaacttaatctccaaaatttttttcaatacgaaaaattaaaaaaaaaaaaaaaaaaaaactatacctcccaaaaactaatatataatatcaacccgaaccgataactccaaccgcaacccccgaactccgaccgctaaaaaaaactaacctaacgccctaaaaataaccaacgaacctcgtccgaatacgaataaaaaataaaaaaacttcgaataacccgaatataaataactcaccctaccccctacgcgatcataaaaaacgctttatacaatacaaaaaatatctaaaaaacccgaaaacaaaaacgaaaacccccaaaaacctaccttaaataaccgaaccgatcaatactaaaattataaaatatttcttttaaaaatctacccacgtctcgccgaaaaaaaaaccgcttaaaaacgccgaaacccttaacccgcgataatcttcaataccacttccccccccaaattctcacccacactatataaaccccttaaaaaacgaacccctaacccccactcctacgaatttccctctttaccctaaaaaatcccgacgtcttcgtcaaacgtaaaaaaaaacaaaaatcataacaaaaacaacgaaactaaactaccaaacgcgaaaatccaaaatcgcacgaaaaatccaaaatactccgaatctaatacaaactacgcgcgacctccaaacgcctaacgcgcttctctctccccctccccccaatacacgatctaactaccgaaacgccccctcaaaactcaaactccaaatccccgaaaccctcgaccgacgaatcaccgaacaataacaaaaaaaccccgaacgacgaaaaaaacgccgacaaaaaacgaaaacgacgaatacttttctccaaaacgcaaacctacgaactaaaacgacgctttcgacaacaacgatacctatcgacgcccgaacgcgaacacctaaccaacctcatccgcctcacgcccacgcaaatcaaaatctaattccaaaaccaccgctacaaaataaaacgcgcccgaaccgaaaaaaatataaaaataacgcccctaccctcgccgcgccgaataaccgtacccgtcttaatcaaaaacgacaaaccatatcacgcgctcaaaacccaaaacctaacaaccgccaccttccaaacgaacattcccttttctacctacaacgcgcaatcgctacaacacatacaatacaacgcccaatacaactcgaccaacaccccccaatacccgacaacacaccccctaatccaaacccaacaataaacttaataaacgccgccccaacgaaactcgcgaccccaaacccaaaccccaccccgacgacgataacgacgaaaaaacctcgatccttataataattattattattattataattattattataaaatcgaattaactctcgactccactaaaaaaacgccgaaaaattacctacgtctccttaaaataacaaattccacccacccaactctacccatacctctccttctaaaccttaaaaaaaaactaaactctacgccgtatttacaaaatatttacgcaacttcgcttctttacctctccccgaaaaaaccaaaccgtcccaacgttaatatcgtcacttaaaaacgaaaaaaaaaccgaccccccacccctactttcgtacattttataaaatatatttatataaataacgatattatcaaccgtcttctaaaacaaataaaaaacactttaaaaatacaaaaaatttcttcctttttttaaaaaaaaataaaaaaatactaaatatttataaccatataaacgttctaacaactaataacaaatttcgcttttcgttataaatatcgataataattattaccaaaataaccttcaaaaccgacctatttcccgtctaaatccaactcctttctttataacttatttaaatttattttttattttatttttatttttacgttttcccctactttcttcctttctctttttattttattatacaaacatttctcaaatataaaaaaaaaaaccctataaacaaaaaaccctctaccctatcctccgaaccttcaaccccgaacttaaaactcaactattcgacgcgattccccaacaacgccgaacgcaaaaaactttcgattttttaaataaaaattttaataaaaatcctatatttaaaaaaaaaaaaaaaaaaaaatccctaccctcccgtttatcctactacgccgaaccgaaaacctaaacctaaaaaccgaacccgaccgcccgcgaaactcccacaaacgattcctctcgaacgaaaaaaccgaaaataccgaaaccctaaaacgaacgaaactcccgatacaaacccctccgaccacaaactacaccccgcgcctaaaattaaaacgaaaaaaaaaacgctcgacctccgacctacgaacgatcgaccgaaaacccaaaaccgcccaccctcccccgcatctcccctaatctcgcgcgctatctaaaccccgaacgacaacgtccgactacgaaaaaaaaaataacttcgaaaaccgcaaaaacga

SEQ ID NO: 129 - DNA, 3088 bp, Artificial Sequence
  converted
tcgcctttacgactcccgaaatcacttttcctcccgtaaccgaacgctaccgcccgaaattcaaacaacgcgcgaaaccaaaaaaaatacgaaaaaaaataaacgaccctaaacttccgaccgaccgctcgcaaaccgaaaaccgaacgttctcctccccgccccaatcctaaacgcgaaatacaacctataatcgaaaaaacctacaccgaaaaccccgcccgccccaaaaccccgatacccccgacctccccgcccgaaaaaaaccgcctataaaaaccccgcgaacgaccgaacccgactcccaaacccaaacctccgacccgacgcaataaaacaaacgaaaaaacaaaaattttttctttttttcttttttaaacacaaaatttttattaaaattcttatttaaaaaatcgaaaactttctacgcccgacgctattaaaaaaccgcgccgaataactaaactccaaattcgaaactaaaaacccgaaaaacaaaacaaaaaactccctacctacaaaattttcttttccatatttaaaaaatatttacacaataaaataaaaaaaaaaaaaaaaaaaacaaaaaaaaacgcaaaaacaaaaacaaaacaaaaaacaaacccaaacaaaccacaaaaaaaaaaattaaacccaaacgaaaaacaaaccgatcctaaaaatcattttaacaacaatcaccaccgatatttacaacgaaaaacgaaatctaccaccaattatcaaaacgtttacataaccataaatatttaacattttcttatttttttttaaaaaaaaaaaaaaattctctatatttttaaaatattctccacttactttaaaaaacgactaacaatatcgctactcacacaaacatattttacaaaatacacgaaaacaaaaataaaaaatcgatctttttctcgttttcaaataacgacattaacgctaaaacgatttaatccccccgaaaaaaaacaaaaaaacgaaactacgcaaacattctataaacacgacgtaaaattcaaccctctcccaaaattcaaaaaaaaaaacataaacaaaactaaataaataaaatctaccactccaaaaaaacgcaaacaacctcccgacgcctccctaataaaaccgaaaatcaactcgactccataataataattataataataataataaccaccataaaaaccgaaacctcctcgccgccaccgccgccgaaataaaacctaaacctaaaaccgcgaatctcgttaaaacgacgctcaccaaatccactactaaacctaaaccaaaaaatatactatcgaatactaaaaaatactaaccgaactatactaaacgttatactacatatactacaacgactacgcgctataaacaaaaaaaaaaatacccgcctaaaaaataacgactaccaaatcctaaactttaaacgcgtaacataatttaccgtccctaaccaaaacgaacacgaccacccgacgcgacgaaaacaaaaacgtcacctccatacctttctcgacccgaacgcgcttcatcttataacgataattctaaaaccaaatcttaacctacgtaaacgtaaaacgaataaaactaaccaaatattcgcgctcgaacgccgacaaataccgctactaccgaaaacgccgctccaactcgtaaatctacgccttaaaaaaaaacactcgccgctttcgcttcttaccgacgtcccccccgccgcccgaaatctccttatcattatccgataactcgtcgaccgaaaactccgaaaacttaaaacttaaatcctaaaaaaacgccccgacaaccaaaccgtacactaaaaaaaaaaaaaaaaaaaaacgcgtcaaacgtctaaaaaccgcgcgcaacctacaccaaactcgaaacaccctaaatcctccgtacgaccctaaacctccgcgcctaacaacccaaccccgctacctttaccataacccctaccttcctctacgcctaacgaaaacgtcgaaacctcccaaaataaaaaaaaaaatccgtaaaaataaaaactaaaaactcgcctttcaaaaaactcacataatataaataaaaatttaaaaaaaaaaataacactaaaaatcatcgcgaattaaaaactccgacgcccttaaacgatttcctctccgacgaaacgtaaacaaatttttaaaaaaaataccccacaatcccaacattaatcgactcgaccatttaaaacaaatttttaaaaactttcgtttctactttcgaaccttctaaacaccccctacactacacaaaacgtcctctataatcgcgcaaaaaacaaaataaactatttatacccgaaccacccgaaacctccccaccctccacccgtacccgaacgaaactcgctaactacctccaaaacgccaaaccaatccctcccaacgaccgaaatccgaaaactacgactaaaaccatcgatccgaattaacatcatataccaacccctaaaaaatataacccttctccctctttctccttaacttctcgcattaaaaaaaattttaaaaaccaaattctttcccgaaactaaaaaaacttaaaaaaaaaaacaaaaaacatcctatacaaatattaaaaatctttctacgaatccgaataaaaaaatccgaacttacataaccccttcccctttcactcccaacgtccaacccgaactacgactacaaaaataaaaaaaaccacgacctcgacaaccaaattttactacttacgaaaatactaaaaaccctcgatactaaccaaccaacgcgtatacgaattatcgctactatcgtaaaaaaaattcttcaaaaacaaactctacaccgcgtccaaaacgccctaccccaacgacccgaccctcttaactaactcgaacccctcgttctcttcctccgaaccttcgaccacaaaaccctcctcatcgttaatatccgacaaatctaaaatatccttaaccgaaaaccccgtctttatattaatcaacgacataattcgaaaccccaaaatttatatcgcaaaattataacttcacttaatcaattcgtaacgctcccctaccccg

SEQ ID NO: 130 - DNA, 3088 bp, Artificial Sequence
  converted
cggggtaggggagcgttacgaattgattaagtgaagttataattttgcgatataaattttggggtttcgaattatgtcgttgattaatataaagacggggttttcggttaaggatattttagatttgtcggatattaacgatgaggagggttttgtggtcgaaggttcggaggaagagaacgaggggttcgagttagttaagagggtcgggtcgttggggtagggcgttttggacgcggtgtagagtttgtttttgaagaattttttttacgatagtagcgataattcgtatacgcgttggttggttagtatcgagggtttttagtattttcgtaagtagtaaaatttggttgtcgaggtcgtggttttttttatttttgtagtcgtagttcgggttggacgttgggagtgaaaggggaaggggttatgtaagttcggatttttttattcggattcgtagaaagatttttaatatttgtataggatgttttttgtttttttttttaagtttttttagtttcgggaaagaatttggtttttaaaattttttttaatgcgagaagttaaggagaaagagggagaagggttatattttttaggggttggtatatgatgttaattcggatcgatggttttagtcgtagttttcggatttcggtcgttgggagggattggtttggcgttttggaggtagttagcgagtttcgttcgggtacgggtggagggtggggaggtttcgggtggttcgggtataaatagtttattttgttttttgcgcgattatagaggacgttttgtgtagtgtagggggtgtttagaaggttcgaaagtagaaacgaaagtttttaaaaatttgttttaaatggtcgagtcgattaatgttgggattgtggggtattttttttaaaaatttgtttacgtttcgtcggagaggaaatcgtttaagggcgtcggagtttttaattcgcgatgatttttagtgttatttttttttttaaatttttatttatattatgtgagttttttgaaaggcgagtttttagtttttatttttacggatttttttttttattttgggaggtttcgacgttttcgttaggcgtagaggaaggtaggggttatggtaaaggtagcggggttgggttgttaggcgcggaggtttagggtcgtacggaggatttagggtgtttcgagtttggtgtaggttgcgcgcggtttttagacgtttgacgcgtttttttttttttttttttttagtgtacggtttggttgtcggggcgtttttttaggatttaagttttaagttttcggagttttcggtcgacgagttatcggataatgataaggagatttcgggcggcgggggggacgtcggtaagaagcgaaagcggcgagtgtttttttttaaggcgtagatttacgagttggagcggcgttttcggtagtagcggtatttgtcggcgttcgagcgcgaatatttggttagttttattcgttttacgtttacgtaggttaagatttggttttagaattatcgttataagatgaagcgcgttcgggtcgagaaaggtatggaggtgacgtttttgttttcgtcgcgtcgggtggtcgtgttcgttttggttagggacggtaaattatgttacgcgtttaaagtttaggatttggtagtcgttattttttaggcgggtatttttttttttgtttatagcgcgtagtcgttgtagtatatgtagtataacgtttagtatagttcggttagtattttttagtattcgatagtatattttttggtttaggtttagtagtggatttggtgagcgtcgttttaacgagattcgcggttttaggtttaggttttatttcggcggcggtggcggcgaggaggtttcggtttttatggtggttattattattattataattattattatggagtcgagttgattttcggttttattagggaggcgtcgggaggttgtttgcgtttttttggagtggtagattttatttatttagttttgtttatgtttttttttttgaattttgggagagggttgaattttacgtcgtgtttatagaatgtttgcgtagtttcgtttttttgttttttttcggggggattaaatcgttttagcgttaatgtcgttatttgaaaacgagaaaaagatcgattttttatttttgttttcgtgtattttgtaaaatatgtttgtgtgagtagcgatattgttagtcgttttttaaagtaagtggagaatattttaaaaatatagagaatttttttttttttttaaaaaaaaataagaaaatgttaaatatttatggttatgtaaacgttttgataattggtggtagatttcgtttttcgttgtaaatatcggtggtgattgttgttaaaatgatttttaggatcggtttgtttttcgtttgggtttaatttttttttttgtggtttgtttgggtttgttttttgttttgtttttgtttttgcgtttttttttgttttttttttttttttttttattttattgtgtaaatattttttaaatatggaaaagaaaattttgtaggtagggagttttttgttttgtttttcgggtttttagtttcgaatttggagtttagttattcggcgcggttttttaatagcgtcgggcgtagaaagttttcgattttttaaataagaattttaataaaaattttgtgtttaaaaaagaaaaaaagaaaaaatttttgttttttcgtttgttttattgcgtcgggtcggaggtttgggtttgggagtcgggttcggtcgttcgcggggtttttataggcggtttttttcgggcggggaggtcgggggtatcggggttttggggcgggcggggttttcggtgtaggttttttcgattataggttgtatttcgcgtttaggattggggcggggaggagaacgttcggttttcggtttgcgagcggtcggtcggaagtttagggtcgtttattttttttcgtattttttttggtttcgcgcgttgtttgaatttcgggcggtagcgttcggttacgggaggaaaagtgatttcgggagtcgtaaaggcga

SEQ ID NO: 131 - DNA, 1067 bp, Homo sapiens
ggctggccaggtgttcgcgctcgggcgccgacaggtaccgctgctgccgaaagcgccgctccagctcgtaggtctgcgccttggagaaaagcactcgccgctttcgcttcttgccggcgtcccccccgccgcccggggtctccttgtcattgtccggtgactcgtcggccgagggctccggggacttggagcttgagtcctgagggggcgccccggcagccagaccgtgcactggggggagggggagagagaagcgcgtcaggcgtctggaggccgcgcgcagcctgcaccagactcggagcaccctggatcctccgtgcgaccctggacctccgcgcctggcagcccagccccgctgcctttgccatgacccctgccttcctctacgcctgacgaagacgtcgggacctcccagggtaaagagggaaatccgtaggagtgggggctaggggctcgcctttcaaggggctcacatagtgtgggtgagaatttggggggggaagtggcactgaagatcatcgcgggttaagggctccggcgcccttaagcggtttcctctccggcgagacgtgggcagatttttaaaagaaataccccacaatcccagcattgatcggctcggccatttaaggcaggtttttgggggctttcgtttctgctttcgggccttctagacaccccctgcactgcacaaagcgtcctctatgatcgcgcagggggcagggtgagctatttatacccgggccacccgaagcctccccaccctccacccgtacccggacgaggctcgctggctacctccagggcgccaggccagtccctcccagcggccggagtccgggggctgcggctggagccatcggtccgggttgacatcatataccagcccctgggaggtatagcccttctccctctttctccttaacttctcgcattgaaaaaaattttggagaccaagttctttcccggaactaaggaggcttgaagaggagggcagaggacatcctatacaggtgttaaaaatctttctacggatccgagtgagggggtccgggcttacatggccc

SEQ ID NO: 132 - DNA, 1067 bp, Artificial Sequence
  converted
ggttggttaggtgttcgcgttcgggcgtcgataggtatcgttgttgtcgaaagcgtcgttttagttcgtaggtttgcgttttggagaaaagtattcgtcgttttcgttttttgtcggcgtttttttcgtcgttcggggtttttttgttattgttcggtgattcgtcggtcgagggtttcggggatttggagtttgagttttgagggggcgtttcggtagttagatcgtgtattggggggagggggagagagaagcgcgttaggcgtttggaggtcgcgcgtagtttgtattagattcggagtattttggatttttcgtgcgattttggattttcgcgtttggtagtttagtttcgttgtttttgttatgatttttgtttttttttacgtttgacgaagacgtcgggattttttagggtaaagagggaaattcgtaggagtgggggttaggggttcgttttttaaggggtttatatagtgtgggtgagaatttggggggggaagtggtattgaagattatcgcgggttaagggtttcggcgtttttaagcggtttttttttcggcgagacgtgggtagatttttaaaagaaatattttataattttagtattgatcggttcggttatttaaggtaggtttttgggggttttcgtttttgttttcgggttttttagatattttttgtattgtataaagcgttttttatgatcgcgtagggggtagggtgagttatttatattcgggttattcgaagtttttttattttttattcgtattcggacgaggttcgttggttatttttagggcgttaggttagttttttttagcggtcggagttcgggggttgcggttggagttatcggttcgggttgatattatatattagtttttgggaggtatagtttttttttttttttttttttaatttttcgtattgaaaaaaattttggagattaagttttttttcggaattaaggaggtttgaagaggagggtagaggatattttatataggtgttaaaaatttttttacggattcgagtgagggggttcgggtttatatggttt

SEQ ID NO: 133 - DNA, 1067 bp, Artificial Sequence
  converted
aaaccatataaacccgaaccccctcactcgaatccgtaaaaaaatttttaacacctatataaaatatcctctaccctcctcttcaaacctccttaattccgaaaaaaaacttaatctccaaaatttttttcaatacgaaaaattaaaaaaaaaaaaaaaaaaaaactatacctcccaaaaactaatatataatatcaacccgaaccgataactccaaccgcaacccccgaactccgaccgctaaaaaaaactaacctaacgccctaaaaataaccaacgaacctcgtccgaatacgaataaaaaataaaaaaacttcgaataacccgaatataaataactcaccctaccccctacgcgatcataaaaaacgctttatacaatacaaaaaatatctaaaaaacccgaaaacaaaaacgaaaacccccaaaaacctaccttaaataaccgaaccgatcaatactaaaattataaaatatttcttttaaaaatctacccacgtctcgccgaaaaaaaaaccgcttaaaaacgccgaaacccttaacccgcgataatcttcaataccacttccccccccaaattctcacccacactatataaaccccttaaaaaacgaacccctaacccccactcctacgaatttccctctttaccctaaaaaatcccgacgtcttcgtcaaacgtaaaaaaaaacaaaaatcataacaaaaacaacgaaactaaactaccaaacgcgaaaatccaaaatcgcacgaaaaatccaaaatactccgaatctaatacaaactacgcgcgacctccaaacgcctaacgcgcttctctctccccctccccccaatacacgatctaactaccgaaacgccccctcaaaactcaaactccaaatccccgaaaccctcgaccgacgaatcaccgaacaataacaaaaaaaccccgaacgacgaaaaaaacgccgacaaaaaacgaaaacgacgaatacttttctccaaaacgcaaacctacgaactaaaacgacgctttcgacaacaacgatacctatcgacgcccgaacgcgaacacctaaccaacc

SEQ ID NO: 134 - DNA, 1067 bp, Artificial Sequence
  converted
aactaaccaaatattcgcgctcgaacgccgacaaataccgctactaccgaaaacgccgctccaactcgtaaatctacgccttaaaaaaaaacactcgccgctttcgcttcttaccgacgtcccccccgccgcccgaaatctccttatcattatccgataactcgtcgaccgaaaactccgaaaacttaaaacttaaatcctaaaaaaacgccccgacaaccaaaccgtacactaaaaaaaaaaaaaaaaaaaaacgcgtcaaacgtctaaaaaccgcgcgcaacctacaccaaactcgaaacaccctaaatcctccgtacgaccctaaacctccgcgcctaacaacccaaccccgctacctttaccataacccctaccttcctctacgcctaacgaaaacgtcgaaacctcccaaaataaaaaaaaaaatccgtaaaaataaaaactaaaaactcgcctttcaaaaaactcacataatataaataaaaatttaaaaaaaaaaataacactaaaaatcatcgcgaattaaaaactccgacgcccttaaacgatttcctctccgacgaaacgtaaacaaatttttaaaaaaaataccccacaatcccaacattaatcgactcgaccatttaaaacaaatttttaaaaactttcgtttctactttcgaaccttctaaacaccccctacactacacaaaacgtcctctataatcgcgcaaaaaacaaaataaactatttatacccgaaccacccgaaacctccccaccctccacccgtacccgaacgaaactcgctaactacctccaaaacgccaaaccaatccctcccaacgaccgaaatccgaaaactacgactaaaaccatcgatccgaattaacatcatataccaacccctaaaaaatataacccttctccctctttctccttaacttctcgcattaaaaaaaattttaaaaaccaaattctttcccgaaactaaaaaaacttaaaaaaaaaaacaaaaaacatcctatacaaatattaaaaatctttctacgaatccgaataaaaaaatccgaacttacataaccc

SEQ ID NO: 135 - DNA, 1067 bp, Artificial Sequence
  converted
gggttatgtaagttcggatttttttattcggattcgtagaaagatttttaatatttgtataggatgttttttgtttttttttttaagtttttttagtttcgggaaagaatttggtttttaaaattttttttaatgcgagaagttaaggagaaagagggagaagggttatattttttaggggttggtatatgatgttaattcggatcgatggttttagtcgtagttttcggatttcggtcgttgggagggattggtttggcgttttggaggtagttagcgagtttcgttcgggtacgggtggagggtggggaggtttcgggtggttcgggtataaatagtttattttgttttttgcgcgattatagaggacgttttgtgtagtgtagggggtgtttagaaggttcgaaagtagaaacgaaagtttttaaaaatttgttttaaatggtcgagtcgattaatgttgggattgtggggtattttttttaaaaatttgtttacgtttcgtcggagaggaaatcgtttaagggcgtcggagtttttaattcgcgatgatttttagtgttatttttttttttaaatttttatttatattatgtgagttttttgaaaggcgagtttttagtttttatttttacggatttttttttttattttgggaggtttcgacgttttcgttaggcgtagaggaaggtaggggttatggtaaaggtagcggggttgggttgttaggcgcggaggtttagggtcgtacggaggatttagggtgtttcgagtttggtgtaggttgcgcgcggtttttagacgtttgacgcgtttttttttttttttttttttagtgtacggtttggttgtcggggcgtttttttaggatttaagttttaagttttcggagttttcggtcgacgagttatcggataatgataaggagatttcgggcggcgggggggacgtcggtaagaagcgaaagcggcgagtgtttttttttaaggcgtagatttacgagttggagcggcgttttcggtagtagcggtatttgtcggcgttcgagcgcgaatatttggttagtt

SEQ ID NO: 136 - DNA, 67 bp, Homo sapiens
aagtggcactgaagatcatcgcgggttaagggctccggcgcccttaagcggtttcctctccggcgag

SEQ ID NO: 137 - DNA, 67 bp, Artificial Sequence
  converted
aagtggtattgaagattatcgcgggttaagggtttcggcgtttttaagcggtttttttttcggcgag

SEQ ID NO: 138 - DNA, 67 bp, Artificial Sequence
  converted
ctcgccgaaaaaaaaaccgcttaaaaacgccgaaacccttaacccgcgataatcttcaataccactt

SEQ ID NO: 139 - DNA, 67 bp, Artificial Sequence
  converted
aaataacactaaaaatcatcgcgaattaaaaactccgacgcccttaaacgatttcctctccgacgaa

SEQ ID NO: 140 - DNA, 67 bp, Artificial Sequence
  converted
ttcgtcggagaggaaatcgtttaagggcgtcggagtttttaattcgcgatgatttttagtgttattt

SEQ ID NO: 141 - DNA, 1120 bp, Homo sapiens
ccgaaaaacacgttctaggcgccgggattccagatacctgggaaatagagtgcacgcagctgttgagaggcctcgcgcttggcttctcctatcactgaggcgcagaggtgctgtggacagcccagacccacacggcgcccgaggtgaaacagaaccctcagtctccctatgaggccactggcactctcggctgtccccagagctctccgacttagagctgaatgcaaagtaagcgctcgaaatgcagaagtagccggggccgcccacggcacctgcctcgctcggggcgagagaagacgccaggctgaggtcccagcgacctcaggcaccagctccgaaggagggcggggagaccgcaaaggggaagtgcccggagggccaacggcccccgcgcaccctgcgcccctctgaagcgcgccgcctccccgcgccggggactgggacctgcctctggggaatccgcctagaagacggcggcggactggggtcgggcactctccagggctgtcaggccctccccagccctgcacctgccgcgccgccccacctcgccaggaagtctcagagaccccggggatggggtgggagcgccttcccatcgcgggctcaaaaagaaggaaggacgcccccaggggtcgtagaaggaggactagctccaagccacaactttcttcggacccaaggcaggccggctggggctccgcgcctacacggcccctggcgggggtccgcgcgccccgggagccccgcggctcggggaggaaagaggagacaagagacaggcgaggattacggggctgacccagccggggtagggaccatcgtggaaaaactttggcgaggtggggggacgcggaaagagagcggcccgcgccctgcaccttgcgccgggcatcccgcgccagtgcctcgctcccagtgccccgcgccccgcgccccgcgccttgccttcaccccgggccagctgcatcgcgcccgcgccgcaggaaccgtggagttggaaagtgggggcgccgcggctggggggctgcttcagctgcgcctcggccagcgatcggcgggccgggctcaaatccagccaggctgggcaggcggtggccgcgcgactggggaccgggcg

SEQ ID NO: 142 - DNA, 1120 bp, Artificial Sequence
  converted
tcgaaaaatacgttttaggcgtcgggattttagatatttgggaaatagagtgtacgtagttgttgagaggtttcgcgtttggttttttttattattgaggcgtagaggtgttgtggatagtttagatttatacggcgttcgaggtgaaatagaatttttagtttttttatgaggttattggtattttcggttgtttttagagtttttcgatttagagttgaatgtaaagtaagcgttcgaaatgtagaagtagtcggggtcgtttacggtatttgtttcgttcggggcgagagaagacgttaggttgaggttttagcgattttaggtattagtttcgaaggagggcggggagatcgtaaaggggaagtgttcggagggttaacggttttcgcgtattttgcgtttttttgaagcgcgtcgttttttcgcgtcggggattgggatttgtttttggggaattcgtttagaagacggcggcggattggggtcgggtattttttagggttgttaggttttttttagttttgtatttgtcgcgtcgttttatttcgttaggaagttttagagatttcggggatggggtgggagcgtttttttatcgcgggtttaaaaagaaggaaggacgtttttaggggtcgtagaaggaggattagttttaagttataatttttttcggatttaaggtaggtcggttggggtttcgcgtttatacggtttttggcgggggttcgcgcgtttcgggagtttcgcggttcggggaggaaagaggagataagagataggcgaggattacggggttgatttagtcggggtagggattatcgtggaaaaattttggcgaggtggggggacgcggaaagagagcggttcgcgttttgtattttgcgtcgggtatttcgcgttagtgtttcgtttttagtgtttcgcgtttcgcgtttcgcgttttgtttttatttcgggttagttgtatcgcgttcgcgtcgtaggaatcgtggagttggaaagtgggggcgtcgcggttggggggttgttttagttgcgtttcggttagcgatcggcgggtcgggtttaaatttagttaggttgggtaggcggtggtcgcgcgattggggatcgggcg

SEQ ID NO: 143 - DNA, 1120 bp, Artificial Sequence
  converted
cgcccgatccccaatcgcgcgaccaccgcctacccaacctaactaaatttaaacccgacccgccgatcgctaaccgaaacgcaactaaaacaaccccccaaccgcgacgcccccactttccaactccacgattcctacgacgcgaacgcgatacaactaacccgaaataaaaacaaaacgcgaaacgcgaaacgcgaaacactaaaaacgaaacactaacgcgaaatacccgacgcaaaatacaaaacgcgaaccgctctctttccgcgtccccccacctcgccaaaatttttccacgataatccctaccccgactaaatcaaccccgtaatcctcgcctatctcttatctcctctttcctccccgaaccgcgaaactcccgaaacgcgcgaacccccgccaaaaaccgtataaacgcgaaaccccaaccgacctaccttaaatccgaaaaaaattataacttaaaactaatcctccttctacgacccctaaaaacgtccttccttctttttaaacccgcgataaaaaaacgctcccaccccatccccgaaatctctaaaacttcctaacgaaataaaacgacgcgacaaatacaaaactaaaaaaaacctaacaaccctaaaaaatacccgaccccaatccgccgccgtcttctaaacgaattccccaaaaacaaatcccaatccccgacgcgaaaaaacgacgcgcttcaaaaaaacgcaaaatacgcgaaaaccgttaaccctccgaacacttcccctttacgatctccccgccctccttcgaaactaatacctaaaatcgctaaaacctcaacctaacgtcttctctcgccccgaacgaaacaaataccgtaaacgaccccgactacttctacatttcgaacgcttactttacattcaactctaaatcgaaaaactctaaaaacaaccgaaaataccaataacctcataaaaaaactaaaaattctatttcacctcgaacgccgtataaatctaaactatccacaacacctctacgcctcaataataaaaaaaaccaaacgcgaaacctctcaacaactacgtacactctatttcccaaatatctaaaatcccgacgcctaaaacgtatttttcga

SEQ ID NO: 144 - DNA, 1120 bp, Artificial Sequence
  converted
ccgaaaaacacgttctaaacgccgaaattccaaatacctaaaaaataaaatacacgcaactattaaaaaacctcgcgcttaacttctcctatcactaaaacgcaaaaatactataaacaacccaaacccacacgacgcccgaaataaaacaaaaccctcaatctccctataaaaccactaacactctcgactatccccaaaactctccgacttaaaactaaatacaaaataaacgctcgaaatacaaaaataaccgaaaccgcccacgacacctacctcgctcgaaacgaaaaaaaacgccaaactaaaatcccaacgacctcaaacaccaactccgaaaaaaaacgaaaaaaccgcaaaaaaaaaatacccgaaaaaccaacgacccccgcgcaccctacgcccctctaaaacgcgccgcctccccgcgccgaaaactaaaacctacctctaaaaaatccgcctaaaaaacgacgacgaactaaaatcgaacactctccaaaactatcaaaccctccccaaccctacacctaccgcgccgccccacctcgccaaaaaatctcaaaaaccccgaaaataaaataaaaacgccttcccatcgcgaactcaaaaaaaaaaaaaaacgcccccaaaaatcgtaaaaaaaaaactaactccaaaccacaactttcttcgaacccaaaacaaaccgactaaaactccgcgcctacacgacccctaacgaaaatccgcgcgccccgaaaaccccgcgactcgaaaaaaaaaaaaaaaacaaaaaacaaacgaaaattacgaaactaacccaaccgaaataaaaaccatcgtaaaaaaactttaacgaaataaaaaaacgcgaaaaaaaaacgacccgcgccctacaccttacgccgaacatcccgcgccaatacctcgctcccaataccccgcgccccgcgccccgcgccttaccttcaccccgaaccaactacatcgcgcccgcgccgcaaaaaccgtaaaattaaaaaataaaaacgccgcgactaaaaaactacttcaactacgcctcgaccaacgatcgacgaaccgaactcaaatccaaccaaactaaacaaacgataaccgcgcgactaaaaaccgaacg

SEQ ID NO: 145 - DNA, 1120 bp, Artificial Sequence
  converted
cgttcggtttttagtcgcgcggttatcgtttgtttagtttggttggatttgagttcggttcgtcgatcgttggtcgaggcgtagttgaagtagttttttagtcgcggcgtttttattttttaattttacggtttttgcggcgcgggcgcgatgtagttggttcggggtgaaggtaaggcgcggggcgcggggcgcggggtattgggagcgaggtattggcgcgggatgttcggcgtaaggtgtagggcgcgggtcgtttttttttcgcgtttttttatttcgttaaagtttttttacgatggtttttatttcggttgggttagtttcgtaattttcgtttgttttttgttttttttttttttttcgagtcgcggggttttcggggcgcgcggattttcgttaggggtcgtgtaggcgcggagttttagtcggtttgttttgggttcgaagaaagttgtggtttggagttagttttttttttacgatttttgggggcgtttttttttttttttgagttcgcgatgggaaggcgtttttattttattttcggggtttttgagattttttggcgaggtggggcggcgcggtaggtgtagggttggggagggtttgatagttttggagagtgttcgattttagttcgtcgtcgttttttaggcggattttttagaggtaggttttagttttcggcgcggggaggcggcgcgttttagaggggcgtagggtgcgcgggggtcgttggtttttcgggtattttttttttgcggttttttcgttttttttcggagttggtgtttgaggtcgttgggattttagtttggcgttttttttcgtttcgagcgaggtaggtgtcgtgggcggtttcggttatttttgtatttcgagcgtttattttgtatttagttttaagtcggagagttttggggatagtcgagagtgttagtggttttatagggagattgagggttttgttttatttcgggcgtcgtgtgggtttgggttgtttatagtatttttgcgttttagtgataggagaagttaagcgcgaggttttttaatagttgcgtgtattttattttttaggtatttggaatttcggcgtttagaacgtgtttttcgg

SEQ ID NO: 146 - DNA, 1077 bp, Homo sapiens
cctggtaagtaaatgtggatttattagtaaaataagcctagctgcagattcctcatccagatgttcctgaaagaaggcccctggctctgtcttcccggctgtttaaaagaatcattgtttctaattctttcctcccattgtactaagatcaactgtggcaggaactgggtccccctccaggacaaattgttgtttgtcagctgattggttcatacattcattcatcatttacaaggtacttactatatccccaggctgccgtagacacagcctttgctctcccgaaaaacacgttctaggcgccgggattccagatacctgggaaatagagtgcacgcagctgttgagaggcctcgcgcttggcttctcctatcactgaggcgcagaggtgctgtggacagcccagacccacacggcgcccgaggtgaaacagaaccctcagtctccctatgaggccactggcactctcggctgtccccagagctctccgacttagagctgaatgcaaagtaagcgctcgaaatgcagaagtagccggggccgcccacggcacctgcctcgctcggggcgagagaagacgccaggctgaggtcccagcgacctcaggcaccagctccgaaggagggcggggagaccgcaaaggggaagtgcccggagggccaacggcccccgcgcaccctgcgcccctctgaagcgcgccgcctccccgcgccggggactgggacctgcctctggggaatccgcctagaagacggcggcggactggggtcgggcactctccagggctgtcaggccctccccagccctgcacctgccgcgccgccccacctcgccaggaagtctcagagaccccggggatggggtgggagcgccttcccatcgcgggctcaaaaagaaggaaggacgcccccaggggtcgtagaaggaggactagctccaagccacaactttcttcggacccaaggcaggccggctggggctccgcgcctacacggcccctggcgggggtccgcgcgccccgggagccccgcggctcggggaggaaagaggagacaagagacaggcgaggattacggg

SEQ ID NO: 147 - DNA, 1077 bp, Artificial Sequence
  converted
tttggtaagtaaatgtggatttattagtaaaataagtttagttgtagattttttatttagatgtttttgaaagaaggtttttggttttgttttttcggttgtttaaaagaattattgtttttaattttttttttttattgtattaagattaattgtggtaggaattgggtttttttttaggataaattgttgtttgttagttgattggtttatatatttatttattatttataaggtatttattatatttttaggttgtcgtagatatagtttttgttttttcgaaaaatacgttttaggcgtcgggattttagatatttgggaaatagagtgtacgtagttgttgagaggtttcgcgtttggttttttttattattgaggcgtagaggtgttgtggatagtttagatttatacggcgttcgaggtgaaatagaatttttagtttttttatgaggttattggtattttcggttgtttttagagtttttcgatttagagttgaatgtaaagtaagcgttcgaaatgtagaagtagtcggggtcgtttacggtatttgtttcgttcggggcgagagaagacgttaggttgaggttttagcgattttaggtattagtttcgaaggagggcggggagatcgtaaaggggaagtgttcggagggttaacggttttcgcgtattttgcgtttttttgaagcgcgtcgttttttcgcgtcggggattgggatttgtttttggggaattcgtttagaagacggcggcggattggggtcgggtattttttagggttgttaggttttttttagttttgtatttgtcgcgtcgttttatttcgttaggaagttttagagatttcggggatggggtgggagcgtttttttatcgcgggtttaaaaagaaggaaggacgtttttaggggtcgtagaaggaggattagttttaagttataatttttttcggatttaaggtaggtcggttggggtttcgcgtttatacggtttttggcgggggttcgcgcgtttcgggagtttcgcggttcggggaggaaagaggagataagagataggcgaggattacggg

SEQ ID NO: 148 - DNA, 1077 bp, Artificial Sequence
  converted
cccgtaatcctcgcctatctcttatctcctctttcctccccgaaccgcgaaactcccgaaacgcgcgaacccccgccaaaaaccgtataaacgcgaaaccccaaccgacctaccttaaatccgaaaaaaattataacttaaaactaatcctccttctacgacccctaaaaacgtccttccttctttttaaacccgcgataaaaaaacgctcccaccccatccccgaaatctctaaaacttcctaacgaaataaaacgacgcgacaaatacaaaactaaaaaaaacctaacaaccctaaaaaatacccgaccccaatccgccgccgtcttctaaacgaattccccaaaaacaaatcccaatccccgacgcgaaaaaacgacgcgcttcaaaaaaacgcaaaatacgcgaaaaccgttaaccctccgaacacttcccctttacgatctccccgccctccttcgaaactaatacctaaaatcgctaaaacctcaacctaacgtcttctctcgccccgaacgaaacaaataccgtaaacgaccccgactacttctacatttcgaacgcttactttacattcaactctaaatcgaaaaactctaaaaacaaccgaaaataccaataacctcataaaaaaactaaaaattctatttcacctcgaacgccgtataaatctaaactatccacaacacctctacgcctcaataataaaaaaaaccaaacgcgaaacctctcaacaactacgtacactctatttcccaaatatctaaaatcccgacgcctaaaacgtatttttcgaaaaaacaaaaactatatctacgacaacctaaaaatataataaataccttataaataataaataaatatataaaccaatcaactaacaaacaacaatttatcctaaaaaaaaacccaattcctaccacaattaatcttaatacaataaaaaaaaaaaattaaaaacaataattcttttaaacaaccgaaaaaacaaaaccaaaaaccttctttcaaaaacatctaaataaaaaatctacaactaaacttattttactaataaatccacatttacttaccaaa

SEQ ID NO: 149 - DNA, 1077 bp, Artificial Sequence
  converted
cctaataaataaatataaatttattaataaaataaacctaactacaaattcctcatccaaatattcctaaaaaaaaacccctaactctatcttcccgactatttaaaaaaatcattatttctaattctttcctcccattatactaaaatcaactataacaaaaactaaatccccctccaaaacaaattattatttatcaactaattaattcatacattcattcatcatttacaaaatacttactatatccccaaactaccgtaaacacaacctttactctcccgaaaaacacgttctaaacgccgaaattccaaatacctaaaaaataaaatacacgcaactattaaaaaacctcgcgcttaacttctcctatcactaaaacgcaaaaatactataaacaacccaaacccacacgacgcccgaaataaaacaaaaccctcaatctccctataaaaccactaacactctcgactatccccaaaactctccgacttaaaactaaatacaaaataaacgctcgaaatacaaaaataaccgaaaccgcccacgacacctacctcgctcgaaacgaaaaaaaacgccaaactaaaatcccaacgacctcaaacaccaactccgaaaaaaaacgaaaaaaccgcaaaaaaaaaatacccgaaaaaccaacgacccccgcgcaccctacgcccctctaaaacgcgccgcctccccgcgccgaaaactaaaacctacctctaaaaaatccgcctaaaaaacgacgacgaactaaaatcgaacactctccaaaactatcaaaccctccccaaccctacacctaccgcgccgccccacctcgccaaaaaatctcaaaaaccccgaaaataaaataaaaacgccttcccatcgcgaactcaaaaaaaaaaaaaaacgcccccaaaaatcgtaaaaaaaaaactaactccaaaccacaactttcttcgaacccaaaacaaaccgactaaaactccgcgcctacacgacccctaacgaaaatccgcgcgccccgaaaaccccgcgactcgaaaaaaaaaaaaaaaacaaaaaacaaacgaaaattacgaa

SEQ ID NO: 150 - DNA, 1077 bp, Artificial Sequence
  converted
ttcgtaattttcgtttgttttttgttttttttttttttttcgagtcgcggggttttcggggcgcgcggattttcgttaggggtcgtgtaggcgcggagttttagtcggtttgttttgggttcgaagaaagttgtggtttggagttagttttttttttacgatttttgggggcgtttttttttttttttgagttcgcgatgggaaggcgtttttattttattttcggggtttttgagattttttggcgaggtggggcggcgcggtaggtgtagggttggggagggtttgatagttttggagagtgttcgattttagttcgtcgtcgttttttaggcggattttttagaggtaggttttagttttcggcgcggggaggcggcgcgttttagaggggcgtagggtgcgcgggggtcgttggtttttcgggtattttttttttgcggttttttcgttttttttcggagttggtgtttgaggtcgttgggattttagtttggcgttttttttcgtttcgagcgaggtaggtgtcgtgggcggtttcggttatttttgtatttcgagcgtttattttgtatttagttttaagtcggagagttttggggatagtcgagagtgttagtggttttatagggagattgagggttttgttttatttcgggcgtcgtgtgggtttgggttgtttatagtatttttgcgttttagtgataggagaagttaagcgcgaggttttttaatagttgcgtgtattttattttttaggtatttggaatttcggcgtttagaacgtgtttttcgggagagtaaaggttgtgtttacggtagtttggggatatagtaagtattttgtaaatgatgaatgaatgtatgaattaattagttgataaataataatttgttttggagggggatttagtttttgttatagttgattttagtataatgggaggaaagaattagaaataatgatttttttaaatagtcgggaagatagagttaggggtttttttttaggaatatttggatgaggaatttgtagttaggtttattttattaataaatttatatttatttattagg

SEQ ID NO: 151 - DNA, 77 bp, Homo sapiens
gaatgcaaagtaagcgctcgaaatgcagaagtagccggggccgcccacggcacctgcctcgctcggggcgagagaag

SEQ ID NO: 152 - DNA, 77 bp, Artificial Sequence
  converted
gaatgtaaagtaagcgttcgaaatgtagaagtagtcggggtcgtttacggtatttgtttcgttcggggcgagagaag

SEQ ID NO: 153 - DNA, 77 bp, Artificial Sequence
  converted
cttctctcgccccgaacgaaacaaataccgtaaacgaccccgactacttctacatttcgaacgcttactttacattc

SEQ ID NO: 154 - DNA, 77 bp, Artificial Sequence
  converted
aaatacaaaataaacgctcgaaatacaaaaataaccgaaaccgcccacgacacctacctcgctcgaaacgaaaaaaa

SEQ ID NO: 155 - DNA, 77 bp, Artificial Sequence
  converted
tttttttcgtttcgagcgaggtaggtgtcgtgggcggtttcggttatttttgtatttcgagcgtttattttgtattt

SEQ ID NO: 156 - DNA, 361 bp, Homo sapiens
ccgcttcggcccccagcgcagctgcagaggggcccccctcgacgcatacactcaagagcccgaccgcgcggctgaaatcgcggagctcggagccgcggctggctgagcgatcgcggttcctgggctgcgtgcgcgccccttggagctgaaaggagcgccaggatcgggggcgctgcaccgggctgggcccctcaacgctcgcagaccgggccgggctgcagctggagatggcagcaatcccgggaggtctccgggcctcttcagggtgcgtccaggaggcgggttccgtgcgacgcggcgcagcccacccccacgagaccgcttaacttcgcgggggcagcctcgggcgctcggagacgcg

SEQ ID NO: 157 - DNA, 361 bp, Artificial Sequence
  converted
tcgtttcggtttttagcgtagttgtagaggggtttttttcgacgtatatatttaagagttcgatcgcgcggttgaaatcgcggagttcggagtcgcggttggttgagcgatcgcggtttttgggttgcgtgcgcgttttttggagttgaaaggagcgttaggatcgggggcgttgtatcgggttgggttttttaacgttcgtagatcgggtcgggttgtagttggagatggtagtaatttcgggaggttttcgggtttttttagggtgcgtttaggaggcgggtttcgtgcgacgcggcgtagtttatttttacgagatcgtttaatttcgcgggggtagtttcgggcgttcggagacgcg

SEQ ID NO: 158 - DNA, 361 bp, Artificial Sequence
  converted
cgcgtctccgaacgcccgaaactacccccgcgaaattaaacgatctcgtaaaaataaactacgccgcgtcgcacgaaacccgcctcctaaacgcaccctaaaaaaacccgaaaacctcccgaaattactaccatctccaactacaacccgacccgatctacgaacgttaaaaaacccaacccgatacaacgcccccgatcctaacgctcctttcaactccaaaaaacgcgcacgcaacccaaaaaccgcgatcgctcaaccaaccgcgactccgaactccgcgatttcaaccgcgcgatcgaactcttaaatatatacgtcgaaaaaaacccctctacaactacgctaaaaaccgaaacga

SEQ ID NO: 159 - DNA, 361 bp, Artificial Sequence
  converted
ccgcttcgacccccaacgcaactacaaaaaaacccccctcgacgcatacactcaaaaacccgaccgcgcgactaaaatcgcgaaactcgaaaccgcgactaactaaacgatcgcgattcctaaactacgtacgcgccccttaaaactaaaaaaaacgccaaaatcgaaaacgctacaccgaactaaacccctcaacgctcgcaaaccgaaccgaactacaactaaaaataacaacaatcccgaaaaatctccgaacctcttcaaaatacgtccaaaaaacgaattccgtacgacgcgacgcaacccacccccacgaaaccgcttaacttcgcgaaaacaacctcgaacgctcgaaaacgcg

SEQ ID NO: 160 - DNA, 361 bp, Artificial Sequence
  converted
cgcgttttcgagcgttcgaggttgttttcgcgaagttaagcggtttcgtgggggtgggttgcgtcgcgtcgtacggaattcgttttttggacgtattttgaagaggttcggagatttttcgggattgttgttatttttagttgtagttcggttcggtttgcgagcgttgaggggtttagttcggtgtagcgttttcgattttggcgttttttttagttttaaggggcgcgtacgtagtttaggaatcgcgatcgtttagttagtcgcggtttcgagtttcgcgattttagtcgcgcggtcgggtttttgagtgtatgcgtcgaggggggtttttttgtagttgcgttgggggtcgaagcgg

SEQ ID NO: 161 - DNA, 1097 bp, Homo sapiens
ggcagtgcggatgctgcctatgggggaggcagccaaggacggagggcgagaggcggttcttccaaggtcaccctcttccgggttgcaagcaaaggtcaggggatcccggaatggttagtgcaggagcttctctgtgccttccacgtcctagatcctcagagcctcagaaacggagatcatcgtccccacccccattttacagatgaagaaactgagccgaggaaaggaagcgacttggccaaggtcggagagctcattctttgcagggcggggtttggaacccggggtctggctctcggcaacgcgccctcggcccgcagcctcctgccccctgtgccccgcttcggcccccagcgcagctgcagaggggcccccctcgacgcatacactcaagagcccgaccgcgcggctgaaatcgcggagctcggagccgcggctggctgagcgatcgcggttcctgggctgcgtgcgcgccccttggagctgaaaggagcgccaggatcgggggcgctgcaccgggctgggcccctcaacgctcgcagaccgggccgggctgcagctggagatggcagcaatcccgggaggtctccgggcctcttcagggtgcgtccaggaggcgggttccgtgcgacgcggcgcagcccacccccacgagaccgcttaacttcgcgggggcagcctcgggcgctcggagacgcggaggcccagactgcagcctccggatgctggaagcccagactccctggggtcaccggctctcccgccaccccagctgcaaagagtcccattgcttcaccgtccggagcttagtctccttgttcctctaccagtccctccctccgcaggtctctggggacttctgaccgcctgttcttactctccccctgcccccatacttcccgcccttgtctcaggaacggtgatacagtcatcggattgctctccatctcctgttagtctacactgcacacaactcaataatccgctcccttcatccgggtgacagagacacagataatctgagctagtggtgctcaaagtaccggtcccagaacagcagcatcagcatctcttgggaacttgttaaaaatgagaat

SEQ ID NO: 162 - DNA, 1097 bp, Artificial Sequence
  converted
ggtagtgcggatgttgtttatgggggaggtagttaaggacggagggcgagaggcggtttttttaaggttattttttttcgggttgtaagtaaaggttaggggatttcggaatggttagtgtaggagtttttttgtgttttttacgttttagatttttagagttttagaaacggagattatcgtttttatttttattttatagatgaagaaattgagtcgaggaaaggaagcgatttggttaaggtcggagagtttattttttgtagggcggggtttggaattcggggtttggttttcggtaacgcgttttcggttcgtagttttttgttttttgtgtttcgtttcggtttttagcgtagttgtagaggggtttttttcgacgtatatatttaagagttcgatcgcgcggttgaaatcgcggagttcggagtcgcggttggttgagcgatcgcggtttttgggttgcgtgcgcgttttttggagttgaaaggagcgttaggatcgggggcgttgtatcgggttgggttttttaacgttcgtagatcgggtcgggttgtagttggagatggtagtaatttcgggaggttttcgggtttttttagggtgcgtttaggaggcgggtttcgtgcgacgcggcgtagtttatttttacgagatcgtttaatttcgcgggggtagtttcgggcgttcggagacgcggaggtttagattgtagttttcggatgttggaagtttagattttttggggttatcggttttttcgttattttagttgtaaagagttttattgttttatcgttcggagtttagtttttttgtttttttattagttttttttttcgtaggtttttggggatttttgatcgtttgtttttattttttttttgtttttatatttttcgtttttgttttaggaacggtgatatagttatcggattgttttttattttttgttagtttatattgtatataatttaataattcgttttttttattcgggtgatagagatatagataatttgagttagtggtgtttaaagtatcggttttagaatagtagtattagtattttttgggaatttgttaaaaatgagaat

SEQ ID NO: 163 - DNA, 1097 bp, Artificial Sequence
  converted
attctcatttttaacaaattcccaaaaaatactaatactactattctaaaaccgatactttaaacaccactaactcaaattatctatatctctatcacccgaataaaaaaaacgaattattaaattatatacaatataaactaacaaaaaataaaaaacaatccgataactatatcaccgttcctaaaacaaaaacgaaaaatataaaaacaaaaaaaaaataaaaacaaacgatcaaaaatccccaaaaacctacgaaaaaaaaaactaataaaaaaacaaaaaaactaaactccgaacgataaaacaataaaactctttacaactaaaataacgaaaaaaccgataaccccaaaaaatctaaacttccaacatccgaaaactacaatctaaacctccgcgtctccgaacgcccgaaactacccccgcgaaattaaacgatctcgtaaaaataaactacgccgcgtcgcacgaaacccgcctcctaaacgcaccctaaaaaaacccgaaaacctcccgaaattactaccatctccaactacaacccgacccgatctacgaacgttaaaaaacccaacccgatacaacgcccccgatcctaacgctcctttcaactccaaaaaacgcgcacgcaacccaaaaaccgcgatcgctcaaccaaccgcgactccgaactccgcgatttcaaccgcgcgatcgaactcttaaatatatacgtcgaaaaaaacccctctacaactacgctaaaaaccgaaacgaaacacaaaaaacaaaaaactacgaaccgaaaacgcgttaccgaaaaccaaaccccgaattccaaaccccgccctacaaaaaataaactctccgaccttaaccaaatcgcttcctttcctcgactcaatttcttcatctataaaataaaaataaaaacgataatctccgtttctaaaactctaaaaatctaaaacgtaaaaaacacaaaaaaactcctacactaaccattccgaaatcccctaacctttacttacaacccgaaaaaaaataaccttaaaaaaaccgcctctcgccctccgtccttaactacctcccccataaacaacatccgcactacc

SEQ ID NO: 164 - DNA, 1097 bp, Artificial Sequence
  converted
aacaatacgaatactacctataaaaaaaacaaccaaaaacgaaaaacgaaaaacgattcttccaaaatcaccctcttccgaattacaaacaaaaatcaaaaaatcccgaaataattaatacaaaaacttctctataccttccacgtcctaaatcctcaaaacctcaaaaacgaaaatcatcgtccccacccccattttacaaataaaaaaactaaaccgaaaaaaaaaaacgacttaaccaaaatcgaaaaactcattctttacaaaacgaaatttaaaacccgaaatctaactctcgacaacgcgccctcgacccgcaacctcctaccccctataccccgcttcgacccccaacgcaactacaaaaaaacccccctcgacgcatacactcaaaaacccgaccgcgcgactaaaatcgcgaaactcgaaaccgcgactaactaaacgatcgcgattcctaaactacgtacgcgccccttaaaactaaaaaaaacgccaaaatcgaaaacgctacaccgaactaaacccctcaacgctcgcaaaccgaaccgaactacaactaaaaataacaacaatcccgaaaaatctccgaacctcttcaaaatacgtccaaaaaacgaattccgtacgacgcgacgcaacccacccccacgaaaccgcttaacttcgcgaaaacaacctcgaacgctcgaaaacgcgaaaacccaaactacaacctccgaatactaaaaacccaaactccctaaaatcaccgactctcccgccaccccaactacaaaaaatcccattacttcaccgtccgaaacttaatctccttattcctctaccaatccctccctccgcaaatctctaaaaacttctaaccgcctattcttactctccccctacccccatacttcccgcccttatctcaaaaacgataatacaatcatcgaattactctccatctcctattaatctacactacacacaactcaataatccgctcccttcatccgaataacaaaaacacaaataatctaaactaataatactcaaaataccgatcccaaaacaacaacatcaacatctcttaaaaacttattaaaaataaaaat

SEQ ID NO: 165 - DNA, 1097 bp, Artificial Sequence
  converted
atttttatttttaataagtttttaagagatgttgatgttgttgttttgggatcggtattttgagtattattagtttagattatttgtgtttttgttattcggatgaagggagcggattattgagttgtgtgtagtgtagattaataggagatggagagtaattcgatgattgtattatcgtttttgagataagggcgggaagtatgggggtagggggagagtaagaataggcggttagaagtttttagagatttgcggagggagggattggtagaggaataaggagattaagtttcggacggtgaagtaatgggattttttgtagttggggtggcgggagagtcggtgattttagggagtttgggtttttagtattcggaggttgtagtttgggttttcgcgttttcgagcgttcgaggttgttttcgcgaagttaagcggtttcgtgggggtgggttgcgtcgcgtcgtacggaattcgttttttggacgtattttgaagaggttcggagatttttcgggattgttgttatttttagttgtagttcggttcggtttgcgagcgttgaggggtttagttcggtgtagcgttttcgattttggcgttttttttagttttaaggggcgcgtacgtagtttaggaatcgcgatcgtttagttagtcgcggtttcgagtttcgcgattttagtcgcgcggtcgggtttttgagtgtatgcgtcgaggggggtttttttgtagttgcgttgggggtcgaagcggggtatagggggtaggaggttgcgggtcgagggcgcgttgtcgagagttagatttcgggttttaaatttcgttttgtaaagaatgagtttttcgattttggttaagtcgtttttttttttcggtttagtttttttatttgtaaaatgggggtggggacgatgattttcgtttttgaggttttgaggatttaggacgtggaaggtatagagaagtttttgtattaattatttcgggattttttgatttttgtttgtaattcggaagagggtgattttggaagaatcgtttttcgtttttcgtttttggttgttttttttataggtagtattcgtattgtt

SEQ ID NO: 166 - DNA, 97 bp, Homo sapiens
atcgggggcgctgcaccgggctgggcccctcaacgctcgcagaccgggccgggctgcagctggagatggcagcaatcccgggaggtctccgggcctc

SEQ ID NO: 167 - DNA, 97 bp, Artificial Sequence
  converted
atcgggggcgttgtatcgggttgggttttttaacgttcgtagatcgggtcgggttgtagttggagatggtagtaatttcgggaggttttcgggtttt

SEQ ID NO: 168 - DNA, 97 bp, Artificial Sequence
  converted
aaaacccgaaaacctcccgaaattactaccatctccaactacaacccgacccgatctacgaacgttaaaaaacccaacccgatacaacgcccccgat

SEQ ID NO: 169 - DNA, 97 bp, Artificial Sequence
  converted
atcgaaaacgctacaccgaactaaacccctcaacgctcgcaaaccgaaccgaactacaactaaaaataacaacaatcccgaaaaatctccgaacctc

SEQ ID NO: 170 - DNA, 97 bp, Artificial Sequence
  converted
gaggttcggagatttttcgggattgttgttatttttagttgtagttcggttcggtttgcgagcgttgaggggtttagttcggtgtagcgttttcgat

SEQ ID NO: 171 - DNA, 1431 bp, Homo sapiens
ccgggagaggcgcgcgggcagcgtccaccccacccgctgggcctccgcagggccaaggccccagcagaaaaggcttcacgccgccgccggggtctgggacgcttgcccgacggagtcagaggagctcccgggtccagagtccccagtgcaaactccgacgcaacctgcgcctcgaagcgcaagcagcaaaagcgcccggactctggtcccaagagcctgggcctccttaagccataagcgtctgcggcgcctcgctttgggccttcttttgggccgggccggaggcatcttctagaaggctcttcagaccccgctttcgccaaactcccggcgccctgcgcttccagcccagcagagaaaagtgtgaaaagcaagcccgcggtcgccgtcggccttggcagagaaatcaagaggagaagggaagggaaccgctcaactacccttcgggaaaccaagtttccaaatatgccgccctcttcctggtttgcacaaacggtttagggcattcgttccggtttcagggtggggtatgccgtcgctcccctcctccccgccctgtgcttttaaaagttaggaaacaaaaaagagcacccattggctggaaccccaagggaggcagatgcaggaagcacagagctgcaccgctaggcgcagcaaacagccgcggccgaaggcgcgggtcgccgagtgggcggcggccgcccgcgaggcgagcctggaagcagtatctgaaccgtctggagggtgaccaggttgcgcggcgagctttgcgactgacacggccaatgcgctagtggctgagtggccttggccatgtagggacgcggcgcgcacgcacacccacagactcgcagaagttcaggttcgcgccggagagaagttataggcaggaaggggcccactcacccgcggctccgcgctccaggtctgcgccactccgagccggcccagacgtcgccccagccggcggcgggagcgcagcgccttcgtctccctcagaagaacccttttcgcccgcgccgccgccgcggctgcagcctccgtcgtccacggcccccgccgcctcttcggcgcccagttttcgccgcttcttctgaagctgttgctgcttctcggcgccgatcagcgcctccaccgagaaagcgtgcgccttgaggcttagcatgctgcacggcgagccccttcgcttctcggccatcccccccgccccgcgcccgcccgcccctctctcatatacactcacgcgggcacacgcgcgctctcgctcttcccccaccaaaaactaaaaggctctcggggcctcccgagatctgcccccttccccaccgcgggcaaaaaacagatttggcgtttccgctttctcgcttgtgttgggatccaggaaccggcgacgcgccggccaagtctcctttcctgggtctctctcgcgcg

SEQ ID NO: 172 - DNA, 1431 bp, Artificial Sequence
  converted
tcgggagaggcgcgcgggtagcgtttattttattcgttgggttttcgtagggttaaggttttagtagaaaaggttttacgtcgtcgtcggggtttgggacgtttgttcgacggagttagaggagttttcgggtttagagtttttagtgtaaatttcgacgtaatttgcgtttcgaagcgtaagtagtaaaagcgttcggattttggttttaagagtttgggtttttttaagttataagcgtttgcggcgtttcgttttgggtttttttttgggtcgggtcggaggtattttttagaaggttttttagatttcgttttcgttaaattttcggcgttttgcgtttttagtttagtagagaaaagtgtgaaaagtaagttcgcggtcgtcgtcggttttggtagagaaattaagaggagaagggaagggaatcgtttaattatttttcgggaaattaagtttttaaatatgtcgttttttttttggtttgtataaacggtttagggtattcgtttcggttttagggtggggtatgtcgtcgtttttttttttttcgttttgtgtttttaaaagttaggaaataaaaaagagtatttattggttggaattttaagggaggtagatgtaggaagtatagagttgtatcgttaggcgtagtaaatagtcgcggtcgaaggcgcgggtcgtcgagtgggcggcggtcgttcgcgaggcgagtttggaagtagtatttgaatcgtttggagggtgattaggttgcgcggcgagttttgcgattgatacggttaatgcgttagtggttgagtggttttggttatgtagggacgcggcgcgtacgtatatttatagattcgtagaagtttaggttcgcgtcggagagaagttataggtaggaaggggtttatttattcgcggtttcgcgttttaggtttgcgttatttcgagtcggtttagacgtcgttttagtcggcggcgggagcgtagcgttttcgttttttttagaagaatttttttcgttcgcgtcgtcgtcgcggttgtagttttcgtcgtttacggttttcgtcgttttttcggcgtttagttttcgtcgttttttttgaagttgttgttgtttttcggcgtcgattagcgtttttatcgagaaagcgtgcgttttgaggtttagtatgttgtacggcgagttttttcgtttttcggttatttttttcgtttcgcgttcgttcgttttttttttatatatatttacgcgggtatacgcgcgttttcgttttttttttattaaaaattaaaaggttttcggggtttttcgagatttgtttttttttttatcgcgggtaaaaaatagatttggcgttttcgttttttcgtttgtgttgggatttaggaatcggcgacgcgtcggttaagtttttttttttgggtttttttcgcgcg

SEQ ID NO: 173 - DNA, 1431 bp, Artificial Sequence
  converted
cgcgcgaaaaaaacccaaaaaaaaaaacttaaccgacgcgtcgccgattcctaaatcccaacacaaacgaaaaaacgaaaacgccaaatctattttttacccgcgataaaaaaaaaaacaaatctcgaaaaaccccgaaaaccttttaatttttaataaaaaaaaaacgaaaacgcgcgtatacccgcgtaaatatatataaaaaaaaaacgaacgaacgcgaaacgaaaaaaataaccgaaaaacgaaaaaactcgccgtacaacatactaaacctcaaaacgcacgctttctcgataaaaacgctaatcgacgccgaaaaacaacaacaacttcaaaaaaaacgacgaaaactaaacgccgaaaaaacgacgaaaaccgtaaacgacgaaaactacaaccgcgacgacgacgcgaacgaaaaaaattcttctaaaaaaaacgaaaacgctacgctcccgccgccgactaaaacgacgtctaaaccgactcgaaataacgcaaacctaaaacgcgaaaccgcgaataaataaaccccttcctacctataacttctctccgacgcgaacctaaacttctacgaatctataaatatacgtacgcgccgcgtccctacataaccaaaaccactcaaccactaacgcattaaccgtatcaatcgcaaaactcgccgcgcaacctaatcaccctccaaacgattcaaatactacttccaaactcgcctcgcgaacgaccgccgcccactcgacgacccgcgccttcgaccgcgactatttactacgcctaacgatacaactctatacttcctacatctacctcccttaaaattccaaccaataaatactcttttttatttcctaacttttaaaaacacaaaacgaaaaaaaaaaaaacgacgacataccccaccctaaaaccgaaacgaataccctaaaccgtttatacaaaccaaaaaaaaaacgacatatttaaaaacttaatttcccgaaaaataattaaacgattcccttcccttctcctcttaatttctctaccaaaaccgacgacgaccgcgaacttacttttcacacttttctctactaaactaaaaacgcaaaacgccgaaaatttaacgaaaacgaaatctaaaaaaccttctaaaaaatacctccgacccgacccaaaaaaaaacccaaaacgaaacgccgcaaacgcttataacttaaaaaaacccaaactcttaaaaccaaaatccgaacgcttttactacttacgcttcgaaacgcaaattacgtcgaaatttacactaaaaactctaaacccgaaaactcctctaactccgtcgaacaaacgtcccaaaccccgacgacgacgtaaaaccttttctactaaaaccttaaccctacgaaaacccaacgaataaaataaacgctacccgcgcgcctctcccga

SEQ ID NO: 174 - DNA, 1431 bp, Artificial Sequence
  converted
ccgaaaaaaacgcgcgaacaacgtccaccccacccgctaaacctccgcaaaaccaaaaccccaacaaaaaaaacttcacgccgccgccgaaatctaaaacgcttacccgacgaaatcaaaaaaactcccgaatccaaaatccccaatacaaactccgacgcaacctacgcctcgaaacgcaaacaacaaaaacgcccgaactctaatcccaaaaacctaaacctccttaaaccataaacgtctacgacgcctcgctttaaaccttcttttaaaccgaaccgaaaacatcttctaaaaaactcttcaaaccccgctttcgccaaactcccgacgccctacgcttccaacccaacaaaaaaaaatataaaaaacaaacccgcgatcgccgtcgaccttaacaaaaaaatcaaaaaaaaaaaaaaaaaaaaccgctcaactacccttcgaaaaaccaaatttccaaatataccgccctcttcctaatttacacaaacgatttaaaacattcgttccgatttcaaaataaaatataccgtcgctcccctcctccccgccctatacttttaaaaattaaaaaacaaaaaaaaacacccattaactaaaaccccaaaaaaaacaaatacaaaaaacacaaaactacaccgctaaacgcaacaaacaaccgcgaccgaaaacgcgaatcgccgaataaacgacgaccgcccgcgaaacgaacctaaaaacaatatctaaaccgtctaaaaaataaccaaattacgcgacgaactttacgactaacacgaccaatacgctaataactaaataaccttaaccatataaaaacgcgacgcgcacgcacacccacaaactcgcaaaaattcaaattcgcgccgaaaaaaaattataaacaaaaaaaaacccactcacccgcgactccgcgctccaaatctacgccactccgaaccgacccaaacgtcgccccaaccgacgacgaaaacgcaacgccttcgtctccctcaaaaaaacccttttcgcccgcgccgccgccgcgactacaacctccgtcgtccacgacccccgccgcctcttcgacgcccaattttcgccgcttcttctaaaactattactacttctcgacgccgatcaacgcctccaccgaaaaaacgtacgccttaaaacttaacatactacacgacgaaccccttcgcttctcgaccatcccccccgccccgcgcccgcccgcccctctctcatatacactcacgcgaacacacgcgcgctctcgctcttcccccaccaaaaactaaaaaactctcgaaacctcccgaaatctacccccttccccaccgcgaacaaaaaacaaatttaacgtttccgctttctcgcttatattaaaatccaaaaaccgacgacgcgccgaccaaatctcctttcctaaatctctctcgcgcg

SEQ ID NO: 175 - DNA, 1431 bp, Artificial Sequence
  converted
cgcgcgagagagatttaggaaaggagatttggtcggcgcgtcgtcggtttttggattttaatataagcgagaaagcggaaacgttaaatttgttttttgttcgcggtggggaagggggtagatttcgggaggtttcgagagttttttagtttttggtgggggaagagcgagagcgcgcgtgtgttcgcgtgagtgtatatgagagaggggcgggcgggcgcggggcgggggggatggtcgagaagcgaaggggttcgtcgtgtagtatgttaagttttaaggcgtacgttttttcggtggaggcgttgatcggcgtcgagaagtagtaatagttttagaagaagcggcgaaaattgggcgtcgaagaggcggcgggggtcgtggacgacggaggttgtagtcgcggcggcggcgcgggcgaaaagggtttttttgagggagacgaaggcgttgcgttttcgtcgtcggttggggcgacgtttgggtcggttcggagtggcgtagatttggagcgcggagtcgcgggtgagtgggtttttttttgtttataattttttttcggcgcgaatttgaatttttgcgagtttgtgggtgtgcgtgcgcgtcgcgtttttatatggttaaggttatttagttattagcgtattggtcgtgttagtcgtaaagttcgtcgcgtaatttggttattttttagacggtttagatattgtttttaggttcgtttcgcgggcggtcgtcgtttattcggcgattcgcgttttcggtcgcggttgtttgttgcgtttagcggtgtagttttgtgttttttgtatttgttttttttggggttttagttaatgggtgtttttttttgttttttaatttttaaaagtatagggcggggaggaggggagcgacggtatattttattttgaaatcggaacgaatgttttaaatcgtttgtgtaaattaggaagagggcggtatatttggaaatttggtttttcgaagggtagttgagcggttttttttttttttttttttgatttttttgttaaggtcgacggcgatcgcgggtttgttttttatatttttttttgttgggttggaagcgtagggcgtcgggagtttggcgaaagcggggtttgaagagttttttagaagatgttttcggttcggtttaaaagaaggtttaaagcgaggcgtcgtagacgtttatggtttaaggaggtttaggtttttgggattagagttcgggcgtttttgttgtttgcgtttcgaggcgtaggttgcgtcggagtttgtattggggattttggattcgggagtttttttgatttcgtcgggtaagcgttttagatttcggcggcggcgtgaagttttttttgttggggttttggttttgcggaggtttagcgggtggggtggacgttgttcgcgcgtttttttcgg

SEQ ID NO: 176 - DNA, 1087 bp, Homo sapiens
cagaccccgctttcgccaaactcccggcgccctgcgcttccagcccagcagagaaaagtgtgaaaagcaagcccgcggtcgccgtcggccttggcagagaaatcaagaggagaagggaagggaaccgctcaactacccttcgggaaaccaagtttccaaatatgccgccctcttcctggtttgcacaaacggtttagggcattcgttccggtttcagggtggggtatgccgtcgctcccctcctccccgccctgtgcttttaaaagttaggaaacaaaaaagagcacccattggctggaaccccaagggaggcagatgcaggaagcacagagctgcaccgctaggcgcagcaaacagccgcggccgaaggcgcgggtcgccgagtgggcggcggccgcccgcgaggcgagcctggaagcagtatctgaaccgtctggagggtgaccaggttgcgcggcgagctttgcgactgacacggccaatgcgctagtggctgagtggccttggccatgtagggacgcggcgcgcacgcacacccacagactcgcagaagttcaggttcgcgccggagagaagttataggcaggaaggggcccactcacccgcggctccgcgctccaggtctgcgccactccgagccggcccagacgtcgccccagccggcggcgggagcgcagcgccttcgtctccctcagaagaacccttttcgcccgcgccgccgccgcggctgcagcctccgtcgtccacggcccccgccgcctcttcggcgcccagttttcgccgcttcttctgaagctgttgctgcttctcggcgccgatcagcgcctccaccgagaaagcgtgcgccttgaggcttagcatgctgcacggcgagccccttcgcttctcggccatcccccccgccccgcgcccgcccgcccctctctcatatacactcacgcgggcacacgcgcgctctcgctcttcccccaccaaaaactaaaaggctctcggggcctcccgagatctgcccccttccccaccgcgggcaaaaaacagatttggcgtttccgctttctcgcttgtgttgggatccaggaaccggcga

SEQ ID NO: 177 - DNA, 1087 bp, Artificial Sequence
  converted
tagatttcgttttcgttaaattttcggcgttttgcgtttttagtttagtagagaaaagtgtgaaaagtaagttcgcggtcgtcgtcggttttggtagagaaattaagaggagaagggaagggaatcgtttaattatttttcgggaaattaagtttttaaatatgtcgttttttttttggtttgtataaacggtttagggtattcgtttcggttttagggtggggtatgtcgtcgtttttttttttttcgttttgtgtttttaaaagttaggaaataaaaaagagtatttattggttggaattttaagggaggtagatgtaggaagtatagagttgtatcgttaggcgtagtaaatagtcgcggtcgaaggcgcgggtcgtcgagtgggcggcggtcgttcgcgaggcgagtttggaagtagtatttgaatcgtttggagggtgattaggttgcgcggcgagttttgcgattgatacggttaatgcgttagtggttgagtggttttggttatgtagggacgcggcgcgtacgtatatttatagattcgtagaagtttaggttcgcgtcggagagaagttataggtaggaaggggtttatttattcgcggtttcgcgttttaggtttgcgttatttcgagtcggtttagacgtcgttttagtcggcggcgggagcgtagcgttttcgttttttttagaagaatttttttcgttcgcgtcgtcgtcgcggttgtagttttcgtcgtttacggttttcgtcgttttttcggcgtttagttttcgtcgttttttttgaagttgttgttgtttttcggcgtcgattagcgtttttatcgagaaagcgtgcgttttgaggtttagtatgttgtacggcgagttttttcgtttttcggttatttttttcgtttcgcgttcgttcgttttttttttatatatatttacgcgggtatacgcgcgttttcgttttttttttattaaaaattaaaaggttttcggggtttttcgagatttgtttttttttttatcgcgggtaaaaaatagatttggcgttttcgttttttcgtttgtgttgggatttaggaatcggcga

SEQ ID NO: 178 - DNA, 1087 bp, Artificial Sequence
  converted
tcgccgattcctaaatcccaacacaaacgaaaaaacgaaaacgccaaatctattttttacccgcgataaaaaaaaaaacaaatctcgaaaaaccccgaaaaccttttaatttttaataaaaaaaaaacgaaaacgcgcgtatacccgcgtaaatatatataaaaaaaaaacgaacgaacgcgaaacgaaaaaaataaccgaaaaacgaaaaaactcgccgtacaacatactaaacctcaaaacgcacgctttctcgataaaaacgctaatcgacgccgaaaaacaacaacaacttcaaaaaaaacgacgaaaactaaacgccgaaaaaacgacgaaaaccgtaaacgacgaaaactacaaccgcgacgacgacgcgaacgaaaaaaattcttctaaaaaaaacgaaaacgctacgctcccgccgccgactaaaacgacgtctaaaccgactcgaaataacgcaaacctaaaacgcgaaaccgcgaataaataaaccccttcctacctataacttctctccgacgcgaacctaaacttctacgaatctataaatatacgtacgcgccgcgtccctacataaccaaaaccactcaaccactaacgcattaaccgtatcaatcgcaaaactcgccgcgcaacctaatcaccctccaaacgattcaaatactacttccaaactcgcctcgcgaacgaccgccgcccactcgacgacccgcgccttcgaccgcgactatttactacgcctaacgatacaactctatacttcctacatctacctcccttaaaattccaaccaataaatactcttttttatttcctaacttttaaaaacacaaaacgaaaaaaaaaaaaacgacgacataccccaccctaaaaccgaaacgaataccctaaaccgtttatacaaaccaaaaaaaaaacgacatatttaaaaacttaatttcccgaaaaataattaaacgattcccttcccttctcctcttaatttctctaccaaaaccgacgacgaccgcgaacttacttttcacacttttctctactaaactaaaaacgcaaaacgccgaaaatttaacgaaaacgaaatcta

SEQ ID NO: 179 - DNA, 1087 bp, Artificial Sequence
  converted
caaaccccgctttcgccaaactcccgacgccctacgcttccaacccaacaaaaaaaaatataaaaaacaaacccgcgatcgccgtcgaccttaacaaaaaaatcaaaaaaaaaaaaaaaaaaaaccgctcaactacccttcgaaaaaccaaatttccaaatataccgccctcttcctaatttacacaaacgatttaaaacattcgttccgatttcaaaataaaatataccgtcgctcccctcctccccgccctatacttttaaaaattaaaaaacaaaaaaaaacacccattaactaaaaccccaaaaaaaacaaatacaaaaaacacaaaactacaccgctaaacgcaacaaacaaccgcgaccgaaaacgcgaatcgccgaataaacgacgaccgcccgcgaaacgaacctaaaaacaatatctaaaccgtctaaaaaataaccaaattacgcgacgaactttacgactaacacgaccaatacgctaataactaaataaccttaaccatataaaaacgcgacgcgcacgcacacccacaaactcgcaaaaattcaaattcgcgccgaaaaaaaattataaacaaaaaaaaacccactcacccgcgactccgcgctccaaatctacgccactccgaaccgacccaaacgtcgccccaaccgacgacgaaaacgcaacgccttcgtctccctcaaaaaaacccttttcgcccgcgccgccgccgcgactacaacctccgtcgtccacgacccccgccgcctcttcgacgcccaattttcgccgcttcttctaaaactattactacttctcgacgccgatcaacgcctccaccgaaaaaacgtacgccttaaaacttaacatactacacgacgaaccccttcgcttctcgaccatcccccccgccccgcgcccgcccgcccctctctcatatacactcacgcgaacacacgcgcgctctcgctcttcccccaccaaaaactaaaaaactctcgaaacctcccgaaatctacccccttccccaccgcgaacaaaaaacaaatttaacgtttccgctttctcgcttatattaaaatccaaaaaccgacga

SEQ ID NO: 180 - DNA, 1087 bp, Artificial Sequence
  converted
tcgtcggtttttggattttaatataagcgagaaagcggaaacgttaaatttgttttttgttcgcggtggggaagggggtagatttcgggaggtttcgagagttttttagtttttggtgggggaagagcgagagcgcgcgtgtgttcgcgtgagtgtatatgagagaggggcgggcgggcgcggggcgggggggatggtcgagaagcgaaggggttcgtcgtgtagtatgttaagttttaaggcgtacgttttttcggtggaggcgttgatcggcgtcgagaagtagtaatagttttagaagaagcggcgaaaattgggcgtcgaagaggcggcgggggtcgtggacgacggaggttgtagtcgcggcggcggcgcgggcgaaaagggtttttttgagggagacgaaggcgttgcgttttcgtcgtcggttggggcgacgtttgggtcggttcggagtggcgtagatttggagcgcggagtcgcgggtgagtgggtttttttttgtttataattttttttcggcgcgaatttgaatttttgcgagtttgtgggtgtgcgtgcgcgtcgcgtttttatatggttaaggttatttagttattagcgtattggtcgtgttagtcgtaaagttcgtcgcgtaatttggttattttttagacggtttagatattgtttttaggttcgtttcgcgggcggtcgtcgtttattcggcgattcgcgttttcggtcgcggttgtttgttgcgtttagcggtgtagttttgtgttttttgtatttgttttttttggggttttagttaatgggtgtttttttttgttttttaatttttaaaagtatagggcggggaggaggggagcgacggtatattttattttgaaatcggaacgaatgttttaaatcgtttgtgtaaattaggaagagggcggtatatttggaaatttggtttttcgaagggtagttgagcggttttttttttttttttttttgatttttttgttaaggtcgacggcgatcgcgggtttgttttttatatttttttttgttgggttggaagcgtagggcgtcgggagtttggcgaaagcggggtttg

SEQ ID NO: 181 - DNA, 87 bp, Homo sapiens
gccttggccatgtagggacgcggcgcgcacgcacacccacagactcgcagaagttcaggttcgcgccggagagaagttataggcagg

SEQ ID NO: 182 - DNA, 87 bp, Artificial Sequence
  converted
gttttggttatgtagggacgcggcgcgtacgtatatttatagattcgtagaagtttaggttcgcgtcggagagaagttataggtagg

SEQ ID NO: 183 - DNA, 87 bp, Artificial Sequence
  converted
cctacctataacttctctccgacgcgaacctaaacttctacgaatctataaatatacgtacgcgccgcgtccctacataaccaaaac

SEQ ID NO: 184 - DNA, 87 bp, Artificial Sequence
  converted
accttaaccatataaaaacgcgacgcgcacgcacacccacaaactcgcaaaaattcaaattcgcgccgaaaaaaaattataaacaaa

SEQ ID NO: 185 - DNA, 87 bp, Artificial Sequence
  converted
tttgtttataattttttttcggcgcgaatttgaatttttgcgagtttgtgggtgtgcgtgcgcgtcgcgtttttatatggttaaggt

SEQ ID NO: 186 - DNA, 1013 bp, Homo sapiens
gcggcgcgctggctccctggagcggggcgggacgcggccgcgcggactcacgtgcacaaccgcgcgggacggggccacgcggactcacgtgcacaaccgcgggaccccagcgccagcgggaccccagcgccagcgggaccccagcgccagcgggaccccagcgccagcgggaccccagcgccagcgggaccccagcgccagcgggaccccagcgccagcgggtctgtggcccagtggagcgagtggagcgctggcgacctgagcggagactgcgccctggacgccccagcctagacgtcaagttacagcccgcgcagcagcagcaaaggggaaggggcaggagccgggcacagttggatccggaggtcgtgacccaggggaaagcgtgggcggtcgacccagggcagctgcggcggcgaggcaggtgggctccttgctccctggagccgcccctccccacacctgccctcggcgcccccagcagttttcaccttggccctccgcggtcactgcgggattcggcgttgccgccagcccagtggggagtgaattagcgccctccttcgtcctcggcccttccgacggcacgaggaactcctgtcctgccccacagaccttcggcctccgccgagtgcggtactggagcctgccccgccagggccctggaatcagagaaagtcgctctttggccacctgaagcgtcggatccctacagtgcctcccagcctgggcgggagcggcggctgcgtcgctgaaggttggggtccttggtgcgaaagggaggcagctgcagcctcagccccaccccagaagcggccttcgcatcgctgcggtgggcgttctcgggcttcgacttcgccagcgccgcggggcagaggcacctggagctcgcagggcccagacctgggttggaaaagcttcgctgactgcaggcaagcgtccgggaggggcggccaggcgaagccccggcgctttaccacacacttccgggtcccatgccagttgcatccgcg

SEQ ID NO: 187 - DNA, 1013 bp, Artificial Sequence
  converted
gcggcgcgttggttttttggagcggggcgggacgcggtcgcgcggatttacgtgtataatcgcgcgggacggggttacgcggatttacgtgtataatcgcgggattttagcgttagcgggattttagcgttagcgggattttagcgttagcgggattttagcgttagcgggattttagcgttagcgggattttagcgttagcgggattttagcgttagcgggtttgtggtttagtggagcgagtggagcgttggcgatttgagcggagattgcgttttggacgttttagtttagacgttaagttatagttcgcgtagtagtagtaaaggggaaggggtaggagtcgggtatagttggattcggaggtcgtgatttaggggaaagcgtgggcggtcgatttagggtagttgcggcggcgaggtaggtgggttttttgttttttggagtcgtttttttttatatttgttttcggcgtttttagtagtttttattttggtttttcgcggttattgcgggattcggcgttgtcgttagtttagtggggagtgaattagcgttttttttcgttttcggttttttcgacggtacgaggaatttttgttttgttttatagattttcggttttcgtcgagtgcggtattggagtttgtttcgttagggttttggaattagagaaagtcgttttttggttatttgaagcgtcggatttttatagtgttttttagtttgggcgggagcggcggttgcgtcgttgaaggttggggtttttggtgcgaaagggaggtagttgtagttttagttttattttagaagcggttttcgtatcgttgcggtgggcgttttcgggtttcgatttcgttagcgtcgcggggtagaggtatttggagttcgtagggtttagatttgggttggaaaagtttcgttgattgtaggtaagcgttcgggaggggcggttaggcgaagtttcggcgttttattatatattttcgggttttatgttagttgtattcgcg

SEQ ID NO: 188 - DNA, 1013 bp, Artificial Sequence
  converted
cgcgaatacaactaacataaaacccgaaaatatataataaaacgccgaaacttcgcctaaccgcccctcccgaacgcttacctacaatcaacgaaacttttccaacccaaatctaaaccctacgaactccaaatacctctaccccgcgacgctaacgaaatcgaaacccgaaaacgcccaccgcaacgatacgaaaaccgcttctaaaataaaactaaaactacaactacctccctttcgcaccaaaaaccccaaccttcaacgacgcaaccgccgctcccgcccaaactaaaaaacactataaaaatccgacgcttcaaataaccaaaaaacgactttctctaattccaaaaccctaacgaaacaaactccaataccgcactcgacgaaaaccgaaaatctataaaacaaaacaaaaattcctcgtaccgtcgaaaaaaccgaaaacgaaaaaaaacgctaattcactccccactaaactaacgacaacgccgaatcccgcaataaccgcgaaaaaccaaaataaaaactactaaaaacgccgaaaacaaatataaaaaaaaacgactccaaaaaacaaaaaacccacctacctcgccgccgcaactaccctaaatcgaccgcccacgctttcccctaaatcacgacctccgaatccaactatacccgactcctaccccttcccctttactactactacgcgaactataacttaacgtctaaactaaaacgtccaaaacgcaatctccgctcaaatcgccaacgctccactcgctccactaaaccacaaacccgctaacgctaaaatcccgctaacgctaaaatcccgctaacgctaaaatcccgctaacgctaaaatcccgctaacgctaaaatcccgctaacgctaaaatcccgctaacgctaaaatcccgcgattatacacgtaaatccgcgtaaccccgtcccgcgcgattatacacgtaaatccgcgcgaccgcgtcccgccccgctccaaaaaaccaacgcgccgc

SEQ ID NO: 189 - DNA, 1013 bp, Artificial Sequence
  converted
acgacgcgctaactccctaaaacgaaacgaaacgcgaccgcgcgaactcacgtacacaaccgcgcgaaacgaaaccacgcgaactcacgtacacaaccgcgaaaccccaacgccaacgaaaccccaacgccaacgaaaccccaacgccaacgaaaccccaacgccaacgaaaccccaacgccaacgaaaccccaacgccaacgaaaccccaacgccaacgaatctataacccaataaaacgaataaaacgctaacgacctaaacgaaaactacgccctaaacgccccaacctaaacgtcaaattacaacccgcgcaacaacaacaaaaaaaaaaaaacaaaaaccgaacacaattaaatccgaaaatcgtaacccaaaaaaaaacgtaaacgatcgacccaaaacaactacgacgacgaaacaaataaactccttactccctaaaaccgcccctccccacacctaccctcgacgcccccaacaattttcaccttaaccctccgcgatcactacgaaattcgacgttaccgccaacccaataaaaaataaattaacgccctccttcgtcctcgacccttccgacgacacgaaaaactcctatcctaccccacaaaccttcgacctccgccgaatacgatactaaaacctaccccgccaaaaccctaaaatcaaaaaaaatcgctctttaaccacctaaaacgtcgaatccctacaatacctcccaacctaaacgaaaacgacgactacgtcgctaaaaattaaaatccttaatacgaaaaaaaaacaactacaacctcaaccccaccccaaaaacgaccttcgcatcgctacgataaacgttctcgaacttcgacttcgccaacgccgcgaaacaaaaacacctaaaactcgcaaaacccaaacctaaattaaaaaaacttcgctaactacaaacaaacgtccgaaaaaaacgaccaaacgaaaccccgacgctttaccacacacttccgaatcccataccaattacatccgcg

SEQ ID NO: 190 - DNA, 1013 bp, Artificial Sequence
  converted
cgcggatgtaattggtatgggattcggaagtgtgtggtaaagcgtcggggtttcgtttggtcgtttttttcggacgtttgtttgtagttagcgaagtttttttaatttaggtttgggttttgcgagttttaggtgtttttgtttcgcggcgttggcgaagtcgaagttcgagaacgtttatcgtagcgatgcgaaggtcgtttttggggtggggttgaggttgtagttgtttttttttcgtattaaggattttaatttttagcgacgtagtcgtcgttttcgtttaggttgggaggtattgtagggattcgacgttttaggtggttaaagagcgattttttttgattttagggttttggcggggtaggttttagtatcgtattcggcggaggtcgaaggtttgtggggtaggataggagtttttcgtgtcgtcggaagggtcgaggacgaaggagggcgttaatttattttttattgggttggcggtaacgtcgaatttcgtagtgatcgcggagggttaaggtgaaaattgttgggggcgtcgagggtaggtgtggggaggggcggttttagggagtaaggagtttatttgtttcgtcgtcgtagttgttttgggtcgatcgtttacgtttttttttgggttacgattttcggatttaattgtgttcggtttttgtttttttttttttgttgttgttgcgcgggttgtaatttgacgtttaggttggggcgtttagggcgtagttttcgtttaggtcgttagcgttttattcgttttattgggttatagattcgttggcgttggggtttcgttggcgttggggtttcgttggcgttggggtttcgttggcgttggggtttcgttggcgttggggtttcgttggcgttggggtttcgttggcgttggggtttcgcggttgtgtacgtgagttcgcgtggtttcgtttcgcgcggttgtgtacgtgagttcgcgcggtcgcgtttcgtttcgttttagggagttagcgcgtcgt

SEQ ID NO: 191 - DNA, 1069 bp, Homo sapiens
ggcaggtgggctccttgctccctggagccgcccctccccacacctgccctcggcgcccccagcagttttcaccttggccctccgcggtcactgcgggattcggcgttgccgccagcccagtggggagtgaattagcgccctccttcgtcctcggcccttccgacggcacgaggaactcctgtcctgccccacagaccttcggcctccgccgagtgcggtactggagcctgccccgccagggccctggaatcagagaaagtcgctctttggccacctgaagcgtcggatccctacagtgcctcccagcctgggcgggagcggcggctgcgtcgctgaaggttggggtccttggtgcgaaagggaggcagctgcagcctcagccccaccccagaagcggccttcgcatcgctgcggtgggcgttctcgggcttcgacttcgccagcgccgcggggcagaggcacctggagctcgcagggcccagacctgggttggaaaagcttcgctgactgcaggcaagcgtccgggaggggcggccaggcgaagccccggcgctttaccacacacttccgggtcccatgccagttgcatccgcggtattgggcaggaaatggcagggctgaggccgaccctaggagtataagggagccctccatttcctgcccacatttgtcacctccagttttgcaacctatcccagacacacagaaagcaagcaggactggtggggagacggagcttaacaggaatattttccagcagtgagcaggggctgtatgggacgcgggaggagctcagaggaggcgcggagagtgcccgaggttgggtgagtgcctagaggggagatagttgaaccgggttcaagaggtgcttagtgggtgtttgttgaatgaatgagtgatgggctttgaagtctgagtgcattgaaagagggggtgtgtaaaaagggctcctttcatcacacaggacacagcatatgcaaatcctctccctgtggaaaagccagacaggttaaaaaggttacaaacaaattagccgggcatggtggtgcgcgtctgtagtcccagctac

SEQ ID NO: 192 - DNA, 1069 bp, Artificial Sequence
  converted
ggtaggtgggttttttgttttttggagtcgtttttttttatatttgttttcggcgtttttagtagtttttattttggtttttcgcggttattgcgggattcggcgttgtcgttagtttagtggggagtgaattagcgttttttttcgttttcggttttttcgacggtacgaggaatttttgttttgttttatagattttcggttttcgtcgagtgcggtattggagtttgtttcgttagggttttggaattagagaaagtcgttttttggttatttgaagcgtcggatttttatagtgttttttagtttgggcgggagcggcggttgcgtcgttgaaggttggggtttttggtgcgaaagggaggtagttgtagttttagttttattttagaagcggttttcgtatcgttgcggtgggcgttttcgggtttcgatttcgttagcgtcgcggggtagaggtatttggagttcgtagggtttagatttgggttggaaaagtttcgttgattgtaggtaagcgttcgggaggggcggttaggcgaagtttcggcgttttattatatattttcgggttttatgttagttgtattcgcggtattgggtaggaaatggtagggttgaggtcgattttaggagtataagggagttttttattttttgtttatatttgttatttttagttttgtaatttattttagatatatagaaagtaagtaggattggtggggagacggagtttaataggaatattttttagtagtgagtaggggttgtatgggacgcgggaggagtttagaggaggcgcggagagtgttcgaggttgggtgagtgtttagaggggagatagttgaatcgggtttaagaggtgtttagtgggtgtttgttgaatgaatgagtgatgggttttgaagtttgagtgtattgaaagagggggtgtgtaaaaagggttttttttattatataggatatagtatatgtaaattttttttttgtggaaaagttagataggttaaaaaggttataaataaattagtcgggtatggtggtgcgcgtttgtagttttagttat

SEQ ID NO: 193 - DNA, 1069 bp, Artificial Sequence
  converted
ataactaaaactacaaacgcgcaccaccatacccgactaatttatttataacctttttaacctatctaacttttccacaaaaaaaaaatttacatatactatatcctatataataaaaaaaaccctttttacacaccccctctttcaatacactcaaacttcaaaacccatcactcattcattcaacaaacacccactaaacacctcttaaacccgattcaactatctcccctctaaacactcacccaacctcgaacactctccgcgcctcctctaaactcctcccgcgtcccatacaacccctactcactactaaaaaatattcctattaaactccgtctccccaccaatcctacttactttctatatatctaaaataaattacaaaactaaaaataacaaatataaacaaaaaataaaaaactcccttatactcctaaaatcgacctcaaccctaccatttcctacccaataccgcgaatacaactaacataaaacccgaaaatatataataaaacgccgaaacttcgcctaaccgcccctcccgaacgcttacctacaatcaacgaaacttttccaacccaaatctaaaccctacgaactccaaatacctctaccccgcgacgctaacgaaatcgaaacccgaaaacgcccaccgcaacgatacgaaaaccgcttctaaaataaaactaaaactacaactacctccctttcgcaccaaaaaccccaaccttcaacgacgcaaccgccgctcccgcccaaactaaaaaacactataaaaatccgacgcttcaaataaccaaaaaacgactttctctaattccaaaaccctaacgaaacaaactccaataccgcactcgacgaaaaccgaaaatctataaaacaaaacaaaaattcctcgtaccgtcgaaaaaaccgaaaacgaaaaaaaacgctaattcactccccactaaactaacgacaacgccgaatcccgcaataaccgcgaaaaaccaaaataaaaactactaaaaacgccgaaaacaaatataaaaaaaaacgactccaaaaaacaaaaaacccacctacc

SEQ ID NO: 194 - DNA, 1069 bp, Artificial Sequence
  converted
aacaaataaactccttactccctaaaaccgcccctccccacacctaccctcgacgcccccaacaattttcaccttaaccctccgcgatcactacgaaattcgacgttaccgccaacccaataaaaaataaattaacgccctccttcgtcctcgacccttccgacgacacgaaaaactcctatcctaccccacaaaccttcgacctccgccgaatacgatactaaaacctaccccgccaaaaccctaaaatcaaaaaaaatcgctctttaaccacctaaaacgtcgaatccctacaatacctcccaacctaaacgaaaacgacgactacgtcgctaaaaattaaaatccttaatacgaaaaaaaaacaactacaacctcaaccccaccccaaaaacgaccttcgcatcgctacgataaacgttctcgaacttcgacttcgccaacgccgcgaaacaaaaacacctaaaactcgcaaaacccaaacctaaattaaaaaaacttcgctaactacaaacaaacgtccgaaaaaaacgaccaaacgaaaccccgacgctttaccacacacttccgaatcccataccaattacatccgcgatattaaacaaaaaataacaaaactaaaaccgaccctaaaaatataaaaaaaccctccatttcctacccacatttatcacctccaattttacaacctatcccaaacacacaaaaaacaaacaaaactaataaaaaaacgaaacttaacaaaaatattttccaacaataaacaaaaactatataaaacgcgaaaaaaactcaaaaaaaacgcgaaaaatacccgaaattaaataaatacctaaaaaaaaaataattaaaccgaattcaaaaaatacttaataaatatttattaaataaataaataataaactttaaaatctaaatacattaaaaaaaaaaatatataaaaaaaactcctttcatcacacaaaacacaacatatacaaatcctctccctataaaaaaaccaaacaaattaaaaaaattacaaacaaattaaccgaacataataatacgcgtctataatcccaactac

SEQ ID NO: 195 - DNA, 1069 bp, Artificial Sequence
  converted
gtagttgggattatagacgcgtattattatgttcggttaatttgtttgtaatttttttaatttgtttggtttttttatagggagaggatttgtatatgttgtgttttgtgtgatgaaaggagttttttttatatatttttttttttaatgtatttagattttaaagtttattatttatttatttaataaatatttattaagtattttttgaattcggtttaattattttttttttaggtatttatttaatttcgggtatttttcgcgttttttttgagtttttttcgcgttttatatagtttttgtttattgttggaaaatatttttgttaagtttcgtttttttattagttttgtttgttttttgtgtgtttgggataggttgtaaaattggaggtgataaatgtgggtaggaaatggagggtttttttatatttttagggtcggttttagttttgttattttttgtttaatatcgcggatgtaattggtatgggattcggaagtgtgtggtaaagcgtcggggtttcgtttggtcgtttttttcggacgtttgtttgtagttagcgaagtttttttaatttaggtttgggttttgcgagttttaggtgtttttgtttcgcggcgttggcgaagtcgaagttcgagaacgtttatcgtagcgatgcgaaggtcgtttttggggtggggttgaggttgtagttgtttttttttcgtattaaggattttaatttttagcgacgtagtcgtcgttttcgtttaggttgggaggtattgtagggattcgacgttttaggtggttaaagagcgattttttttgattttagggttttggcggggtaggttttagtatcgtattcggcggaggtcgaaggtttgtggggtaggataggagtttttcgtgtcgtcggaagggtcgaggacgaaggagggcgttaatttattttttattgggttggcggtaacgtcgaatttcgtagtgatcgcggagggttaaggtgaaaattgttgggggcgtcgagggtaggtgtggggaggggcggttttagggagtaaggagtttatttgtt

SEQ ID NO: 196 - DNA, 69 bp, Homo sapiens
tcgctgactgcaggcaagcgtccgggaggggcggccaggcgaagccccggcgctttaccacacacttcc

SEQ ID NO: 197 - DNA, 69 bp, Artificial Sequence
  converted
tcgttgattgtaggtaagcgttcgggaggggcggttaggcgaagtttcggcgttttattatatattttt

SEQ ID NO: 198 - DNA, 69 bp, Artificial Sequence
  converted
aaaaatatataataaaacgccgaaacttcgcctaaccgcccctcccgaacgcttacctacaatcaacga

SEQ ID NO: 199 - DNA, 69 bp, Artificial Sequence
  converted
tcgctaactacaaacaaacgtccgaaaaaaacgaccaaacgaaaccccgacgctttaccacacacttcc

SEQ ID NO: 200 - DNA, 69 bp, Artificial Sequence
  converted
ggaagtgtgtggtaaagcgtcggggtttcgtttggtcgtttttttcggacgtttgtttgtagttagcga

SEQ ID NO: 201 - DNA, 1818 bp, Homo sapiens
gcgcgctagggtaggctggtctgtgctggatacgcgtgttcttctgcggagttaaagggtcggggacgggggttctggacttaccagagcaattccagccggtgggcgtttggcagtcacttaaggaggtagggaaagcagcgagcttcaccgggcgggctacgatgagtagcatgacgggcagcagcagcagccagcaaaagccctcgcaaagtgtccagctgctgcactgccgcggggactcccacagcaccatgactagttcgtgcaactctgcagcagcaaacggcttccgaggaacacaggatcgcgggggccgggcagcgggctactgagcatcccgcggacggcggcagcagaggcggcggcggtggcagtggcacccggcggggaagcagcagccaaacccgcgcatgatctcgagagtttcagcaacatccagggactgggctcagccccggagcgagagggtcgtccgctgagaagctgcgccggagacgcgggaagctgctgccataaggagggagctctgggaagccggaggacaggaggagacgggagtccaggggcagacgagtggagcccgaggaggcagggtggagggagagtcaaggcgccccgcagcccggcagccgcctctcgagctctgccgcccgcatccctctggcgtttgggaagcagcaggtcctcagcccgcccggggtcacgtgggaagaggcagtcgggctctgattggtggagcaggatgcaggtcccgggagggaggggtcgacgaggaggtgcaaggatgcaaggaggaggcggccgcggaagccacagatgggctcgctcgccaggcgctggcccgagtggggctaggcggggatggctcaaatgagaagctcgggcttcagggtgggctacccgcacactcatataccattcgcctcactctccgctccaggacgccccctaccgaaggcggggtccggactagcgcccctcttccgcgcgtgaccccgggccgcgagtgcgggccgcggctgggtggcgtctctccgagctggagatggtgggggcggaggtgtcagaggagcagcagcagcagggcagagaggggcgagtcggcgcgggagagggcgtcctgctggcgaccggcgctccagcgtgcgggagcgcgccgcctaggctgtagggggatgcaggctgggaatgtcgcggcggagaggccagggacgtttctctagggatttacaggaaagagggtgagaggcgatggtgttagaaccgctcttgccgacctggaagcaacagcagcatctcccacaagagcgtgcaaccccaaggctgctcgccgaggcagctcagccatcccggcaggcgctctccttccttctctcttctcccctctctcctcccaggccccccgcagctccgacccagcccaagcgttcgcaggtttgaatccctctcctcatcacccgctcctctccagcccgtagcctattagtgtgtccacctgggaggtgcggtcagatgtgtttggaaggtcagattggtcgggacaagtggtctgagagaaagagaaaggctcctctgcatacgccgcgggtgggttgccgggagcatcggccgggcagcggcgtccgggaaggggagagcgggctccatttgttggcccaggcagtgaccctgcgttccttactcgggtctttgccggatggccggtgacctggggcgacgagagaaggtctaactcggcaggagtctctggctctgcgcgtttctttcattctctccagcgggaagggcaaacggcatagcgggacccgccttccg

SEQ ID NO: 202 - DNA, 1818 bp, Artificial Sequence
  converted
gcgcgttagggtaggttggtttgtgttggatacgcgtgtttttttgcggagttaaagggtcggggacgggggttttggatttattagagtaattttagtcggtgggcgtttggtagttatttaaggaggtagggaaagtagcgagttttatcgggcgggttacgatgagtagtatgacgggtagtagtagtagttagtaaaagttttcgtaaagtgtttagttgttgtattgtcgcggggatttttatagtattatgattagttcgtgtaattttgtagtagtaaacggttttcgaggaatataggatcgcgggggtcgggtagcgggttattgagtatttcgcggacggcggtagtagaggcggcggcggtggtagtggtattcggcggggaagtagtagttaaattcgcgtatgatttcgagagttttagtaatatttagggattgggtttagtttcggagcgagagggtcgttcgttgagaagttgcgtcggagacgcgggaagttgttgttataaggagggagttttgggaagtcggaggataggaggagacgggagtttaggggtagacgagtggagttcgaggaggtagggtggagggagagttaaggcgtttcgtagttcggtagtcgtttttcgagttttgtcgttcgtatttttttggcgtttgggaagtagtaggtttttagttcgttcggggttacgtgggaagaggtagtcgggttttgattggtggagtaggatgtaggtttcgggagggaggggtcgacgaggaggtgtaaggatgtaaggaggaggcggtcgcggaagttatagatgggttcgttcgttaggcgttggttcgagtggggttaggcggggatggtttaaatgagaagttcgggttttagggtgggttattcgtatatttatatattattcgttttatttttcgttttaggacgttttttatcgaaggcggggttcggattagcgtttttttttcgcgcgtgatttcgggtcgcgagtgcgggtcgcggttgggtggcgttttttcgagttggagatggtgggggcggaggtgttagaggagtagtagtagtagggtagagaggggcgagtcggcgcgggagagggcgttttgttggcgatcggcgttttagcgtgcgggagcgcgtcgtttaggttgtagggggatgtaggttgggaatgtcgcggcggagaggttagggacgtttttttagggatttataggaaagagggtgagaggcgatggtgttagaatcgtttttgtcgatttggaagtaatagtagtattttttataagagcgtgtaattttaaggttgttcgtcgaggtagtttagttatttcggtaggcgttttttttttttttttttttttttttttttttttttaggtttttcgtagtttcgatttagtttaagcgttcgtaggtttgaatttttttttttattattcgtttttttttagttcgtagtttattagtgtgtttatttgggaggtgcggttagatgtgtttggaaggttagattggtcgggataagtggtttgagagaaagagaaaggtttttttgtatacgtcgcgggtgggttgtcgggagtatcggtcgggtagcggcgttcgggaaggggagagcgggttttatttgttggtttaggtagtgattttgcgttttttattcgggtttttgtcggatggtcggtgatttggggcgacgagagaaggtttaattcggtaggagtttttggttttgcgcgttttttttattttttttagcgggaagggtaaacggtatagcgggattcgtttttcg

SEQ ID NO: 203 - DNA, 1818 bp, Artificial Sequence
  converted
cgaaaaacgaatcccgctataccgtttacccttcccgctaaaaaaaataaaaaaaacgcgcaaaaccaaaaactcctaccgaattaaaccttctctcgtcgccccaaatcaccgaccatccgacaaaaacccgaataaaaaacgcaaaatcactacctaaaccaacaaataaaacccgctctccccttcccgaacgccgctacccgaccgatactcccgacaacccacccgcgacgtatacaaaaaaacctttctctttctctcaaaccacttatcccgaccaatctaaccttccaaacacatctaaccgcacctcccaaataaacacactaataaactacgaactaaaaaaaaacgaataataaaaaaaaaaattcaaacctacgaacgcttaaactaaatcgaaactacgaaaaacctaaaaaaaaaaaaaaaaaaaaaaaaaaaaaaaaaaaacgcctaccgaaataactaaactacctcgacgaacaaccttaaaattacacgctcttataaaaaatactactattacttccaaatcgacaaaaacgattctaacaccatcgcctctcaccctctttcctataaatccctaaaaaaacgtccctaacctctccgccgcgacattcccaacctacatccccctacaacctaaacgacgcgctcccgcacgctaaaacgccgatcgccaacaaaacgccctctcccgcgccgactcgcccctctctaccctactactactactcctctaacacctccgcccccaccatctccaactcgaaaaaacgccacccaaccgcgacccgcactcgcgacccgaaatcacgcgcgaaaaaaaaacgctaatccgaaccccgccttcgataaaaaacgtcctaaaacgaaaaataaaacgaataatatataaatatacgaataacccaccctaaaacccgaacttctcatttaaaccatccccgcctaaccccactcgaaccaacgcctaacgaacgaacccatctataacttccgcgaccgcctcctccttacatccttacacctcctcgtcgacccctccctcccgaaacctacatcctactccaccaatcaaaacccgactacctcttcccacgtaaccccgaacgaactaaaaacctactacttcccaaacgccaaaaaaatacgaacgacaaaactcgaaaaacgactaccgaactacgaaacgccttaactctccctccaccctacctcctcgaactccactcgtctacccctaaactcccgtctcctcctatcctccgacttcccaaaactccctccttataacaacaacttcccgcgtctccgacgcaacttctcaacgaacgaccctctcgctccgaaactaaacccaatccctaaatattactaaaactctcgaaatcatacgcgaatttaactactacttccccgccgaataccactaccaccgccgccgcctctactaccgccgtccgcgaaatactcaataacccgctacccgacccccgcgatcctatattcctcgaaaaccgtttactactacaaaattacacgaactaatcataatactataaaaatccccgcgacaatacaacaactaaacactttacgaaaacttttactaactactactactacccgtcatactactcatcgtaacccgcccgataaaactcgctactttccctacctccttaaataactaccaaacgcccaccgactaaaattactctaataaatccaaaacccccgtccccgaccctttaactccgcaaaaaaacacgcgtatccaacacaaaccaacctaccctaacgcgc

SEQ ID NO: 204 - DNA, 1818 bp, Artificial Sequence
  converted
acgcgctaaaataaactaatctatactaaatacgcgtattcttctacgaaattaaaaaatcgaaaacgaaaattctaaacttaccaaaacaattccaaccgataaacgtttaacaatcacttaaaaaaataaaaaaaacaacgaacttcaccgaacgaactacgataaataacataacgaacaacaacaacaaccaacaaaaaccctcgcaaaatatccaactactacactaccgcgaaaactcccacaacaccataactaattcgtacaactctacaacaacaaacgacttccgaaaaacacaaaatcgcgaaaaccgaacaacgaactactaaacatcccgcgaacgacgacaacaaaaacgacgacgataacaataacacccgacgaaaaaacaacaaccaaacccgcgcataatctcgaaaatttcaacaacatccaaaaactaaactcaaccccgaaacgaaaaaatcgtccgctaaaaaactacgccgaaaacgcgaaaaactactaccataaaaaaaaaactctaaaaaaccgaaaaacaaaaaaaaacgaaaatccaaaaacaaacgaataaaacccgaaaaaacaaaataaaaaaaaaatcaaaacgccccgcaacccgacaaccgcctctcgaactctaccgcccgcatccctctaacgtttaaaaaacaacaaatcctcaacccgcccgaaatcacgtaaaaaaaaacaatcgaactctaattaataaaacaaaatacaaatcccgaaaaaaaaaaatcgacgaaaaaatacaaaaatacaaaaaaaaaacgaccgcgaaaaccacaaataaactcgctcgccaaacgctaacccgaataaaactaaacgaaaataactcaaataaaaaactcgaacttcaaaataaactacccgcacactcatataccattcgcctcactctccgctccaaaacgccccctaccgaaaacgaaatccgaactaacgcccctcttccgcgcgtaaccccgaaccgcgaatacgaaccgcgactaaataacgtctctccgaactaaaaataataaaaacgaaaatatcaaaaaaacaacaacaacaaaacaaaaaaaaacgaatcgacgcgaaaaaaaacgtcctactaacgaccgacgctccaacgtacgaaaacgcgccgcctaaactataaaaaaatacaaactaaaaatatcgcgacgaaaaaaccaaaaacgtttctctaaaaatttacaaaaaaaaaaataaaaaacgataatattaaaaccgctcttaccgacctaaaaacaacaacaacatctcccacaaaaacgtacaaccccaaaactactcgccgaaacaactcaaccatcccgacaaacgctctccttccttctctcttctcccctctctcctcccaaaccccccgcaactccgacccaacccaaacgttcgcaaatttaaatccctctcctcatcacccgctcctctccaacccgtaacctattaatatatccacctaaaaaatacgatcaaatatatttaaaaaatcaaattaatcgaaacaaataatctaaaaaaaaaaaaaaaactcctctacatacgccgcgaataaattaccgaaaacatcgaccgaacaacgacgtccgaaaaaaaaaaaacgaactccatttattaacccaaacaataaccctacgttccttactcgaatctttaccgaataaccgataacctaaaacgacgaaaaaaaatctaactcgacaaaaatctctaactctacgcgtttctttcattctctccaacgaaaaaaacaaacgacataacgaaacccgccttccg

SEQ ID NO: 205 - DNA, 1818 bp, Artificial Sequence
  converted
cggaaggcgggtttcgttatgtcgtttgtttttttcgttggagagaatgaaagaaacgcgtagagttagagatttttgtcgagttagattttttttcgtcgttttaggttatcggttattcggtaaagattcgagtaaggaacgtagggttattgtttgggttaataaatggagttcgttttttttttttcggacgtcgttgttcggtcgatgttttcggtaatttattcgcggcgtatgtagaggagttttttttttttttttagattatttgtttcgattaatttgattttttaaatatatttgatcgtattttttaggtggatatattaataggttacgggttggagaggagcgggtgatgaggagagggatttaaatttgcgaacgtttgggttgggtcggagttgcggggggtttgggaggagagaggggagaagagagaaggaaggagagcgtttgtcgggatggttgagttgtttcggcgagtagttttggggttgtacgtttttgtgggagatgttgttgttgtttttaggtcggtaagagcggttttaatattatcgttttttatttttttttttgtaaatttttagagaaacgtttttggttttttcgtcgcgatatttttagtttgtatttttttatagtttaggcggcgcgttttcgtacgttggagcgtcggtcgttagtaggacgttttttttcgcgtcgattcgtttttttttgttttgttgttgttgtttttttgatattttcgtttttattatttttagttcggagagacgttatttagtcgcggttcgtattcgcggttcggggttacgcgcggaagaggggcgttagttcggatttcgttttcggtagggggcgttttggagcggagagtgaggcgaatggtatatgagtgtgcgggtagtttattttgaagttcgagttttttatttgagttattttcgtttagttttattcgggttagcgtttggcgagcgagtttatttgtggttttcgcggtcgttttttttttgtatttttgtattttttcgtcgatttttttttttcgggatttgtattttgttttattaattagagttcgattgtttttttttacgtgatttcgggcgggttgaggatttgttgttttttaaacgttagagggatgcgggcggtagagttcgagaggcggttgtcgggttgcggggcgttttgattttttttttattttgttttttcgggttttattcgtttgtttttggattttcgtttttttttgtttttcggttttttagagttttttttttatggtagtagtttttcgcgttttcggcgtagttttttagcggacgattttttcgtttcggggttgagtttagtttttggatgttgttgaaattttcgagattatgcgcgggtttggttgttgttttttcgtcgggtgttattgttatcgtcgtcgtttttgttgtcgtcgttcgcgggatgtttagtagttcgttgttcggttttcgcgattttgtgtttttcggaagtcgtttgttgttgtagagttgtacgaattagttatggtgttgtgggagttttcgcggtagtgtagtagttggatattttgcgagggtttttgttggttgttgttgttgttcgttatgttatttatcgtagttcgttcggtgaagttcgttgttttttttatttttttaagtgattgttaaacgtttatcggttggaattgttttggtaagtttagaattttcgttttcgattttttaatttcgtagaagaatacgcgtatttagtatagattagtttattttagcgcgt

SEQ ID NO: 206 - DNA, 1074 bp, Homo sapiens
cagaggagcagcagcagcagggcagagaggggcgagtcggcgcgggagagggcgtcctgctggcgaccggcgctccagcgtgcgggagcgcgccgcctaggctgtagggggatgcaggctgggaatgtcgcggcggagaggccagggacgtttctctagggatttacaggaaagagggtgagaggcgatggtgttagaaccgctcttgccgacctggaagcaacagcagcatctcccacaagagcgtgcaaccccaaggctgctcgccgaggcagctcagccatcccggcaggcgctctccttccttctctcttctcccctctctcctcccaggccccccgcagctccgacccagcccaagcgttcgcaggtttgaatccctctcctcatcacccgctcctctccagcccgtagcctattagtgtgtccacctgggaggtgcggtcagatgtgtttggaaggtcagattggtcgggacaagtggtctgagagaaagagaaaggctcctctgcatacgccgcgggtgggttgccgggagcatcggccgggcagcggcgtccgggaaggggagagcgggctccatttgttggcccaggcagtgaccctgcgttccttactcgggtctttgccggatggccggtgacctggggcgacgagagaaggtctaactcggcaggagtctctggctctgcgcgtttctttcattctctccagcgggaagggcaaacggcatagcgggacccgccttccgtctgctgcattcttcaggcagttagacacactctttagcctaatggaattttagtcgccagtaacgggaccaagagctttcggggacaagggtggagaggaacatctttcttccatgaccggggtcactattgcagtctcagtgttttggatgccccatagggaagagctttctttttggtgtgtgattattcagtgattcctgtttttgtttttgtttatcttctctccgctctccttctctattcctctctgttatccctcctccttcttctccccccgctttcaaaagcctccggatcctccctttttctatttaaattc

SEQ ID NO: 207 - DNA, 1074 bp, Artificial Sequence
  converted
tagaggagtagtagtagtagggtagagaggggcgagtcggcgcgggagagggcgttttgttggcgatcggcgttttagcgtgcgggagcgcgtcgtttaggttgtagggggatgtaggttgggaatgtcgcggcggagaggttagggacgtttttttagggatttataggaaagagggtgagaggcgatggtgttagaatcgtttttgtcgatttggaagtaatagtagtattttttataagagcgtgtaattttaaggttgttcgtcgaggtagtttagttatttcggtaggcgttttttttttttttttttttttttttttttttttttaggtttttcgtagtttcgatttagtttaagcgttcgtaggtttgaatttttttttttattattcgtttttttttagttcgtagtttattagtgtgtttatttgggaggtgcggttagatgtgtttggaaggttagattggtcgggataagtggtttgagagaaagagaaaggtttttttgtatacgtcgcgggtgggttgtcgggagtatcggtcgggtagcggcgttcgggaaggggagagcgggttttatttgttggtttaggtagtgattttgcgttttttattcgggtttttgtcggatggtcggtgatttggggcgacgagagaaggtttaattcggtaggagtttttggttttgcgcgttttttttattttttttagcgggaagggtaaacggtatagcgggattcgtttttcgtttgttgtattttttaggtagttagatatattttttagtttaatggaattttagtcgttagtaacgggattaagagttttcggggataagggtggagaggaatattttttttttatgatcggggttattattgtagttttagtgttttggatgttttatagggaagagttttttttttggtgtgtgattatttagtgatttttgtttttgtttttgtttatttttttttcgttttttttttttatttttttttgttattttttttttttttttttttttcgtttttaaaagttttcggattttttttttttttatttaaattt

SEQ ID NO: 208 - DNA, 1074 bp, Artificial Sequence
  converted
aaatttaaataaaaaaaaaaaaaatccgaaaacttttaaaaacgaaaaaaaaaaaaaaaaaaaaaataacaaaaaaaaataaaaaaaaaaaacgaaaaaaaaataaacaaaaacaaaaacaaaaatcactaaataatcacacaccaaaaaaaaaactcttccctataaaacatccaaaacactaaaactacaataataaccccgatcataaaaaaaaaatattcctctccacccttatccccgaaaactcttaatcccgttactaacgactaaaattccattaaactaaaaaatatatctaactacctaaaaaatacaacaaacgaaaaacgaatcccgctataccgtttacccttcccgctaaaaaaaataaaaaaaacgcgcaaaaccaaaaactcctaccgaattaaaccttctctcgtcgccccaaatcaccgaccatccgacaaaaacccgaataaaaaacgcaaaatcactacctaaaccaacaaataaaacccgctctccccttcccgaacgccgctacccgaccgatactcccgacaacccacccgcgacgtatacaaaaaaacctttctctttctctcaaaccacttatcccgaccaatctaaccttccaaacacatctaaccgcacctcccaaataaacacactaataaactacgaactaaaaaaaaacgaataataaaaaaaaaaattcaaacctacgaacgcttaaactaaatcgaaactacgaaaaacctaaaaaaaaaaaaaaaaaaaaaaaaaaaaaaaaaaaacgcctaccgaaataactaaactacctcgacgaacaaccttaaaattacacgctcttataaaaaatactactattacttccaaatcgacaaaaacgattctaacaccatcgcctctcaccctctttcctataaatccctaaaaaaacgtccctaacctctccgccgcgacattcccaacctacatccccctacaacctaaacgacgcgctcccgcacgctaaaacgccgatcgccaacaaaacgccctctcccgcgccgactcgcccctctctaccctactactactactcctcta

SEQ ID NO: 209 - DNA, 1074 bp, Artificial Sequence
  converted
caaaaaaacaacaacaacaaaacaaaaaaaaacgaatcgacgcgaaaaaaaacgtcctactaacgaccgacgctccaacgtacgaaaacgcgccgcctaaactataaaaaaatacaaactaaaaatatcgcgacgaaaaaaccaaaaacgtttctctaaaaatttacaaaaaaaaaaataaaaaacgataatattaaaaccgctcttaccgacctaaaaacaacaacaacatctcccacaaaaacgtacaaccccaaaactactcgccgaaacaactcaaccatcccgacaaacgctctccttccttctctcttctcccctctctcctcccaaaccccccgcaactccgacccaacccaaacgttcgcaaatttaaatccctctcctcatcacccgctcctctccaacccgtaacctattaatatatccacctaaaaaatacgatcaaatatatttaaaaaatcaaattaatcgaaacaaataatctaaaaaaaaaaaaaaaactcctctacatacgccgcgaataaattaccgaaaacatcgaccgaacaacgacgtccgaaaaaaaaaaaacgaactccatttattaacccaaacaataaccctacgttccttactcgaatctttaccgaataaccgataacctaaaacgacgaaaaaaaatctaactcgacaaaaatctctaactctacgcgtttctttcattctctccaacgaaaaaaacaaacgacataacgaaacccgccttccgtctactacattcttcaaacaattaaacacactctttaacctaataaaattttaatcgccaataacgaaaccaaaaactttcgaaaacaaaaataaaaaaaaacatctttcttccataaccgaaatcactattacaatctcaatattttaaataccccataaaaaaaaactttctttttaatatataattattcaataattcctatttttatttttatttatcttctctccgctctccttctctattcctctctattatccctcctccttcttctccccccgctttcaaaaacctccgaatcctccctttttctatttaaattc

SEQ ID NO: 210 - DNA, 1074 bp, Artificial Sequence
  converted
gaatttaaatagaaaaagggaggattcggaggtttttgaaagcggggggagaagaaggaggagggataatagagaggaatagagaaggagagcggagagaagataaataaaaataaaaataggaattattgaataattatatattaaaaagaaagtttttttttatggggtatttaaaatattgagattgtaatagtgatttcggttatggaagaaagatgtttttttttatttttgttttcgaaagtttttggtttcgttattggcgattaaaattttattaggttaaagagtgtgtttaattgtttgaagaatgtagtagacggaaggcgggtttcgttatgtcgtttgtttttttcgttggagagaatgaaagaaacgcgtagagttagagatttttgtcgagttagattttttttcgtcgttttaggttatcggttattcggtaaagattcgagtaaggaacgtagggttattgtttgggttaataaatggagttcgttttttttttttcggacgtcgttgttcggtcgatgttttcggtaatttattcgcggcgtatgtagaggagttttttttttttttttagattatttgtttcgattaatttgattttttaaatatatttgatcgtattttttaggtggatatattaataggttacgggttggagaggagcgggtgatgaggagagggatttaaatttgcgaacgtttgggttgggtcggagttgcggggggtttgggaggagagaggggagaagagagaaggaaggagagcgtttgtcgggatggttgagttgtttcggcgagtagttttggggttgtacgtttttgtgggagatgttgttgttgtttttaggtcggtaagagcggttttaatattatcgttttttatttttttttttgtaaatttttagagaaacgtttttggttttttcgtcgcgatatttttagtttgtatttttttatagtttaggcggcgcgttttcgtacgttggagcgtcggtcgttagtaggacgttttttttcgcgtcgattcgtttttttttgttttgttgttgttgtttttttg

SEQ ID NO: 211 - DNA, 74 bp, Homo sapiens
aggctcctctgcatacgccgcgggtgggttgccgggagcatcggccgggcagcggcgtccgggaaggggagagc

SEQ ID NO: 212 - DNA, 74 bp, Artificial Sequence
  converted
aggtttttttgtatacgtcgcgggtgggttgtcgggagtatcggtcgggtagcggcgttcgggaaggggagagt

SEQ ID NO: 213 - DNA, 74 bp, Artificial Sequence
  converted
actctccccttcccgaacgccgctacccgaccgatactcccgacaacccacccgcgacgtatacaaaaaaacct

SEQ ID NO: 214 - DNA, 74 bp, Artificial Sequence
  converted
aaactcctctacatacgccgcgaataaattaccgaaaacatcgaccgaacaacgacgtccgaaaaaaaaaaaac

SEQ ID NO: 215 - DNA, 74 bp, Artificial Sequence
  converted
gttttttttttttcggacgtcgttgttcggtcgatgttttcggtaatttattcgcggcgtatgtagaggagttt

SEQ ID NO: 216 - DNA, 1131 bp, Homo sapiens
aggtgtgcaggcgctacaagaaggagccggggccgctgcccccgcagcgcggacttcgccccgtgctgcacacgctttagaagagcgtgtccggaagtgtcctccccggccctcctcgcttcccaagatttgtttgcaacgtctacacccgtcaccaccgcataagcaccagggtgaggaggtctctttgcaggaattaatatcgatttcagtctcctgctccttcaaagtttctctttgacgatggcgagacttatattctaagtcagaagaaaataaaaccttgcctattctgggcccatgattgcacaaaatccaaaatgctgccggcatgaagatgcagctggaagactctagaaacctagcattaggaggcagagactttatagtgcccagagatgagttcctaaataaactaagaattacgcagtgggaaattaaggaggagggtctgaggggccgacctcagagggacagatagagaaatcgagagcgaaacactcaaggaagtggaacaaatcgtcgtagccaggcgtctctgcctctccggaaacccgaaatccgcccaaacctcatcccaggcgctcacggtaccagtcgcagggccccgcactataggggctgccaccctctgcccccggagtccccacgccgagactcatcatttgctggctctttcttccttttcttcttttgtttttttatccttcaaatataagacaaaaatacacctcatttatcatcatagatatatatatttcactctcaccacataaatacaaaacatccaaatatccaaaacattcagaacaaagttagtagcctggattcagtgacacctacccgcgtttgaatcgttcattattttcttcactatttgcctagaaaaaaaaaaaagatgaaaaagagggggagagtttccgacgtaaataatttacaataaaattactattttaaaaaagtgtttttcgcttgcttcagatggatgaatacattctagacactgcggcacctcgcagtctgggtctgtgattttggttaaaatgtgggtggccgtggctgtagggacgatcgccgctgcgcagggtaaggaaagagggtgtcgcggttcgtgttctgatttgggaccac

SEQ ID NO: 217 - DNA, 1131 bp, Artificial Sequence
  converted
aggtgtgtaggcgttataagaaggagtcggggtcgttgttttcgtagcgcggatttcgtttcgtgttgtatacgttttagaagagcgtgttcggaagtgtttttttcggtttttttcgttttttaagatttgtttgtaacgtttatattcgttattatcgtataagtattagggtgaggaggtttttttgtaggaattaatatcgattttagttttttgtttttttaaagtttttttttgacgatggcgagatttatattttaagttagaagaaaataaaattttgtttattttgggtttatgattgtataaaatttaaaatgttgtcggtatgaagatgtagttggaagattttagaaatttagtattaggaggtagagattttatagtgtttagagatgagtttttaaataaattaagaattacgtagtgggaaattaaggaggagggtttgaggggtcgattttagagggatagatagagaaatcgagagcgaaatatttaaggaagtggaataaatcgtcgtagttaggcgtttttgttttttcggaaattcgaaattcgtttaaattttattttaggcgtttacggtattagtcgtagggtttcgtattataggggttgttattttttgttttcggagtttttacgtcgagatttattatttgttggtttttttttttttttttttttttgtttttttattttttaaatataagataaaaatatattttatttattattatagatatatatattttatttttattatataaatataaaatatttaaatatttaaaatatttagaataaagttagtagtttggatttagtgatatttattcgcgtttgaatcgtttattattttttttattatttgtttagaaaaaaaaaaaagatgaaaaagagggggagagttttcgacgtaaataatttataataaaattattattttaaaaaagtgtttttcgtttgttttagatggatgaatatattttagatattgcggtatttcgtagtttgggtttgtgattttggttaaaatgtgggtggtcgtggttgtagggacgatcgtcgttgcgtagggtaaggaaagagggtgtcgcggttcgtgttttgatttgggattat

SEQ ID NO: 218 - DNA, 1131 bp, Artificial Sequence
  converted
ataatcccaaatcaaaacacgaaccgcgacaccctctttccttaccctacgcaacgacgatcgtccctacaaccacgaccacccacattttaaccaaaatcacaaacccaaactacgaaataccgcaatatctaaaatatattcatccatctaaaacaaacgaaaaacacttttttaaaataataattttattataaattatttacgtcgaaaactctccccctctttttcatcttttttttttttctaaacaaataataaaaaaaataataaacgattcaaacgcgaataaatatcactaaatccaaactactaactttattctaaatattttaaatatttaaatattttatatttatataataaaaataaaatatatatatctataataataaataaaatatatttttatcttatatttaaaaaataaaaaaacaaaaaaaaaaaaaaaaaaaaaaaccaacaaataataaatctcgacgtaaaaactccgaaaacaaaaaataacaacccctataatacgaaaccctacgactaataccgtaaacgcctaaaataaaatttaaacgaatttcgaatttccgaaaaaacaaaaacgcctaactacgacgatttattccacttccttaaatatttcgctctcgatttctctatctatccctctaaaatcgacccctcaaaccctcctccttaatttcccactacgtaattcttaatttatttaaaaactcatctctaaacactataaaatctctacctcctaatactaaatttctaaaatcttccaactacatcttcataccgacaacattttaaattttatacaatcataaacccaaaataaacaaaattttattttcttctaacttaaaatataaatctcgccatcgtcaaaaaaaaactttaaaaaaacaaaaaactaaaatcgatattaattcctacaaaaaaacctcctcaccctaatacttatacgataataacgaatataaacgttacaaacaaatcttaaaaaacgaaaaaaaccgaaaaaaacacttccgaacacgctcttctaaaacgtatacaacacgaaacgaaatccgcgctacgaaaacaacgaccccgactccttcttataacgcctacacacct

SEQ ID NO: 219 - DNA, 1131 bp, Artificial Sequence
  converted
aaatatacaaacgctacaaaaaaaaaccgaaaccgctacccccgcaacgcgaacttcgccccgtactacacacgctttaaaaaaacgtatccgaaaatatcctccccgaccctcctcgcttcccaaaatttatttacaacgtctacacccgtcaccaccgcataaacaccaaaataaaaaaatctctttacaaaaattaatatcgatttcaatctcctactccttcaaaatttctctttaacgataacgaaacttatattctaaatcaaaaaaaaataaaaccttacctattctaaacccataattacacaaaatccaaaatactaccgacataaaaatacaactaaaaaactctaaaaacctaacattaaaaaacaaaaactttataatacccaaaaataaattcctaaataaactaaaaattacgcaataaaaaattaaaaaaaaaaatctaaaaaaccgacctcaaaaaaacaaataaaaaaatcgaaaacgaaacactcaaaaaaataaaacaaatcgtcgtaaccaaacgtctctacctctccgaaaacccgaaatccgcccaaacctcatcccaaacgctcacgataccaatcgcaaaaccccgcactataaaaactaccaccctctacccccgaaatccccacgccgaaactcatcatttactaactctttcttccttttcttcttttatttttttatccttcaaatataaaacaaaaatacacctcatttatcatcataaatatatatatttcactctcaccacataaatacaaaacatccaaatatccaaaacattcaaaacaaaattaataacctaaattcaataacacctacccgcgtttaaatcgttcattattttcttcactatttacctaaaaaaaaaaaaaaaataaaaaaaaaaaaaaaaatttccgacgtaaataatttacaataaaattactattttaaaaaaatatttttcgcttacttcaaataaataaatacattctaaacactacgacacctcgcaatctaaatctataattttaattaaaatataaataaccgtaactataaaaacgatcgccgctacgcaaaataaaaaaaaaaaatatcgcgattcgtattctaatttaaaaccac

SEQ ID NO: 220 - DNA, 1131 bp, Artificial Sequence
  converted
gtggttttaaattagaatacgaatcgcgatattttttttttttattttgcgtagcggcgatcgtttttatagttacggttatttatattttaattaaaattatagatttagattgcgaggtgtcgtagtgtttagaatgtatttatttatttgaagtaagcgaaaaatatttttttaaaatagtaattttattgtaaattatttacgtcggaaatttttttttttttttttattttttttttttttttaggtaaatagtgaagaaaataatgaacgatttaaacgcgggtaggtgttattgaatttaggttattaattttgttttgaatgttttggatatttggatgttttgtatttatgtggtgagagtgaaatatatatatttatgatgataaatgaggtgtatttttgttttatatttgaaggataaaaaaataaaagaagaaaaggaagaaagagttagtaaatgatgagtttcggcgtggggatttcgggggtagagggtggtagtttttatagtgcggggttttgcgattggtatcgtgagcgtttgggatgaggtttgggcggatttcgggttttcggagaggtagagacgtttggttacgacgatttgttttatttttttgagtgtttcgttttcgatttttttatttgtttttttgaggtcggttttttagatttttttttttaattttttattgcgtaatttttagtttatttaggaatttatttttgggtattataaagtttttgttttttaatgttaggtttttagagttttttagttgtatttttatgtcggtagtattttggattttgtgtaattatgggtttagaataggtaaggttttatttttttttgatttagaatataagtttcgttatcgttaaagagaaattttgaaggagtaggagattgaaatcgatattaatttttgtaaagagatttttttattttggtgtttatgcggtggtgacgggtgtagacgttgtaaataaattttgggaagcgaggagggtcggggaggatattttcggatacgtttttttaaagcgtgtgtagtacggggcgaagttcgcgttgcgggggtagcggtttcggtttttttttgtagcgtttgtatattt

SEQ ID NO: 221 - DNA, 131 bp, Homo sapiens
ctcaaggaagtggaacaaatcgtcgtagccaggcgtctctgcctctccggaaacccgaaatccgcccaaacctcatcccaggcgctcacggtaccagtcgcagggccccgcactataggggctgccaccct

SEQ ID NO: 222 - DNA, 131 bp, Artificial Sequence
  converted
tttaaggaagtggaataaatcgtcgtagttaggcgtttttgttttttcggaaattcgaaattcgtttaaattttattttaggcgtttacggtattagtcgtagggtttcgtattataggggttgttatttt

SEQ ID NO: 223 - DNA, 131 bp, Artificial Sequence
  converted
aaaataacaacccctataatacgaaaccctacgactaataccgtaaacgcctaaaataaaatttaaacgaatttcgaatttccgaaaaaacaaaaacgcctaactacgacgatttattccacttccttaaa

SEQ ID NO: 224 - DNA, 131 bp, Artificial Sequence
  converted
ctcaaaaaaataaaacaaatcgtcgtaaccaaacgtctctacctctccgaaaacccgaaatccgcccaaacctcatcccaaacgctcacgataccaatcgcaaaaccccgcactataaaaactaccaccct

SEQ ID NO: 225 - DNA, 131 bp, Artificial Sequence
  converted
agggtggtagtttttatagtgcggggttttgcgattggtatcgtgagcgtttgggatgaggtttgggcggatttcgggttttcggagaggtagagacgtttggttacgacgatttgttttatttttttgag

SEQ ID NO: 226 - DNA, 21 bp, Artificial Sequence
  converted
ggtgttttaaggttattaaac

SEQ ID NO: 227 - DNA, 20 bp, Artificial Sequence
  converted
gagaaggagtattgttgttc

SEQ ID NO: 228 - DNA, 21 bp, Artificial Sequence
  converted
tgtaggttgtagtaggttttc

SEQ ID NO: 229 - DNA, 18 bp, Artificial Sequence
  converted
ttttttgtcgggattttc

SEQ ID NO: 230 - DNA, 19 bp, Artificial Sequence
  converted
atttaaattttcgagttcg

SEQ ID NO: 231 - DNA, 26 bp, Artificial Sequence
  converted
gagttgagagaatttttaaatttttc

SEQ ID NO: 232 - DNA, 20 bp, Artificial Sequence
  converted
agtttatagagttagggttc

SEQ ID NO: 233 - DNA, 17 bp, Artificial Sequence
  converted
ggttgaaattagagcgc

SEQ ID NO: 234 - DNA, 20 bp, Artificial Sequence
  converted
aagtggtattgaagattatc

SEQ ID NO: 235 - DNA, 19 bp, Artificial Sequence
  converted
gaatgtaaagtaagcgttc

SEQ ID NO: 236 - DNA, 18 bp, Artificial Sequence
  converted
atcgaaaacgctacaccg

SEQ ID NO: 237 - DNA, 19 bp, Artificial Sequence
  converted
gttttggttatgtagggac

SEQ ID NO: 238 - DNA, 19 bp, Artificial Sequence
  converted
tcgctaactacaaacaaac

SEQ ID NO: 239 - DNA, 19 bp, Artificial Sequence
  converted
aggtttttttgtatacgtc

SEQ ID NO: 240 - DNA, 21 bp, Artificial Sequence
  converted
tttaaggaagtggaataaatc

SEQ ID NO: 241 - DNA, 19 bp, Artificial Sequence
  converted
ctcgatccccctcctcgta

SEQ ID NO: 242 - DNA, 22 bp, Artificial Sequence
  converted
actatccccctaaaaatactcg

SEQ ID NO: 243 - DNA, 19 bp, Artificial Sequence
  converted
atacctcctctaacaaacg

SEQ ID NO: 244 - DNA, 18 bp, Artificial Sequence
  converted
tccaatctccaaccgccg

SEQ ID NO: 245 - DNA, 19 bp, Artificial Sequence
  converted
attccctaatactaatccc

SEQ ID NO: 246 - DNA, 22 bp, Artificial Sequence
  converted
tcttctaaaacaaaaaacaccg

SEQ ID NO: 247 - DNA, 21 bp, Artificial Sequence
  converted
tccaaaaaaaacttcctttcg

SEQ ID NO: 248 - DNA, 17 bp, Artificial Sequence
  converted
tacctccattaccgacg

SEQ ID NO: 249 - DNA, 19 bp, Artificial Sequence
  converted
ctcgccgaaaaaaaaaccg

SEQ ID NO: 250 - DNA, 18 bp, Artificial Sequence
  converted
cttctctcgccccgaacg

SEQ ID NO: 251 - DNA, 19 bp, Artificial Sequence
  converted
gaggttcggagatttttcg

SEQ ID NO: 252 - DNA, 21 bp, Artificial Sequence
  converted
cctacctataacttctctccg

SEQ ID NO: 253 - DNA, 19 bp, Artificial Sequence
  converted
ggaagtgtgtggtaaagcg

SEQ ID NO: 254 - DNA, 19 bp, Artificial Sequence
  converted
gctctccccttcccgaacg

SEQ ID NO: 255 - DNA, 23 bp, Artificial Sequence
  converted
aaaataacaacccctataatacg